(19) United States
(12) Patent Application Publication
(10) Pub. No.: US 2025/0243203 A1
(43) Pub. Date: Jul. 31, 2025

(54) NICOTINAMIDE- AND BENZAMIDE-BASED COMPOUNDS, CONJUGATES, AND COMPOSITIONS AS INHIBITORS OF TRANSLATIONAL- AND TRANSCRIPTIONAL-RELATED KINASES

(71) Applicant: PURDUE RESEARCH FOUNDATION, West Lafayette, IN (US)

(72) Inventors: Herman Sintim, Westfield, IN (US); Neetu Dayal, Agra (IN)

(21) Appl. No.: 18/854,427

(22) PCT Filed: Apr. 6, 2023
(86) PCT No.: PCT/US23/65476

§ 371 Date: Oct. 4, 2024

Related U.S. Application Data
(60) Provisional application No. 63/327,984, filed on Apr. 6, 2022.

(51) Int. Cl. C07D 487/04; A61K 31/5025; A61K 31/5377; A61K 31/5386; A61K 31/541; A61K 31/551; A61K 31/553; A61K 47/55; A61P 35/00; C07D 513/04; C07D 519/00

(52) U.S. Cl. CPC C07D 487/04; A61K 31/5025; A61K 31/5377; A61K 31/5386; A61K 31/541; A61K 31/551; A61K 31/553; A61K 47/551; A61P 35/00; C07D 513/04; C07D 519/00

(57) ABSTRACT

Compounds comprising nicotinamide or benzamide linked to a bicyclic heterocycle such as an alkynyl imidazo-[1,2-b]-pyridazine, substituted with one or more ring moieties, as well as conjugates and compositions comprising the same. Methods for treatment of kinase-associated diseases and disorders are also provided.

### PRIORITY

This application is related to and claims the priority benefit of U.S. Provisional Patent Application No. 63/327,984 filed Apr. 6, 2022. The content of the aforementioned application is hereby incorporated by reference in its entirety into this disclosure.

### TECHNICAL FIELD

The present disclosure generally relates to compounds comprising nicotinamide or benzamide-based compounds comprising a bicyclic heterocycle such as an alkynyl imidazo-[1,2-b]-pyridazine substituted with one or more ring moieties that can inhibit certain kinases (e.g., tyrosine-protein kinase ABL1), compositions comprising the same, and methods for use of such compounds and compositions for treating cancer (e.g., solid tumor cancer) and other disease states.

### BACKGROUND

This section introduces aspects that may help facilitate a better understanding of the disclosure. Accordingly, these statements are to be read in this light and are not to be understood as admissions about what is or is not prior art.

There are over 500 kinases in human cells that regulate important processes, such as the cell cycle, cell proliferation, apoptosis and migration. Inhibitors of protein kinases have the potential to treat many diseases that can be controlled by the dysregulation of protein kinases. To date, over twenty kinase inhibitors have been approved by the U.S. Food and Drug Administration (FDA) to treat various diseases.

Ponatinib, an oral drug developed by ARIAD Pharmaceuticals, Inc. for the treatment of chronic myeloid leukemia and Philadelphia chromosome-positive acute lymphoblastic leukemia, is a multi-targeted tyrosine-kinase inhibitor and was approved by the FDA in 2012. The kinases targeted by ponatinib can include, for example, non-receptor tyrosine-protein kinase ABL1 (ABL1), T3151 mutant kinase, Fms-like tyrosine kinase 3 (FLT3), members of the vascular endothelial growth factor receptor (VEGF) family of kinases, members of the platelet-derived growth factor receptor (PDGFR) family of kinases, members of the ephrin (EPH) receptor family of kinases, members of the Src family of kinases, fibroblast growth factor receptor 1, 2, 3, and 4 (FGFR1-4), and rearranged during transfection (RET).

Ponatinib has been shown to potently inhibit various cancers, including chronic myelogenous leukemia (CML), acute myeloid leukemia (AML), and various FGFR- and RET-driven cancers (e.g., such as non-small cell lung cancer and thyroid cancer). Currently, ponatinib is one of only a few drugs approved by the FDA for the treatment of imatinib-resistant CML that harbor the T315I mutation, and is also undergoing various clinical trials for the treatment of AML, lung, and several other cancers; however, ponatinib and its nicotinamide analogs that have a linear substituent have not been effective at inhibiting the growth of solid tumor cancers and/or kinases related thereto such as ribosomal protein S6 kinase beta-1 (p70S6K), AXL receptor tyrosine kinase (AXL), and MER Proto-Oncogene tyrosine kinase (MERTK).

Despite ponatinib's potential efficacy against several cancer type, the drug is relatively toxic and has been associated with cardiovascular adverse events (e.g., the induction of cardiac inflammation, the reduction in cardiac function, and the like) at least in part because it is a potent human Ether-A-go-go-Related Gene (hERG) inhibitor. Patients taking ponatinib have also shown side effects of hypertension, platelet dysfunction and peripheral arterial occlusive disease, as well as myocardial infraction, stroke, and liver failure. Additionally, about 40% of patients on ponatinib develop some form of thrombosis, and the drug was even temporarily pulled from the market in 2014 due to these adverse side effects. Currently, ponatinib is generally only given as a drug of last resort for CML patients with the ABL (T3151) mutation that have not responded to any other therapy. Gainor et al., Ponatinib: Accelerated Disapproval, Oncologist 20(8): 847-848 (2015); Talbert et al., A Multi-Parameter In Vitro Screen in Human Stem Cell-Derived Cardiomyocytes Identifies Ponatinib-Induced Structural and Functional Cardiac Toxicity, Toxicology Sci 143(1): 147-155 (2015). These unfavorable cardiovascular toxicity effects are likely related to ponatinib's simultaneous inhibition of cardiovascular-related kinases such as hERG.

There remains a need for a compound (e.g., a drug) that is an effective inhibitor of disease-related kinases, such as p70S6K, but does not induce adverse side effects such as cardiovascular toxicity as is seen with conventional kinase inhibitors.

### SUMMARY

Generally, the present disclosure provides that appending one or more ring moieties (e.g., morpholine or piperazine-type moieties) to the imidazo[1,2-b]pyridazine portion of nicotinamide ponatinib analog can dramatically improve the inhibitory activities of these new compounds against at least ribosomal protein S6 kinase beta-1 (p70S6K), AXL receptor tyrosine kinase (AXL), and MER Proto-Oncogene tyrosine kinase (MERTK), which are either not inhibited or, at best only weakly inhibited, by both ponatinib and a nicotinamide analog, HSN748, having a linear substituent. The compounds hereof, such as HSND80, are orally bioavailable and potently inhibit several solid tumors, such as breast, ovarian, lung, colon etc.

In certain embodiments, the compound has a structure of Formula (XX):

R₄-L-Z—R₃ (XX)

or is a pharmaceutically acceptable salt thereof, wherein: R₄ is a nicotinamide or benzamide, each of the nicotinamide or benzamide optionally substituted; L is a linker comprising at least one atom; Z is a bicyclic heterocycle substituted with R₃; and R₃ comprises one or more ring moieties.

The compound can have a structure of Formula (I):

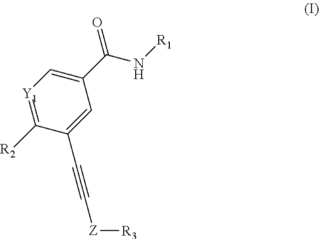

or be a pharmaceutically acceptable salt thereof, wherein: R₁ is a pyridine, an alkyl, an isoxazole, a pyrazole, or a phenyl group, each optionally substituted with one or more of a trifluoromethyl group, a piperazine (e.g., an alkyl piperazine), a pyrazine (e.g., an alkyl pyrazine), an imidazole (e.g., an alkyl imidazole), a cyanide, an amine, a halogen, an N-containing heterocycle, and/or an alkyl; Y₁ is N or C; R₂ is H, an alkyl, a heteroalkyl, or a halogen; Z is imidazo[1,2-b]pyridazine, imidazo[1,2-b]pyrazole, or imidazo[2,1-b][1,3,4]thiadiazole; and R₃ has a structure of Formula (II), Formula (III), or Formula (IV):

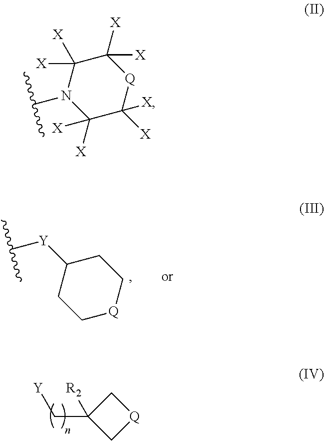

wherein:

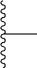

is a point of attachment; each X is independently an alkyl or an H, with the proviso that all X are not H; Q is O, S, N, or C, and is optionally substituted with one or more of an alkyl, a halogen, an O-alkyl, an amine, an —OH group, an alkoxy, a piperazine, a morpholine, an aziridine, a carbocycle or heterocycle, and/or a carbonyl; Y is n or an amine, with the proviso that Y is not NH; and n is 0 or 1. The linker (L) can be an alkyne.

R₃ can be a substituted morpholine. R₃ can be an azetidine, optionally substituted with one or more of an alkyl, a halogen, an amine, a O-alkyl, and/or an —OH group. R₃ can be a pyrolidine, optionally substituted with an oxetane. R₃ can be or comprise

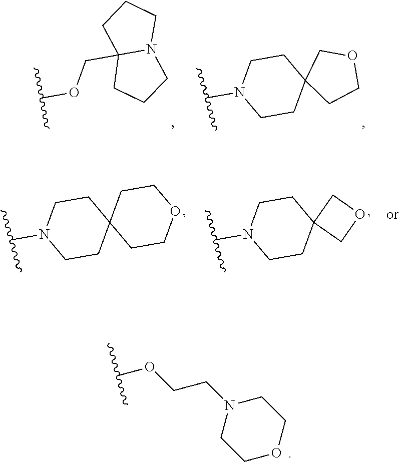

Z can have a structure of

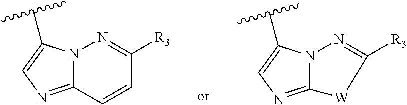

wherein

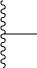

is a point of attachment, and W is C or S.i

In certain embodiments, the compound has a structure of Formula (V):

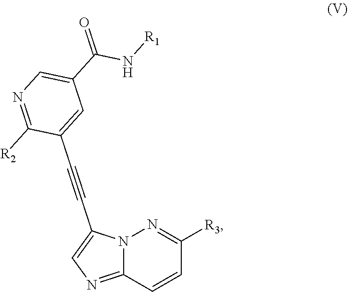

or is a pharmaceutically acceptable salt thereof.

In certain embodiments where R₃ has a structure of Formula (III), Y is a methylamine (—NMe) or an ethylamine (—NEt). Q can be O. R₃ can have the structure of Formula (II) and at least two X can be linked together to form a bicyclic heterocycle. R₃ can comprise morpholine. R₃ can comprise unsubstituted morpholine. R₃ can comprise morpholine substituted with at least two methyl groups. R₃ can comprise a 6-membered heterocycle. R₃ can comprise a 4-6-membered heterocycle. R₃ can comprise an oxygen-containing heterocycle.

The compound can have a structure:

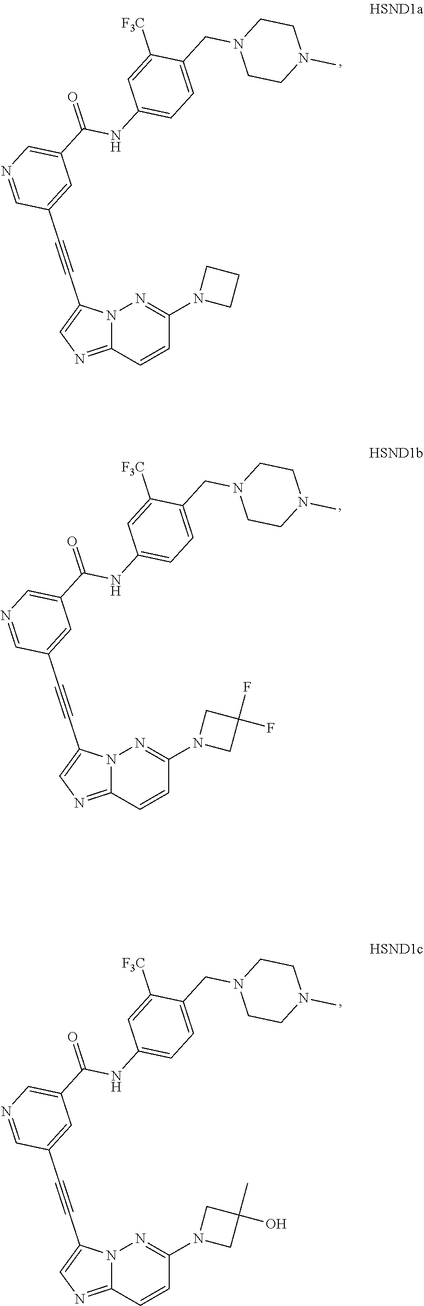 [SMILES: CN1CCN(Cc2ccc(NC(=O)c3cncc(C#Cc4cnc5ccc(N6CCC6)nn45)c3)cc2C(F)(F)F)CC1 ; CN1CCN(Cc2ccc(NC(=O)c3cncc(C#Cc4cnc5ccc(N6CC(F)(F)C6)nn45)c3)cc2C(F)(F)F)CC1 ; CN1CCN(Cc2ccc(NC(=O)c3cncc(C#Cc4cnc5ccc(N6CC(C)(O)C6)nn45)c3)cc2C(F)(F)F)CC1]

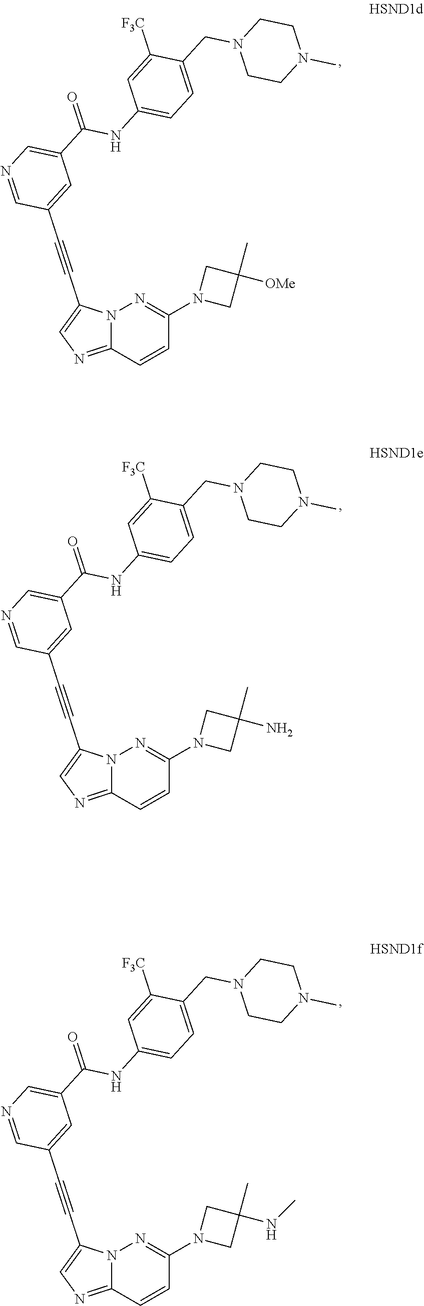 [SMILES: COC1(C)CN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CC(C)(N)C6)nn45)c3)cc2C(F)(F)F)CC1 ; CNC1(C)CN(c2ccc3ncc(C#Cc4cncc(C(=O)Nc5ccc(CN6CCN(C)CC6)c(C(F)(F)F)c5)c4)n3n2)C1]

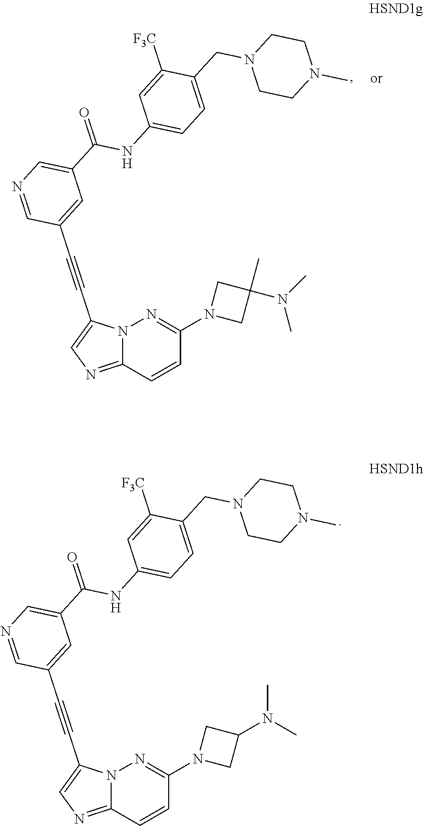 [SMILES: CN1CCN(Cc2ccc(NC(=O)c3cncc(C#Cc4cnc5ccc(N6CC(C)(N(C)C)C6)nn45)c3)cc2C(F)(F)F)CC1 ; CN1CCN(Cc2ccc(NC(=O)c3cncc(C#Cc4cnc5ccc(N6CC(N(C)C)C6)nn45)c3)cc2C(F)(F)F)CC1]

The compound can have a structure:

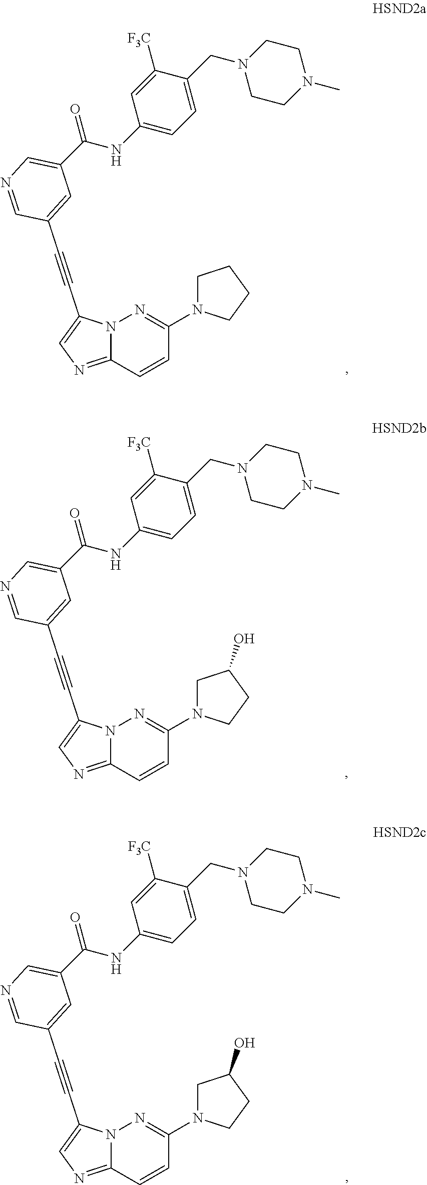 [SMILES: CN1CCN(Cc2ccc(NC(=O)c3cncc(C#Cc4cnc5ccc(N6CCCC6)nn45)c3)cc2C(F)(F)F)CC1 ; CN1CCN(Cc2ccc(NC(=O)c3cncc(C#Cc4cnc5ccc(N6CC[C@@H](O)C6)nn45)c3)cc2C(F)(F)F)CC1 ; CN1CCN(Cc2ccc(NC(=O)c3cncc(C#Cc4cnc5ccc(N6CC[C@H](O)C6)nn45)c3)cc2C(F)(F)F)CC1]

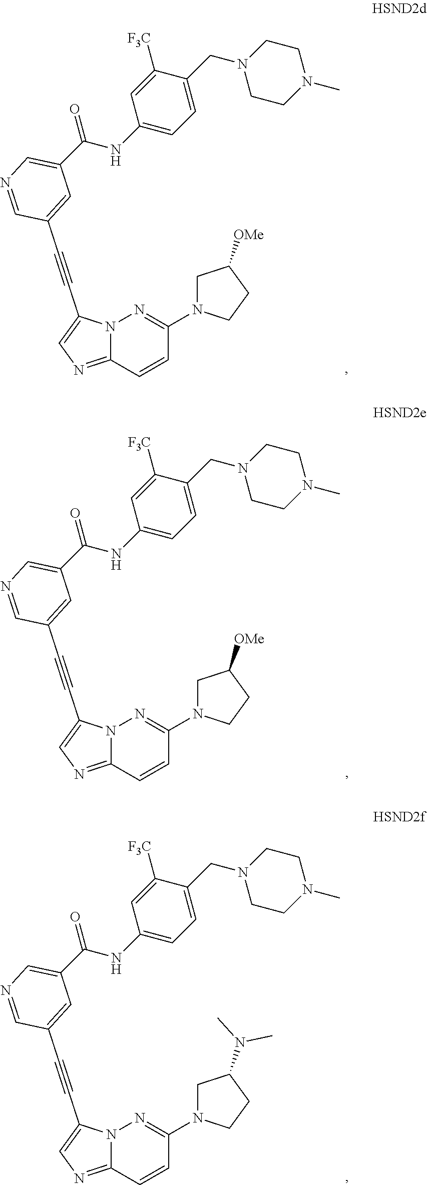 [SMILES: CO[C@@H]1CCN(c2ccc3ncc(C#Cc4cncc(C(=O)Nc5ccc(CN6CCN(C)CC6)c(C(F)(F)F)c5)c4)n3n2)C1 ; CO[C@H]1CCN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CC[C@@H](N(C)C)C6)nn45)c3)cc2C(F)(F)F)CC1]

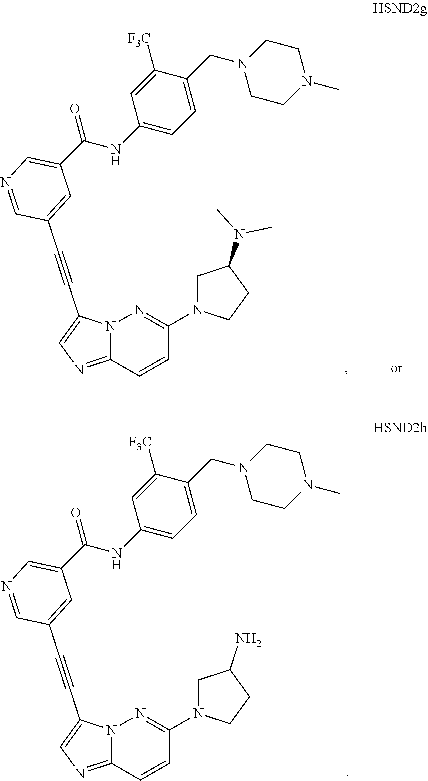 [SMILES: CN1CCN(Cc2ccc(NC(=O)c3cncc(C#Cc4cnc5ccc(N6CC[C@H](N(C)C)C6)nn45)c3)cc2C(F)(F)F)CC1 ; CN1CCN(Cc2ccc(NC(=O)c3cncc(C#Cc4cnc5ccc(N6CCC(N)C6)nn45)c3)cc2C(F)(F)F)CC1]

The compound can have a structure:

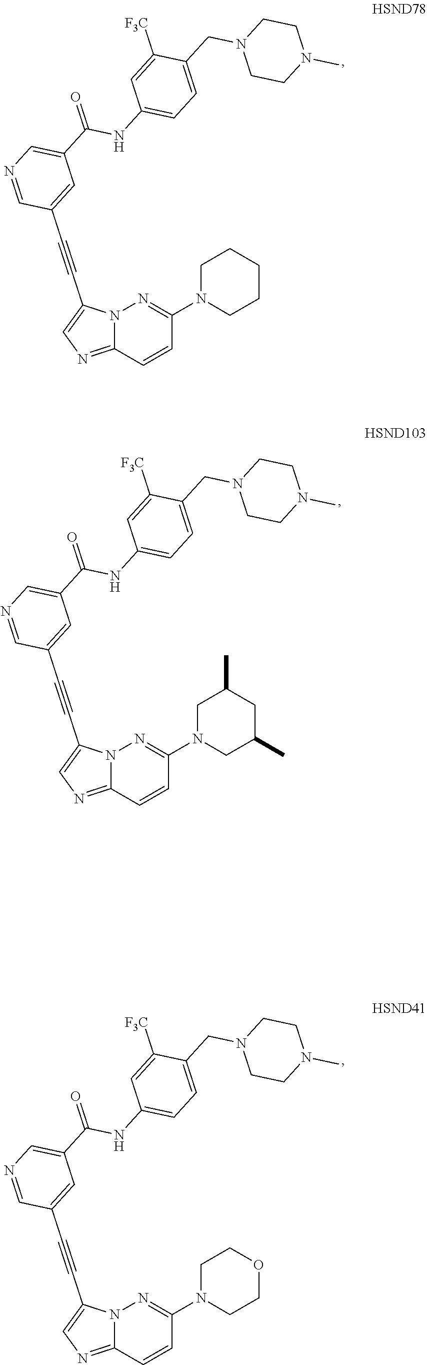 [SMILES: CN1CCN(Cc2ccc(NC(=O)c3cncc(C#Cc4cnc5ccc(N6CCCCC6)nn45)c3)cc2C(F)(F)F)CC1 ; C[C@@H]1C[C@H](C)CN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CCOCC6)nn45)c3)cc2C(F)(F)F)CC1]

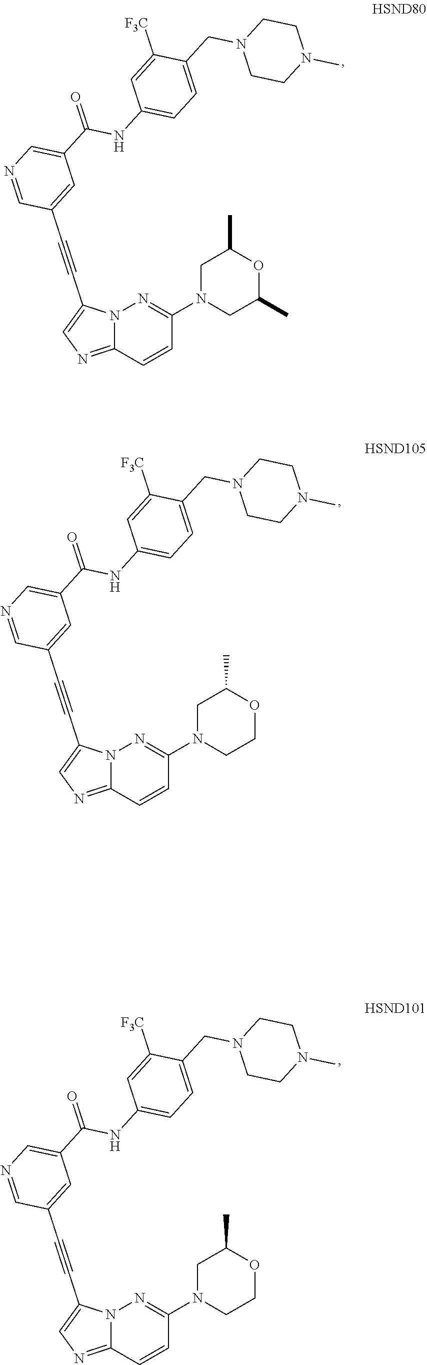 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)O1 ; C[C@H]1CN(c2ccc3ncc(C#Cc4cncc(C(=O)Nc5ccc(CN6CCN(C)CC6)c(C(F)(F)F)c5)c4)n3n2)CCO1 ; C[C@@H]1CN(c2ccc3ncc(C#Cc4cncc(C(=O)Nc5ccc(CN6CCN(C)CC6)c(C(F)(F)F)c5)c4)n3n2)CCO1]

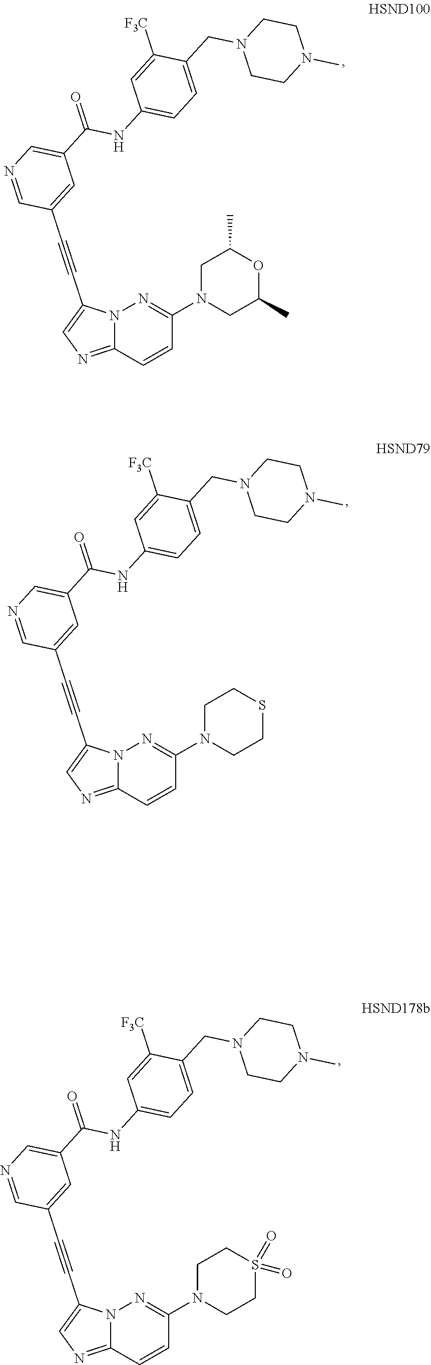 [SMILES: C[C@H]1CN(c2ccc3ncc(C#Cc4cncc(C(=O)Nc5ccc(CN6CCN(C)CC6)c(C(F)(F)F)c5)c4)n3n2)C[C@H](C)O1 ; CN1CCN(Cc2ccc(NC(=O)c3cncc(C#Cc4cnc5ccc(N6CCSCC6)nn45)c3)cc2C(F)(F)F)CC1 ; CN1CCN(Cc2ccc(NC(=O)c3cncc(C#Cc4cnc5ccc(N6CCS(=O)(=O)CC6)nn45)c3)cc2C(F)(F)F)CC1]

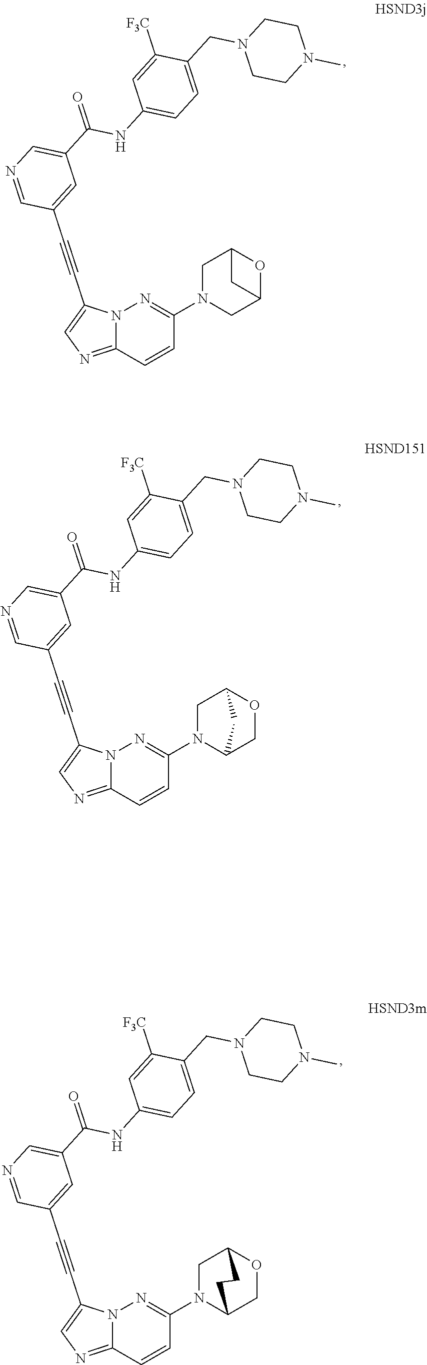

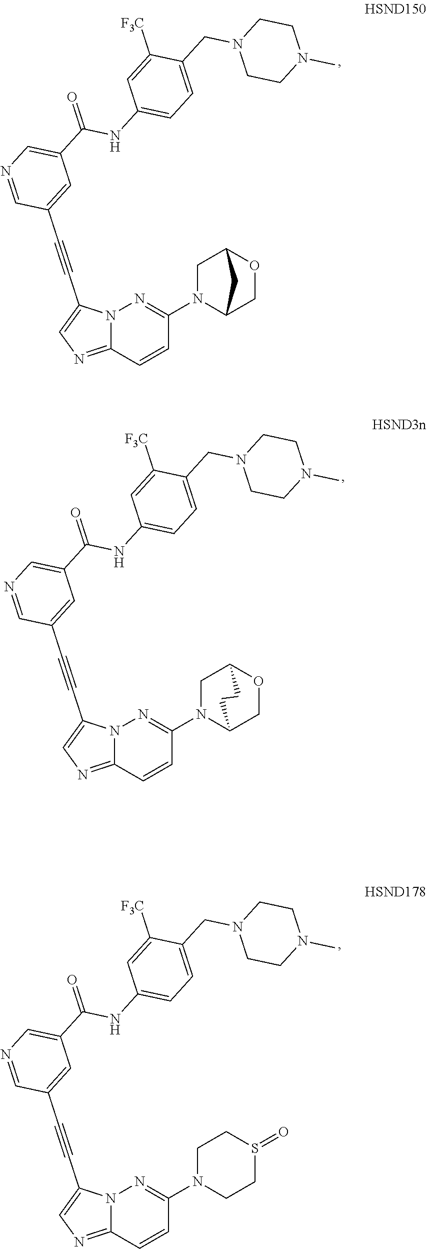 [SMILES: CN1CCN(Cc2ccc(NC(=O)c3cncc(C#Cc4cnc5ccc(N6C[C@H]7C[C@@H]6CO7)nn45)c3)cc2C(F)(F)F)CC1 ; CN1CCN(Cc2ccc(NC(=O)c3cncc(C#Cc4cnc5ccc(N6C[C@@H]7CC[C@H]6CO7)nn45)c3)cc2C(F)(F)F)CC1 ; CN1CCN(Cc2ccc(NC(=O)c3cncc(C#Cc4cnc5ccc(N6CCS(=O)CC6)nn45)c3)cc2C(F)(F)F)CC1]

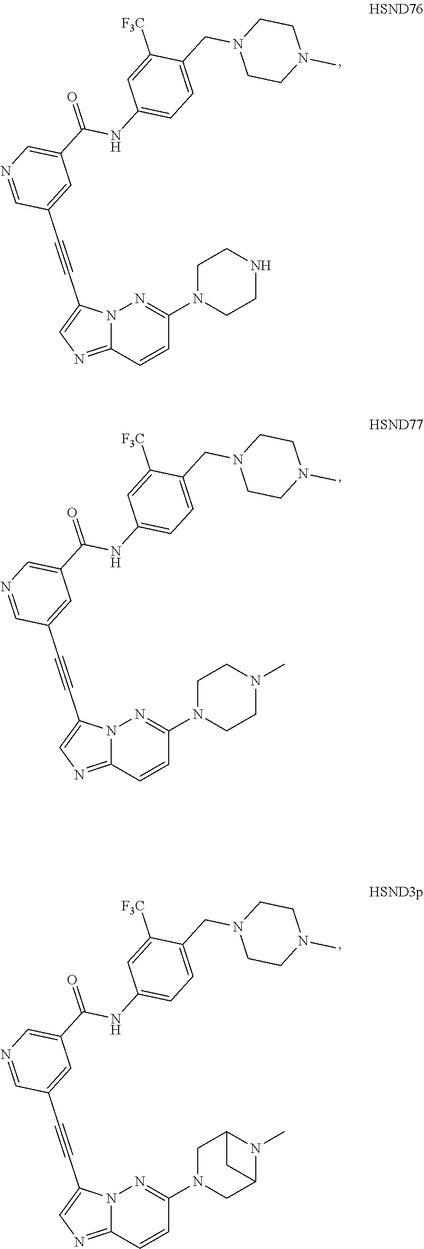 [SMILES: CN1CCN(Cc2ccc(NC(=O)c3cncc(C#Cc4cnc5ccc(N6CCNCC6)nn45)c3)cc2C(F)(F)F)CC1 ; CN1CCN(Cc2ccc(NC(=O)c3cncc(C#Cc4cnc5ccc(N6CCN(C)CC6)nn45)c3)cc2C(F)(F)F)CC1 ; CN1CCN(Cc2ccc(NC(=O)c3cncc(C#Cc4cnc5ccc(N6CC7CC(C6)N7C)nn45)c3)cc2C(F)(F)F)CC1]

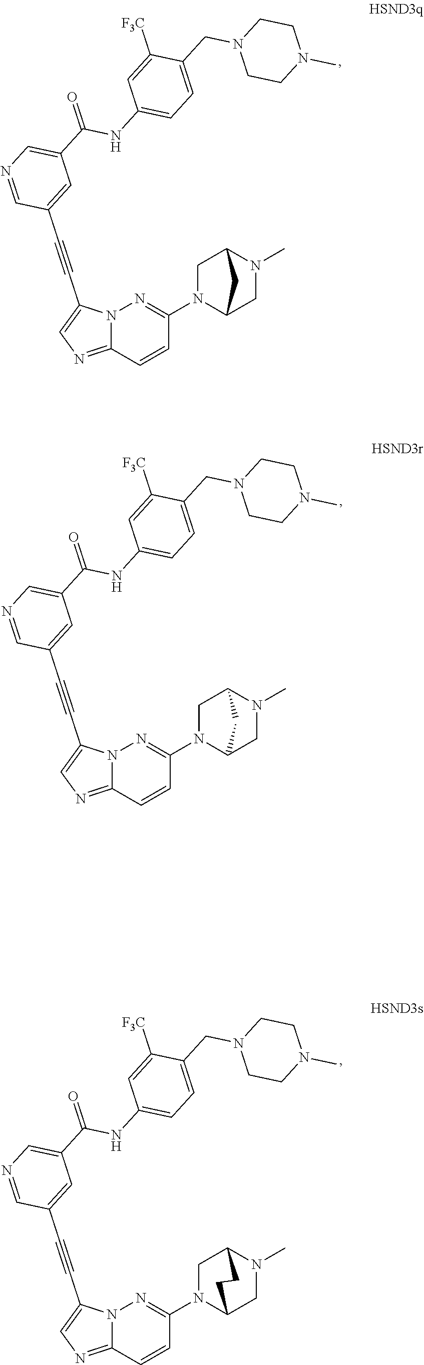

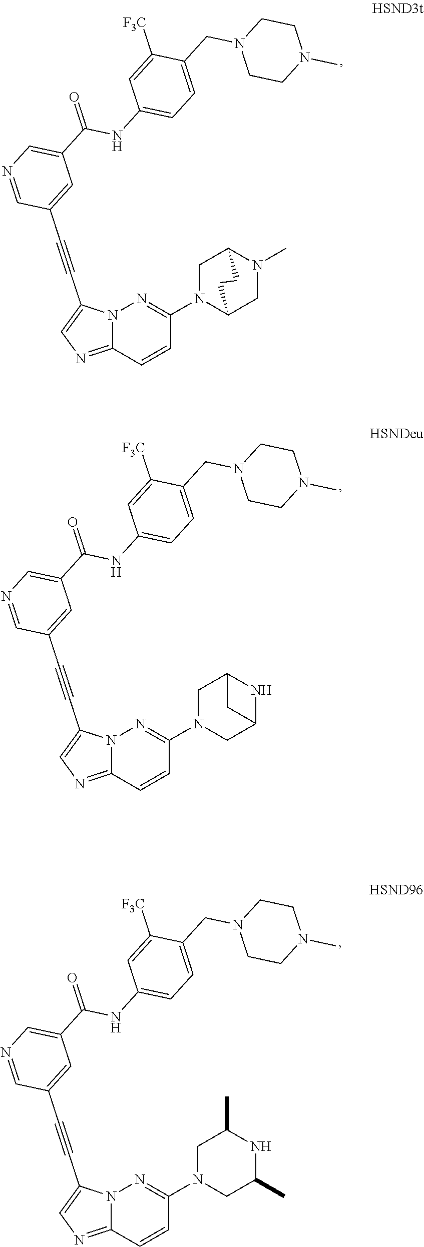 [SMILES: CN1CCN(Cc2ccc(NC(=O)c3cncc(C#Cc4cnc5ccc(N6C[C@@H]7CC[C@H]6CN7C)nn45)c3)cc2C(F)(F)F)CC1 ; CN1CCN(Cc2ccc(NC(=O)c3cncc(C#Cc4cnc5ccc(N6CC7CC(C6)N7)nn45)c3)cc2C(F)(F)F)CC1 ; C[C@@H]1CN(c2ccc3ncc(C#Cc4cncc(C(=O)Nc5ccc(CN6CCN(C)CC6)c(C(F)(F)F)c5)c4)n3n2)C[C@H](C)N1]

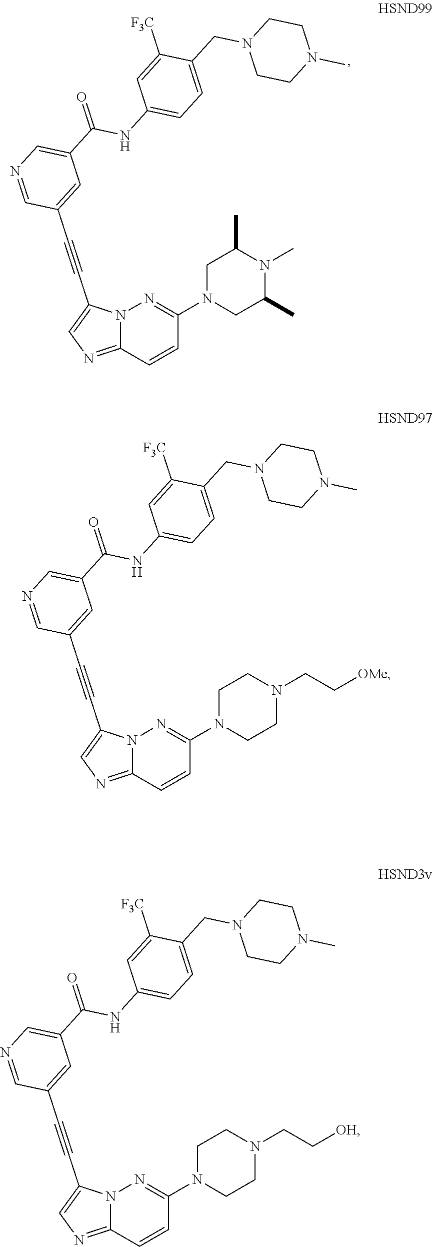 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)N1C ; COCCN1CCN(c2ccc3ncc(C#Cc4cncc(C(=O)Nc5ccc(CN6CCN(C)CC6)c(C(F)(F)F)c5)c4)n3n2)CC1 ; CN1CCN(Cc2ccc(NC(=O)c3cncc(C#Cc4cnc5ccc(N6CCN(CCO)CC6)nn45)c3)cc2C(F)(F)F)CC1]

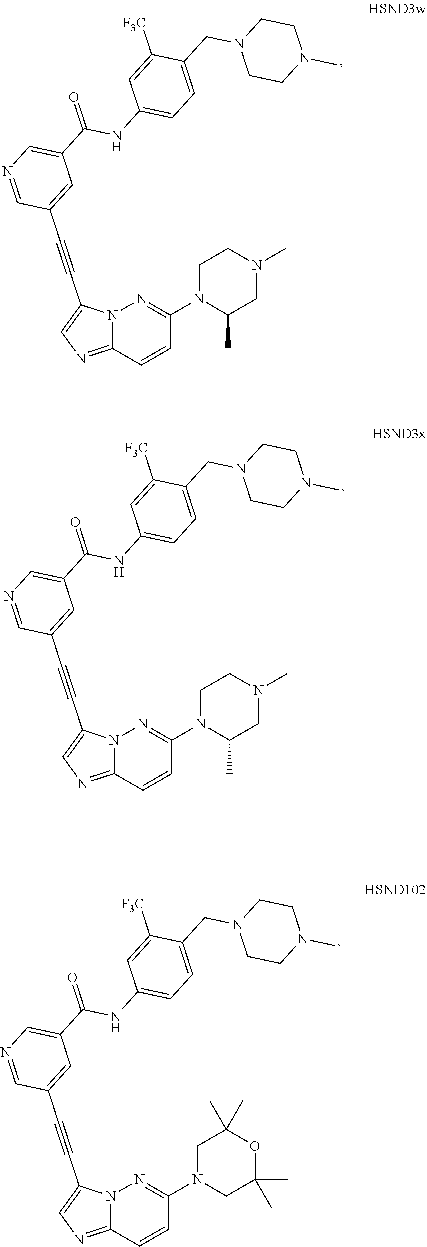 [SMILES: C[C@@H]1CN(C)CCN1c1ccc2ncc(C#Cc3cncc(C(=O)Nc4ccc(CN5CCN(C)CC5)c(C(F)(F)F)c4)c3)n2n1 ; C[C@H]1CN(C)CCN1c1ccc2ncc(C#Cc3cncc(C(=O)Nc4ccc(CN5CCN(C)CC5)c(C(F)(F)F)c4)c3)n2n1 ; CN1CCN(Cc2ccc(NC(=O)c3cncc(C#Cc4cnc5ccc(N6CC(C)(C)OC(C)(C)C6)nn45)c3)cc2C(F)(F)F)CC1]

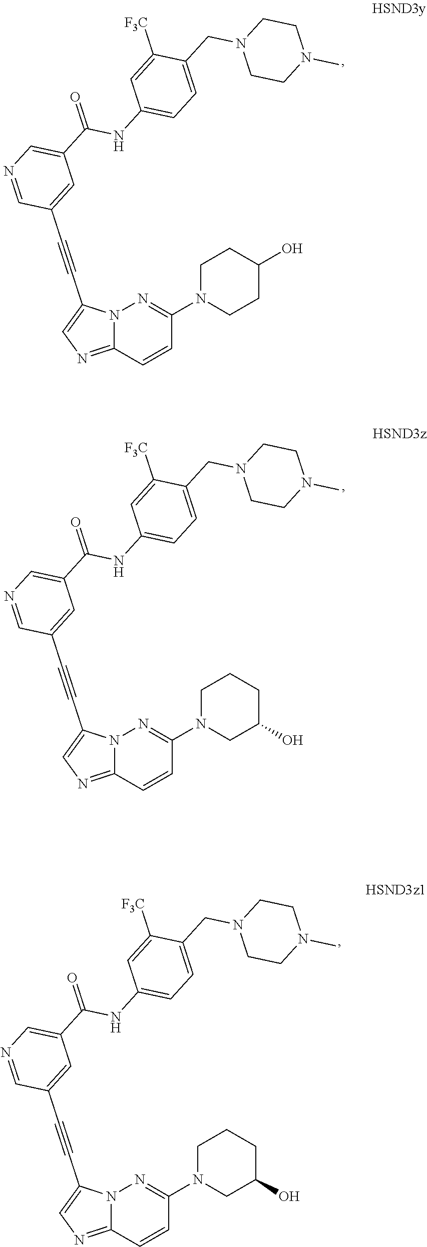 [SMILES: CN1CCN(Cc2ccc(NC(=O)c3cncc(C#Cc4cnc5ccc(N6CCC(O)CC6)nn45)c3)cc2C(F)(F)F)CC1 ; CN1CCN(Cc2ccc(NC(=O)c3cncc(C#Cc4cnc5ccc(N6CCC[C@H](O)C6)nn45)c3)cc2C(F)(F)F)CC1 ; CN1CCN(Cc2ccc(NC(=O)c3cncc(C#Cc4cnc5ccc(N6CCC[C@@H](O)C6)nn45)c3)cc2C(F)(F)F)CC1]

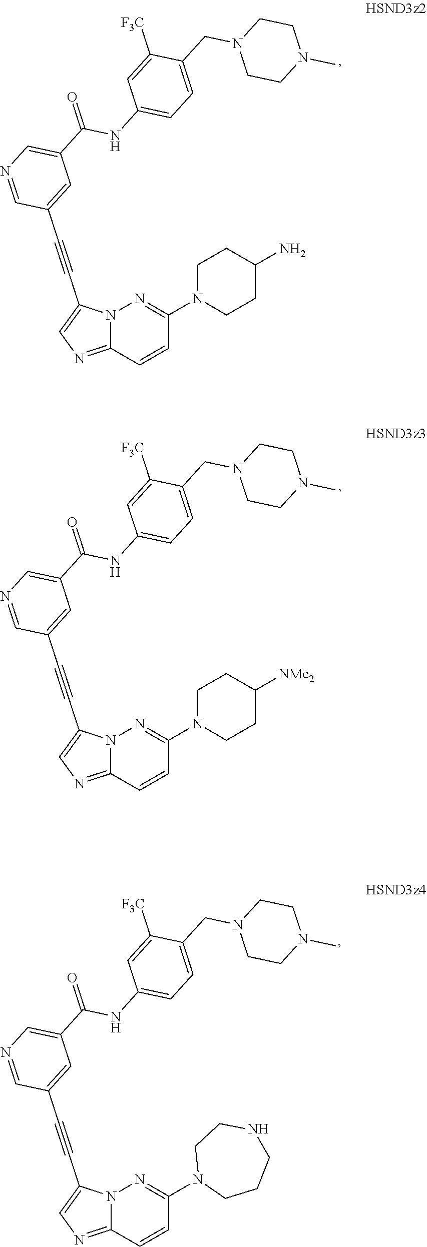 [SMILES: CN1CCN(Cc2ccc(NC(=O)c3cncc(C#Cc4cnc5ccc(N6CCC(N)CC6)nn45)c3)cc2C(F)(F)F)CC1 ; CN1CCN(Cc2ccc(NC(=O)c3cncc(C#Cc4cnc5ccc(N6CCC(N(C)C)CC6)nn45)c3)cc2C(F)(F)F)CC1 ; CN1CCN(Cc2ccc(NC(=O)c3cncc(C#Cc4cnc5ccc(N6CCCNCC6)nn45)c3)cc2C(F)(F)F)CC1]

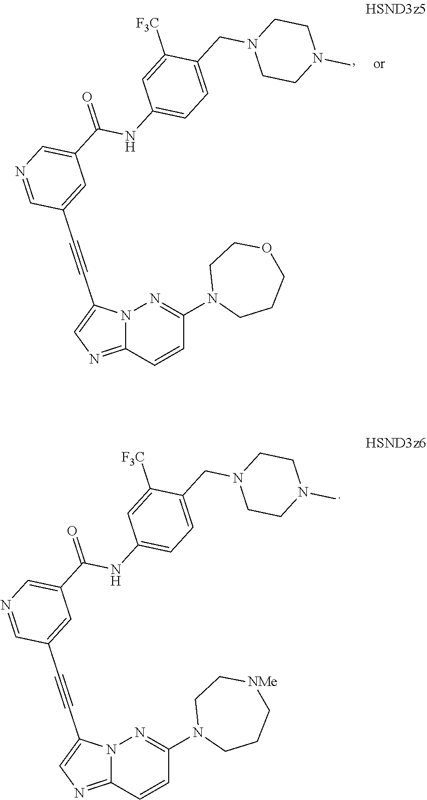 [SMILES: CN1CCN(Cc2ccc(NC(=O)c3cncc(C#Cc4cnc5ccc(N6CCCOCC6)nn45)c3)cc2C(F)(F)F)CC1 ; CN1CCN(Cc2ccc(NC(=O)c3cncc(C#Cc4cnc5ccc(N6CCCN(C)CC6)nn45)c3)cc2C(F)(F)F)CC1]

The compound can have the structure:

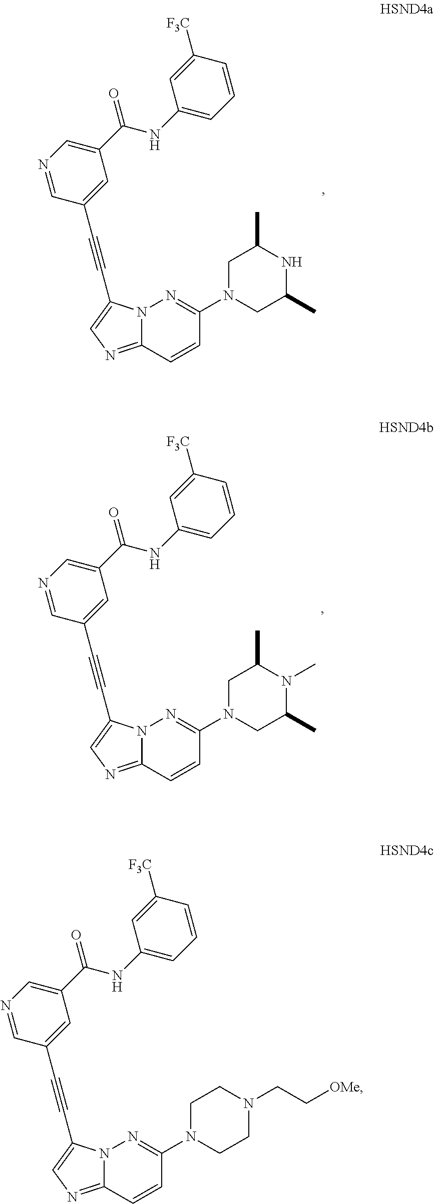 [SMILES: C[C@@H]1CN(c2ccc3ncc(C#Cc4cncc(C(=O)Nc5cccc(C(F)(F)F)c5)c4)n3n2)C[C@H](C)N1 ; C[C@@H]1CN(c2ccc3ncc(C#Cc4cncc(C(=O)Nc5cccc(C(F)(F)F)c5)c4)n3n2)C[C@H](C)N1C ; COCCN1CCN(c2ccc3ncc(C#Cc4cncc(C(=O)Nc5cccc(C(F)(F)F)c5)c4)n3n2)CC1]

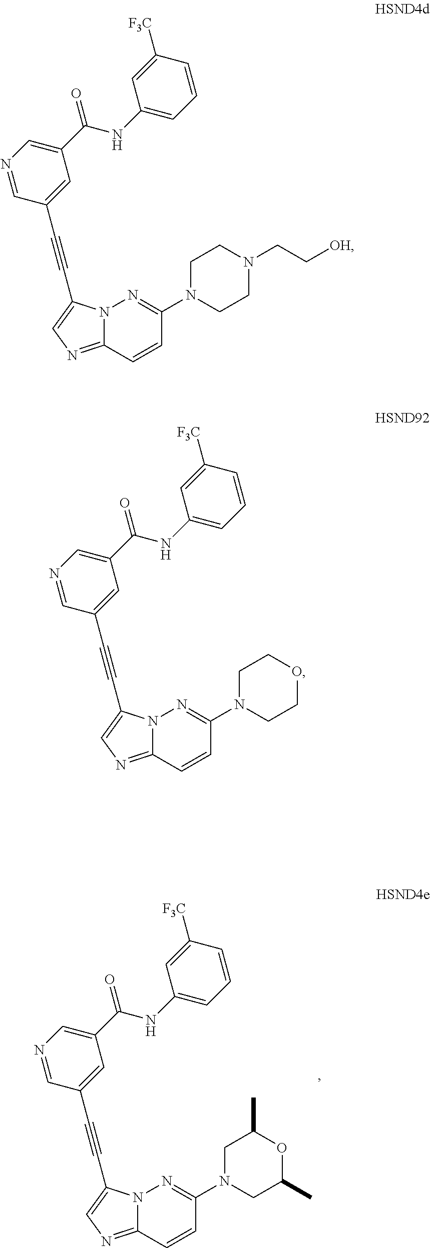 [SMILES: O=C(Nc1cccc(C(F)(F)F)c1)c1cncc(C#Cc2cnc3ccc(N4CCN(CCO)CC4)nn23)c1 ; O=C(Nc1cccc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5cccc(C(F)(F)F)c5)c4)n3n2)C[C@H](C)O1]

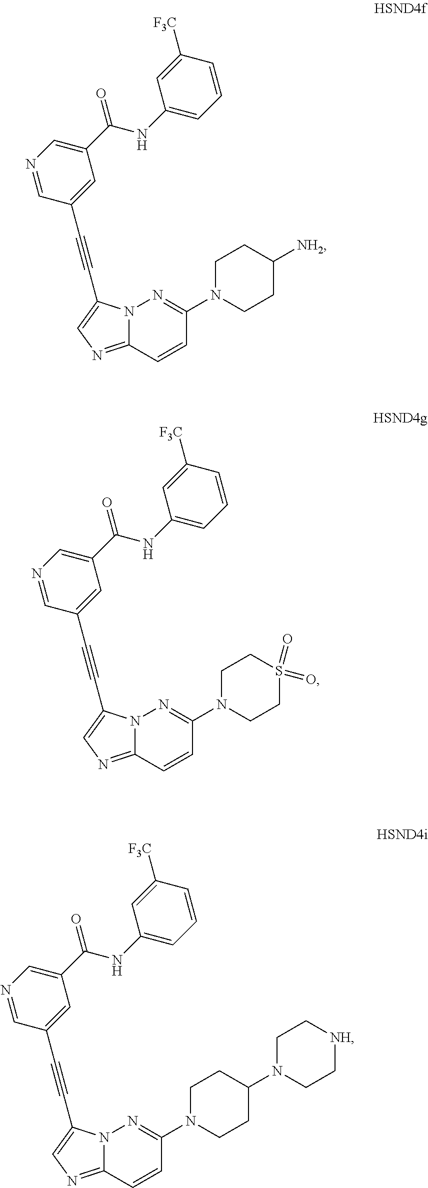 [SMILES: NC1CCN(c2ccc3ncc(C#Cc4cncc(C(=O)Nc5cccc(C(F)(F)F)c5)c4)n3n2)CC1 ; O=C(Nc1cccc(C(F)(F)F)c1)c1cncc(C#Cc2cnc3ccc(N4CCS(=O)(=O)CC4)nn23)c1 ; O=C(Nc1cccc(C(F)(F)F)c1)c1cncc(C#Cc2cnc3ccc(N4CCC(N5CCNCC5)CC4)nn23)c1]

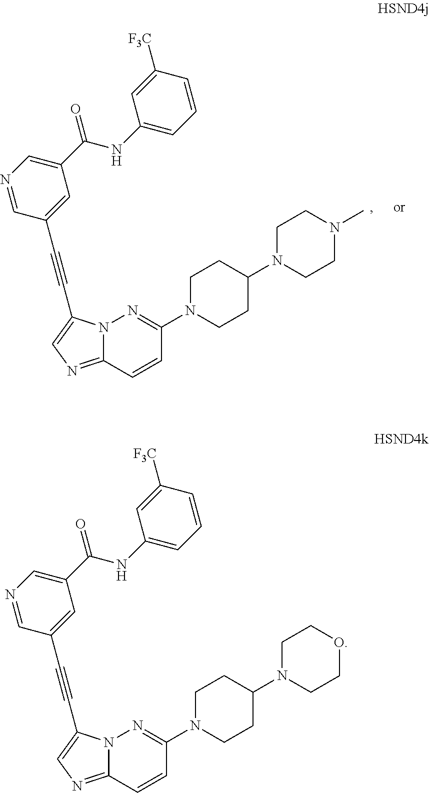 [SMILES: CN1CCN(C2CCN(c3ccc4ncc(C#Cc5cncc(C(=O)Nc6cccc(C(F)(F)F)c6)c5)n4n3)CC2)CC1 ; O=C(Nc1cccc(C(F)(F)F)c1)c1cncc(C#Cc2cnc3ccc(N4CCC(N5CCOCC5)CC4)nn23)c1]

The compound can have a structure:

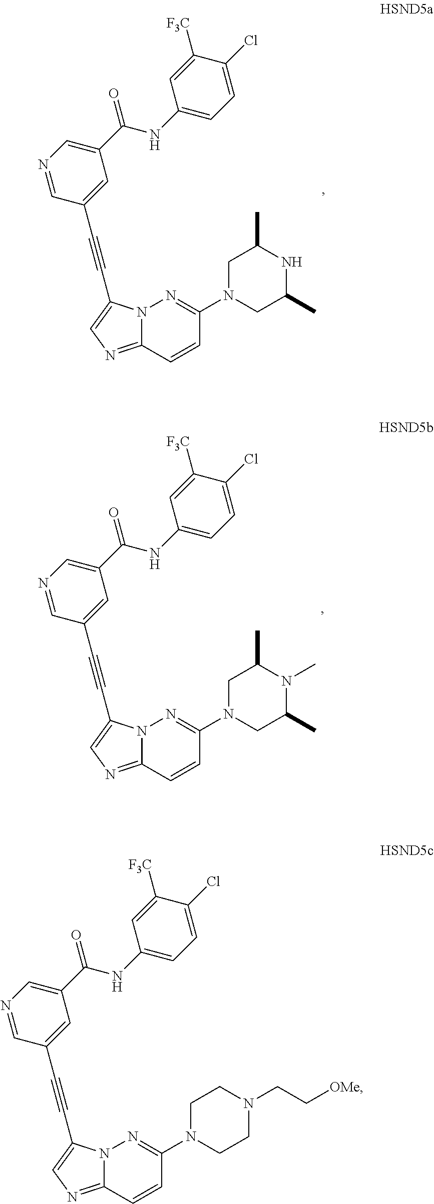 [SMILES: C[C@@H]1CN(c2ccc3ncc(C#Cc4cncc(C(=O)Nc5ccc(Cl)c(C(F)(F)F)c5)c4)n3n2)C[C@H](C)N1 ; C[C@@H]1CN(c2ccc3ncc(C#Cc4cncc(C(=O)Nc5ccc(Cl)c(C(F)(F)F)c5)c4)n3n2)C[C@H](C)N1C ; COCCN1CCN(c2ccc3ncc(C#Cc4cncc(C(=O)Nc5ccc(Cl)c(C(F)(F)F)c5)c4)n3n2)CC1]

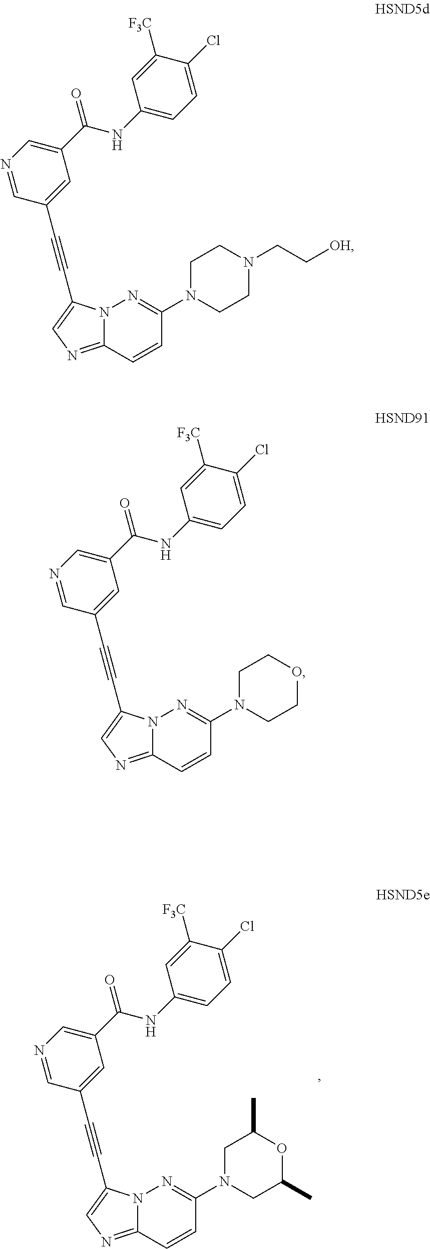 [SMILES: O=C(Nc1ccc(Cl)c(C(F)(F)F)c1)c1cncc(C#Cc2cnc3ccc(N4CCN(CCO)CC4)nn23)c1 ; O=C(Nc1ccc(Cl)c(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5ccc(Cl)c(C(F)(F)F)c5)c4)n3n2)C[C@H](C)O1]

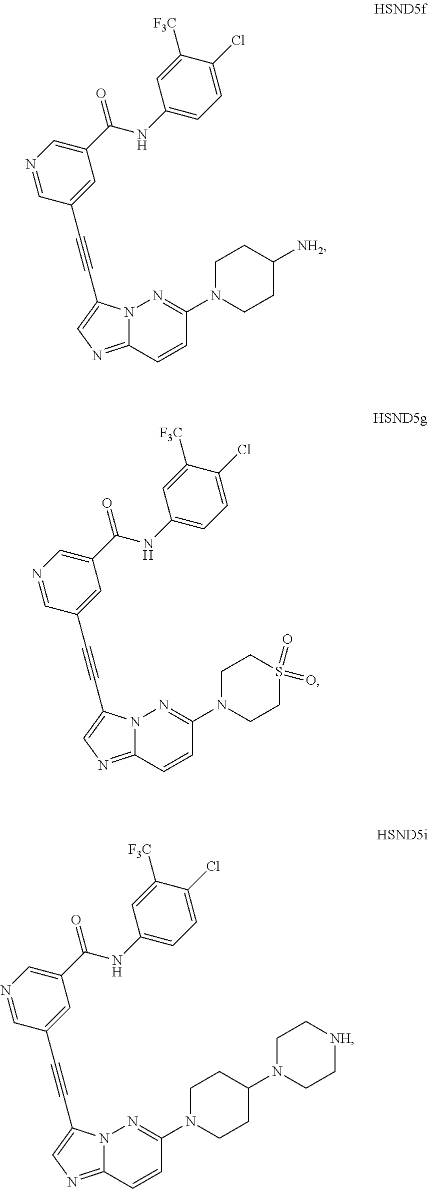 [SMILES: NC1CCN(c2ccc3ncc(C#Cc4cncc(C(=O)Nc5ccc(Cl)c(C(F)(F)F)c5)c4)n3n2)CC1 ; O=C(Nc1ccc(Cl)c(C(F)(F)F)c1)c1cncc(C#Cc2cnc3ccc(N4CCS(=O)(=O)CC4)nn23)c1 ; O=C(Nc1ccc(Cl)c(C(F)(F)F)c1)c1cncc(C#Cc2cnc3ccc(N4CCC(N5CCNCC5)CC4)nn23)c1]

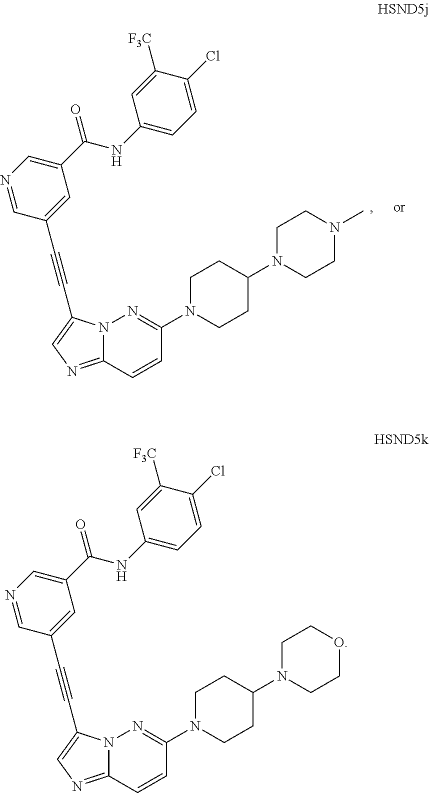 [SMILES: CN1CCN(C2CCN(c3ccc4ncc(C#Cc5cncc(C(=O)Nc6ccc(Cl)c(C(F)(F)F)c6)c5)n4n3)CC2)CC1 ; O=C(Nc1ccc(Cl)c(C(F)(F)F)c1)c1cncc(C#Cc2cnc3ccc(N4CCC(N5CCOCC5)CC4)nn23)c1]

A compound can have a structure:

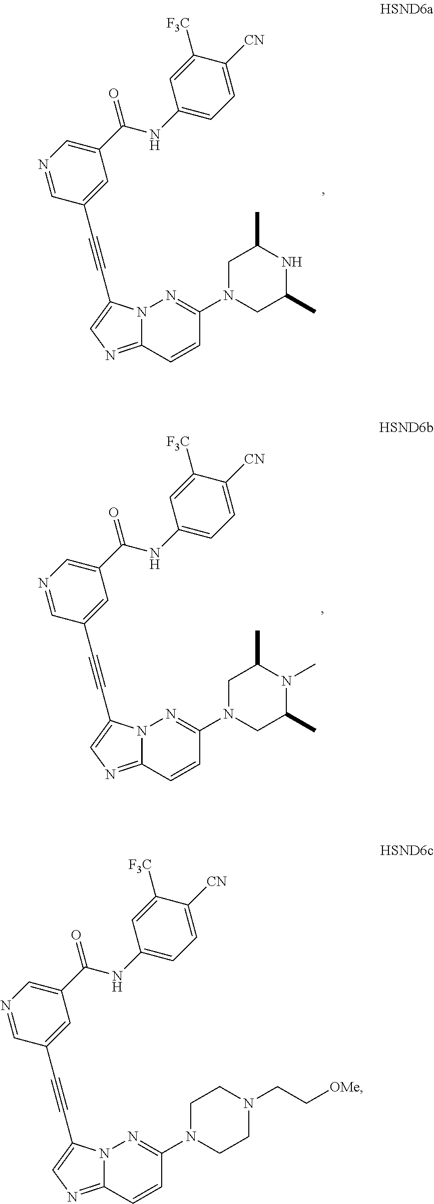 [SMILES: C[C@@H]1CN(c2ccc3ncc(C#Cc4cncc(C(=O)Nc5ccc(C#N)c(C(F)(F)F)c5)c4)n3n2)C[C@H](C)N1 ; C[C@@H]1CN(c2ccc3ncc(C#Cc4cncc(C(=O)Nc5ccc(C#N)c(C(F)(F)F)c5)c4)n3n2)C[C@H](C)N1C ; COCCN1CCN(c2ccc3ncc(C#Cc4cncc(C(=O)Nc5ccc(C#N)c(C(F)(F)F)c5)c4)n3n2)CC1]

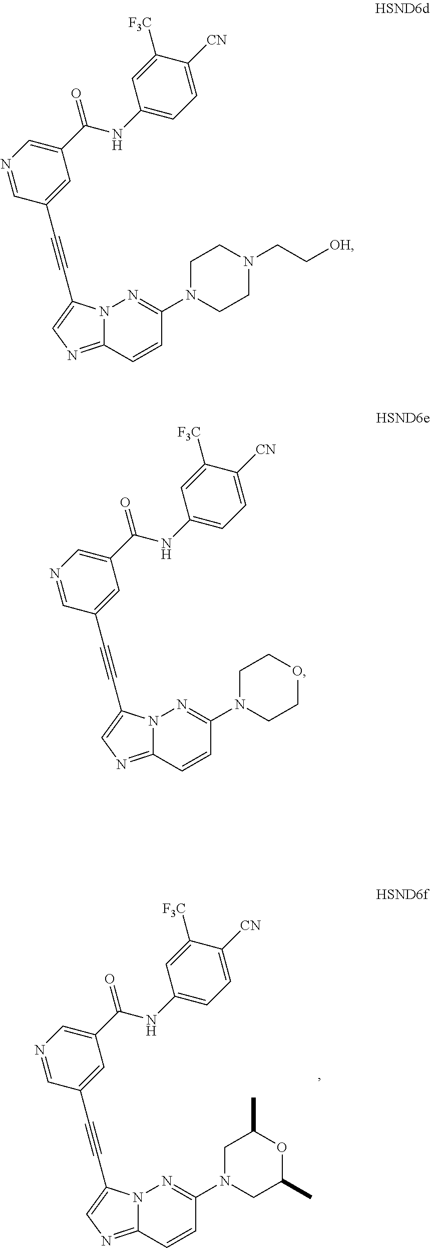 [SMILES: N#Cc1ccc(NC(=O)c2cncc(C#Cc3cnc4ccc(N5CCN(CCO)CC5)nn34)c2)cc1C(F)(F)F ; N#Cc1ccc(NC(=O)c2cncc(C#Cc3cnc4ccc(N5CCOCC5)nn34)c2)cc1C(F)(F)F ; C[C@@H]1CN(c2ccc3ncc(C#Cc4cncc(C(=O)Nc5ccc(C#N)c(C(F)(F)F)c5)c4)n3n2)C[C@H](C)O1]

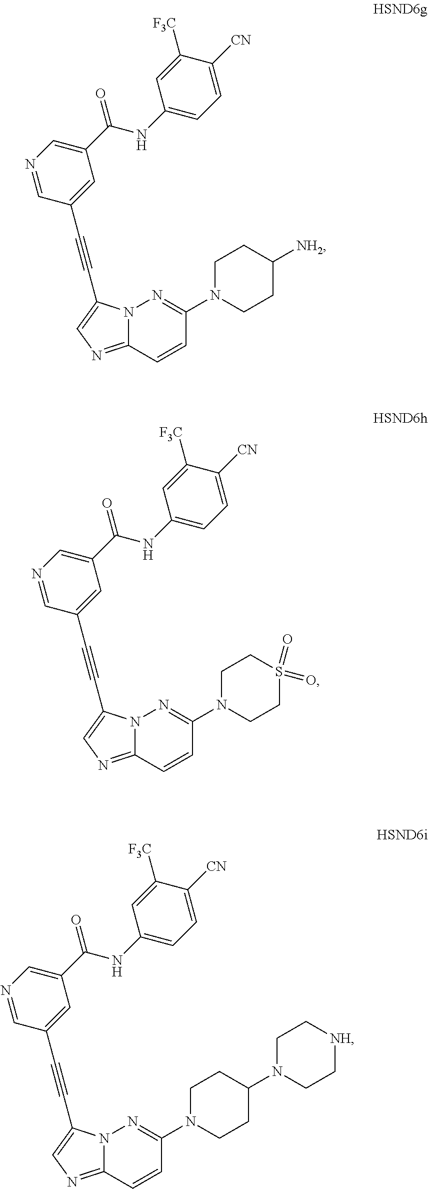 [SMILES: N#Cc1ccc(NC(=O)c2cncc(C#Cc3cnc4ccc(N5CCC(N)CC5)nn34)c2)cc1C(F)(F)F ; N#Cc1ccc(NC(=O)c2cncc(C#Cc3cnc4ccc(N5CCS(=O)(=O)CC5)nn34)c2)cc1C(F)(F)F ; N#Cc1ccc(NC(=O)c2cncc(C#Cc3cnc4ccc(N5CCC(N6CCNCC6)CC5)nn34)c2)cc1C(F)(F)F]

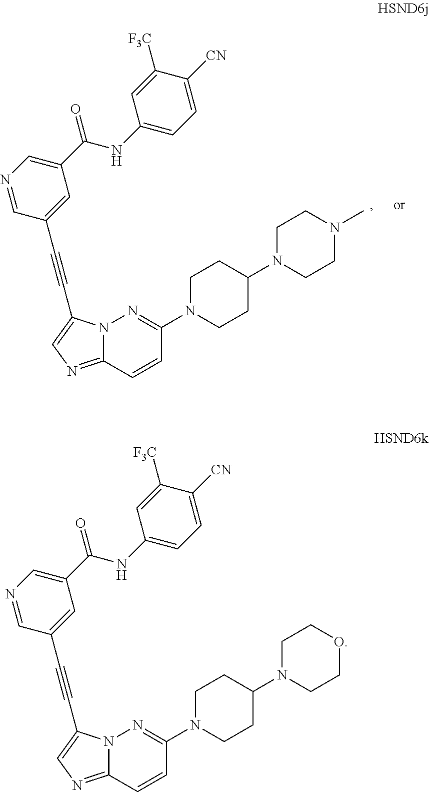 [SMILES: CN1CCN(C2CCN(c3ccc4ncc(C#Cc5cncc(C(=O)Nc6ccc(C#N)c(C(F)(F)F)c6)c5)n4n3)CC2)CC1 ; N#Cc1ccc(NC(=O)c2cncc(C#Cc3cnc4ccc(N5CCC(N6CCOCC6)CC5)nn34)c2)cc1C(F)(F)F]

The compound can have a structure:

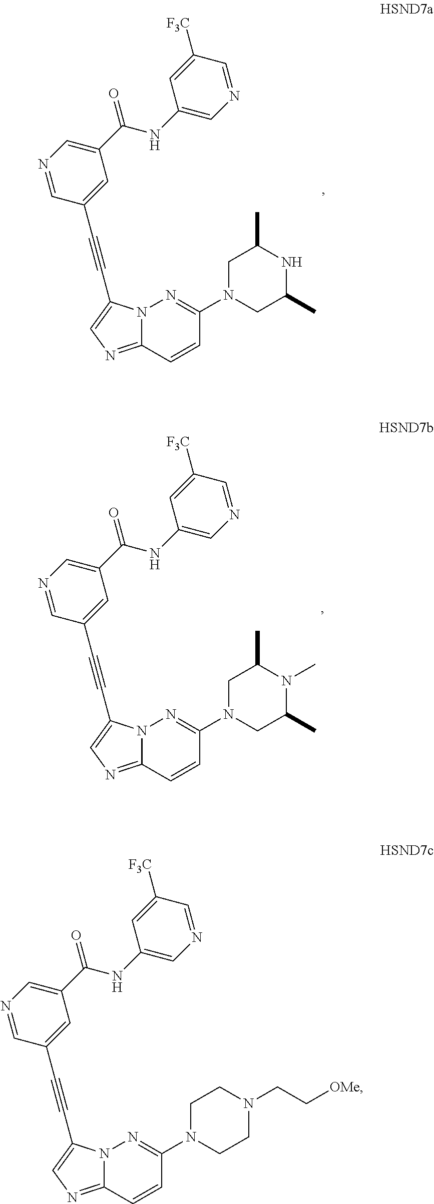 [SMILES: C[C@@H]1CN(c2ccc3ncc(C#Cc4cncc(C(=O)Nc5cncc(C(F)(F)F)c5)c4)n3n2)C[C@H](C)N1 ; C[C@@H]1CN(c2ccc3ncc(C#Cc4cncc(C(=O)Nc5cncc(C(F)(F)F)c5)c4)n3n2)C[C@H](C)N1C ; COCCN1CCN(c2ccc3ncc(C#Cc4cncc(C(=O)Nc5cncc(C(F)(F)F)c5)c4)n3n2)CC1]

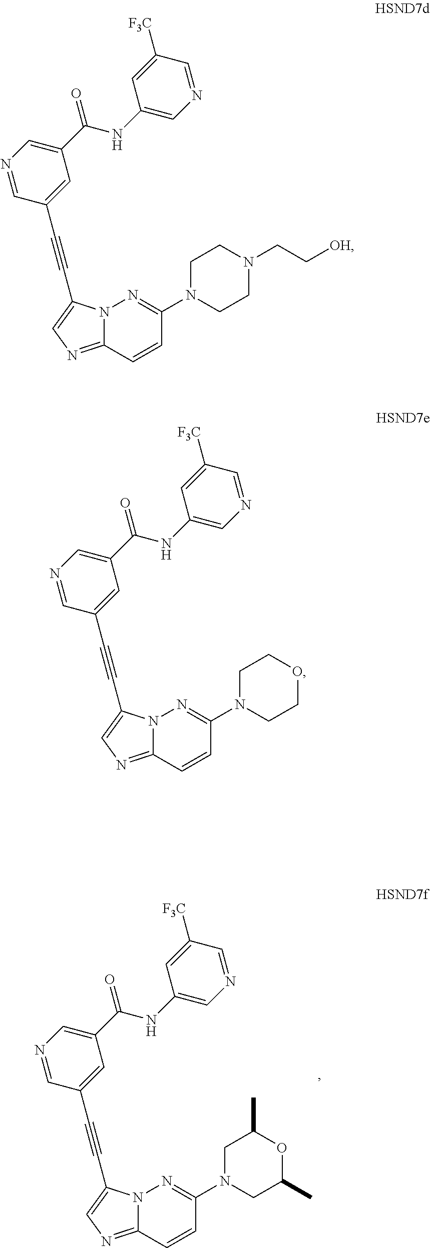 [SMILES: O=C(Nc1cncc(C(F)(F)F)c1)c1cncc(C#Cc2cnc3ccc(N4CCN(CCO)CC4)nn23)c1 ; O=C(Nc1cncc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5cncc(C(F)(F)F)c5)c4)n3n2)C[C@H](C)O1]

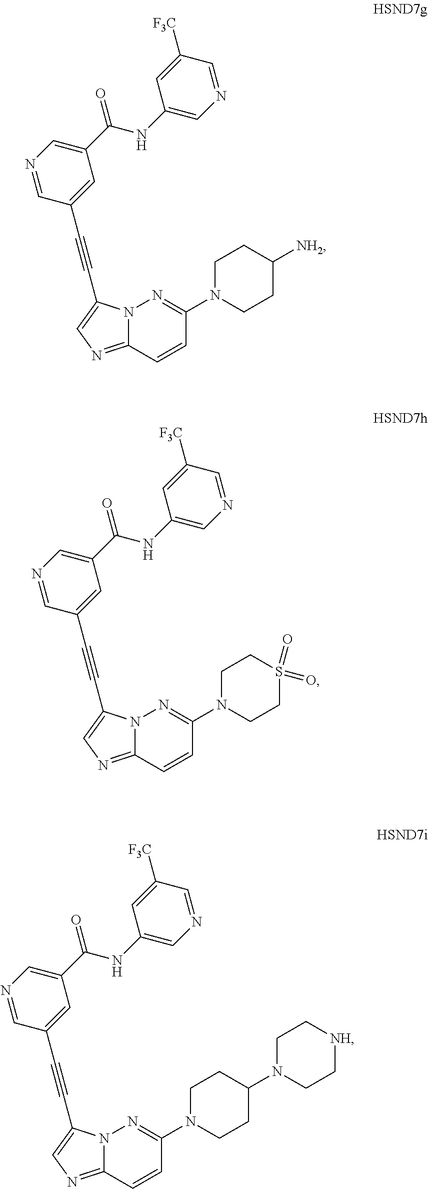 [SMILES: NC1CCN(c2ccc3ncc(C#Cc4cncc(C(=O)Nc5cncc(C(F)(F)F)c5)c4)n3n2)CC1 ; O=C(Nc1cncc(C(F)(F)F)c1)c1cncc(C#Cc2cnc3ccc(N4CCS(=O)(=O)CC4)nn23)c1 ; O=C(Nc1cncc(C(F)(F)F)c1)c1cncc(C#Cc2cnc3ccc(N4CCC(N5CCNCC5)CC4)nn23)c1]

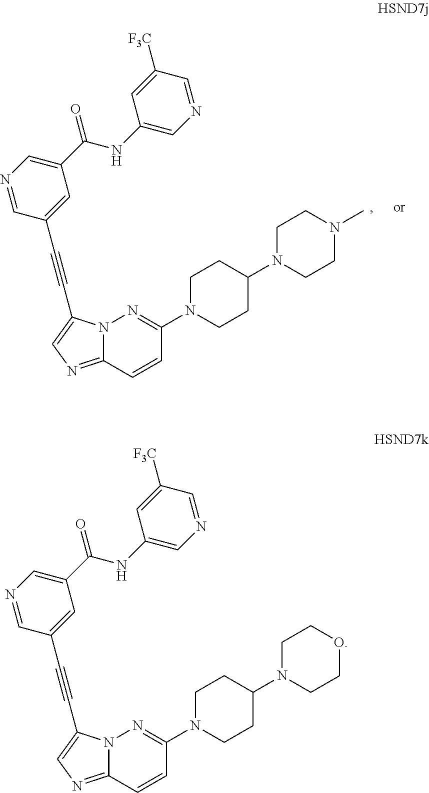 [SMILES: CN1CCN(C2CCN(c3ccc4ncc(C#Cc5cncc(C(=O)Nc6cncc(C(F)(F)F)c6)c5)n4n3)CC2)CC1 ; O=C(Nc1cncc(C(F)(F)F)c1)c1cncc(C#Cc2cnc3ccc(N4CCC(N5CCOCC5)CC4)nn23)c1]

The compound can have a structure:

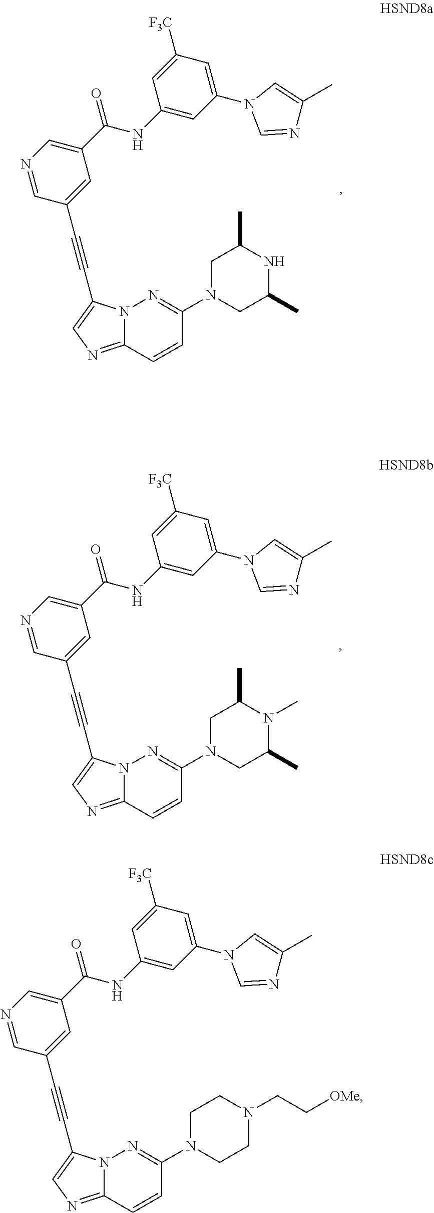 [SMILES: Cc1cn(-c2cc(NC(=O)c3cncc(C#Cc4cnc5ccc(N6C[C@@H](C)N[C@@H](C)C6)nn45)c3)cc(C(F)(F)F)c2)cn1 ; Cc1cn(-c2cc(NC(=O)c3cncc(C#Cc4cnc5ccc(N6C[C@@H](C)N(C)[C@@H](C)C6)nn45)c3)cc(C(F)(F)F)c2)cn1 ; COCCN1CCN(c2ccc3ncc(C#Cc4cncc(C(=O)Nc5cc(-n6cnc(C)c6)cc(C(F)(F)F)c5)c4)n3n2)CC1]

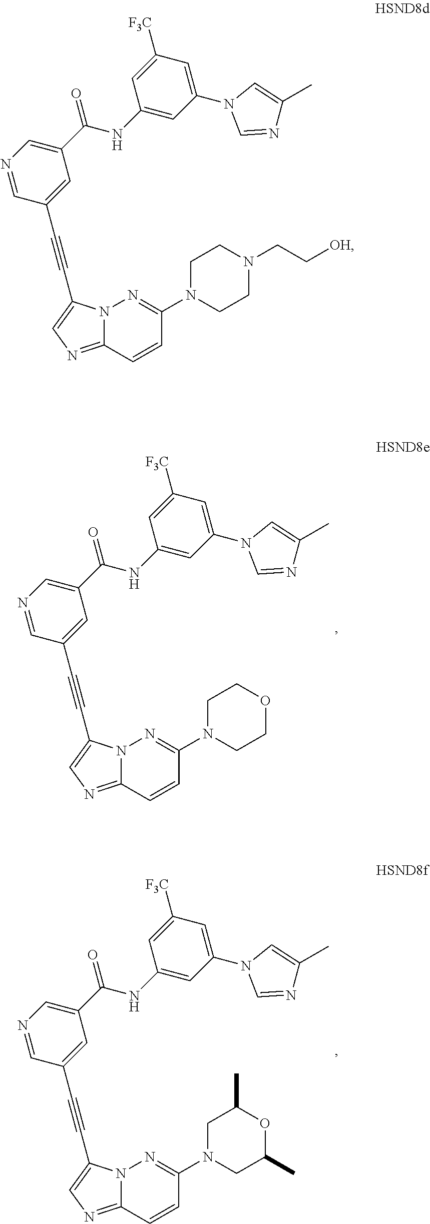 [SMILES: Cc1cn(-c2cc(NC(=O)c3cncc(C#Cc4cnc5ccc(N6CCN(CCO)CC6)nn45)c3)cc(C(F)(F)F)c2)cn1 ; Cc1cn(-c2cc(NC(=O)c3cncc(C#Cc4cnc5ccc(N6CCOCC6)nn45)c3)cc(C(F)(F)F)c2)cn1 ; Cc1cn(-c2cc(NC(=O)c3cncc(C#Cc4cnc5ccc(N6C[C@@H](C)O[C@@H](C)C6)nn45)c3)cc(C(F)(F)F)c2)cn1]

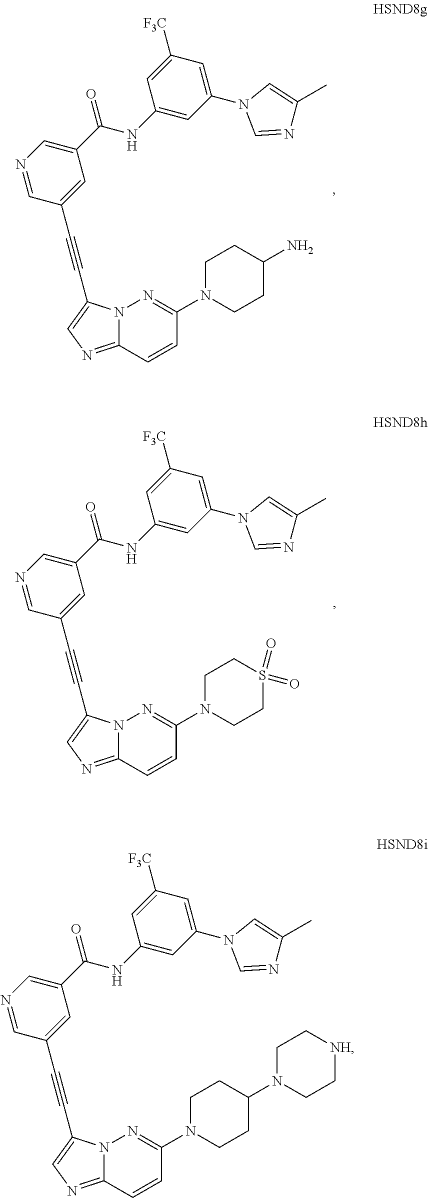 [SMILES: Cc1cn(-c2cc(NC(=O)c3cncc(C#Cc4cnc5ccc(N6CCC(N)CC6)nn45)c3)cc(C(F)(F)F)c2)cn1 ; Cc1cn(-c2cc(NC(=O)c3cncc(C#Cc4cnc5ccc(N6CCS(=O)(=O)CC6)nn45)c3)cc(C(F)(F)F)c2)cn1 ; Cc1cn(-c2cc(NC(=O)c3cncc(C#Cc4cnc5ccc(N6CCC(N7CCNCC7)CC6)nn45)c3)cc(C(F)(F)F)c2)cn1]

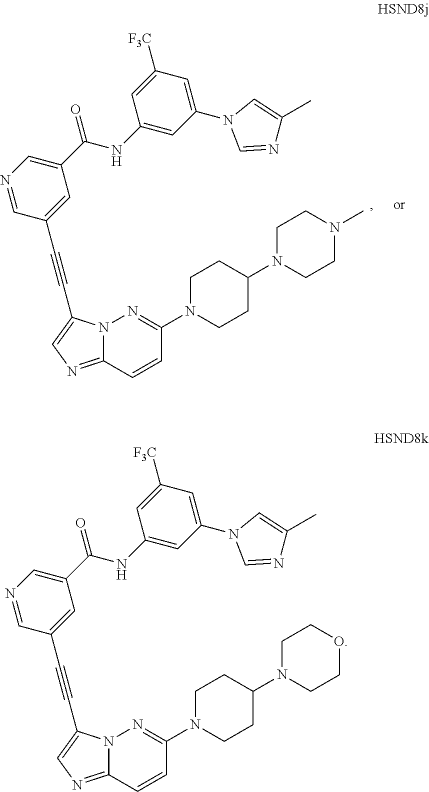 [SMILES: Cc1cn(-c2cc(NC(=O)c3cncc(C#Cc4cnc5ccc(N6CCC(N7CCN(C)CC7)CC6)nn45)c3)cc(C(F)(F)F)c2)cn1 ; Cc1cn(-c2cc(NC(=O)c3cncc(C#Cc4cnc5ccc(N6CCC(N7CCOCC7)CC6)nn45)c3)cc(C(F)(F)F)c2)cn1]

The compound can have the structure:

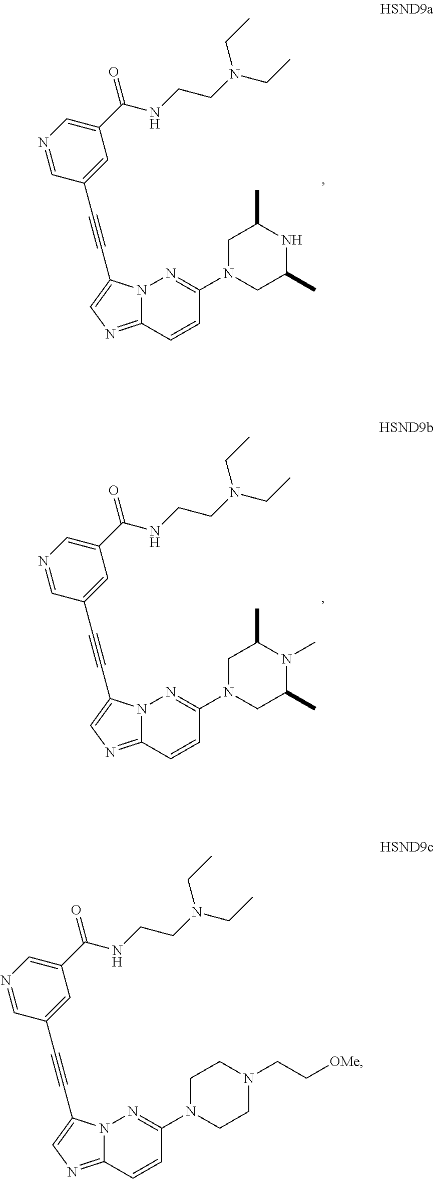 [SMILES: CCN(CC)CCNC(=O)c1cncc(C#Cc2cnc3ccc(N4C[C@@H](C)N[C@@H](C)C4)nn23)c1 ; CCN(CC)CCNC(=O)c1cncc(C#Cc2cnc3ccc(N4C[C@@H](C)N(C)[C@@H](C)C4)nn23)c1 ; CCN(CC)CCNC(=O)c1cncc(C#Cc2cnc3ccc(N4CCN(CCOC)CC4)nn23)c1]

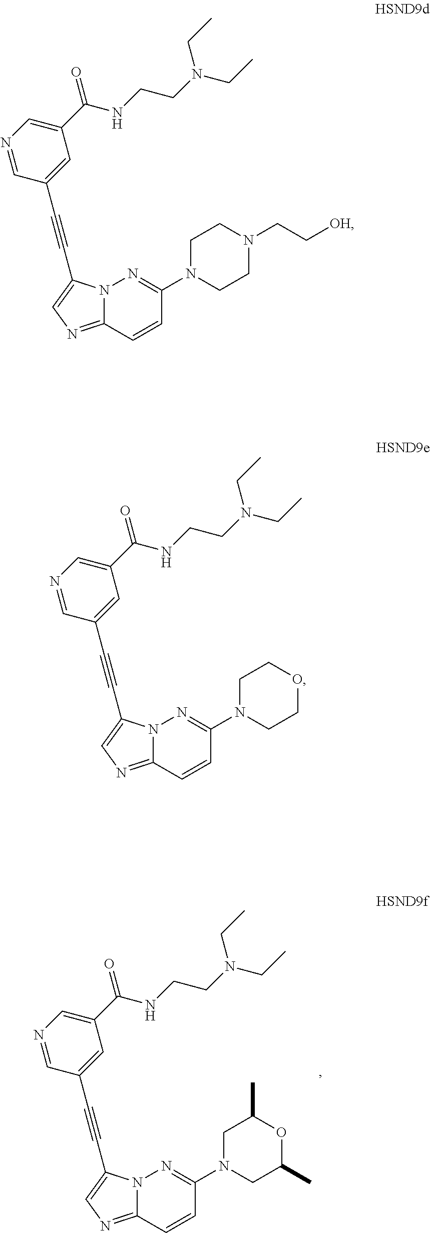 [SMILES: CCN(CC)CCNC(=O)c1cncc(C#Cc2cnc3ccc(N4CCN(CCO)CC4)nn23)c1 ; CCN(CC)CCNC(=O)c1cncc(C#Cc2cnc3ccc(N4CCOCC4)nn23)c1 ; CCN(CC)CCNC(=O)c1cncc(C#Cc2cnc3ccc(N4C[C@@H](C)O[C@@H](C)C4)nn23)c1]

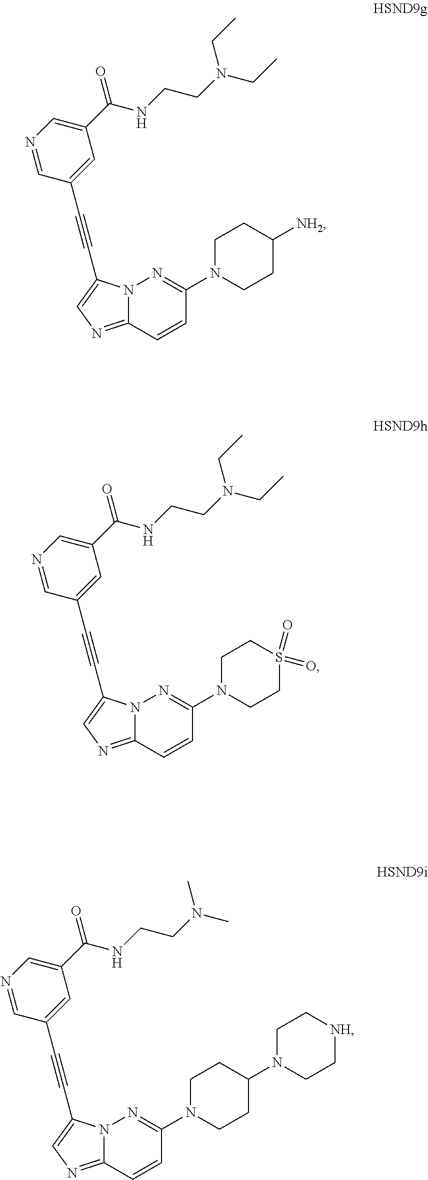 [SMILES: CCN(CC)CCNC(=O)c1cncc(C#Cc2cnc3ccc(N4CCC(N)CC4)nn23)c1 ; CCN(CC)CCNC(=O)c1cncc(C#Cc2cnc3ccc(N4CCS(=O)(=O)CC4)nn23)c1 ; CN(C)CCNC(=O)c1cncc(C#Cc2cnc3ccc(N4CCC(N5CCNCC5)CC4)nn23)c1]

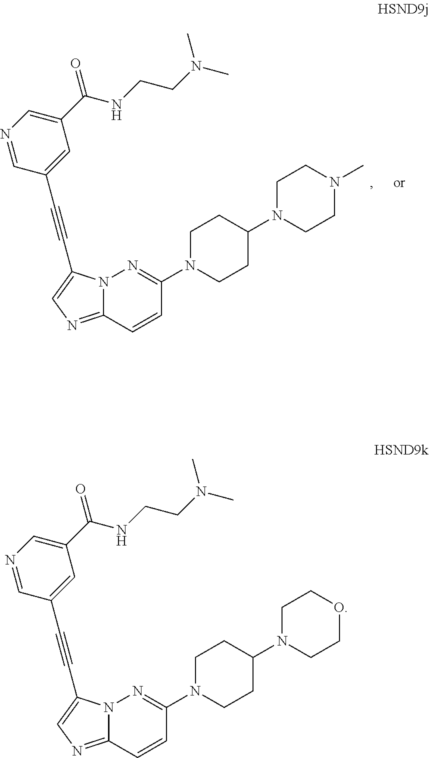 [SMILES: CN(C)CCNC(=O)c1cncc(C#Cc2cnc3ccc(N4CCC(N5CCN(C)CC5)CC4)nn23)c1 ; CN(C)CCNC(=O)c1cncc(C#Cc2cnc3ccc(N4CCC(N5CCOCC5)CC4)nn23)c1]

The compound can have a structure:

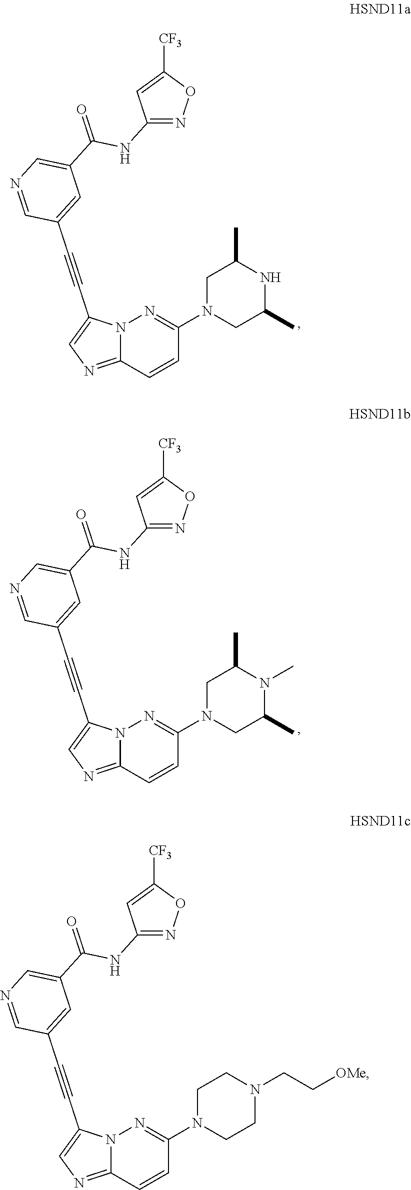 [SMILES: C[C@@H]1CN(c2ccc3ncc(C#Cc4cncc(C(=O)Nc5cc(C(F)(F)F)on5)c4)n3n2)C[C@H](C)N1 ; C[C@@H]1CN(c2ccc3ncc(C#Cc4cncc(C(=O)Nc5cc(C(F)(F)F)on5)c4)n3n2)C[C@H](C)N1C ; COCCN1CCN(c2ccc3ncc(C#Cc4cncc(C(=O)Nc5cc(C(F)(F)F)on5)c4)n3n2)CC1]

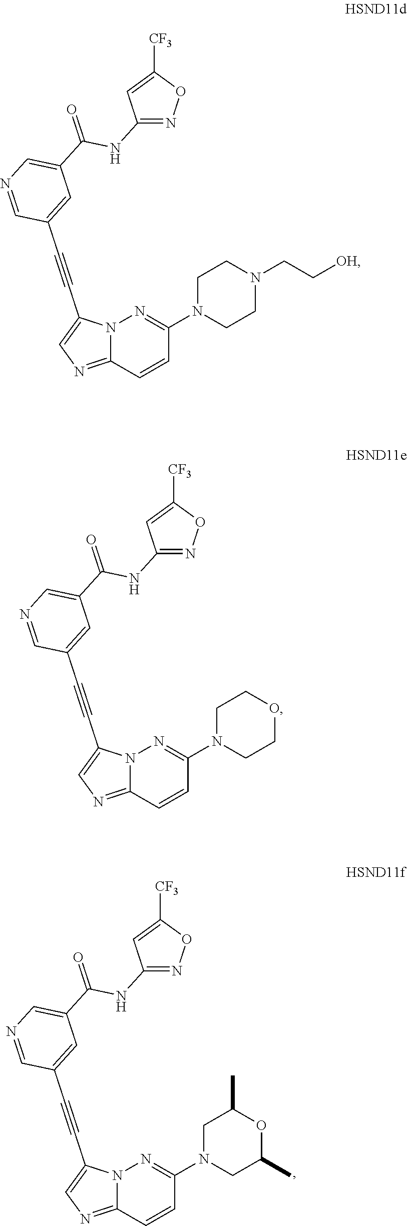 [SMILES: O=C(Nc1cc(C(F)(F)F)on1)c1cncc(C#Cc2cnc3ccc(N4CCN(CCO)CC4)nn23)c1 ; O=C(Nc1cc(C(F)(F)F)on1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)on5)c4)n3n2)C[C@H](C)O1]

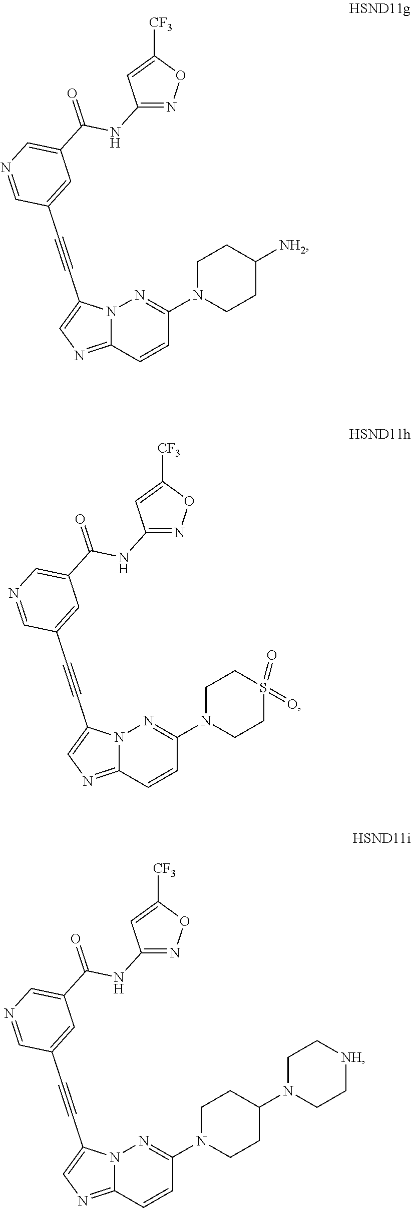 [SMILES: NC1CCN(c2ccc3ncc(C#Cc4cncc(C(=O)Nc5cc(C(F)(F)F)on5)c4)n3n2)CC1 ; O=C(Nc1cc(C(F)(F)F)on1)c1cncc(C#Cc2cnc3ccc(N4CCS(=O)(=O)CC4)nn23)c1 ; O=C(Nc1cc(C(F)(F)F)on1)c1cncc(C#Cc2cnc3ccc(N4CCC(N5CCNCC5)CC4)nn23)c1]

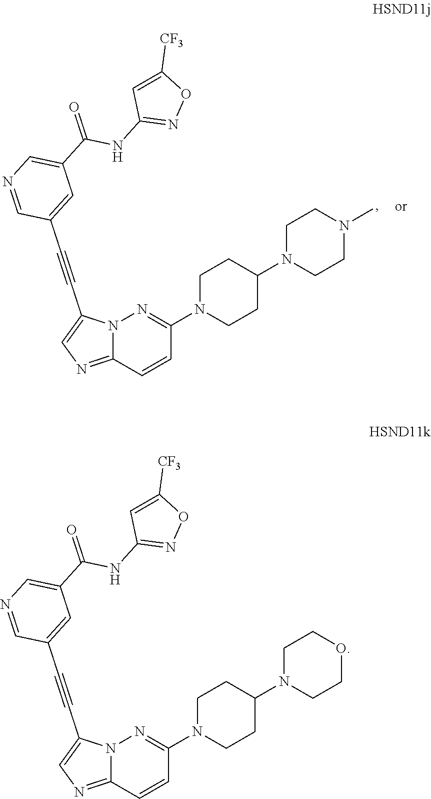 [SMILES: CN1CCN(C2CCN(c3ccc4ncc(C#Cc5cncc(C(=O)Nc6cc(C(F)(F)F)on6)c5)n4n3)CC2)CC1 ; O=C(Nc1cc(C(F)(F)F)on1)c1cncc(C#Cc2cnc3ccc(N4CCC(N5CCOCC5)CC4)nn23)c1]

A compound can have a structure:

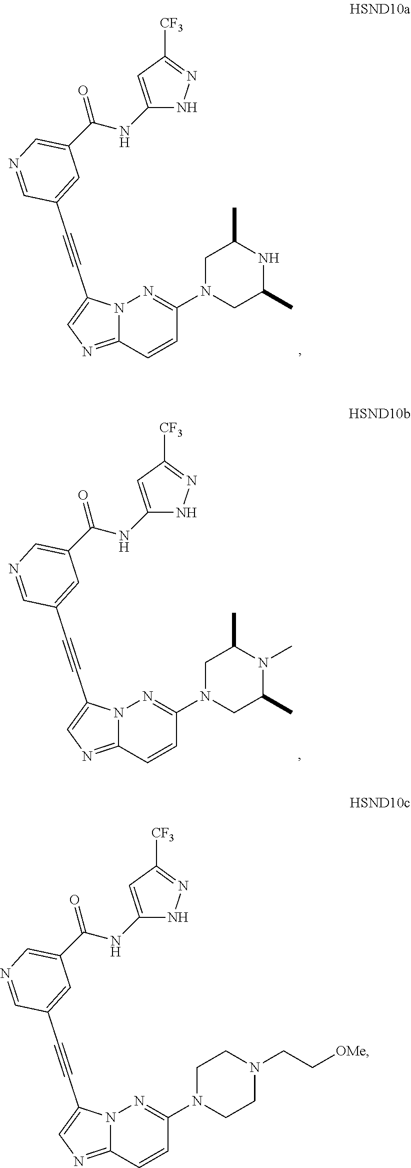 [SMILES: C[C@@H]1CN(c2ccc3ncc(C#Cc4cncc(C(=O)Nc5cc(C(F)(F)F)n[nH]5)c4)n3n2)C[C@H](C)N1 ; C[C@@H]1CN(c2ccc3ncc(C#Cc4cncc(C(=O)Nc5cc(C(F)(F)F)n[nH]5)c4)n3n2)C[C@H](C)N1C ; COCCN1CCN(c2ccc3ncc(C#Cc4cncc(C(=O)Nc5cc(C(F)(F)F)n[nH]5)c4)n3n2)CC1]

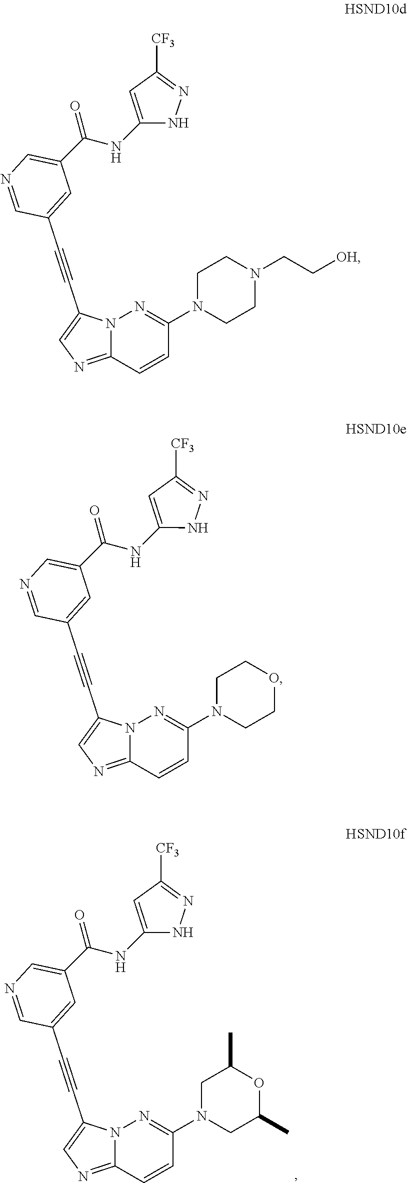 [SMILES: O=C(Nc1cc(C(F)(F)F)n[nH]1)c1cncc(C#Cc2cnc3ccc(N4CCN(CCO)CC4)nn23)c1 ; O=C(Nc1cc(C(F)(F)F)n[nH]1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)n[nH]5)c4)n3n2)C[C@H](C)O1]

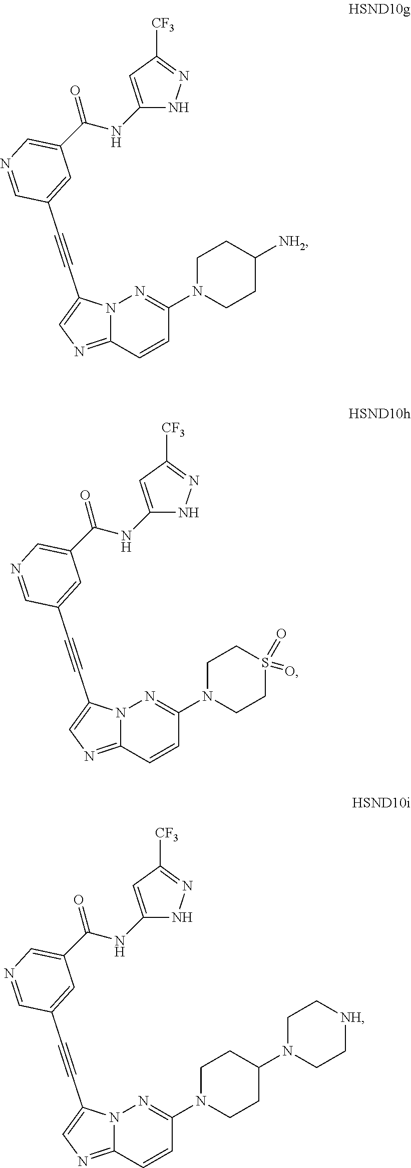 [SMILES: NC1CCN(c2ccc3ncc(C#Cc4cncc(C(=O)Nc5cc(C(F)(F)F)n[nH]5)c4)n3n2)CC1 ; O=C(Nc1cc(C(F)(F)F)n[nH]1)c1cncc(C#Cc2cnc3ccc(N4CCS(=O)(=O)CC4)nn23)c1 ; O=C(Nc1cc(C(F)(F)F)n[nH]1)c1cncc(C#Cc2cnc3ccc(N4CCC(N5CCNCC5)CC4)nn23)c1]

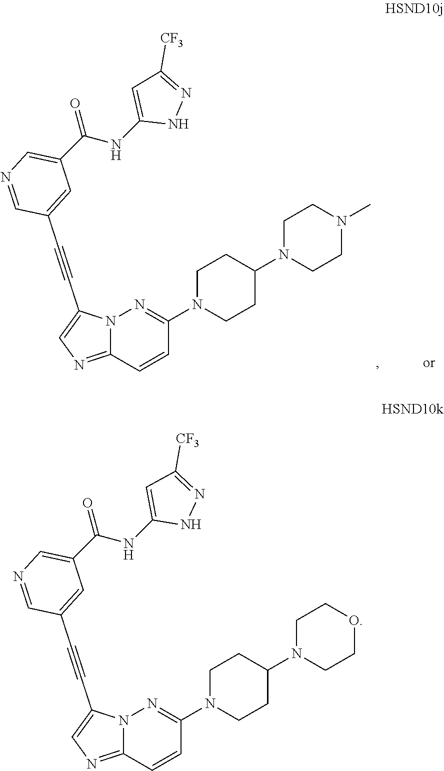 [SMILES: CN1CCN(C2CCN(c3ccc4ncc(C#Cc5cncc(C(=O)Nc6cc(C(F)(F)F)n[nH]6)c5)n4n3)CC2)CC1 ; O=C(Nc1cc(C(F)(F)F)n[nH]1)c1cncc(C#Cc2cnc3ccc(N4CCC(N5CCOCC5)CC4)nn23)c1]

A compound can have a structure:

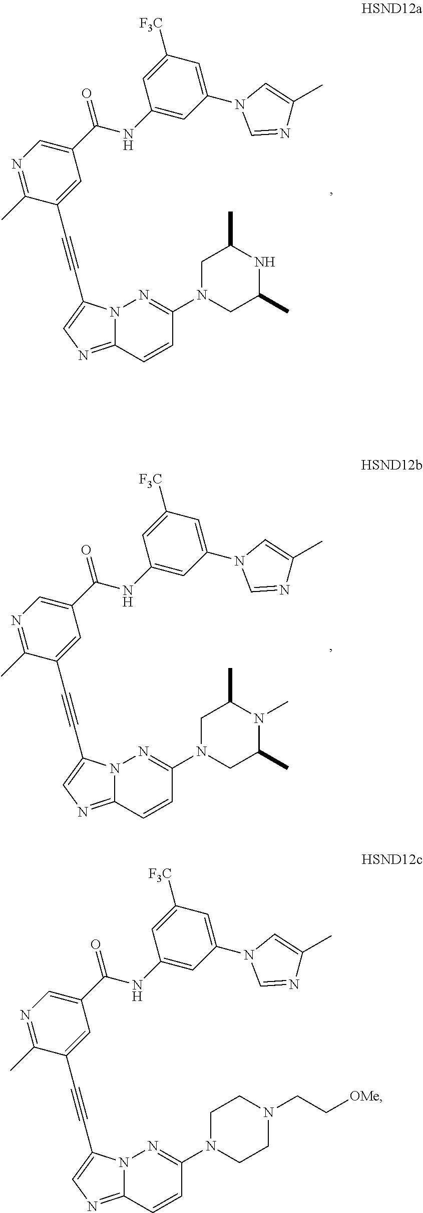

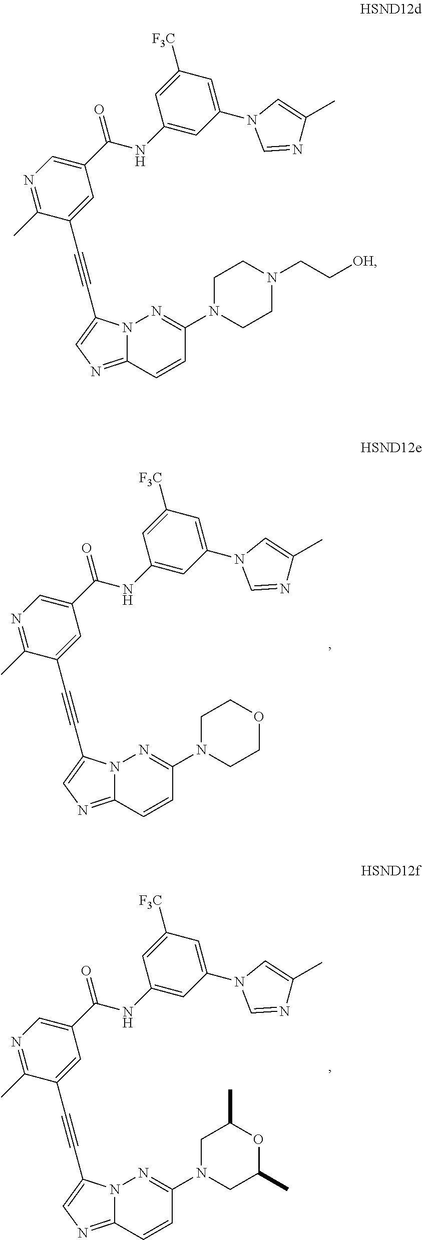

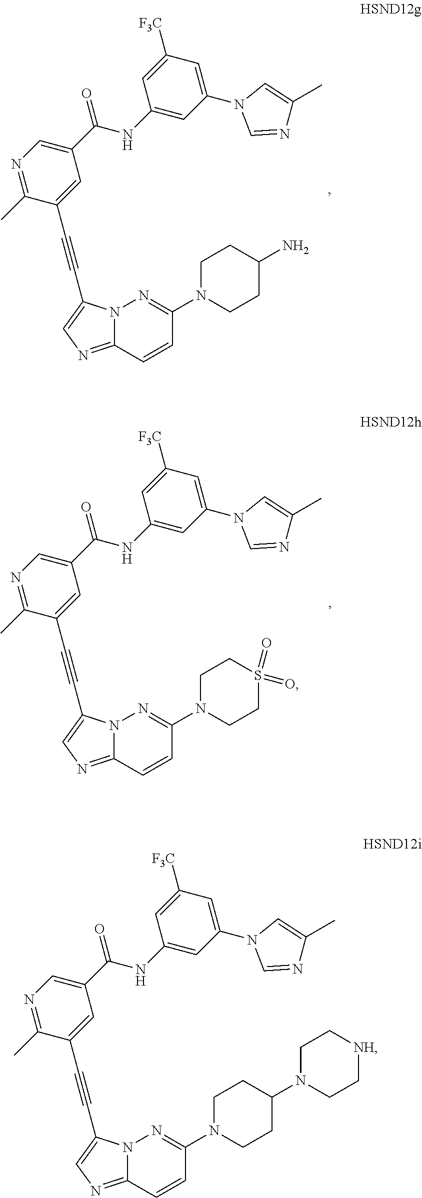

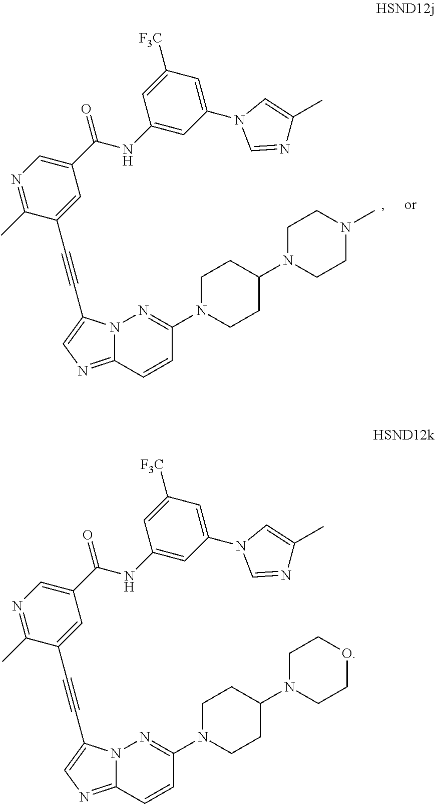

The compound can have a structure:

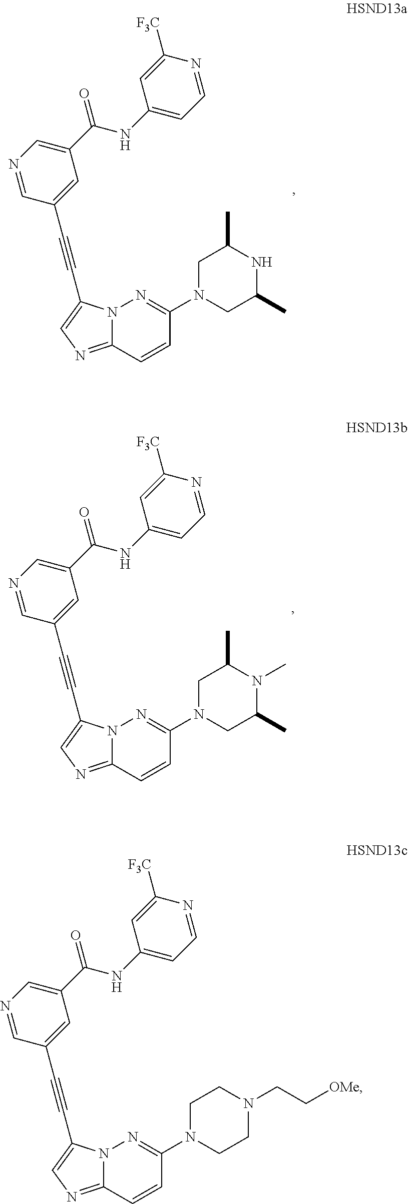 [SMILES: C[C@@H]1CN(c2ccc3ncc(C#Cc4cncc(C(=O)Nc5ccnc(C(F)(F)F)c5)c4)n3n2)C[C@H](C)N1 ; C[C@@H]1CN(c2ccc3ncc(C#Cc4cncc(C(=O)Nc5ccnc(C(F)(F)F)c5)c4)n3n2)C[C@H](C)N1C ; COCCN1CCN(c2ccc3ncc(C#Cc4cncc(C(=O)Nc5ccnc(C(F)(F)F)c5)c4)n3n2)CC1]

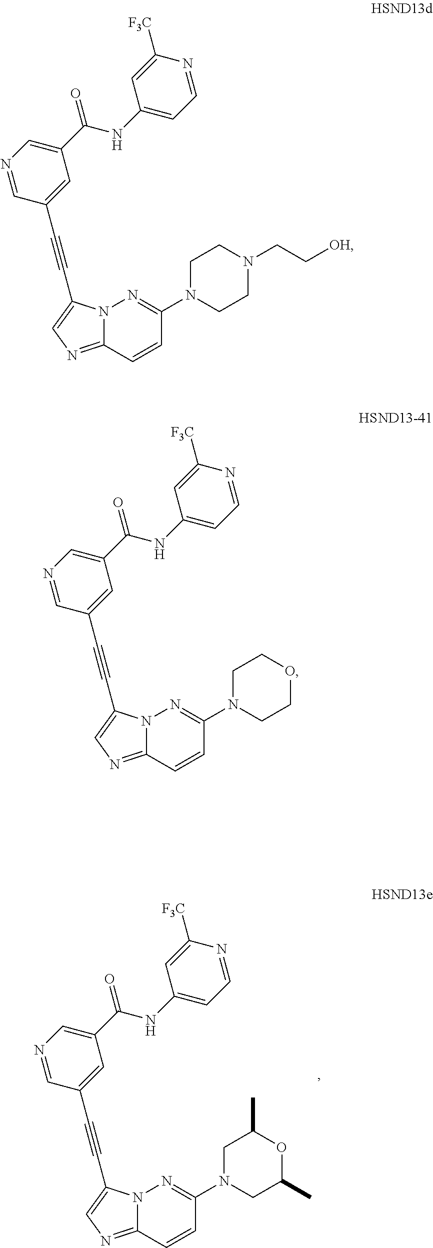 [SMILES: O=C(Nc1ccnc(C(F)(F)F)c1)c1cncc(C#Cc2cnc3ccc(N4CCN(CCO)CC4)nn23)c1 ; O=C(Nc1ccnc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5ccnc(C(F)(F)F)c5)c4)n3n2)C[C@H](C)O1]

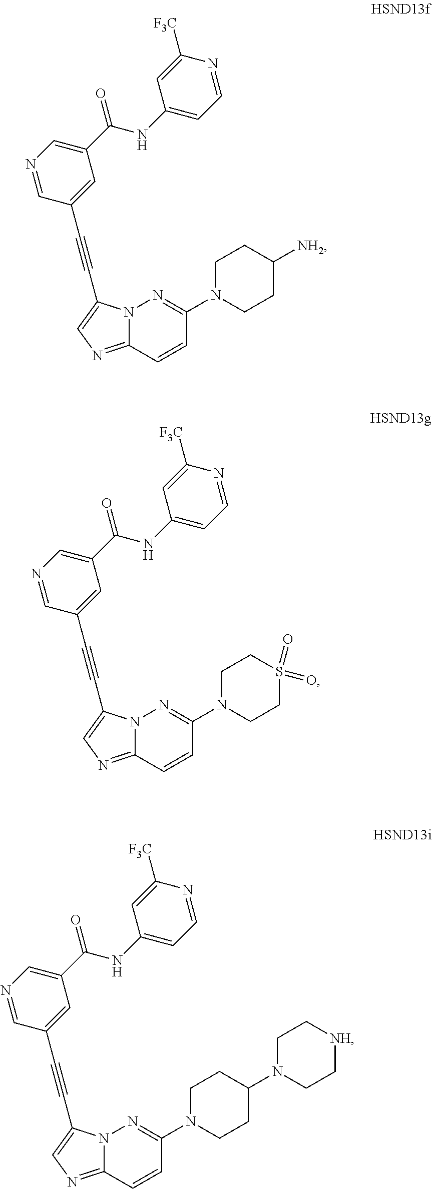 [SMILES: NC1CCN(c2ccc3ncc(C#Cc4cncc(C(=O)Nc5ccnc(C(F)(F)F)c5)c4)n3n2)CC1 ; O=C(Nc1ccnc(C(F)(F)F)c1)c1cncc(C#Cc2cnc3ccc(N4CCS(=O)(=O)CC4)nn23)c1 ; O=C(Nc1ccnc(C(F)(F)F)c1)c1cncc(C#Cc2cnc3ccc(N4CCC(N5CCNCC5)CC4)nn23)c1]

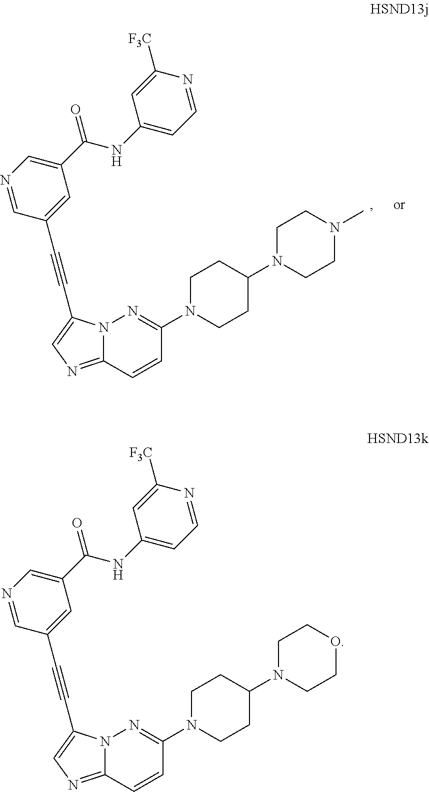 [SMILES: CN1CCN(C2CCN(c3ccc4ncc(C#Cc5cncc(C(=O)Nc6ccnc(C(F)(F)F)c6)c5)n4n3)CC2)CC1 ; O=C(Nc1ccnc(C(F)(F)F)c1)c1cncc(C#Cc2cnc3ccc(N4CCC(N5CCOCC5)CC4)nn23)c1]

A compound can have a structure:

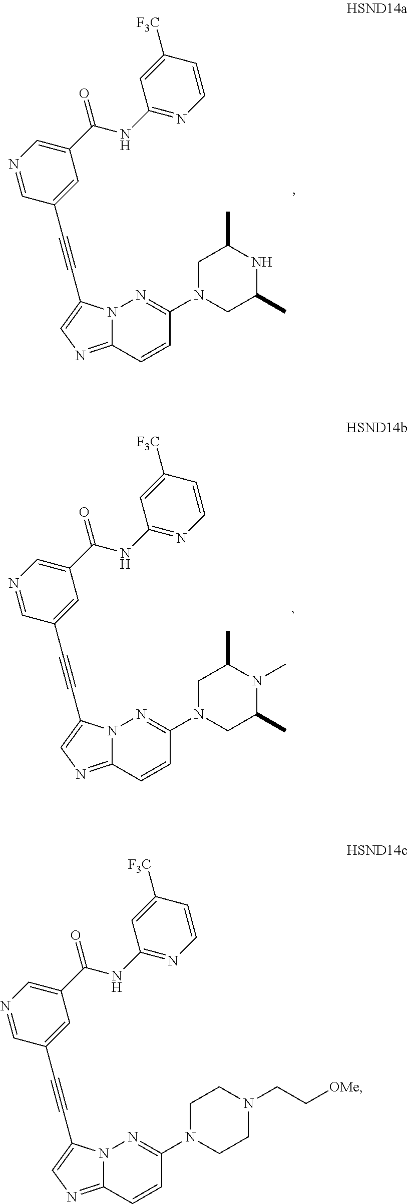 [SMILES: C[C@@H]1CN(c2ccc3ncc(C#Cc4cncc(C(=O)Nc5cc(C(F)(F)F)ccn5)c4)n3n2)C[C@H](C)N1 ; C[C@@H]1CN(c2ccc3ncc(C#Cc4cncc(C(=O)Nc5cc(C(F)(F)F)ccn5)c4)n3n2)C[C@H](C)N1C ; COCCN1CCN(c2ccc3ncc(C#Cc4cncc(C(=O)Nc5cc(C(F)(F)F)ccn5)c4)n3n2)CC1]

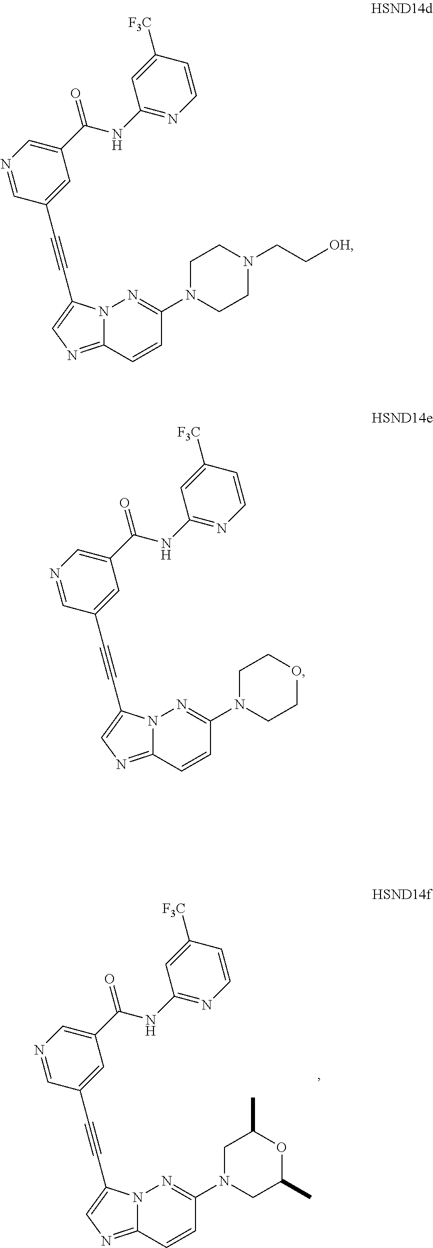 [SMILES: O=C(Nc1cc(C(F)(F)F)ccn1)c1cncc(C#Cc2cnc3ccc(N4CCN(CCO)CC4)nn23)c1 ; O=C(Nc1cc(C(F)(F)F)ccn1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)ccn5)c4)n3n2)C[C@H](C)O1]

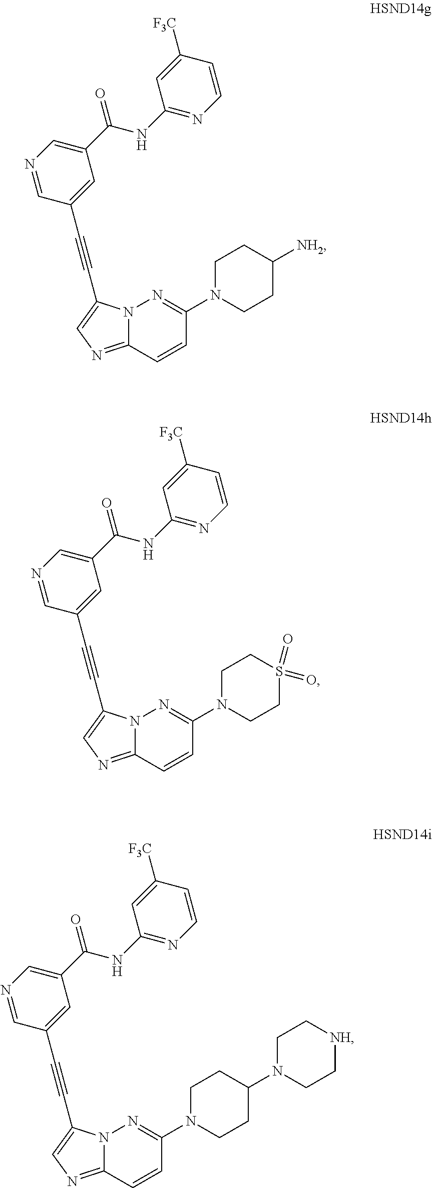 [SMILES: NC1CCN(c2ccc3ncc(C#Cc4cncc(C(=O)Nc5cc(C(F)(F)F)ccn5)c4)n3n2)CC1 ; O=C(Nc1cc(C(F)(F)F)ccn1)c1cncc(C#Cc2cnc3ccc(N4CCS(=O)(=O)CC4)nn23)c1 ; O=C(Nc1cc(C(F)(F)F)ccn1)c1cncc(C#Cc2cnc3ccc(N4CCC(N5CCNCC5)CC4)nn23)c1]

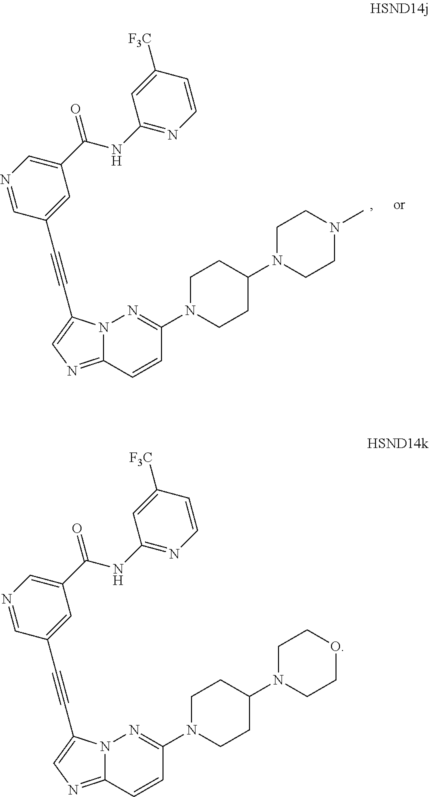 [SMILES: CN1CCN(C2CCN(c3ccc4ncc(C#Cc5cncc(C(=O)Nc6cc(C(F)(F)F)ccn6)c5)n4n3)CC2)CC1 ; O=C(Nc1cc(C(F)(F)F)ccn1)c1cncc(C#Cc2cnc3ccc(N4CCC(N5CCOCC5)CC4)nn23)c1]

The compound can have a structure:

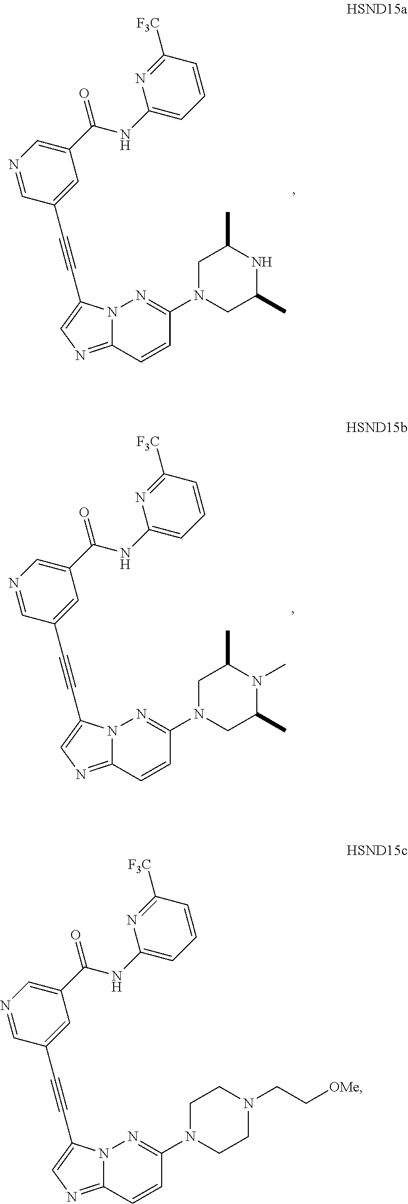 [SMILES: C[C@@H]1CN(c2ccc3ncc(C#Cc4cncc(C(=O)Nc5cccc(C(F)(F)F)n5)c4)n3n2)C[C@H](C)N1 ; C[C@@H]1CN(c2ccc3ncc(C#Cc4cncc(C(=O)Nc5cccc(C(F)(F)F)n5)c4)n3n2)C[C@H](C)N1C ; COCCN1CCN(c2ccc3ncc(C#Cc4cncc(C(=O)Nc5cccc(C(F)(F)F)n5)c4)n3n2)CC1]

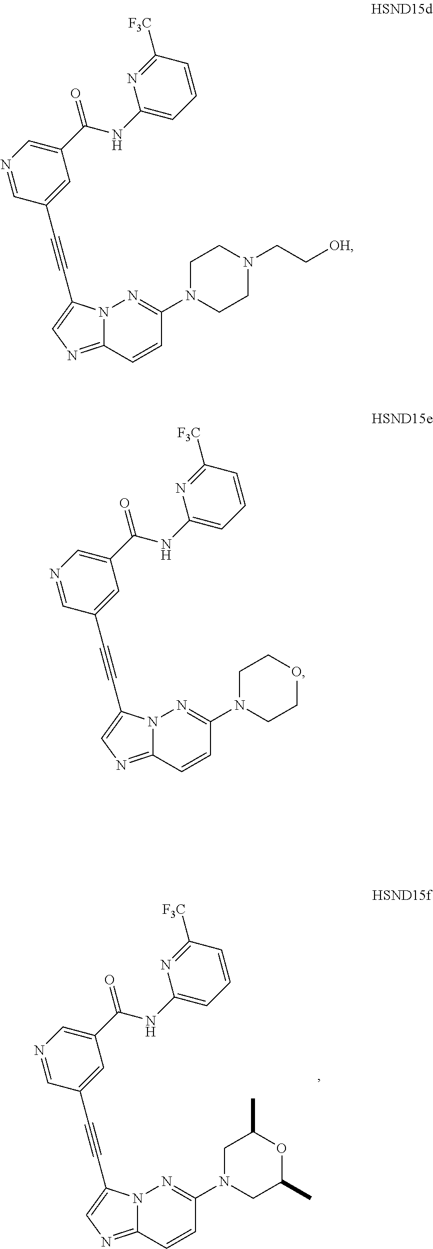 [SMILES: O=C(Nc1cccc(C(F)(F)F)n1)c1cncc(C#Cc2cnc3ccc(N4CCN(CCO)CC4)nn23)c1 ; O=C(Nc1cccc(C(F)(F)F)n1)c1cncc(C#Cc2cnc3ccc(N4CCOCC4)nn23)c1 ; C[C@@H]1CN(c2ccc3ncc(C#Cc4cncc(C(=O)Nc5cccc(C(F)(F)F)n5)c4)n3n2)C[C@H](C)O1]

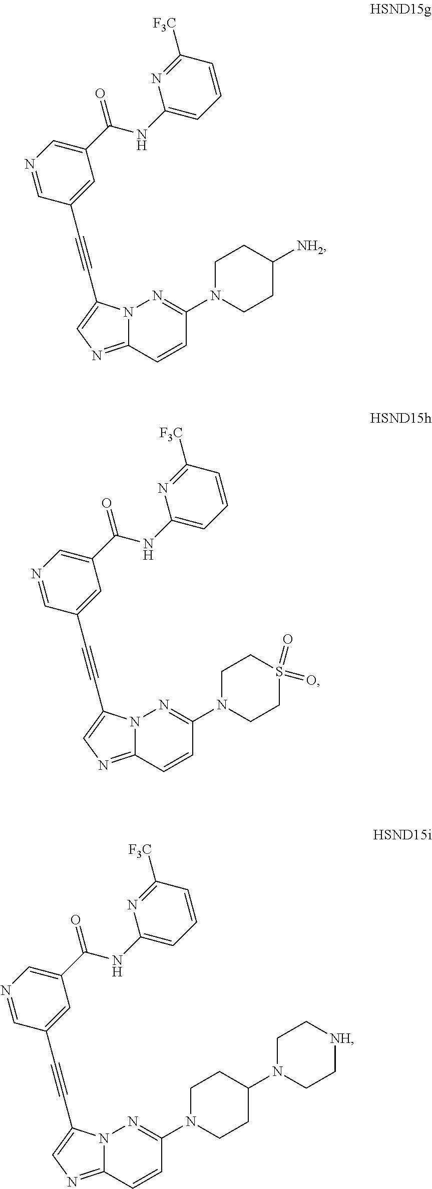 [SMILES: NC1CCN(c2ccc3ncc(C#Cc4cncc(C(=O)Nc5cccc(C(F)(F)F)n5)c4)n3n2)CC1 ; O=C(Nc1cccc(C(F)(F)F)n1)c1cncc(C#Cc2cnc3ccc(N4CCS(=O)(=O)CC4)nn23)c1 ; O=C(Nc1cccc(C(F)(F)F)n1)c1cncc(C#Cc2cnc3ccc(N4CCC(N5CCNCC5)CC4)nn23)c1]

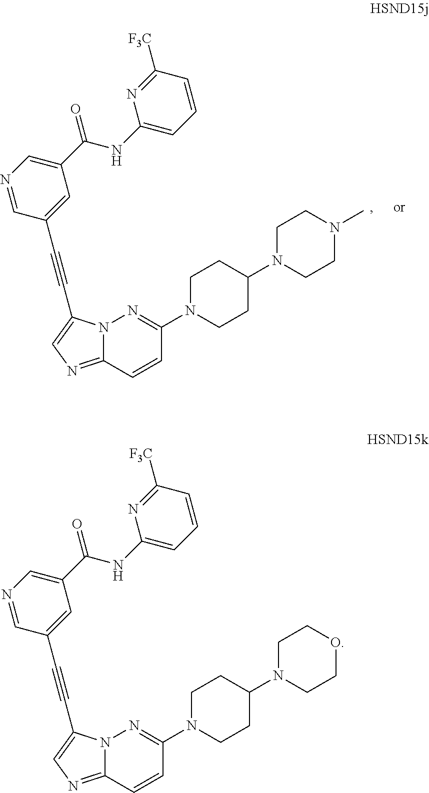 [SMILES: CN1CCN(C2CCN(c3ccc4ncc(C#Cc5cncc(C(=O)Nc6cccc(C(F)(F)F)n6)c5)n4n3)CC2)CC1 ; O=C(Nc1cccc(C(F)(F)F)n1)c1cncc(C#Cc2cnc3ccc(N4CCC(N5CCOCC5)CC4)nn23)c1]

The compound can have a structure:

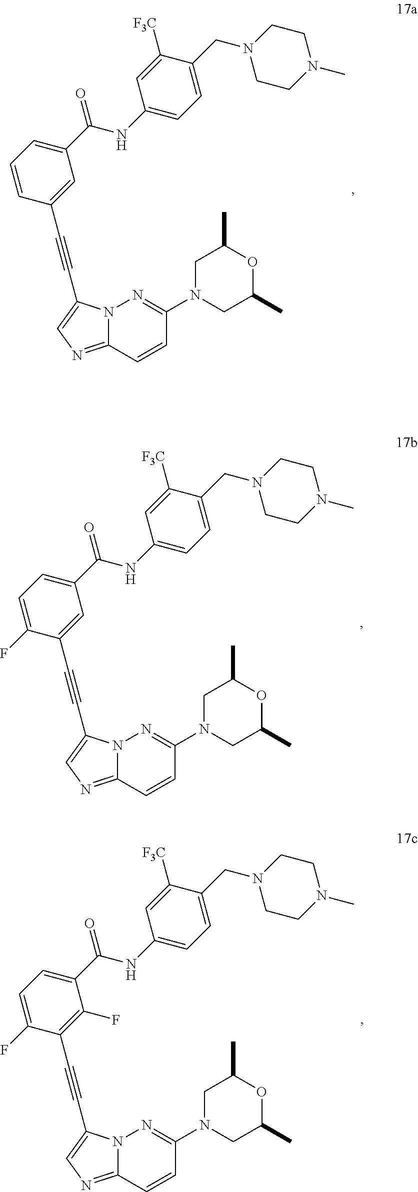

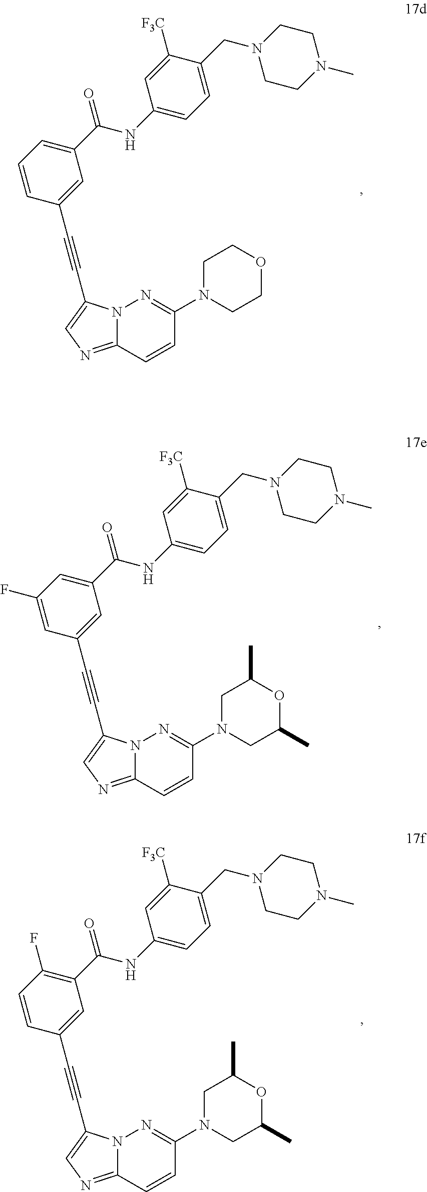

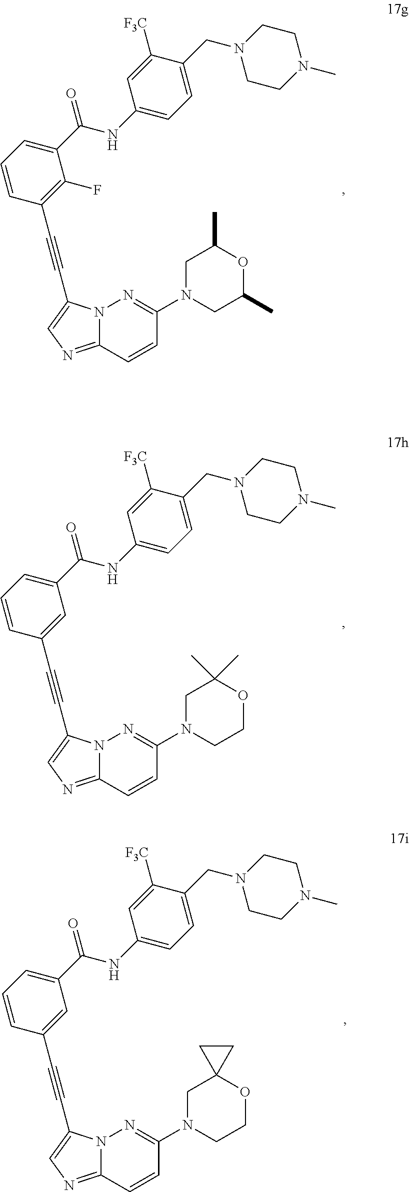

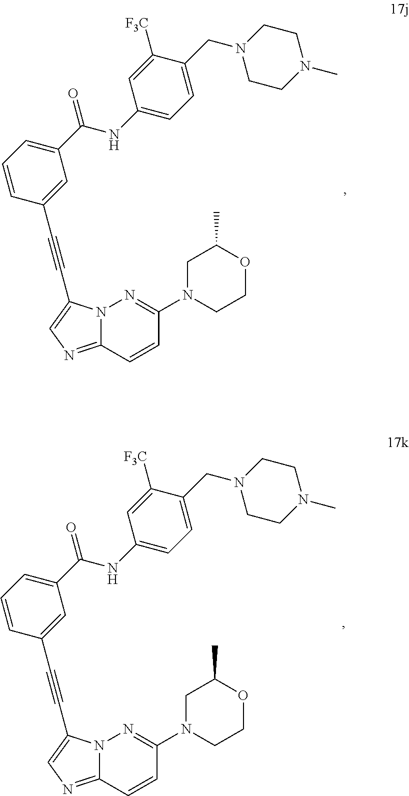

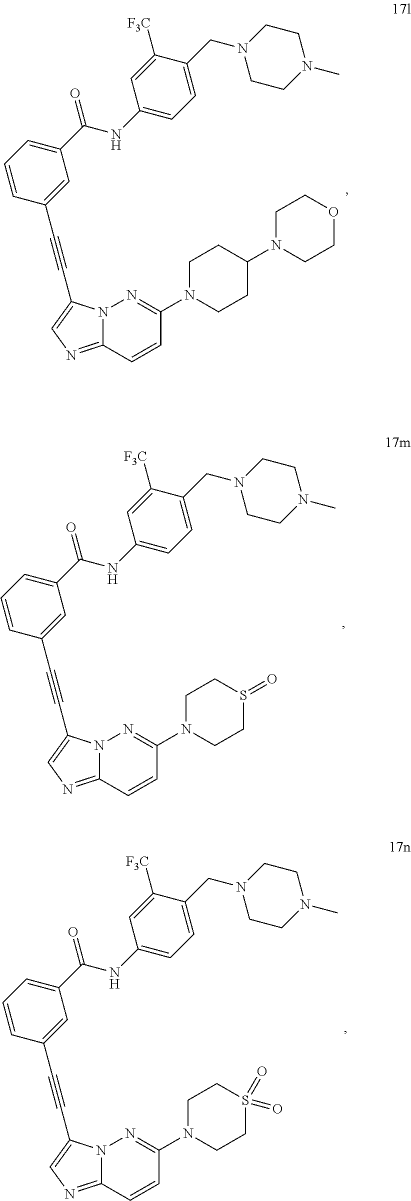

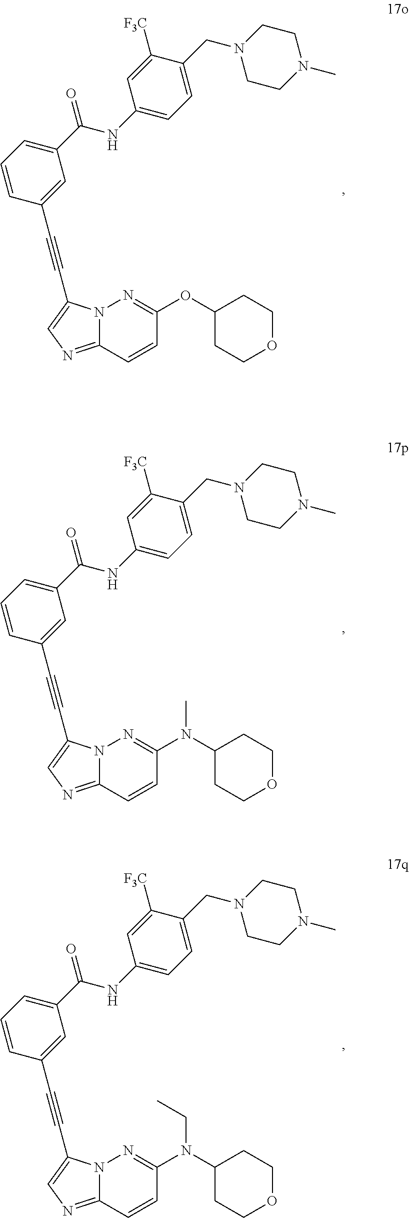

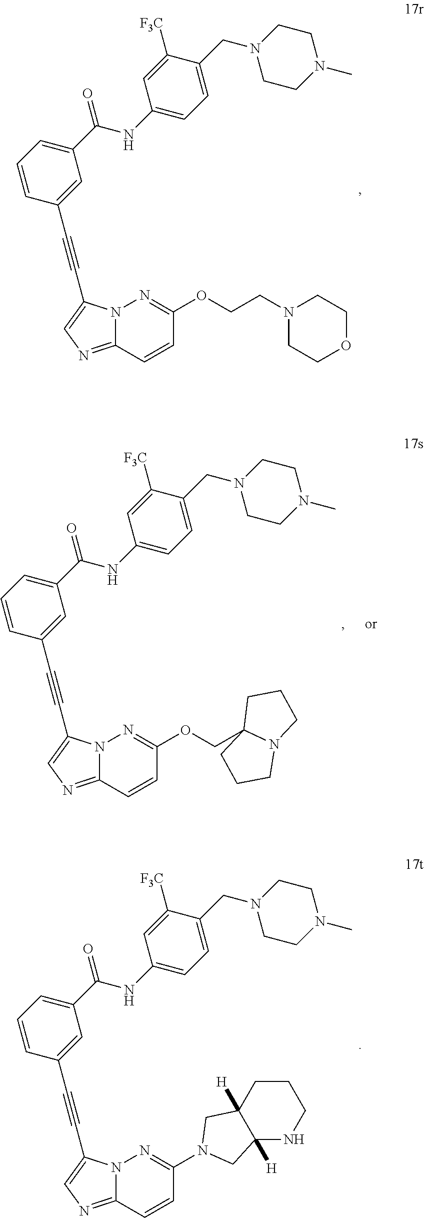

The compound can have a structure:

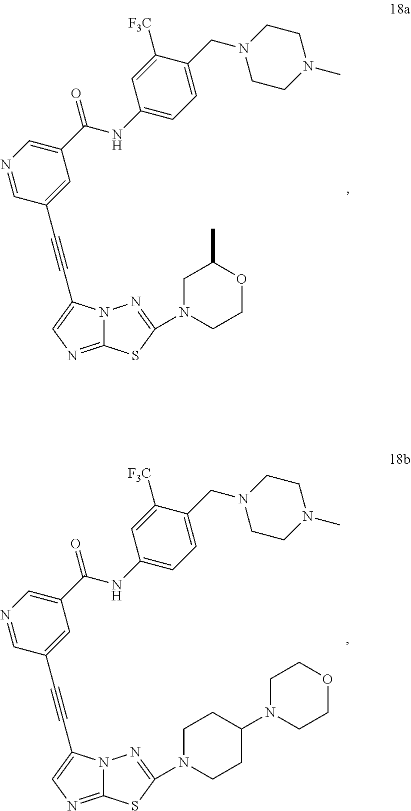

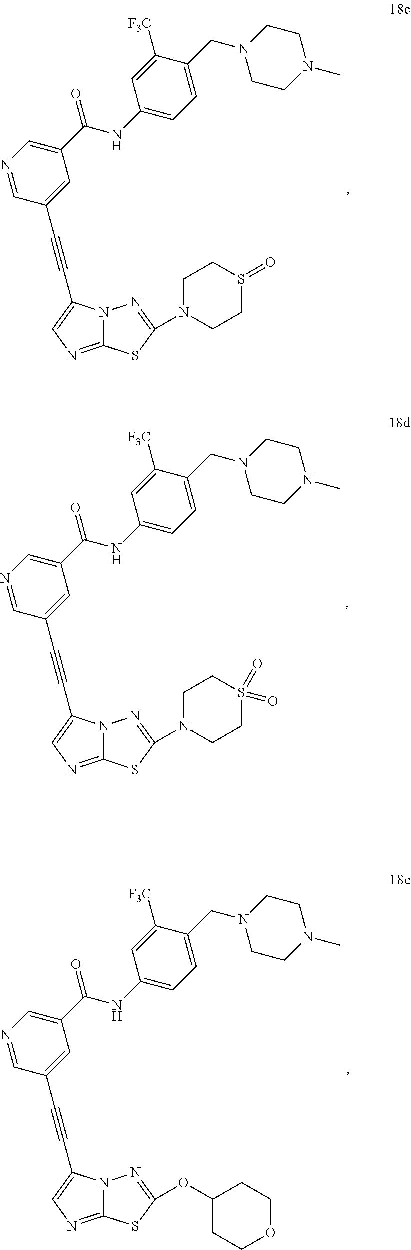

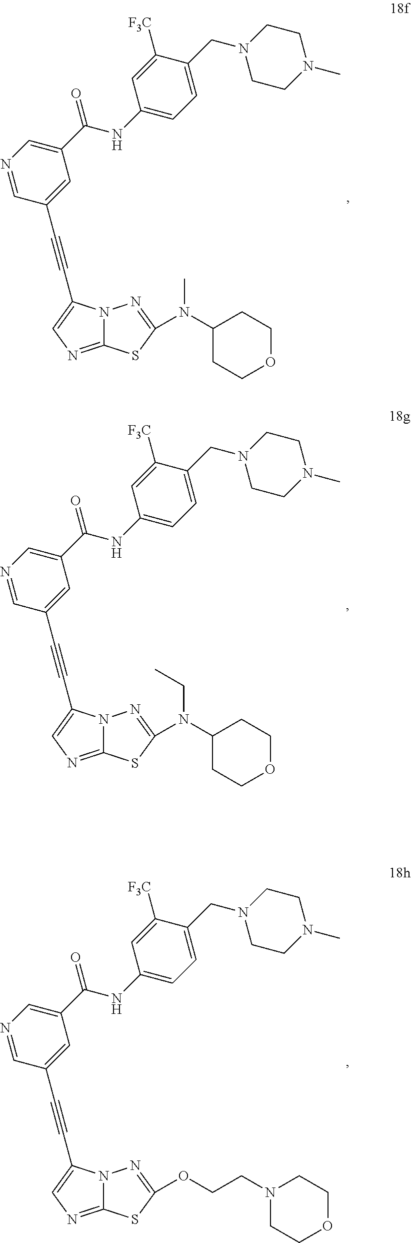

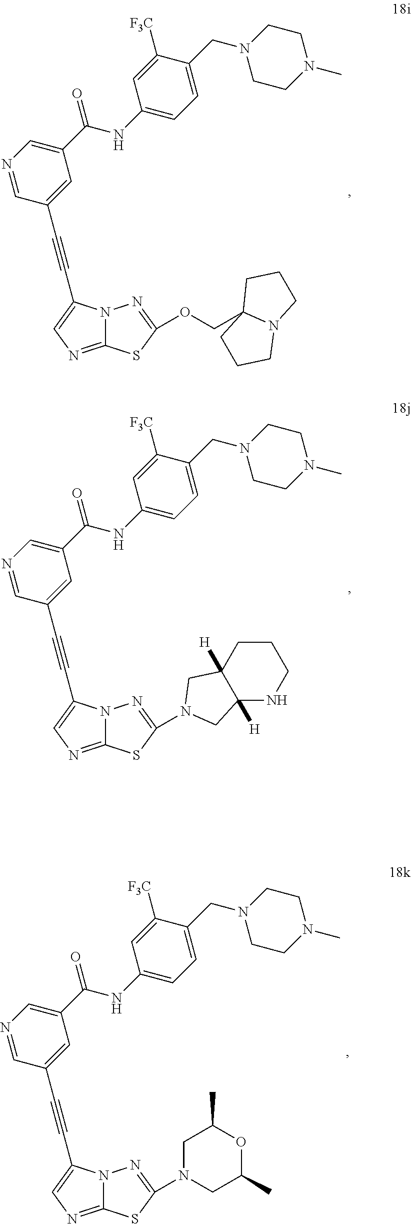

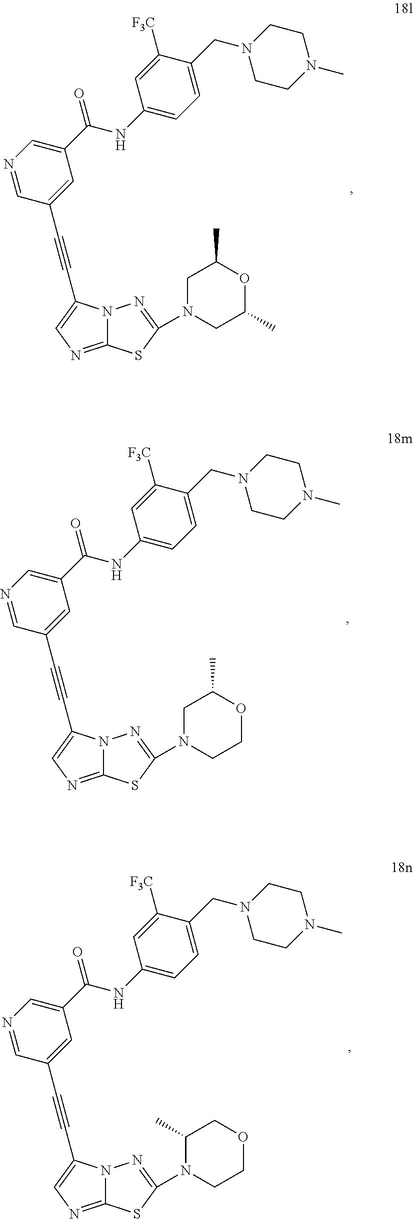

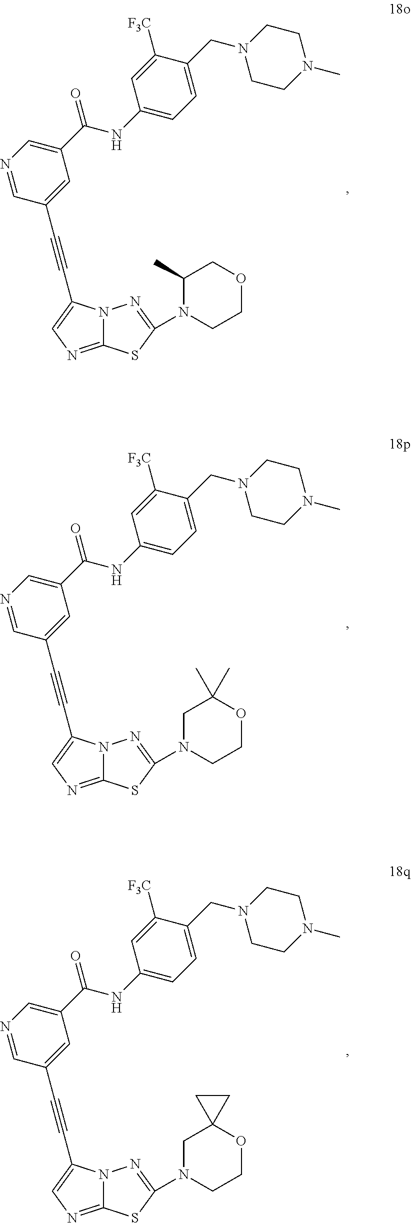

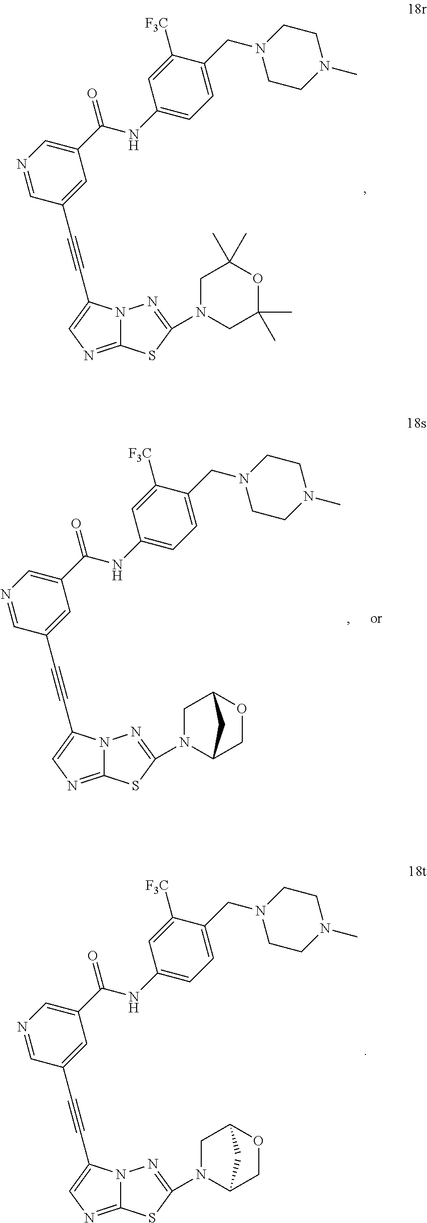

The compound can have the structure:

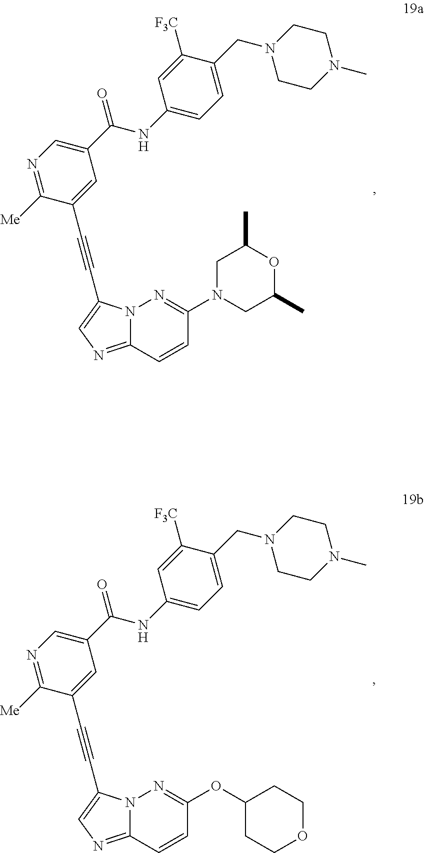

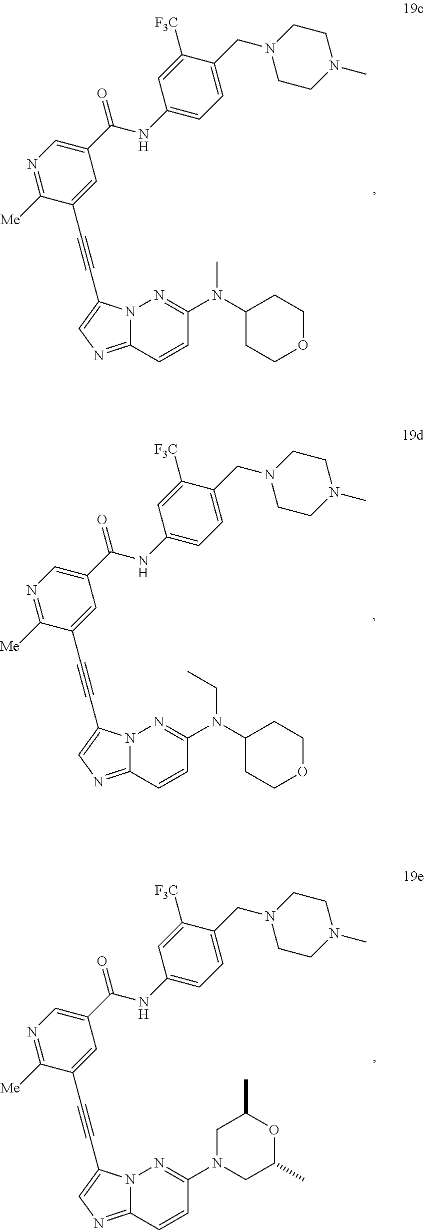

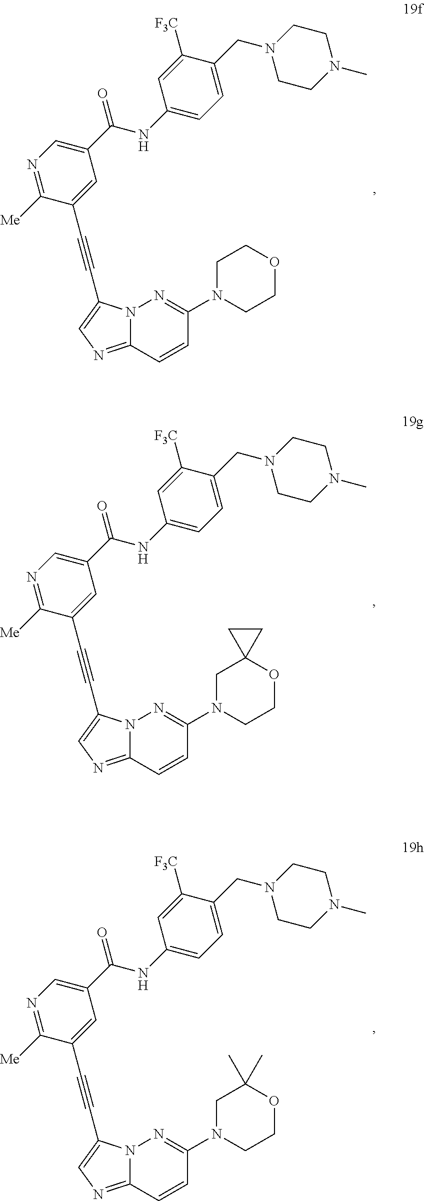

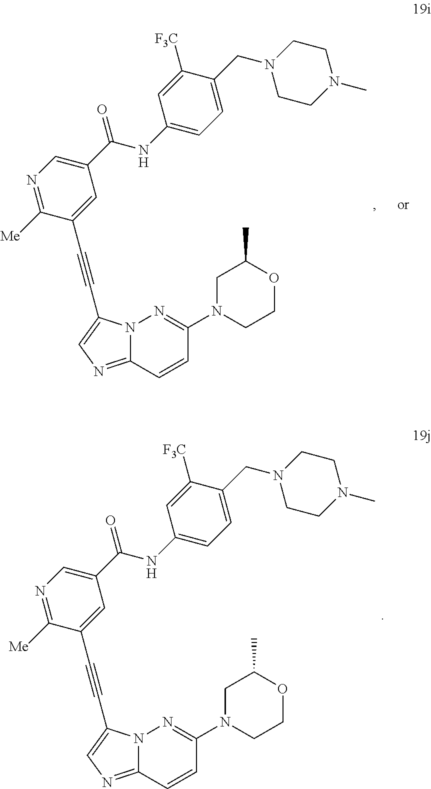

In certain embodiments, R₁ is

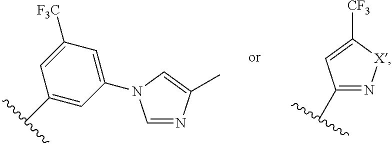

wherein

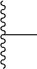

is a point of attachment and X′ is O, C, or N.

A compound can have a structure of

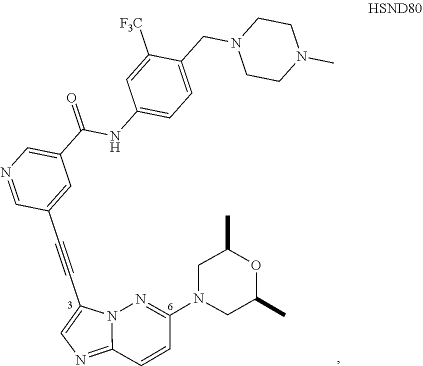 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)O1]

be a pharmaceutically acceptable salt thereof.

A compound can have a structure of

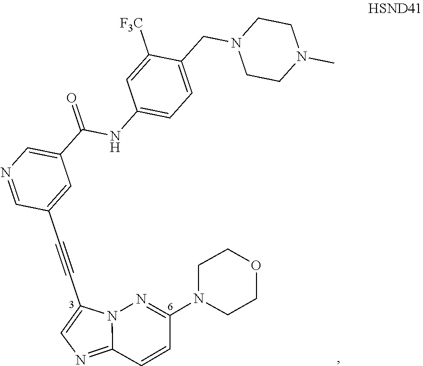 [SMILES: CN1CCN(Cc2ccc(NC(=O)c3cncc(C#Cc4cnc5ccc(N6CCOCC6)nn45)c3)cc2C(F)(F)F)CC1]

or be a pharmaceutically acceptable salt thereof

A compound can have a structure of

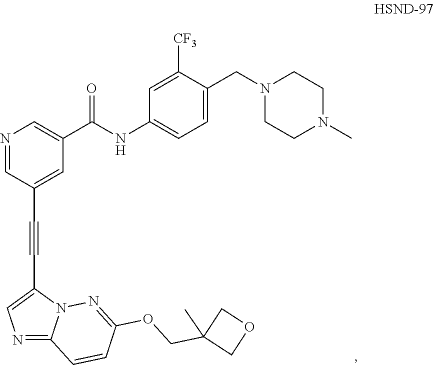 [SMILES: CN1CCN(Cc2ccc(NC(=O)c3cncc(C#Cc4cnc5ccc(OCC6(C)COC6)nn45)c3)cc2C(F)(F)F)CC1]

or be a pharmaceutically acceptable salt thereof.

A compound can have a structure of

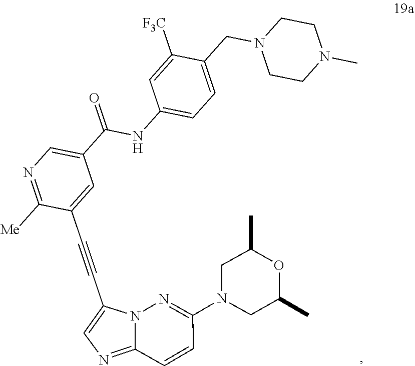 [SMILES: Cc1ncc(C(=O)Nc2ccc(CN3CCN(C)CC3)c(C(F)(F)F)c2)cc1C#Cc1cnc2ccc(N3C[C@@H](C)O[C@@H](C)C3)nn12]

or be a pharmaceutically acceptable salt thereof

A compound can have a structure of

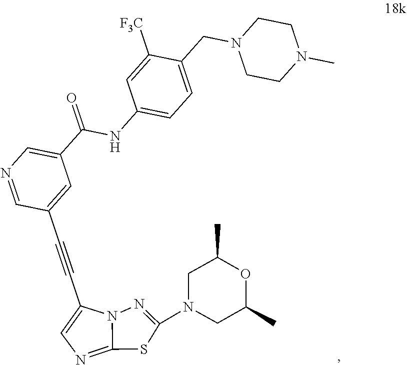 [SMILES: C[C@@H]1CN(c2nn3c(C#Cc4cncc(C(=O)Nc5ccc(CN6CCN(C)CC6)c(C(F)(F)F)c5)c4)cnc3s2)C[C@H](C)O1]

or be a pharmaceutically acceptable salt thereof.

A compound can have a structure of

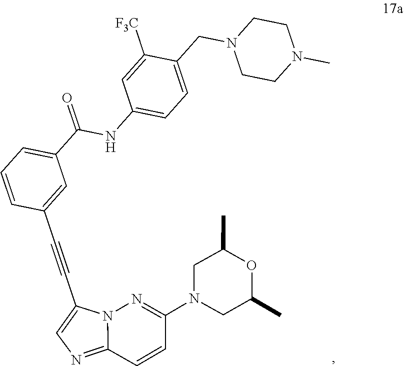 [SMILES: C[C@@H]1CN(c2ccc3ncc(C#Cc4cccc(C(=O)Nc5ccc(CN6CCN(C)CC6)c(C(F)(F)F)c5)c4)n3n2)C[C@H](C)O1]

or be a pharmaceutically acceptable salt thereof

A compound can have a structure of

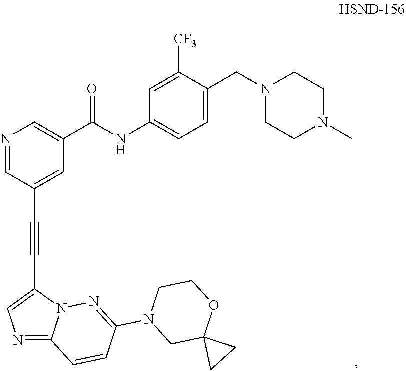 [SMILES: CN1CCN(Cc2ccc(NC(=O)c3cncc(C#Cc4cnc5ccc(N6CCOC7(CC7)C6)nn45)c3)cc2C(F)(F)F)CC1]

or be a pharmaceutically acceptable salt thereof.

A compound can have a structure of

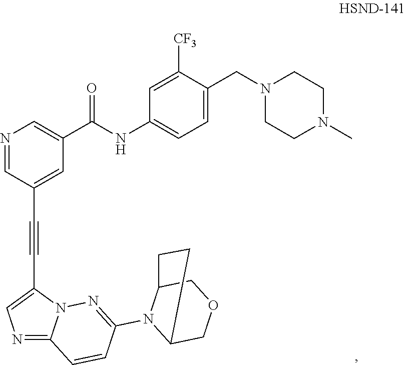 [SMILES: CN1CCN(Cc2ccc(NC(=O)c3cncc(C#Cc4cnc5ccc(N6C7CCC6COC7)nn45)c3)cc2C(F)(F)F)CC1]

or be a pharmaceutically acceptable salt thereof.

A compound can have a structure of

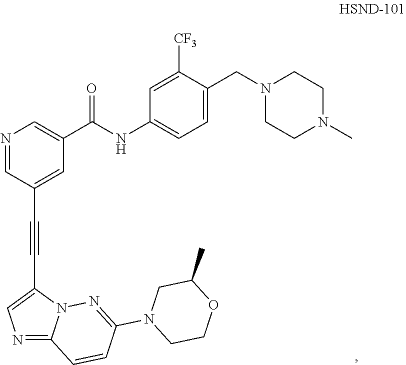 [SMILES: C[C@@H]1CN(c2ccc3ncc(C#Cc4cncc(C(=O)Nc5ccc(CN6CCN(C)CC6)c(C(F)(F)F)c5)c4)n3n2)CCO1]

or be a pharmaceutically acceptable salt thereof.

A compound can have a structure of

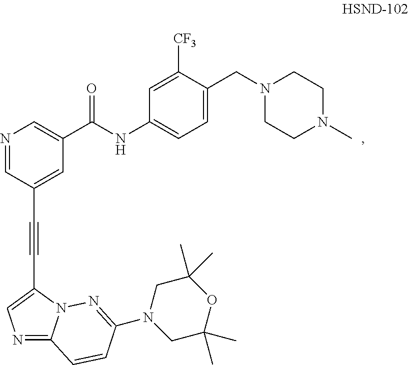 [SMILES: CN1CCN(Cc2ccc(NC(=O)c3cncc(C#Cc4cnc5ccc(N6CC(C)(C)OC(C)(C)C6)nn45)c3)cc2C(F)(F)F)CC1]

or be a pharmaceutically acceptable salt thereof.

A compound can have a structure of

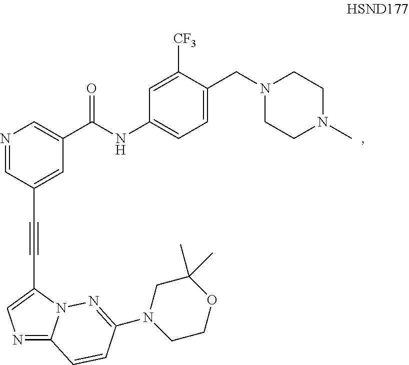 [SMILES: CN1CCN(Cc2ccc(NC(=O)c3cncc(C#Cc4cnc5ccc(N6CCOC(C)(C)C6)nn45)c3)cc2C(F)(F)F)CC1]

or be a pharmaceutically acceptable salt thereof.

A PROTAC conjugate is also provided. In certain embodiments, the PROTAC conjugate has a chemical structure of Formula (X):

A-L′-D (X)

or is a pharmaceutically acceptable salt thereof, wherein: A is a radical of a compound of any one of the compounds hereof; L′ is a linker that binds A and D, or absent; and D is a ubiquitin pathway protein binding moiety.

Still further, pharmaceutical compositions are provided comprising a compound hereof, a conjugate hereof, or a pharmaceutically acceptable salt, N-oxide, hydrate, solvent, tautomer, or optical isomer of the compound or conjugate; and a pharmaceutically acceptable carrier or excipient.

Methods are also provided. In certain embodiments, a method of treating a disease state or disorder in a subject is provided, the method comprising administering to the subject a first therapy comprising an effective amount of a compound hereof, a conjugate hereof, or a pharmaceutically acceptable salt, N-oxide, hydrate, solvate, tautomer, or optical isomer of the compound or conjugate; or a pharmaceutical composition comprising one or more of a compound hereof, a conjugate hereof, or a pharmaceutically acceptable salt, N-oxide, hydrate, solvate, tautomer, or optical isomer of the compound(s) or conjugate.

In certain embodiments, the method further comprises administering to the subject a second therapy comprising: an effective amount of a chemotherapeutic agent, an immunotherapeutic agent, or a hormone therapeutic agent; or radiation therapy.

The disease state or disorder of the subject can be a cancer. The disease state or disorder can be a solid tumor cancer. The cancer can be selected from the group consisting of acute myeloid leukemia, chronic myeloid leukemia, ovarian cancer, cervical cancer, pancreatic cancer, breast cancer, brain cancer, skin cancer, lung cancer, prostate cancer, lymphoma, leukemia, colon cancer, head cancer, neck cancer, thyroid cancer, kidney cancer, liver cancer, and stomach cancer.

The method can further comprise imaging a tumor microenvironment, a population of cancer cells, or a solid tumor in the subject.

Methods for suppressing T cell response in a tumor microenvironment (TME) of a subject are also provided. Such methods can comprise administering to the subject an effective amount of: a compound hereof a conjugate hereof, or a pharmaceutically acceptable salt, N-oxide, hydrate, solvate, tautomer, or optical isomer of the compound or conjugate; or a pharmaceutical composition comprising one or more of a compound hereof, a conjugate hereof, or a pharmaceutically acceptable salt, N-oxide, hydrate, solvate, tautomer, or optical isomer of the compound(s) or conjugate. The subject can have cancer.

In certain embodiments, administering the effective amount to the subject inhibits one or more of MERTK and AXL in the subject.

A compound hereof or a conjugate hereof is also provided for use in the treatment of a disease state modulated by one or more kinases. For example, the disease state can be cancer.

### BRIEF DESCRIPTION OF DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 shows a diagram illustrating an evolution of ponatinib into nicotinamide analogs.

FIG. 2 illustrates various 6-substitutions of compound HSN748 with ring moieties according to the present disclosure, each of which are potent MAP kinase-interacting serine/threonine-protein kinase 2 (MNK2) and ribosomal protein S6 kinase beta-1 (p70S6K) inhibitors.

FIG. 3A shows graphical data representing the anticancer activities of compound HSND41 against NCI-60 (value at 0 represents G150 of 1 μM).

FIG. 3B shows graphical data representing the anticancer activities of compound HSND37 against NCI-60 (value at 0 represents G150 of 1 μM).

FIG. 3C shows graphical data representing the anticancer activities of compound HSN748 against NCI-60 (value at 0 represents G150 of 1 μM).

FIG. 4 shows graphical data representing the anticancer activities of compound HSND80 against NCI-60 (value at 0 represents G150 of 1 μM).

FIG. 5 shows graphical data relating to PK properties of various compounds hereof in rats following administration via oral gavage.

FIGS. 6A and 6B show graphical data related to HSND80 binding affinity to AXL receptor tyrosine kinase (AXL), with data for replicate 1 shown in FIG. 6A (1 Kd=0.77 nM) and data for replicate 2 shown in FIG. 6B (2 Kd=1.3 nM).

FIGS. 7A and 7B show graphical data related to HSND80 binding affinity to MER Proto-Oncogene tyrosine kinase (MERTK), with data for replicate 1 shown in FIG. 7A (1 Kd=1.6 nM) and data for replicate 2 shown in FIG. 7B (2 Kd=2.1 nM).

While the present disclosure is susceptible to various modifications and alternative forms, exemplary embodiments thereof are shown by way of example in the drawings and are herein described in detail.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

The present disclosure generally relates to nicotinamide- or benzamide-based compounds linked to (or substituted with) a bicyclic heterocycle substituted with one or more ring moieties of the 6-position of the nicotinamide or benzamide. Such compounds are kinase inhibitors. In certain embodiments, the bicyclic heterocycle is an alkynyl imidazo[1,2-b]pyridazine, imidazo[1,2-b]pyrazole, or imidazo[2,1-b][1,3,4]thiadiazole.

In certain embodiments, the compounds comprise a modification of the benzamide portion of ponatinib into a nicotinamide and a substitution of the 6-position of nicotinamide alkynyl imidazo[1,2-b]pyridazine with a substituent comprising one or more ring moieties, such as morpholine or piperazine.

The nicotinamide- or benzamide-based compounds hereof have potent activities against MAP kinase-interacting serine/threonine-protein kinase 1 and 2 (MNK1/2) and ribosomal protein S6 kinase β1 (p70S6K) and can exhibit remarkable anticancer activities (e.g., against solid tumor cancers) as compared to analogs without substitution at position 6 or substitution at position 6 with linear heteroalkyl groups. Indeed, p70S6K kinase is only weakly inhibited by both ponatinib and compound HSN748 (a nicotinamide analog having linear heteroalkyl groups instead of the one or more ring moieties of the present compounds), while the compounds hereof dramatically inhibit the activity of p70S6k kinase. Additionally, at least the nicotinamide-based compounds hereof also inhibit TAM kinases, AXL receptor tyrosine kinase (AXL) and MER Proto-Oncogene tyrosine kinase (MERTK), which are not targeted by the parent ponatinib or HSN748 compounds.

Furthermore, the compounds hereof are orally bioavailable and potentially inhibit several solid tumor cancers (e.g., breast cancer, ovarian cancer, lung cancer, colon cancer, and the like), while not inhibiting or only weakly inhibiting human Ether-a-go-go-Related Gene (hERG).

PROTAC conjugates, pharmaceutical compositions, and methods of use thereof (e.g., in treating certain kinase-mediated diseases such as cancer) are also provided. The compounds, compositions, conjugates, and methods are useful for regulating protein activity, and treating diseases and conditions related to protein activity.

Nicotinamide alkynyl imidazo[1,2-b]pyridazine compounds have been described as kinase inhibitors with anticancer activities (International Patent Application No. PCT/IB2019/057711, International Publication No. WO/2020/053812 and International Publication No. WO2013170770A1). However rules governing how various substitutions on the nicotinamide alkynyl imidazo[1,2-b]pyridazine moiety affect both kinase selectivity and anticancer properties have to date not been well described, making it difficult to optimize the biological properties of the said compounds a priori.

Primarily, substituted acetylenic imidazo[1,2-b]pyridazine compounds containing a benzamide unit are known kinase inhibitors. Ponatinib, one of such compounds, is a multi-kinase inhibitor that has been described as anon-receptor tyrosine-protein kinase ABL1 (ABL1) inhibitor. However, ponatinib administration is associated with many adverse toxicities, at least in part due to the concurrent inhibition of many essential kinases (e.g., hERG). Analogs of ponatinib with reduced inhibition of cardiovascular-related kinases, such as VEGFR1-3, c-Src, c-KIT, and the like are predicted to exhibit lower adverse toxicities. Further, ponatinib does not inhibit (or, at best, weakly inhibits) MNK1/2 and p70S6K kinases.

MNK1/2 and p70S6K kinases regulate the translation of oncogenic proteins via the RAS-MEK-ERK and PI3K-AKT-mTORC1 axes, respectively. Xie et al., The MAP Kinase-Interacting Kinases (MNKs) as Targets in Oncology, Expert Opinion on Therapeutic Targets 23(3): 187-199 (2019); Roux & Topisirovic, Signaling Pathways Involved in the Regulation of mRNA Translation, Molecular Cell Biology 38: e00070-18 (2018). MNK is also involved in transcriptional regulation of key oncogenic proteins, such as E2F1, FOXM1 and WEEL. Ke et al., MNK1 and MNK2 Enforce Expression of E2F1, FOXM1, and WEEl to Drive Soft Tissue Sarcoma, Oncogene 40: 1851-1867 (2021). Due to the importance of these proteins in tumorgenesis, several inhibitors of these key kinases (MNK and p70S6K) have been developed and/or evaluated as potential anticancer agents. Jin et al., Progress in Developing MNK Inhibitors, European J Med Chemistry 219: 113420 (2021); Spaan et al., Direct P70S6K1 Inhibition to Replace Dexamethasone in Synergistic Combination with MCL-1 Inhibition in Multiple Myeloma, Blood Advances 5: 2593-2607 (2021); E1-Khoueiry et al., A Phase II, Open-Label Study of Tomivosertib (eFT508) Added on to Continued Checkpoint Inhibitor Therapy in Patients (pts) with Insufficient Response to Single-Agent Treatment, J Clinical Oncology 38(15): suppl DOI: 10.1200/JCO.2020.38.15_suppl.3112 (2020); Tolcher et al., A Phase I Trial of LY2584702 Tosylate, a p70 S6 Kinase Inhibitor, in Patients with Advanced Solid Tumours, European J Cancer 50(5): 867-75, doi: 10.1016/j.ejca.2013.11.039 (2014); Tsimberidou et al., Phase 1 Study of M2698, a p70S6K/AKT Dual Inhibitor, in Patients with Advanced Cancer, JHematology & Oncology 14(1): 127. doi: 10.1186/s13045-021-01132-z (2021).

MNK1 and 2 are immunosuppressive kinases and it has been shown that the inhibition of MNK1 and 2 by tomivosertib (eFT-508), a potent and selective MNK1/2, led to blockage of the expression of programmed cell death protein 1 (PD-1), programmed cell death-ligand 1 (PD-L1) and lymphocyte-activation gene 3 (LAG-3, which are checkpoint proteins. E1-Khoueiry et al. 2020), supra. Tomivosertib, when added to checkpoint therapy, led to objective clinical responses in patients who had poor responses to checkpoint inhibitors alone. Id.

A few p70S6K inhibitors, such as LY2584702 and M2698 have been developed and trialed in the clinic. Tolcher et al. (2014), supra; Tsimberidou et al. (2021), supra. While LY2584702 did not show promising clinical activity, for advanced breast cancer patients, M2698 demonstrated antitumor activity when combined with various treatment regimens (such as trastumab or tamoxifen). Tsimberidou et al. (2021), supra.

Single inhibition of MNKs can lead to tumor growth inhibition, but not tumor elimination so it has been suggested that concurrent inhibition of MNK with other key cancer targets, using drug combination or a single drug with polypharmacology, is a logical approach to anticancer therapy via MNK inhibition. Xie et al. (2019), supra. Considering that both MNK and p70S6K regulate the translation, the data herein supports that the dual inhibition of these kinases translates into the more profound inhibition of cancer cell growth than the inhibition of the individual kinases.

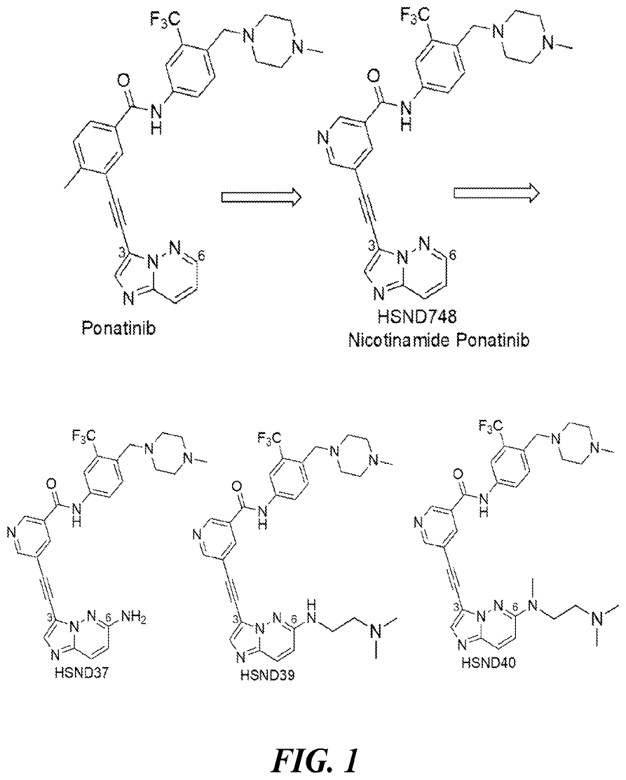

In early studies, the inventors hereof observed that a nicotinamide analog of ponatinib comprising linear heteroalkyl groups (i.e., compound HSN748) was a potent inhibitor of MNK2 (IC50=9 nM; [ATP]=100 μM) but only moderately inhibited p70S6K (IC50=273 nM). Larocque et al., Nicotinamide-Ponatinib Analogues as Potent Anti-CML and Anti-AML Compounds, ACS Omega 5(6), 2690-2698 (2020). FIG. 1 shows the evolution of ponatinib to compound HSN748 and other analogs of HSND748 that comprise a substitution of the 6-position of nicotinamide alkynyl imidazo[1,2-b]pyridazine with linear heteroalkyl groups. Ponatinib also moderately inhibited p70S6K (IC50=273 nM; [ATP]=100 μM), but did not inhibit MNK2. Id. Considering that ponatinib, compound HSN748, and HSN748's analogs are greater than 90% protein bound, it is unlikely that a therapeutic dose could be found that covers the IC90 for the inhibition of both MNK and p70S6K.

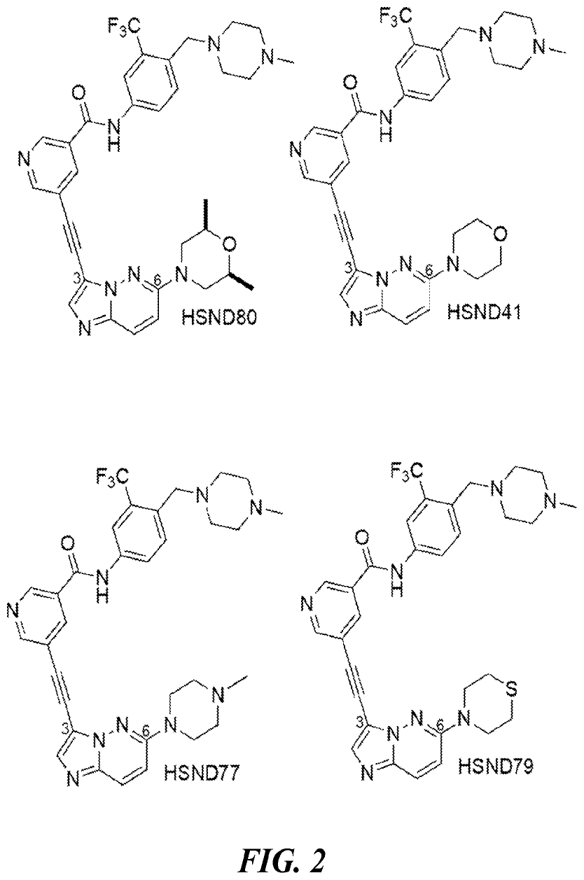

However, the compounds hereof (e.g., comprising a substitution of the 6-position of benzamide or nicotinamide with, for example, an alkynyl imidazo[1,2-b]pyridazine with one or more ring moieties, such as morpholine or piperazine) have potent activities against MNK1/2 and p70S6K. These compounds exhibit remarkable anticancer activities when compared to analogs without substitution at position 6 or substitution with linear heteroalkyl groups. FIG. 2 shows various compounds hereof comprising a substitution of the 6-position of nicotinamide alkynyl imidazo[1,2-b]pyridazine with one or more ring moieties.

### Compounds

The compounds can inhibit kinases. The compounds can selectively and potently inhibit cancer-mediating kinases for killing and/or ameliorating cancer cells (e.g., MNK and p70S6K), while also being poor hERG inhibitors which significantly reduces off-target toxicity (e.g., cardiovascular toxicity).

Based on the discovery that a benzamide or nicotinamide version of ponatinib that further comprises one or more ring moieties imparts different and desirable properties as compared to conventional compounds, the compound hereof can have the general structure of Formula (XX):

R₄-L-Z—R₃ (XX)

or a pharmaceutically acceptable salt thereof, wherein:
R₄ is a nicotinamide or benzamide, each of the nicotinamide or benzamide optionally substituted;
L is a linker (e.g., linked to a 6-position of the nicotinamide or benzamide) comprising at least one atom;
Z is a bicyclic heterocycle substituted with R₃; and
R₃ comprises one or more ring moieties.

The linker (L) can be any suitable linker. The linker can have a backbone that ranges in length, such that there can be as few as one atom in the backbone of the linker to as many as 100 or more contiguous atoms in the backbone of the linker. The “backbone” of the linker is the shortest chain of contiguous atoms forming a covalently bonded connection between R₄ and Z. In some embodiments, the linker is a polyvalent linker with a branched backbone, with each branch serving as a section of backbone linker until reaching a terminus. The linker (L) can be an alkyne, for example, or any other linker comprising one or more atoms.

For example, the linker can have a chain length of at least about 7 atoms. In some embodiments, the linker is at least about 3 atoms in length. In some embodiments, the linker is at least about 10 atoms in length. In some embodiments, the linker is between about 7 and about 31 atoms (such as, about 7 to 31, 7 to about 31, or 7 to 31), between about 7 and about 24 atoms (such as, about 7 to 24, 7 to about 24, or 7 to 24), or between about 7 and about 20 atoms (such as, about 7 to 20, 7 to about 20, or 7 to 20) atoms. In some embodiments, the linker is between about 14 and about 31 atoms (such as, about 14 to 31, 14 to about 31, or 14 to 31), between about 14 and about 24 atoms (such as, about 14 to 24, 14 to about 24, or 14 to 24), or between about 14 and about 20 atoms (such as, about 14 to 20, 14 to about 20, or 14 to 20). In some embodiments, the linker has a chain length of at least 7 atoms, at least 14 atoms, at least 20 atoms, at least 25 atoms, at least 30 atoms, or at least 40 atoms; or from 1 to 15 atoms, 1 to 5 atoms, 5 to 10 atoms, 5 to 20 atoms, 10 to 40 atoms, or 25 to 100 atoms.

The linker can comprise at least one carbon-carbon bond and/or at least one amide bond. The linker can comprise one or more L- or D-configurations, natural or unnatural amino acids, a PEG monomer, a PEG oligomer, a PEG polymer, or a combination of any of the foregoing. For a linker that comprises one or more PEG units, all carbon and oxygen atoms of the PEG units are part of the backbone, unless otherwise specified.

In certain embodiments, the linker is a group comprising one or more covalently connected structural units.

In certain embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, and/or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker is asymmetric. In certain embodiments, the linker is symmetrical.

Alternatively, or in addition to chain length, in some embodiments, the linker can have suitable substituents that affect hydrophobicity or hydrophilicity. Thus, for example, the linker can have a hydrophobic side chain group, such as an alkyl, cycloalkyl, aryl, arylalkyl, or like group, each of which is optionally substituted. If the linker includes one or more amino acids, the linker can contain hydrophobic amino acid side chains, such as one or more amino acid side chains from Phe and Tyr, including substituted variants thereof, and analogs and derivatives of such side chains.

The linker can comprise a spacer (e.g., be conjugated with and/or include a spacer). The spacer can be any suitable spacer. A spacer of the linker can comprise hydrophilic, hydrophobic, amphipathic, non-peptidic, peptidic, and/or aromatic monomers. A length of a spacer can range from 1 to 30 (e.g., 1 to 30 carbon atoms, a PEG with 1-30 units, etc.).

In certain embodiments, the compound has a structure of Formula (I):

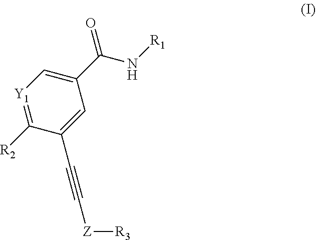

or be a pharmaceutically acceptable salt thereof, wherein:
   R₁ is an alkyl, a pyridine, an isoxazole, a pyrazole, or aryl (e.g., a phenyl group), each optionally substituted with one or more of a piperazine (e.g., an alkyl piperazine), a pyrazine (e.g., an alkyl pyrazine), an imidazole (e.g., an alkyl imidazole), a cyanide, an amine, a halogen, a trifluoromethyl group, an N-containing heterocycle, and/or an alkyl;
   Y₁ is nitrogen (N) or carbon (C);
   R₂ is hydrogen (H), an alkyl, a heteroalkyl, or a halogen;
   Z is a bicyclic heterocycle substituted with R₃, wherein the bicyclic heterocycle is imidazo[1,2-b]pyridazine, imidazo[1,2-b]pyrazole, or imidazo[2,1-][1,3,4]thiadiazole; and
   R₃ is a substituent comprising one or more ring moieties.

R₃ can have a structure of Formula (II), Formula (III), or Formula (IV):

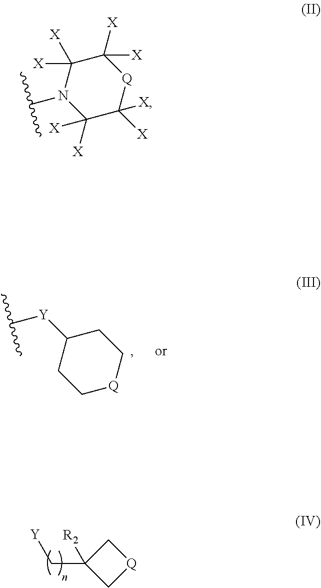

wherein:

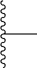

is a point of attachment;
each X is independently an alkyl or an H, with the proviso that all X are not H;
Q is O, S, N, or C, and is optionally substituted with one or more of an alkyl, a halogen, an O-alkyl, an amine, an —OH group, an alkoxy, a piperazine, a morpholine, an aziridine, a carbocycle (e.g., a 3-membered carbocycle) or heterocycle, and/or a carbonyl;
Y is O or an amine, with the proviso that Y is not NH; and
n is 0 or 1.

In certain embodiments, R₃ of the compound has a structure of Formula (II) and Q is O. In certain embodiments, each X is independently an alkyl or an H, with the proviso that not all X are H.

In certain exemplary embodiments, R₃ is a substituted morpholine. In certain embodiments, the linker is an alkyne.

In certain embodiments, Y₁ is C such that the compound is benzamide-based. In certain embodiments, Y₁ is N such that the compound is nicotinamide-based. In certain embodiments, Y₁ is N and, instead of being positioned as shown in Formula (I), the N is in any position on the nicotinamide ring.

The compound can result from a replacement of the methyl benzamide in ponatinib with a nicotinamide moiety, and further incorporating one or more heterocycle groups into R₃.

In certain embodiments, R₁ is

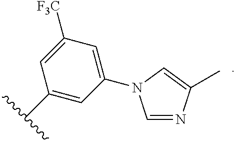 [SMILES: *c1cc(-n2cnc(C)c2)cc(C(F)(F)F)c1]

In certain embodiments, R₁ is

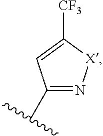

wherein

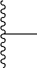

is a point of attachment and X is O, C, or N. In certain embodiments, R₁ is

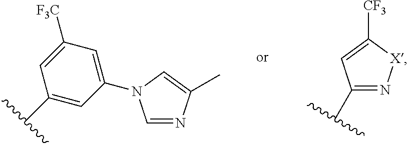

wherein

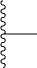

is a point of attachment and X′ is O, C, or N.

Z can have a structure of

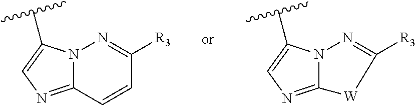

wherein

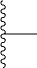

is a point of attachment, and W is C or S.

R₃ can be a heterocycle. R₃ can be a 4-6-membered heterocycle (e.g., a 4-membered heterocycle, a 5-membered heterocycle, or a 6-membered heterocycle). R₃ can be an oxygen (O)-containing heterocycle and/or R₃ can be a nitrogen (N)-containing heterocycle. R₃ can be a sulfur (S)-containing heterocycle.

R₃ can comprise a bicyclic ring moiety. R₃ can comprise a bicyclic ring moiety comprising two 6-membered heterocycles. R₃ can comprise a bicyclic ring moiety comprising a 6-membered heterocycle and a 5-membered heterocycle. R₃ can comprise a bicyclic ring moiety comprising two 5-membered heterocycles. R₃ can comprise a bicyclic ring moiety comprising a 6-membered heterocycle and a 4-membered heterocycle. R₃ can comprise a bicyclic ring moiety comprising a 6-membered heterocycle and a 3-membered carbocycle or heterocycle. R₃ can comprise a bicyclic ring moiety comprising a 5-membered heterocycle and a 4-membered heterocycle or carbocycle.

R₃ can have a structure

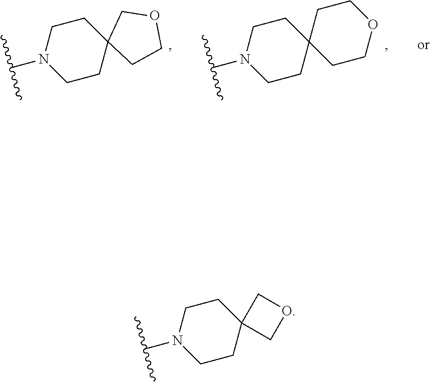 [SMILES: *N1CCC2(CCOC2)CC1 ; *N1CCC2(CCOCC2)CC1 ; *N1CCC2(CC1)COC2]

In certain embodiments, R₃ has a linear alkyl group and a bicyclic ring moiety. For example, R₃ can have a linear heteroalkyl group (e.g., an O- and/or N-containing linear heteroalkyl group) and a bicyclic ring moiety. In certain embodiments, R₃ has a structure

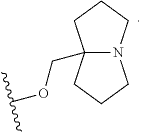 [SMILES: *OCC12CCCN1CCC2]

In certain embodiments, R₃ has a structure of Formula (II):

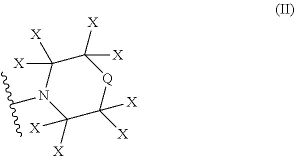

and Q is O. In certain embodiments, R₃ has a structure of Formula (II) and is morpholine (e.g., Q is 0). R₃ can be or comprise unsubstituted morpholine. R₃ can be or comprise substituted morpholine. For example, and without limitation, R₃ can be or comprise morpholine substituted with at least two methyl groups.

In certain embodiments, R₃ has a structure of Formula (II) and is piperdine (e.g., Q is C). In certain embodiments, R₃ has a structure of Formula (III) and Q is N (e.g., R₃ comprises piperdine).

R₃ can have the structure of Formula (II), wherein at least two of the X are an H. R₃ can have the structure of Formula (II), wherein at least three of the X are an H. R₃ can have the structure of Formula (II), wherein at least four of the X are an H. R₃ can have the structure of Formula (II), wherein at least five of the X are an H. R₃ can have the structure of Formula (II), wherein at least six of the X are an H. R₃ can have the structure of Formula (II), wherein seven of the X are an H.

R₃ can have the structure of Formula (II) and at least two of the X can be linked together to form a bicyclic heterocycle. In certain embodiments, R₃ is

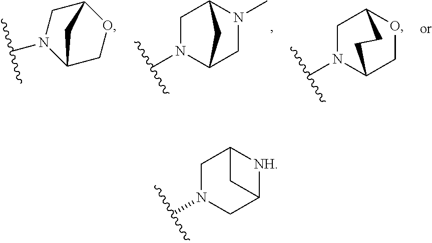

In certain embodiments, R₃ is an azetidine, optionally substituted with one or more of an alkyl, a halogen, an amine, an O-alkyl, and/or an —OH group. In certain embodiments, R₃ is a pyrrolidine, optionally substituted with an oxetane.

R₃ can have the structure of Formula (III):

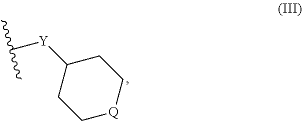

wherein Y is a methylamine (—NMe) or an ethylamine (—NEt). Y of Formula (III) can be a linear alkyl group. Y of Formula (III) can be a linear heteroalkyl group. In certain embodiments, R₃ is or comprises

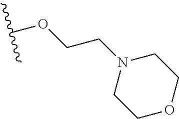 [SMILES: *OCCN1CCOCC1]

(e.g., where Y of Formula (III) is an O-containing linear alkyl group).

In certain embodiments, R₃ is or comprises tetrahydro-2H-pryan.

In certain embodiments, the compound has a structure of Formula (V):

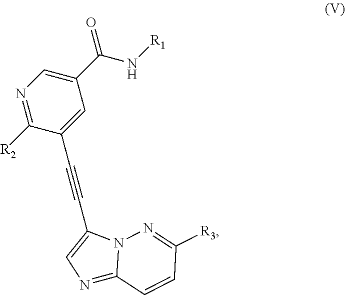

wherein:
R₁ is an alkyl, a pyridine, an isoxazole, a pyrazole, or aryl (e.g., a phenyl group), each optionally substituted with one or more of a piperazine (e.g., an alkyl piperazine), a pyrazine (e.g., an alkyl pyrazine), an imidazole (e.g., an alkyl imidazole), a cyanide, an amine, a halogen, a trifluoromethyl group, an N-containing heterocycle, and/or an alkyl;
R₂ is H, an alkyl, a heteroalkyl, or a halogen; and
R₃ is a substituent comprising one or more ring moieties.

The compound can have the following structure:

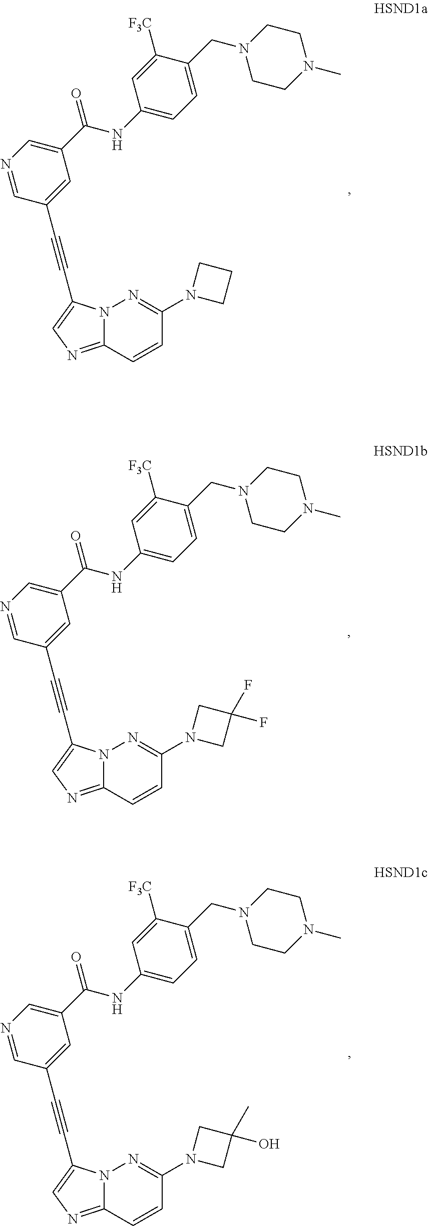 [SMILES: CN1CCN(Cc2ccc(NC(=O)c3cncc(C#Cc4cnc5ccc(N6CCC6)nn45)c3)cc2C(F)(F)F)CC1 ; CN1CCN(Cc2ccc(NC(=O)c3cncc(C#Cc4cnc5ccc(N6CC(F)(F)C6)nn45)c3)cc2C(F)(F)F)CC1 ; CN1CCN(Cc2ccc(NC(=O)c3cncc(C#Cc4cnc5ccc(N6CC(C)(O)C6)nn45)c3)cc2C(F)(F)F)CC1]

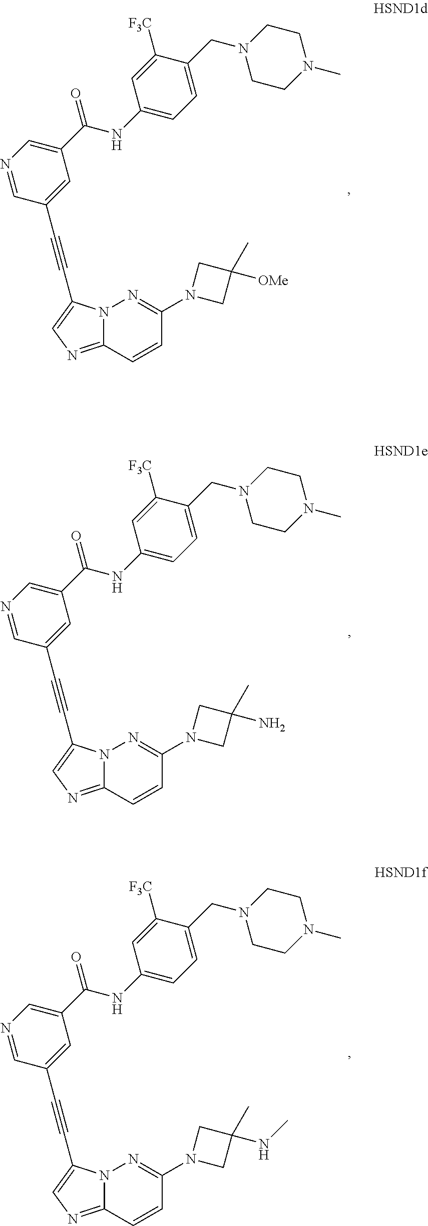 [SMILES: COC1(C)CN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CC(C)(N)C6)nn45)c3)cc2C(F)(F)F)CC1 ; CNC1(C)CN(c2ccc3ncc(C#Cc4cncc(C(=O)Nc5ccc(CN6CCN(C)CC6)c(C(F)(F)F)c5)c4)n3n2)C1]

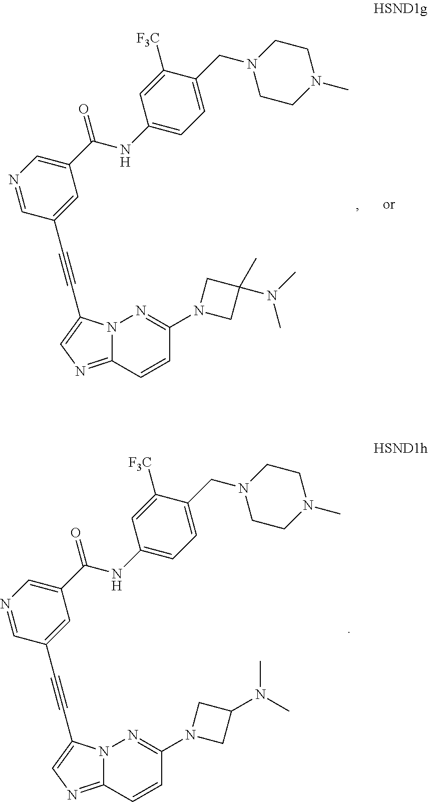 [SMILES: CN1CCN(Cc2ccc(NC(=O)c3cncc(C#Cc4cnc5ccc(N6CC(C)(N(C)C)C6)nn45)c3)cc2C(F)(F)F)CC1 ; CN1CCN(Cc2ccc(NC(=O)c3cncc(C#Cc4cnc5ccc(N6CC(N(C)C)C6)nn45)c3)cc2C(F)(F)F)CC1]

The compound can have the following structure:

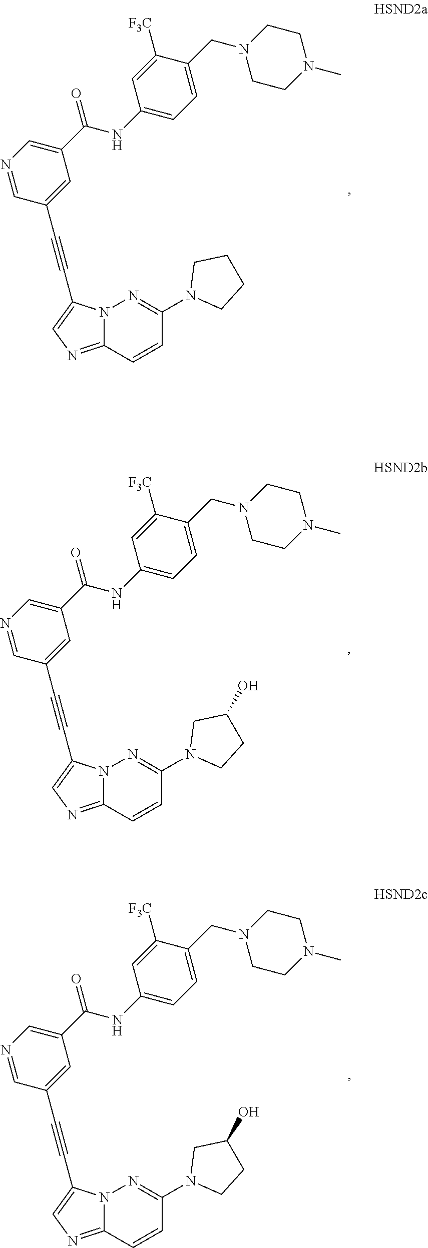 [SMILES: CN1CCN(Cc2ccc(NC(=O)c3cncc(C#Cc4cnc5ccc(N6CCCC6)nn45)c3)cc2C(F)(F)F)CC1 ; CN1CCN(Cc2ccc(NC(=O)c3cncc(C#Cc4cnc5ccc(N6CC[C@@H](O)C6)nn45)c3)cc2C(F)(F)F)CC1 ; CN1CCN(Cc2ccc(NC(=O)c3cncc(C#Cc4cnc5ccc(N6CC[C@H](O)C6)nn45)c3)cc2C(F)(F)F)CC1]

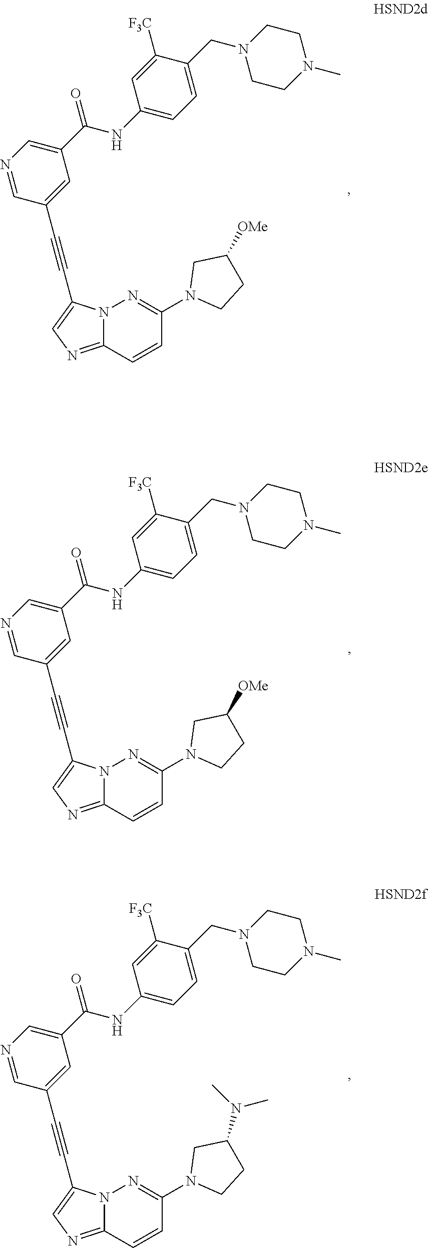 [SMILES: CO[C@@H]1CCN(c2ccc3ncc(C#Cc4cncc(C(=O)Nc5ccc(CN6CCN(C)CC6)c(C(F)(F)F)c5)c4)n3n2)C1 ; CO[C@H]1CCN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CC[C@@H](N(C)C)C6)nn45)c3)cc2C(F)(F)F)CC1]

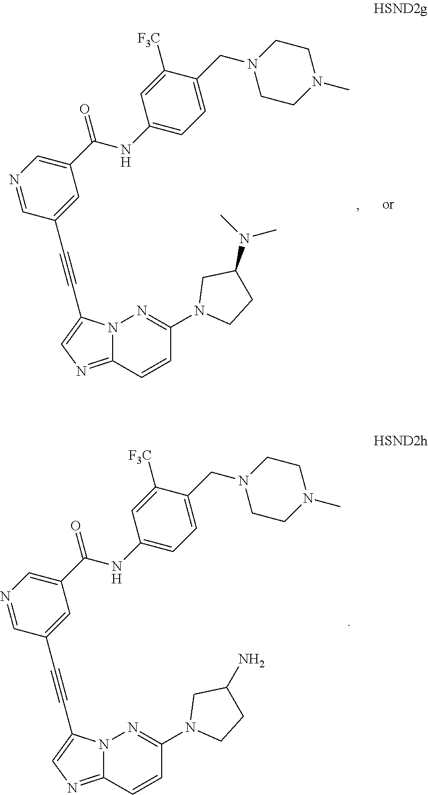 [SMILES: CN1CCN(Cc2ccc(NC(=O)c3cncc(C#Cc4cnc5ccc(N6CC[C@H](N(C)C)C6)nn45)c3)cc2C(F)(F)F)CC1 ; CN1CCN(Cc2ccc(NC(=O)c3cncc(C#Cc4cnc5ccc(N6CCC(N)C6)nn45)c3)cc2C(F)(F)F)CC1]

The compound can have the following structure:

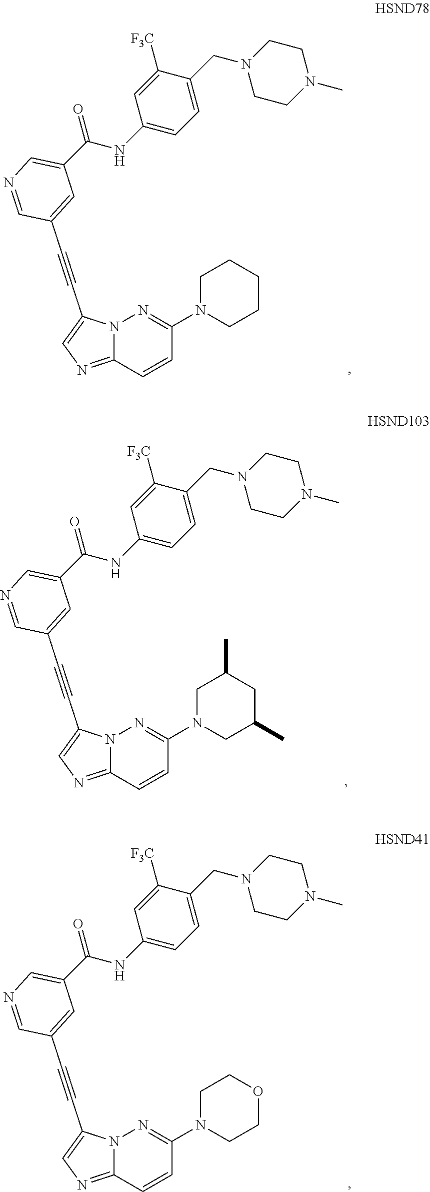 [SMILES: CN1CCN(Cc2ccc(NC(=O)c3cncc(C#Cc4cnc5ccc(N6CCCCC6)nn45)c3)cc2C(F)(F)F)CC1 ; C[C@@H]1C[C@H](C)CN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CCOCC6)nn45)c3)cc2C(F)(F)F)CC1]

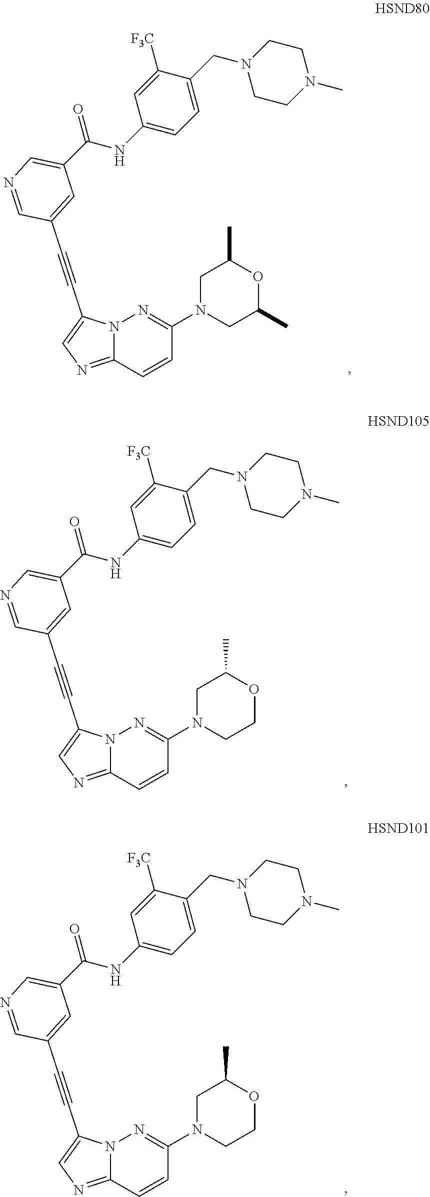 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)O1 ; C[C@H]1CN(c2ccc3ncc(C#Cc4cncc(C(=O)Nc5ccc(CN6CCN(C)CC6)c(C(F)(F)F)c5)c4)n3n2)CCO1 ; C[C@@H]1CN(c2ccc3ncc(C#Cc4cncc(C(=O)Nc5ccc(CN6CCN(C)CC6)c(C(F)(F)F)c5)c4)n3n2)CCO1]

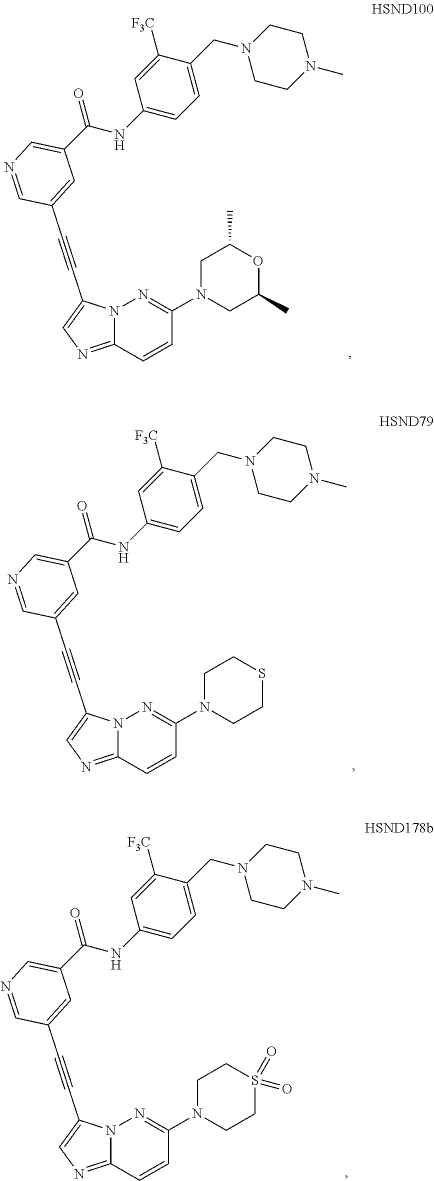 [SMILES: C[C@H]1CN(c2ccc3ncc(C#Cc4cncc(C(=O)Nc5ccc(CN6CCN(C)CC6)c(C(F)(F)F)c5)c4)n3n2)C[C@H](C)O1 ; CN1CCN(Cc2ccc(NC(=O)c3cncc(C#Cc4cnc5ccc(N6CCSCC6)nn45)c3)cc2C(F)(F)F)CC1 ; CN1CCN(Cc2ccc(NC(=O)c3cncc(C#Cc4cnc5ccc(N6CCS(=O)(=O)CC6)nn45)c3)cc2C(F)(F)F)CC1]

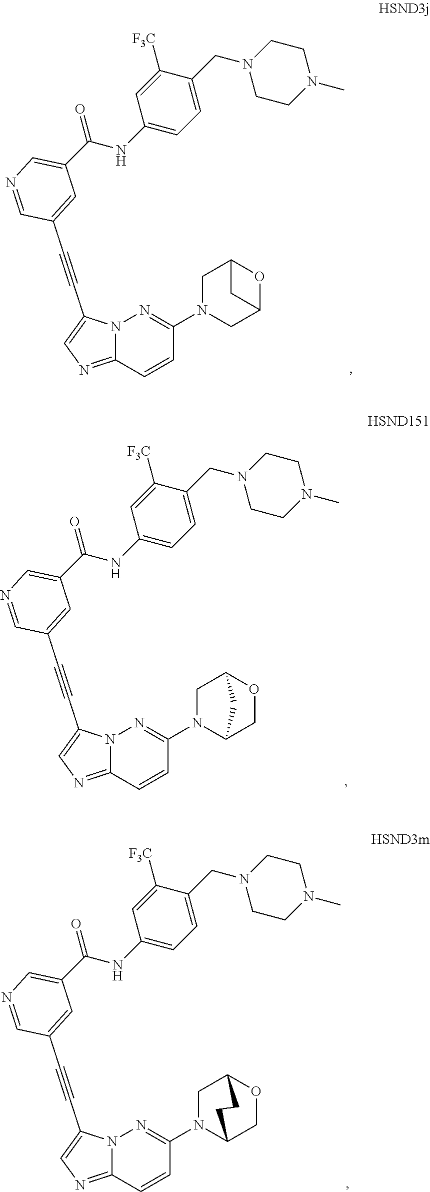 [SMILES: CN1CCN(Cc2ccc(NC(=O)c3cncc(C#Cc4cnc5ccc(N6CC7CC(C6)O7)nn45)c3)cc2C(F)(F)F)CC1 ; CN1CCN(Cc2ccc(NC(=O)c3cncc(C#Cc4cnc5ccc(N6C[C@@H]7C[C@H]6CO7)nn45)c3)cc2C(F)(F)F)CC1 ; CN1CCN(Cc2ccc(NC(=O)c3cncc(C#Cc4cnc5ccc(N6C[C@H]7CC[C@@H]6CO7)nn45)c3)cc2C(F)(F)F)CC1]

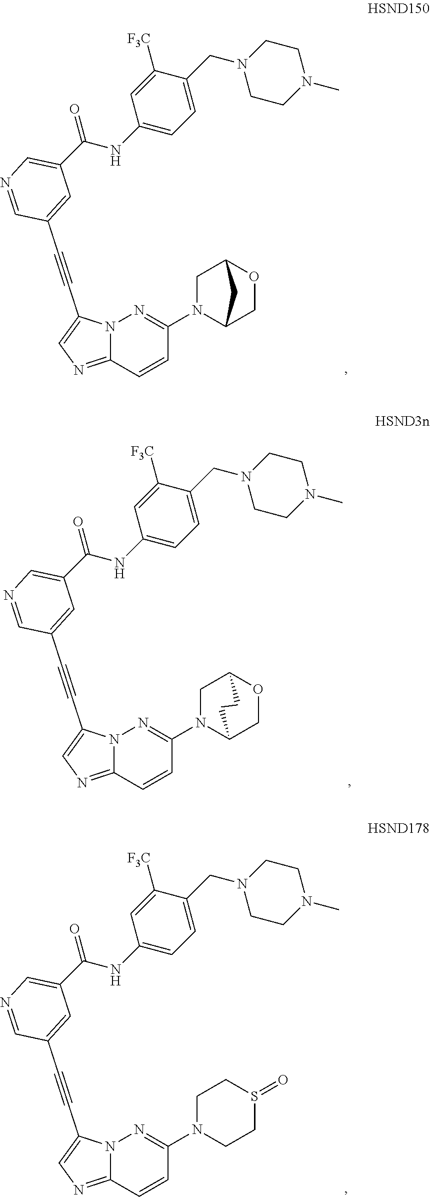 [SMILES: CN1CCN(Cc2ccc(NC(=O)c3cncc(C#Cc4cnc5ccc(N6C[C@H]7C[C@@H]6CO7)nn45)c3)cc2C(F)(F)F)CC1 ; CN1CCN(Cc2ccc(NC(=O)c3cncc(C#Cc4cnc5ccc(N6C[C@@H]7CC[C@H]6CO7)nn45)c3)cc2C(F)(F)F)CC1 ; CN1CCN(Cc2ccc(NC(=O)c3cncc(C#Cc4cnc5ccc(N6CCS(=O)CC6)nn45)c3)cc2C(F)(F)F)CC1]

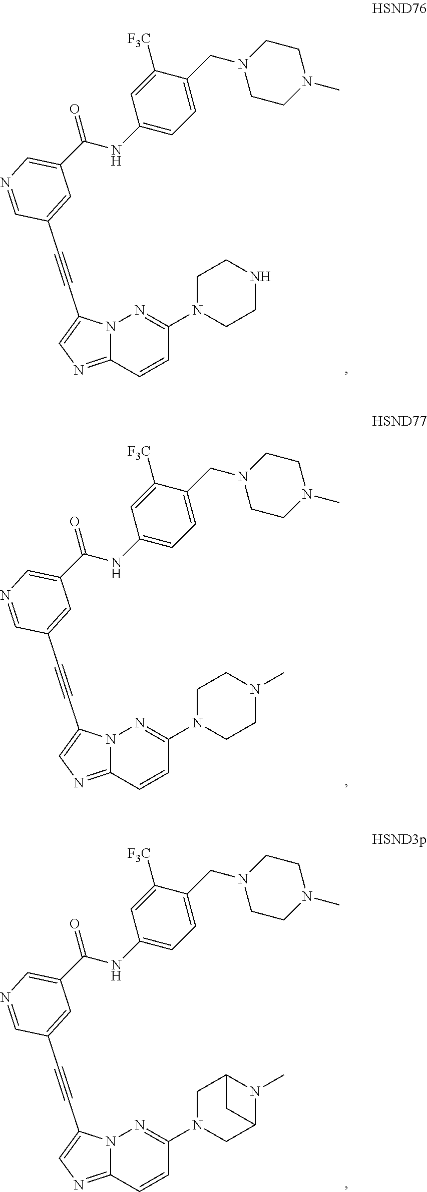 [SMILES: CN1CCN(Cc2ccc(NC(=O)c3cncc(C#Cc4cnc5ccc(N6CCNCC6)nn45)c3)cc2C(F)(F)F)CC1 ; CN1CCN(Cc2ccc(NC(=O)c3cncc(C#Cc4cnc5ccc(N6CCN(C)CC6)nn45)c3)cc2C(F)(F)F)CC1 ; CN1CCN(Cc2ccc(NC(=O)c3cncc(C#Cc4cnc5ccc(N6CC7CC(C6)N7C)nn45)c3)cc2C(F)(F)F)CC1]

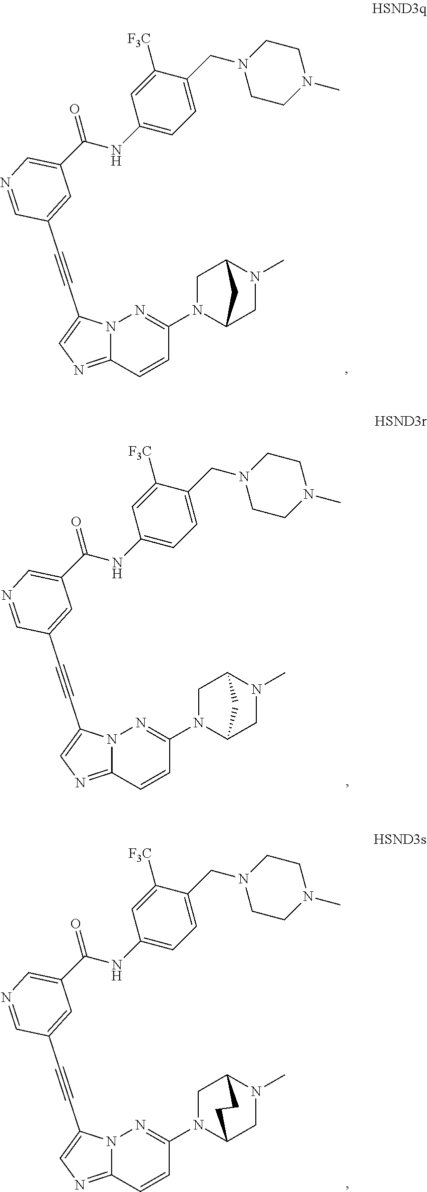 [SMILES: CN1CCN(Cc2ccc(NC(=O)c3cncc(C#Cc4cnc5ccc(N6C[C@H]7C[C@@H]6CN7C)nn45)c3)cc2C(F)(F)F)CC1 ; CN1CCN(Cc2ccc(NC(=O)c3cncc(C#Cc4cnc5ccc(N6C[C@@H]7C[C@H]6CN7C)nn45)c3)cc2C(F)(F)F)CC1 ; CN1CCN(Cc2ccc(NC(=O)c3cncc(C#Cc4cnc5ccc(N6C[C@H]7CC[C@@H]6CN7C)nn45)c3)cc2C(F)(F)F)CC1]

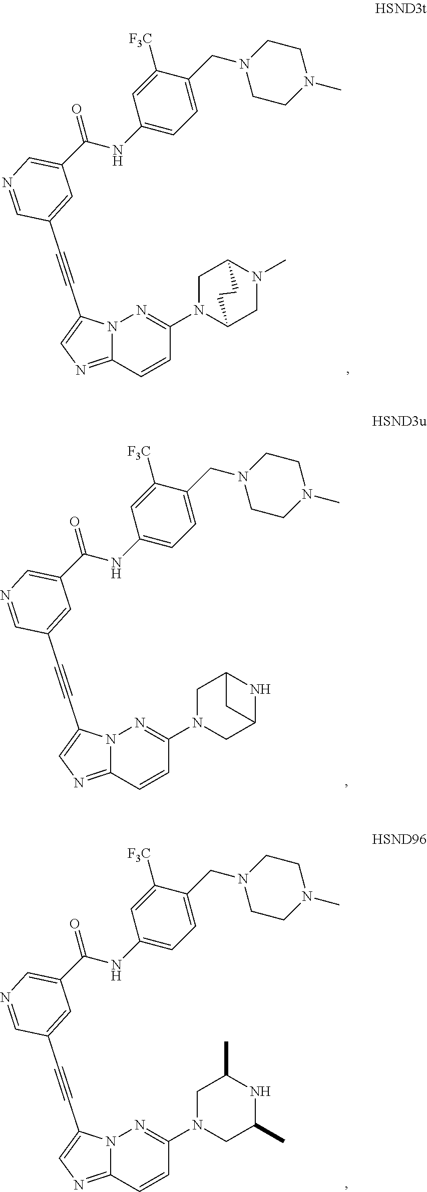 [SMILES: CN1CCN(Cc2ccc(NC(=O)c3cncc(C#Cc4cnc5ccc(N6C[C@@H]7CC[C@H]6CN7C)nn45)c3)cc2C(F)(F)F)CC1 ; CN1CCN(Cc2ccc(NC(=O)c3cncc(C#Cc4cnc5ccc(N6CC7CC(C6)N7)nn45)c3)cc2C(F)(F)F)CC1 ; C[C@@H]1CN(c2ccc3ncc(C#Cc4cncc(C(=O)Nc5ccc(CN6CCN(C)CC6)c(C(F)(F)F)c5)c4)n3n2)C[C@H](C)N1]

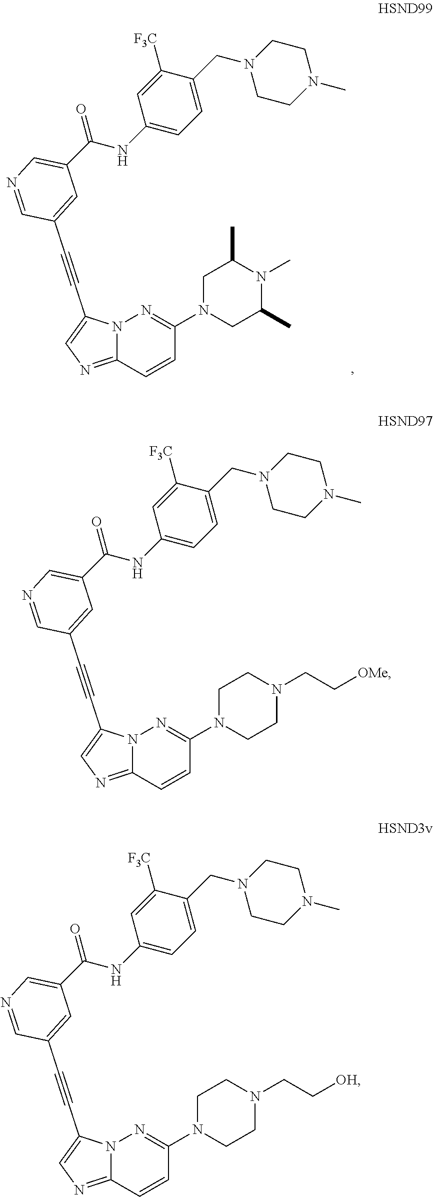 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)N1C ; COCCN1CCN(c2ccc3ncc(C#Cc4cncc(C(=O)Nc5ccc(CN6CCN(C)CC6)c(C(F)(F)F)c5)c4)n3n2)CC1 ; CN1CCN(Cc2ccc(NC(=O)c3cncc(C#Cc4cnc5ccc(N6CCN(CCO)CC6)nn45)c3)cc2C(F)(F)F)CC1]

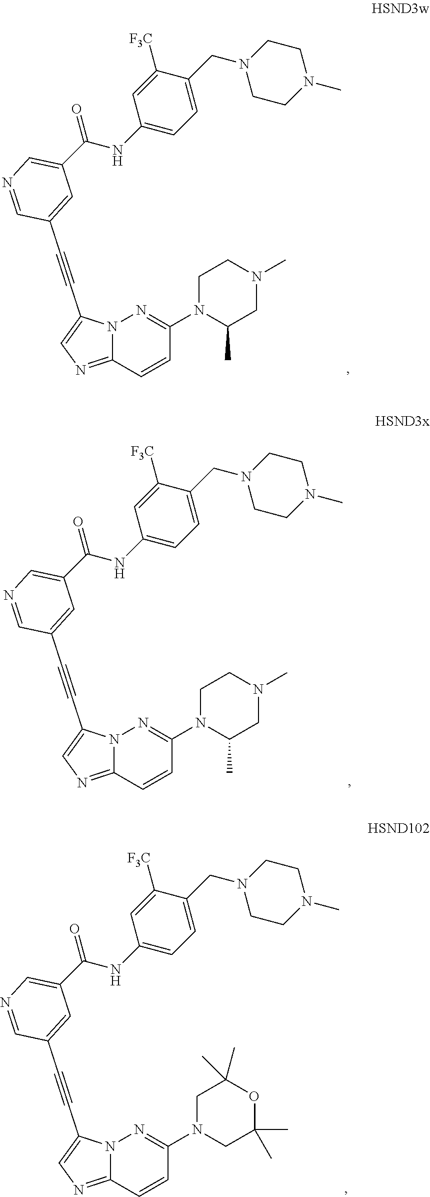 [SMILES: C[C@@H]1CN(C)CCN1c1ccc2ncc(C#Cc3cncc(C(=O)Nc4ccc(CN5CCN(C)CC5)c(C(F)(F)F)c4)c3)n2n1 ; C[C@H]1CN(C)CCN1c1ccc2ncc(C#Cc3cncc(C(=O)Nc4ccc(CN5CCN(C)CC5)c(C(F)(F)F)c4)c3)n2n1 ; CN1CCN(Cc2ccc(NC(=O)c3cncc(C#Cc4cnc5ccc(N6CC(C)(C)OC(C)(C)C6)nn45)c3)cc2C(F)(F)F)CC1]

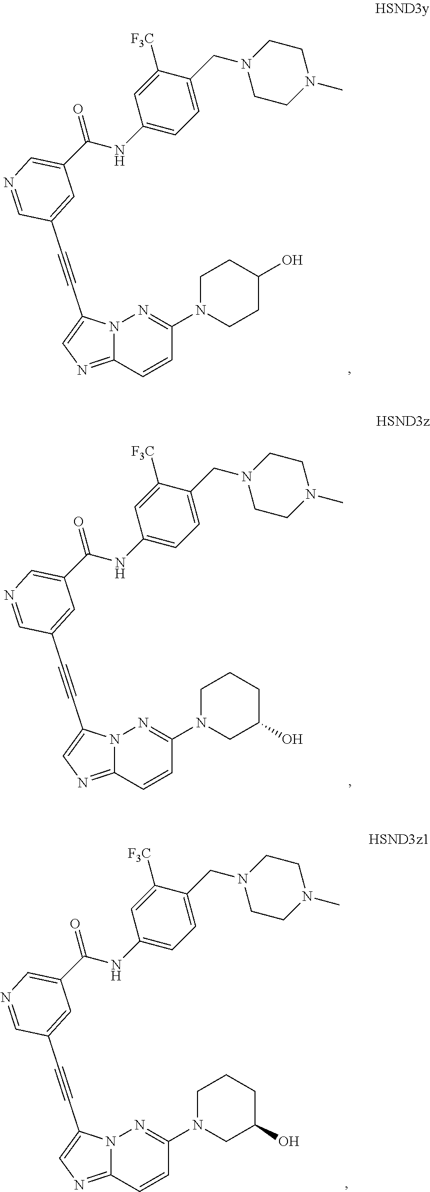 [SMILES: CN1CCN(Cc2ccc(NC(=O)c3cncc(C#Cc4cnc5ccc(N6CCC(O)CC6)nn45)c3)cc2C(F)(F)F)CC1 ; CN1CCN(Cc2ccc(NC(=O)c3cncc(C#Cc4cnc5ccc(N6CCC[C@H](O)C6)nn45)c3)cc2C(F)(F)F)CC1 ; CN1CCN(Cc2ccc(NC(=O)c3cncc(C#Cc4cnc5ccc(N6CCC[C@@H](O)C6)nn45)c3)cc2C(F)(F)F)CC1]

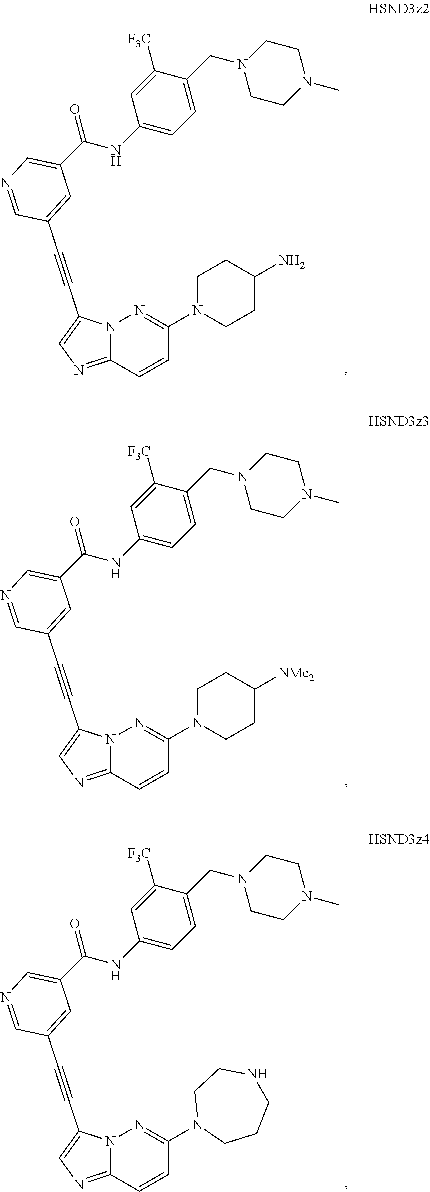 [SMILES: CN1CCN(Cc2ccc(NC(=O)c3cncc(C#Cc4cnc5ccc(N6CCC(N)CC6)nn45)c3)cc2C(F)(F)F)CC1 ; CN1CCN(Cc2ccc(NC(=O)c3cncc(C#Cc4cnc5ccc(N6CCC(N(C)C)CC6)nn45)c3)cc2C(F)(F)F)CC1 ; CN1CCN(Cc2ccc(NC(=O)c3cncc(C#Cc4cnc5ccc(N6CCCNCC6)nn45)c3)cc2C(F)(F)F)CC1]

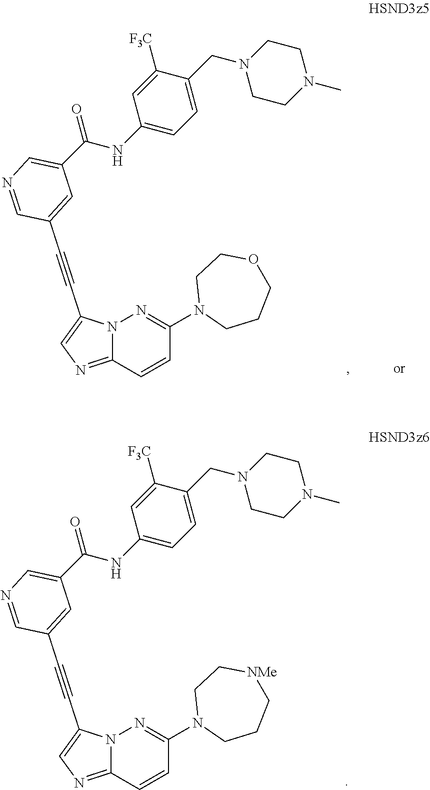 [SMILES: CN1CCN(Cc2ccc(NC(=O)c3cncc(C#Cc4cnc5ccc(N6CCCOCC6)nn45)c3)cc2C(F)(F)F)CC1 ; CN1CCN(Cc2ccc(NC(=O)c3cncc(C#Cc4cnc5ccc(N6CCCN(C)CC6)nn45)c3)cc2C(F)(F)F)CC1]

The compound can have the following structure:

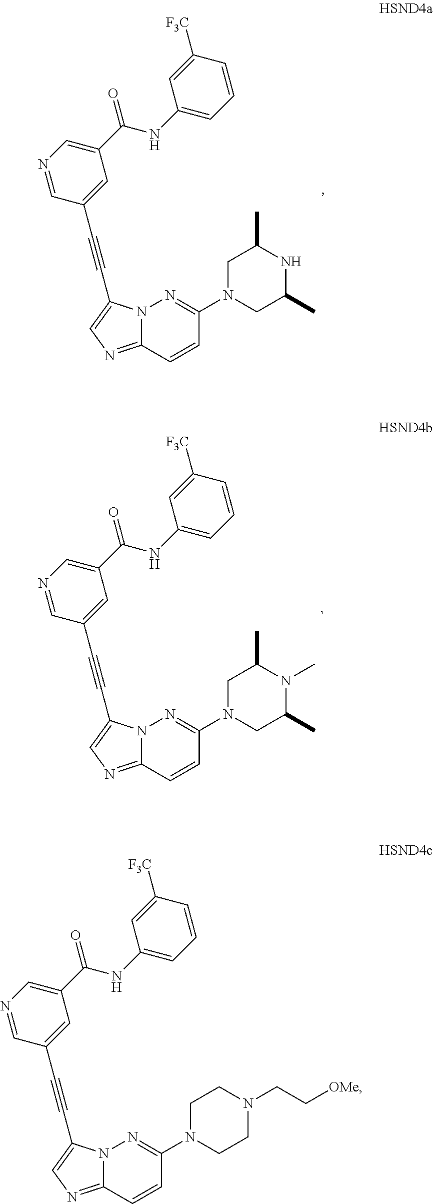 [SMILES: C[C@@H]1CN(c2ccc3ncc(C#Cc4cncc(C(=O)Nc5cccc(C(F)(F)F)c5)c4)n3n2)C[C@H](C)N1 ; C[C@@H]1CN(c2ccc3ncc(C#Cc4cncc(C(=O)Nc5cccc(C(F)(F)F)c5)c4)n3n2)C[C@H](C)N1C ; COCCN1CCN(c2ccc3ncc(C#Cc4cncc(C(=O)Nc5cccc(C(F)(F)F)c5)c4)n3n2)CC1]

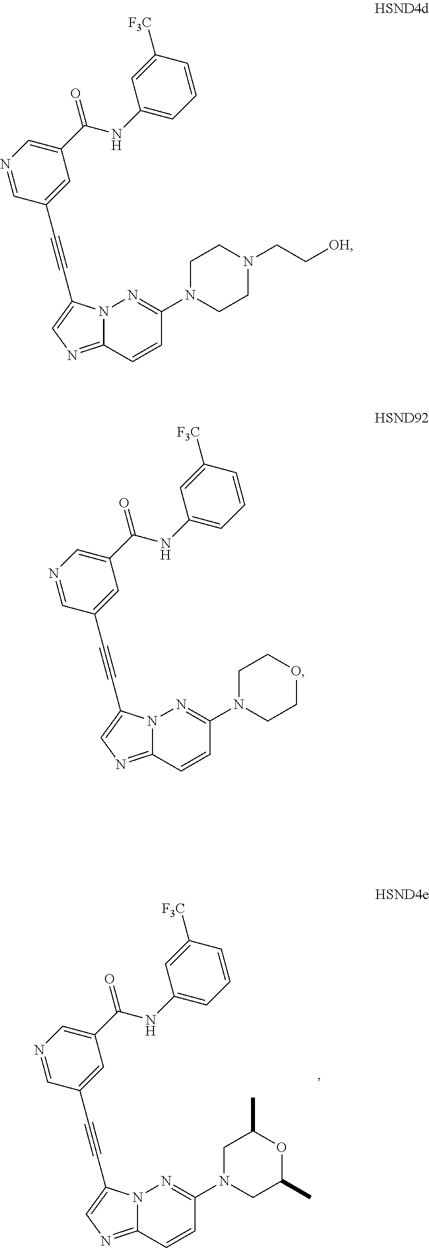 [SMILES: O=C(Nc1cccc(C(F)(F)F)c1)c1cncc(C#Cc2cnc3ccc(N4CCN(CCO)CC4)nn23)c1 ; O=C(Nc1cccc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5cccc(C(F)(F)F)c5)c4)n3n2)C[C@H](C)O1]

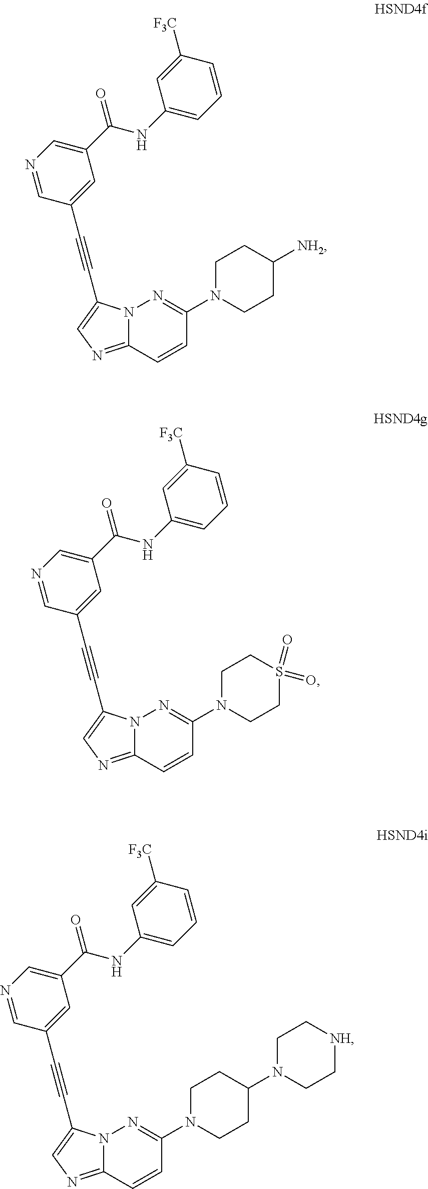 [SMILES: NC1CCN(c2ccc3ncc(C#Cc4cncc(C(=O)Nc5cccc(C(F)(F)F)c5)c4)n3n2)CC1 ; O=C(Nc1cccc(C(F)(F)F)c1)c1cncc(C#Cc2cnc3ccc(N4CCS(=O)(=O)CC4)nn23)c1 ; O=C(Nc1cccc(C(F)(F)F)c1)c1cncc(C#Cc2cnc3ccc(N4CCC(N5CCNCC5)CC4)nn23)c1]

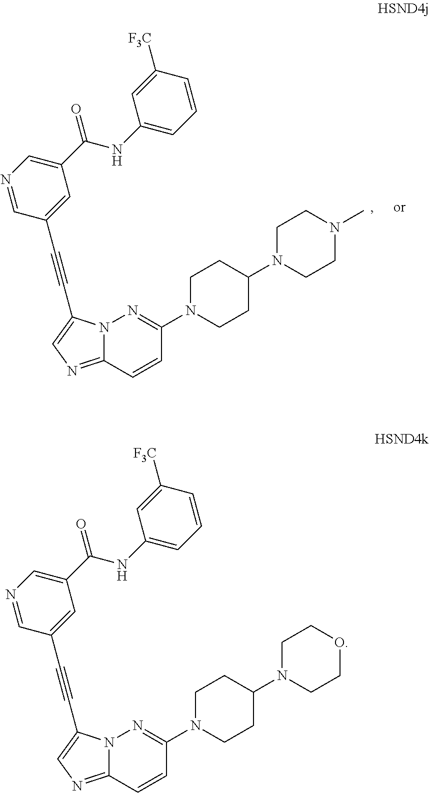 [SMILES: CN1CCN(C2CCN(c3ccc4ncc(C#Cc5cncc(C(=O)Nc6cccc(C(F)(F)F)c6)c5)n4n3)CC2)CC1 ; O=C(Nc1cccc(C(F)(F)F)c1)c1cncc(C#Cc2cnc3ccc(N4CCC(N5CCOCC5)CC4)nn23)c1]

The compound can have the following structure:

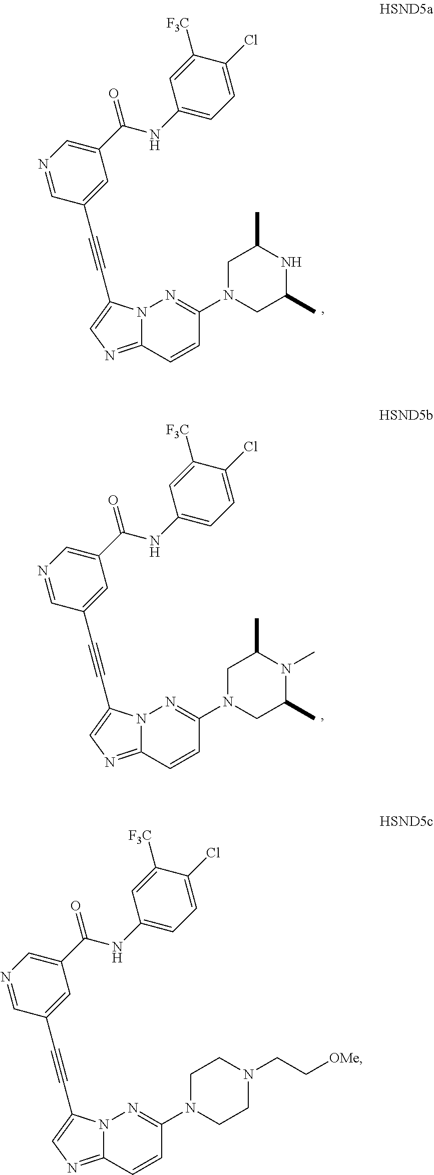 [SMILES: C[C@@H]1CN(c2ccc3ncc(C#Cc4cncc(C(=O)Nc5ccc(Cl)c(C(F)(F)F)c5)c4)n3n2)C[C@H](C)N1 ; C[C@@H]1CN(c2ccc3ncc(C#Cc4cncc(C(=O)Nc5ccc(Cl)c(C(F)(F)F)c5)c4)n3n2)C[C@H](C)N1C ; COCCN1CCN(c2ccc3ncc(C#Cc4cncc(C(=O)Nc5ccc(Cl)c(C(F)(F)F)c5)c4)n3n2)CC1]

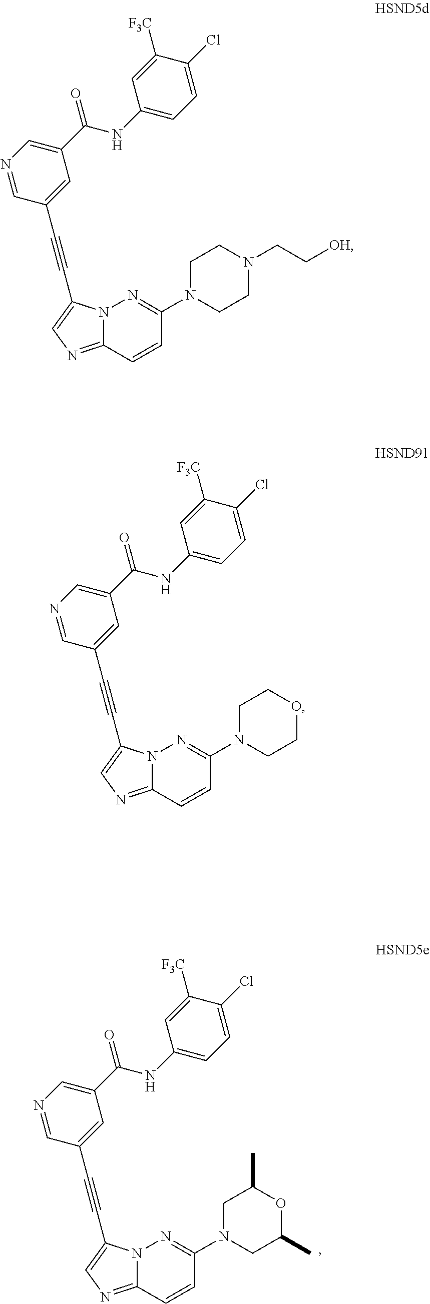 [SMILES: O=C(Nc1ccc(Cl)c(C(F)(F)F)c1)c1cncc(C#Cc2cnc3ccc(N4CCN(CCO)CC4)nn23)c1 ; O=C(Nc1ccc(Cl)c(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5ccc(Cl)c(C(F)(F)F)c5)c4)n3n2)C[C@H](C)O1]

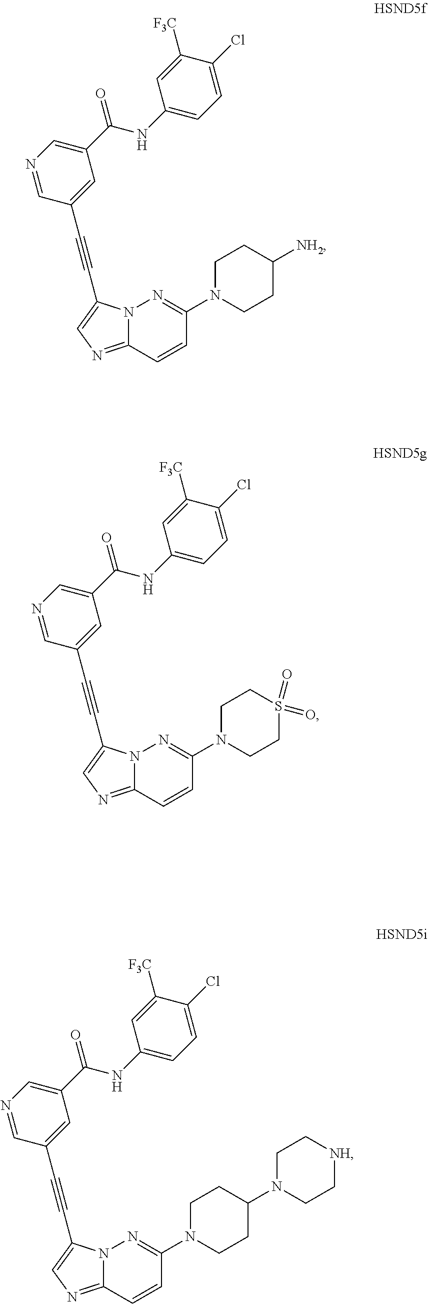 [SMILES: NC1CCN(c2ccc3ncc(C#Cc4cncc(C(=O)Nc5ccc(Cl)c(C(F)(F)F)c5)c4)n3n2)CC1 ; O=C(Nc1ccc(Cl)c(C(F)(F)F)c1)c1cncc(C#Cc2cnc3ccc(N4CCS(=O)(=O)CC4)nn23)c1 ; O=C(Nc1ccc(Cl)c(C(F)(F)F)c1)c1cncc(C#Cc2cnc3ccc(N4CCC(N5CCNCC5)CC4)nn23)c1]

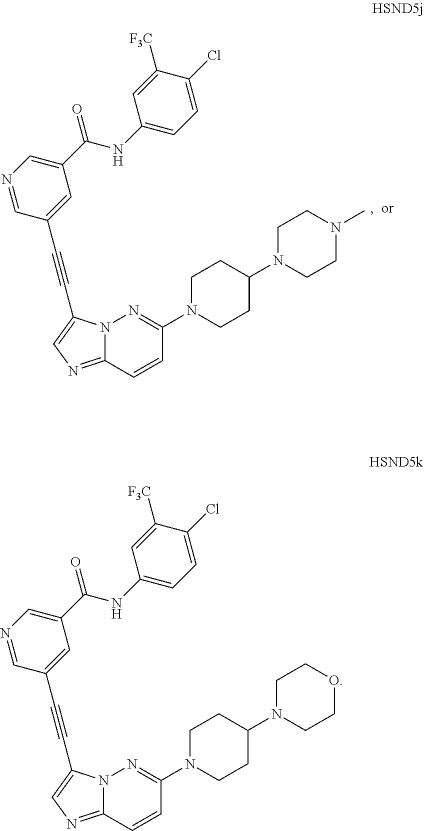 [SMILES: CN1CCN(C2CCN(c3ccc4ncc(C#Cc5cncc(C(=O)Nc6ccc(Cl)c(C(F)(F)F)c6)c5)n4n3)CC2)CC1 ; O=C(Nc1ccc(Cl)c(C(F)(F)F)c1)c1cncc(C#Cc2cnc3ccc(N4CCC(N5CCOCC5)CC4)nn23)c1]

The compound can have the following structure:

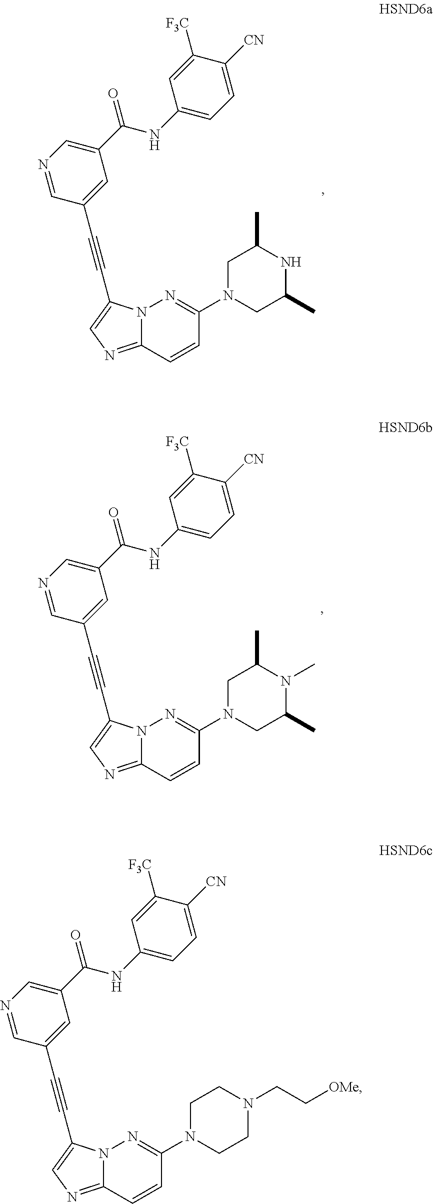 [SMILES: C[C@@H]1CN(c2ccc3ncc(C#Cc4cncc(C(=O)Nc5ccc(C#N)c(C(F)(F)F)c5)c4)n3n2)C[C@H](C)N1 ; C[C@@H]1CN(c2ccc3ncc(C#Cc4cncc(C(=O)Nc5ccc(C#N)c(C(F)(F)F)c5)c4)n3n2)C[C@H](C)N1C ; COCCN1CCN(c2ccc3ncc(C#Cc4cncc(C(=O)Nc5ccc(C#N)c(C(F)(F)F)c5)c4)n3n2)CC1]

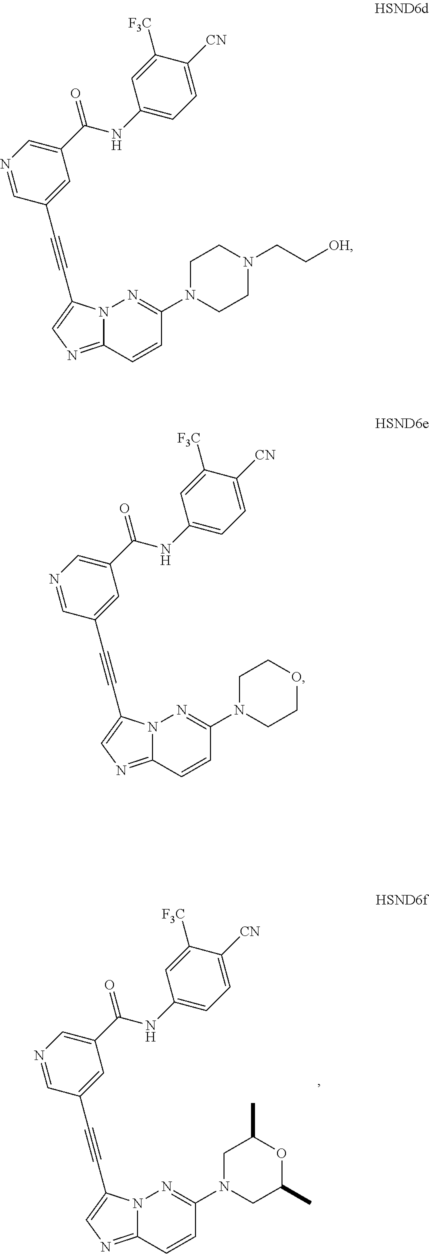 [SMILES: N#Cc1ccc(NC(=O)c2cncc(C#Cc3cnc4ccc(N5CCN(CCO)CC5)nn34)c2)cc1C(F)(F)F ; N#Cc1ccc(NC(=O)c2cncc(C#Cc3cnc4ccc(N5CCOCC5)nn34)c2)cc1C(F)(F)F ; C[C@@H]1CN(c2ccc3ncc(C#Cc4cncc(C(=O)Nc5ccc(C#N)c(C(F)(F)F)c5)c4)n3n2)C[C@H](C)O1]

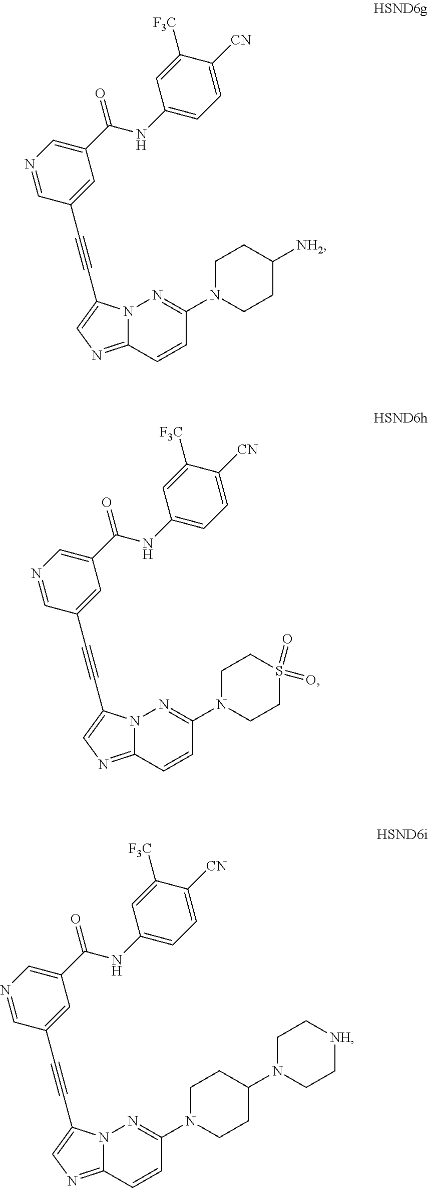 [SMILES: N#Cc1ccc(NC(=O)c2cncc(C#Cc3cnc4ccc(N5CCC(N)CC5)nn34)c2)cc1C(F)(F)F ; N#Cc1ccc(NC(=O)c2cncc(C#Cc3cnc4ccc(N5CCS(=O)(=O)CC5)nn34)c2)cc1C(F)(F)F ; N#Cc1ccc(NC(=O)c2cncc(C#Cc3cnc4ccc(N5CCC(N6CCNCC6)CC5)nn34)c2)cc1C(F)(F)F]

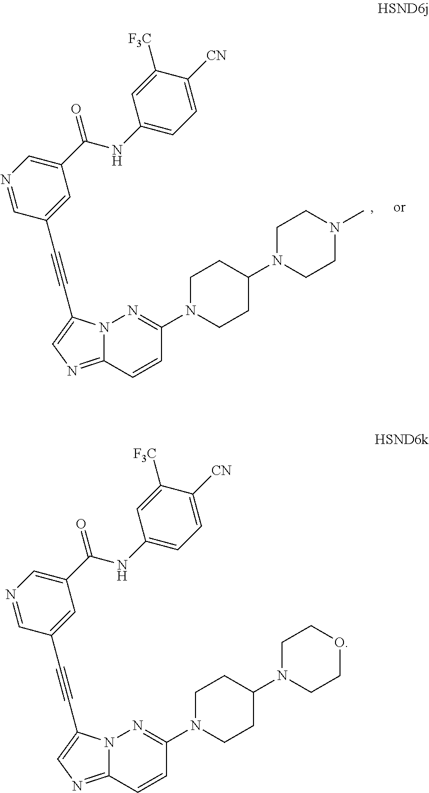 [SMILES: CN1CCN(C2CCN(c3ccc4ncc(C#Cc5cncc(C(=O)Nc6ccc(C#N)c(C(F)(F)F)c6)c5)n4n3)CC2)CC1 ; N#Cc1ccc(NC(=O)c2cncc(C#Cc3cnc4ccc(N5CCC(N6CCOCC6)CC5)nn34)c2)cc1C(F)(F)F]

The compound can have the following structure:

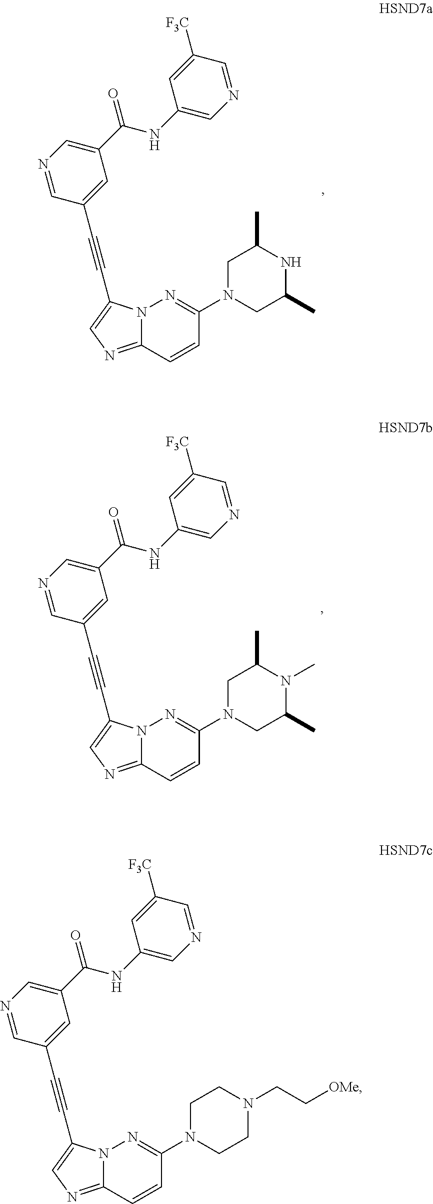 [SMILES: C[C@@H]1CN(c2ccc3ncc(C#Cc4cncc(C(=O)Nc5cncc(C(F)(F)F)c5)c4)n3n2)C[C@H](C)N1 ; C[C@@H]1CN(c2ccc3ncc(C#Cc4cncc(C(=O)Nc5cncc(C(F)(F)F)c5)c4)n3n2)C[C@H](C)N1C ; COCCN1CCN(c2ccc3ncc(C#Cc4cncc(C(=O)Nc5cncc(C(F)(F)F)c5)c4)n3n2)CC1]

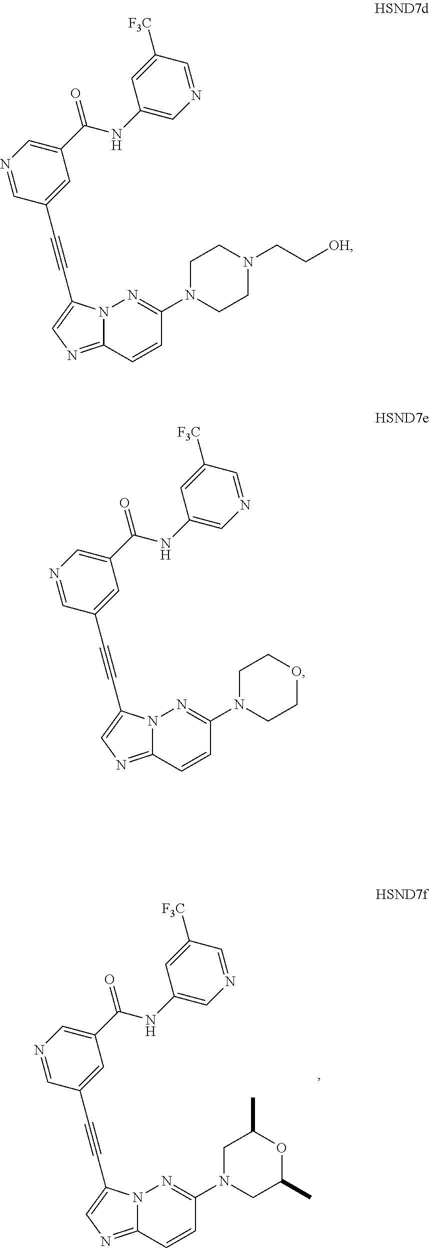 [SMILES: O=C(Nc1cncc(C(F)(F)F)c1)c1cncc(C#Cc2cnc3ccc(N4CCN(CCO)CC4)nn23)c1 ; O=C(Nc1cncc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5cncc(C(F)(F)F)c5)c4)n3n2)C[C@H](C)O1]

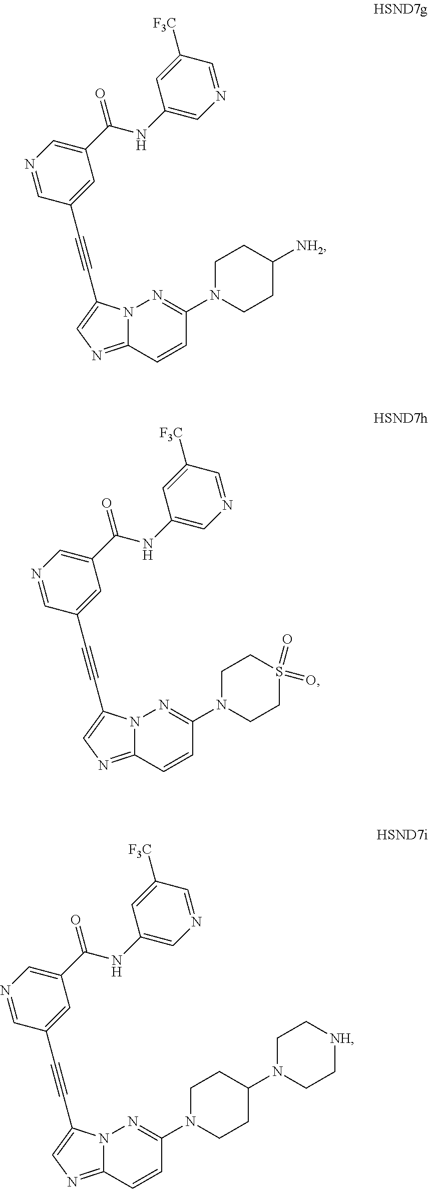 [SMILES: NC1CCN(c2ccc3ncc(C#Cc4cncc(C(=O)Nc5cncc(C(F)(F)F)c5)c4)n3n2)CC1 ; O=C(Nc1cncc(C(F)(F)F)c1)c1cncc(C#Cc2cnc3ccc(N4CCS(=O)(=O)CC4)nn23)c1 ; O=C(Nc1cncc(C(F)(F)F)c1)c1cncc(C#Cc2cnc3ccc(N4CCC(N5CCNCC5)CC4)nn23)c1]

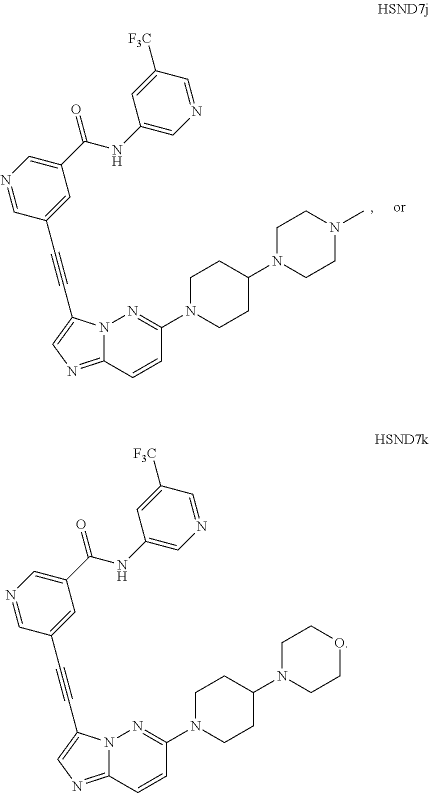 [SMILES: CN1CCN(C2CCN(c3ccc4ncc(C#Cc5cncc(C(=O)Nc6cncc(C(F)(F)F)c6)c5)n4n3)CC2)CC1 ; O=C(Nc1cncc(C(F)(F)F)c1)c1cncc(C#Cc2cnc3ccc(N4CCC(N5CCOCC5)CC4)nn23)c1]

The compound can have the following structure:

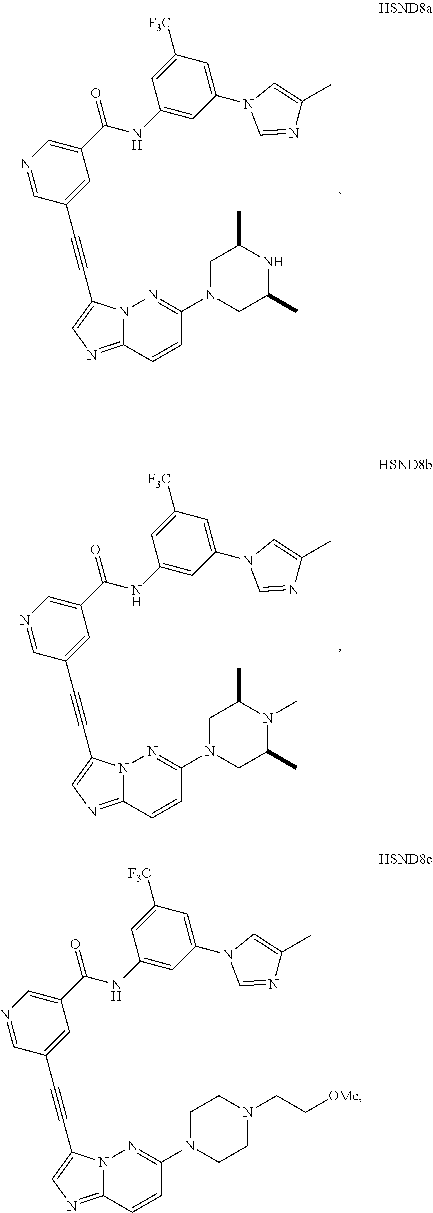 [SMILES: Cc1cn(-c2cc(NC(=O)c3cncc(C#Cc4cnc5ccc(N6C[C@@H](C)N[C@@H](C)C6)nn45)c3)cc(C(F)(F)F)c2)cn1 ; Cc1cn(-c2cc(NC(=O)c3cncc(C#Cc4cnc5ccc(N6C[C@@H](C)N(C)[C@@H](C)C6)nn45)c3)cc(C(F)(F)F)c2)cn1 ; COCCN1CCN(c2ccc3ncc(C#Cc4cncc(C(=O)Nc5cc(-n6cnc(C)c6)cc(C(F)(F)F)c5)c4)n3n2)CC1]

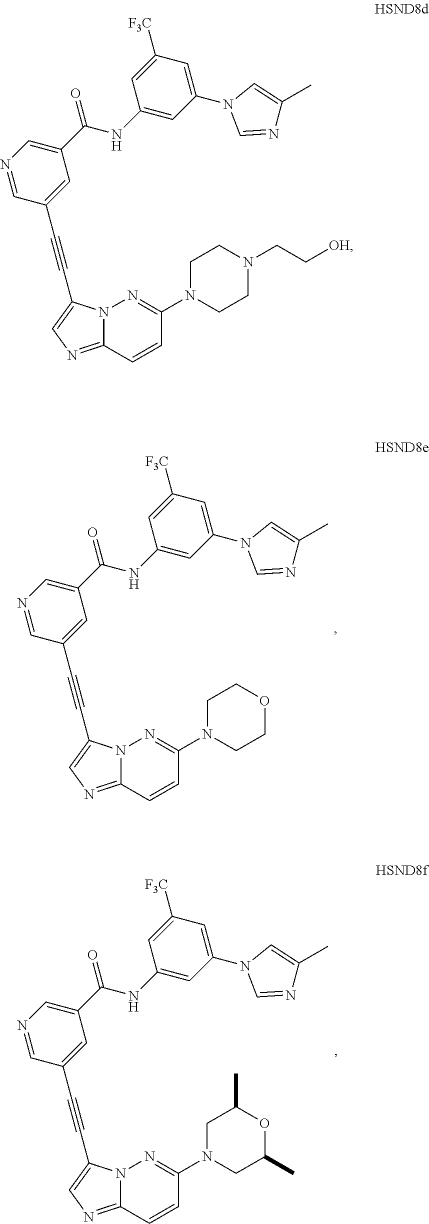 [SMILES: Cc1cn(-c2cc(NC(=O)c3cncc(C#Cc4cnc5ccc(N6CCN(CCO)CC6)nn45)c3)cc(C(F)(F)F)c2)cn1 ; Cc1cn(-c2cc(NC(=O)c3cncc(C#Cc4cnc5ccc(N6CCOCC6)nn45)c3)cc(C(F)(F)F)c2)cn1 ; Cc1cn(-c2cc(NC(=O)c3cncc(C#Cc4cnc5ccc(N6C[C@@H](C)O[C@@H](C)C6)nn45)c3)cc(C(F)(F)F)c2)cn1]

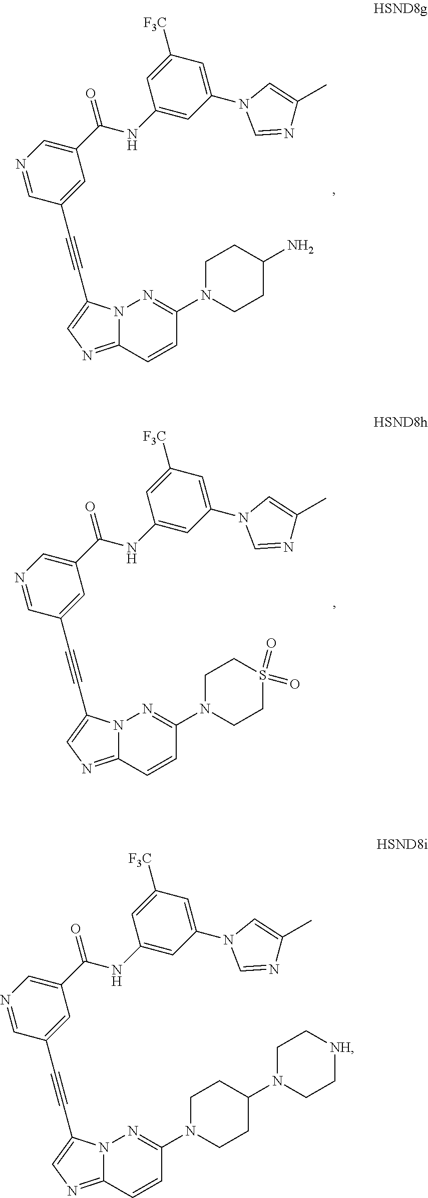 [SMILES: Cc1cn(-c2cc(NC(=O)c3cncc(C#Cc4cnc5ccc(N6CCC(N)CC6)nn45)c3)cc(C(F)(F)F)c2)cn1 ; Cc1cn(-c2cc(NC(=O)c3cncc(C#Cc4cnc5ccc(N6CCS(=O)(=O)CC6)nn45)c3)cc(C(F)(F)F)c2)cn1 ; Cc1cn(-c2cc(NC(=O)c3cncc(C#Cc4cnc5ccc(N6CCC(N7CCNCC7)CC6)nn45)c3)cc(C(F)(F)F)c2)cn1]

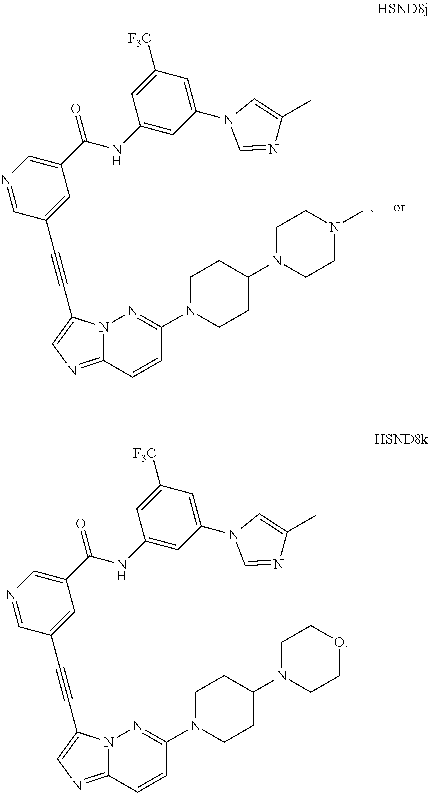 [SMILES: Cc1cn(-c2cc(NC(=O)c3cncc(C#Cc4cnc5ccc(N6CCC(N7CCN(C)CC7)CC6)nn45)c3)cc(C(F)(F)F)c2)cn1 ; Cc1cn(-c2cc(NC(=O)c3cncc(C#Cc4cnc5ccc(N6CCC(N7CCOCC7)CC6)nn45)c3)cc(C(F)(F)F)c2)cn1]

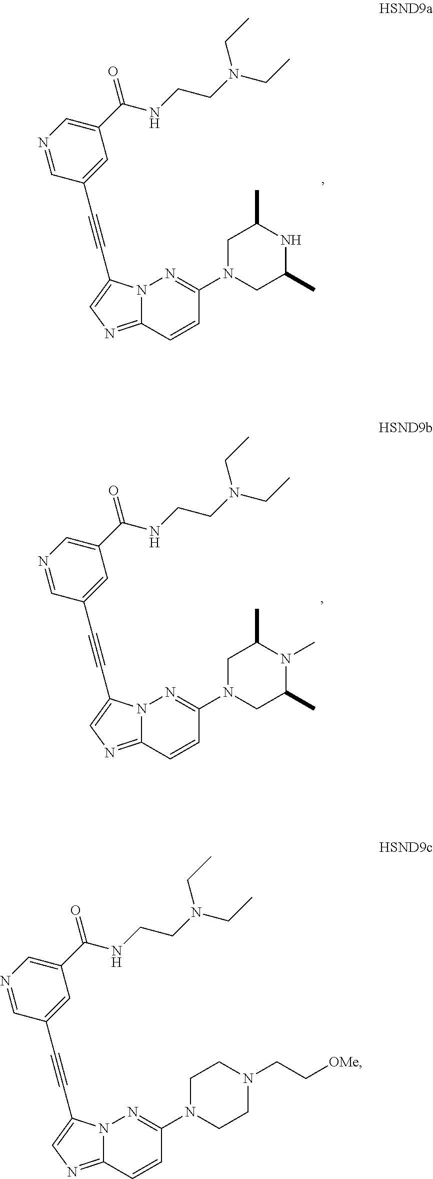 [SMILES: CCN(CC)CCNC(=O)c1cncc(C#Cc2cnc3ccc(N4C[C@@H](C)N[C@@H](C)C4)nn23)c1 ; CCN(CC)CCNC(=O)c1cncc(C#Cc2cnc3ccc(N4C[C@@H](C)N(C)[C@@H](C)C4)nn23)c1 ; CCN(CC)CCNC(=O)c1cncc(C#Cc2cnc3ccc(N4CCN(CCOC)CC4)nn23)c1]

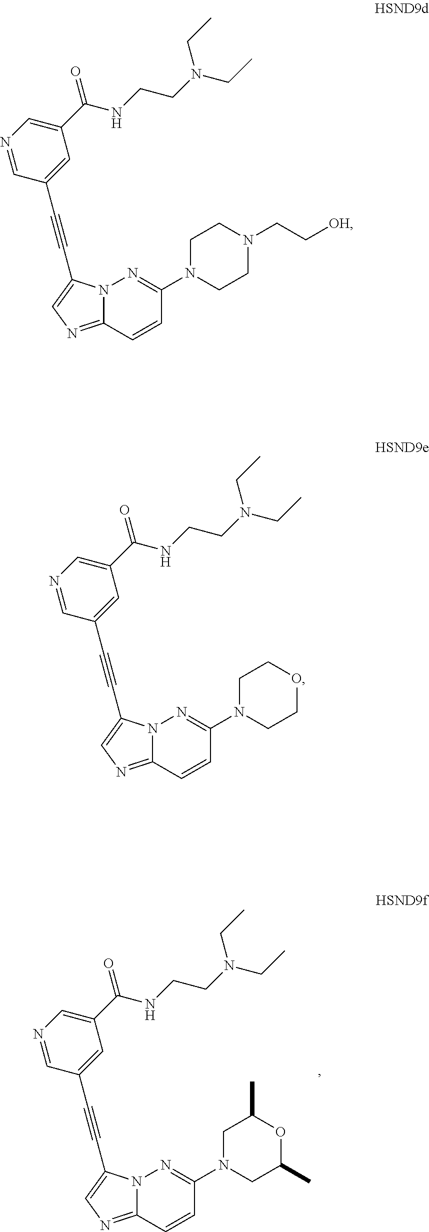 [SMILES: CCN(CC)CCNC(=O)c1cncc(C#Cc2cnc3ccc(N4CCN(CCO)CC4)nn23)c1 ; CCN(CC)CCNC(=O)c1cncc(C#Cc2cnc3ccc(N4CCOCC4)nn23)c1 ; CCN(CC)CCNC(=O)c1cncc(C#Cc2cnc3ccc(N4C[C@@H](C)O[C@@H](C)C4)nn23)c1]

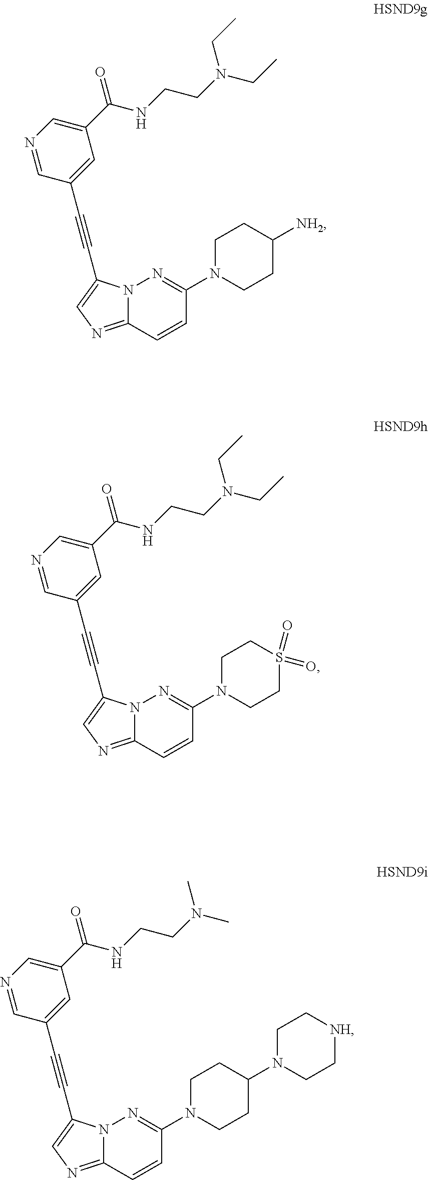 [SMILES: CCN(CC)CCNC(=O)c1cncc(C#Cc2cnc3ccc(N4CCC(N)CC4)nn23)c1 ; CCN(CC)CCNC(=O)c1cncc(C#Cc2cnc3ccc(N4CCS(=O)(=O)CC4)nn23)c1 ; CN(C)CCNC(=O)c1cncc(C#Cc2cnc3ccc(N4CCC(N5CCNCC5)CC4)nn23)c1]

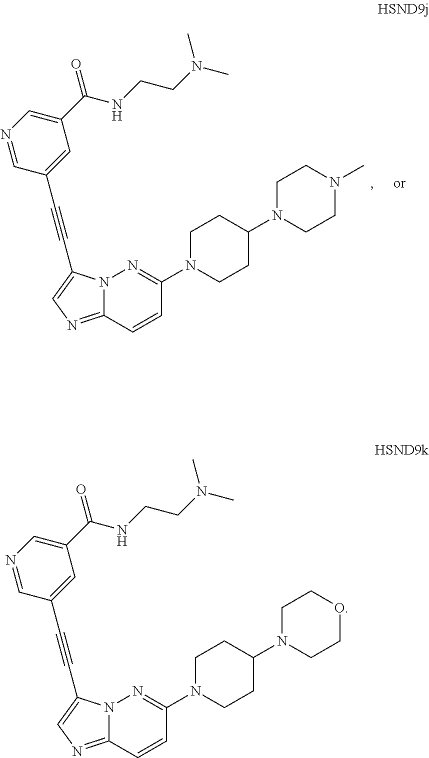 [SMILES: CN(C)CCNC(=O)c1cncc(C#Cc2cnc3ccc(N4CCC(N5CCN(C)CC5)CC4)nn23)c1 ; CN(C)CCNC(=O)c1cncc(C#Cc2cnc3ccc(N4CCC(N5CCOCC5)CC4)nn23)c1]

The compound can have the following structure:

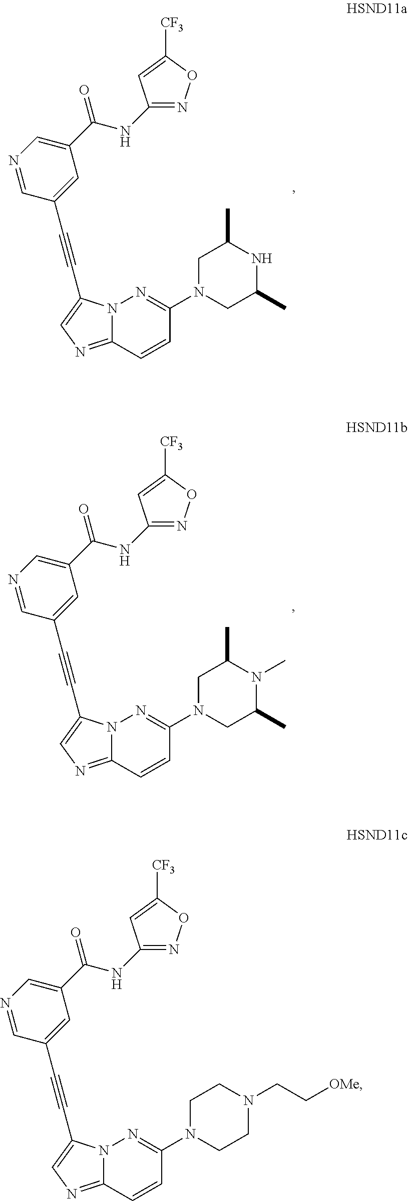 [SMILES: C[C@@H]1CN(c2ccc3ncc(C#Cc4cncc(C(=O)Nc5cc(C(F)(F)F)on5)c4)n3n2)C[C@H](C)N1 ; C[C@@H]1CN(c2ccc3ncc(C#Cc4cncc(C(=O)Nc5cc(C(F)(F)F)on5)c4)n3n2)C[C@H](C)N1C ; COCCN1CCN(c2ccc3ncc(C#Cc4cncc(C(=O)Nc5cc(C(F)(F)F)on5)c4)n3n2)CC1]

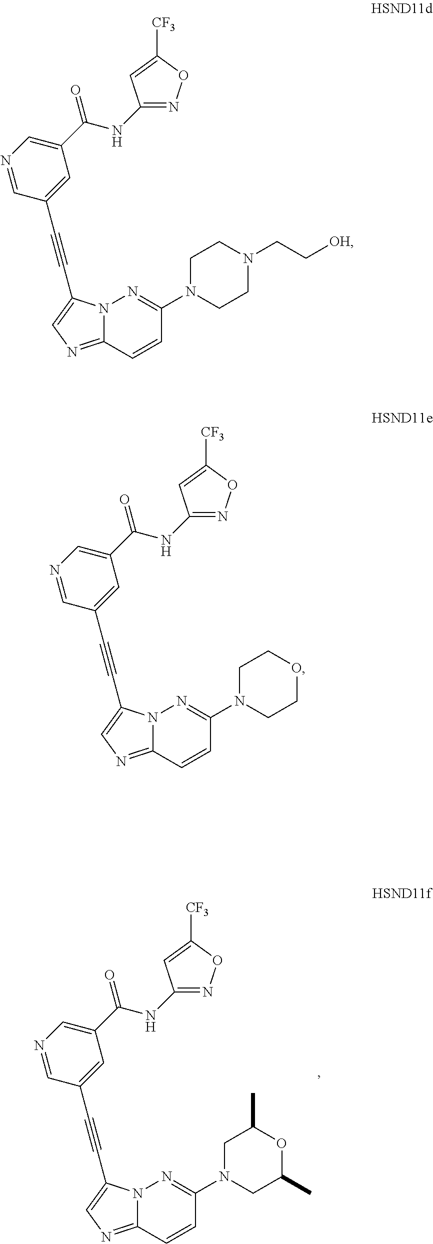 [SMILES: O=C(Nc1cc(C(F)(F)F)on1)c1cncc(C#Cc2cnc3ccc(N4CCN(CCO)CC4)nn23)c1 ; O=C(Nc1cc(C(F)(F)F)on1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)on5)c4)n3n2)C[C@H](C)O1]

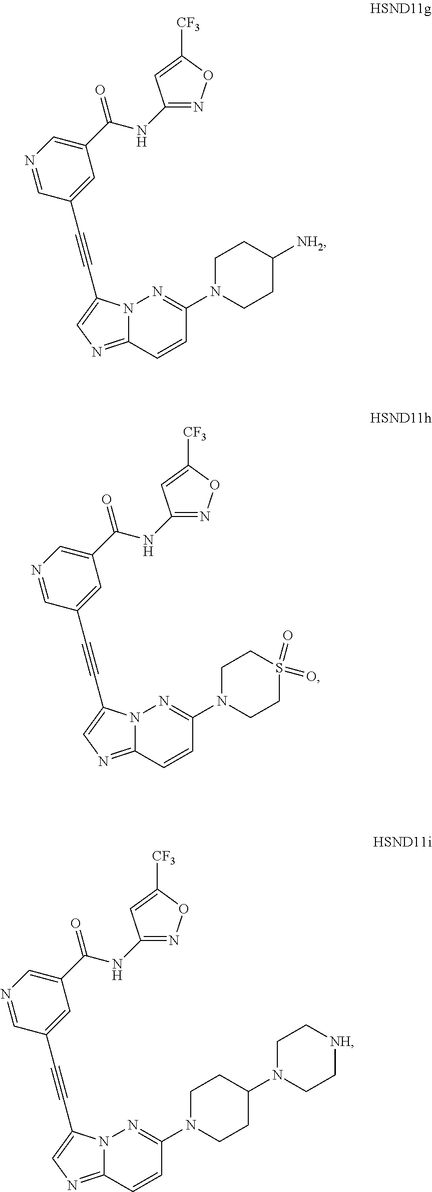 [SMILES: NC1CCN(c2ccc3ncc(C#Cc4cncc(C(=O)Nc5cc(C(F)(F)F)on5)c4)n3n2)CC1 ; O=C(Nc1cc(C(F)(F)F)on1)c1cncc(C#Cc2cnc3ccc(N4CCS(=O)(=O)CC4)nn23)c1 ; O=C(Nc1cc(C(F)(F)F)on1)c1cncc(C#Cc2cnc3ccc(N4CCC(N5CCNCC5)CC4)nn23)c1]

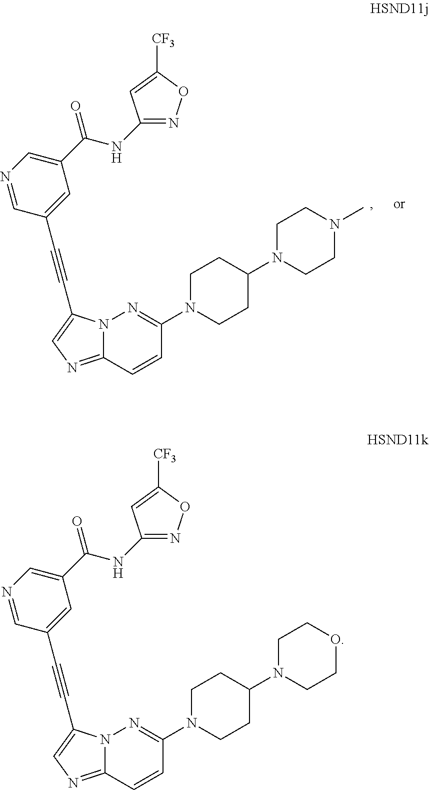 [SMILES: CN1CCN(C2CCN(c3ccc4ncc(C#Cc5cncc(C(=O)Nc6cc(C(F)(F)F)on6)c5)n4n3)CC2)CC1 ; O=C(Nc1cc(C(F)(F)F)on1)c1cncc(C#Cc2cnc3ccc(N4CCC(N5CCOCC5)CC4)nn23)c1]

The compound can have the following structure:

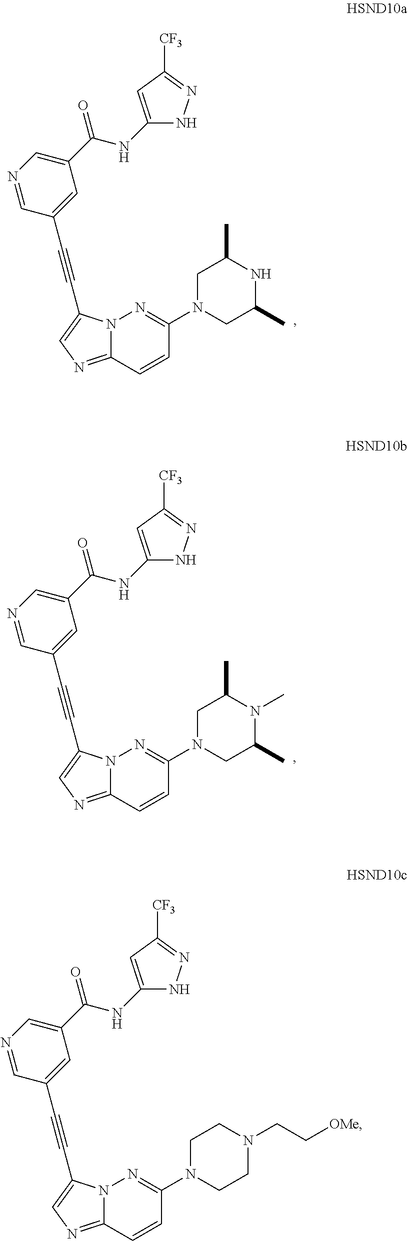 [SMILES: C[C@@H]1CN(c2ccc3ncc(C#Cc4cncc(C(=O)Nc5cc(C(F)(F)F)n[nH]5)c4)n3n2)C[C@H](C)N1 ; C[C@@H]1CN(c2ccc3ncc(C#Cc4cncc(C(=O)Nc5cc(C(F)(F)F)n[nH]5)c4)n3n2)C[C@H](C)N1C ; COCCN1CCN(c2ccc3ncc(C#Cc4cncc(C(=O)Nc5cc(C(F)(F)F)n[nH]5)c4)n3n2)CC1]

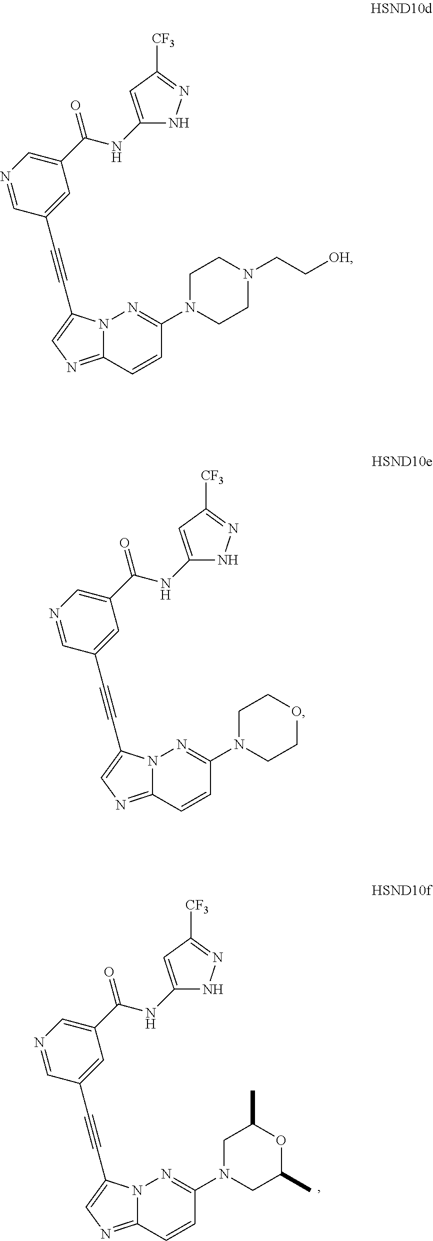 [SMILES: O=C(Nc1cc(C(F)(F)F)n[nH]1)c1cncc(C#Cc2cnc3ccc(N4CCN(CCO)CC4)nn23)c1 ; O=C(Nc1cc(C(F)(F)F)n[nH]1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)n[nH]5)c4)n3n2)C[C@H](C)O1]

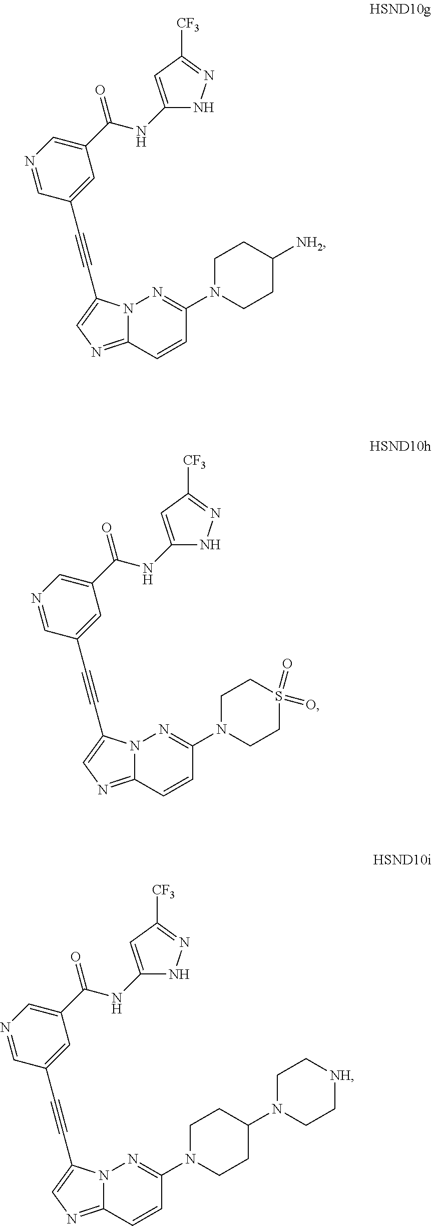 [SMILES: NC1CCN(c2ccc3ncc(C#Cc4cncc(C(=O)Nc5cc(C(F)(F)F)n[nH]5)c4)n3n2)CC1 ; O=C(Nc1cc(C(F)(F)F)n[nH]1)c1cncc(C#Cc2cnc3ccc(N4CCS(=O)(=O)CC4)nn23)c1 ; O=C(Nc1cc(C(F)(F)F)n[nH]1)c1cncc(C#Cc2cnc3ccc(N4CCC(N5CCNCC5)CC4)nn23)c1]

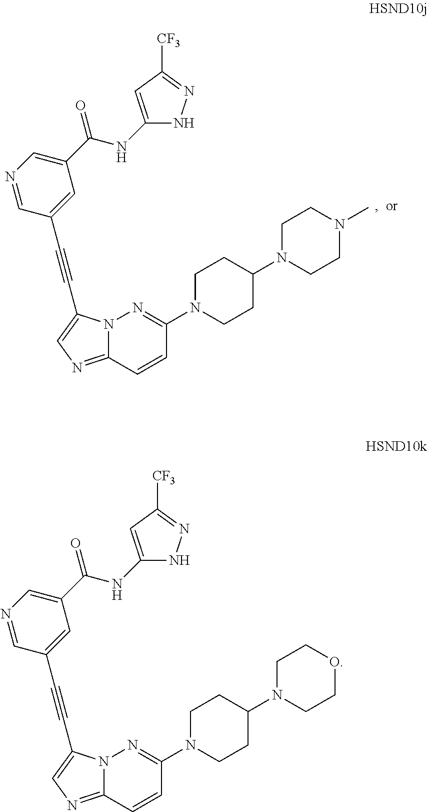 [SMILES: CN1CCN(C2CCN(c3ccc4ncc(C#Cc5cncc(C(=O)Nc6cc(C(F)(F)F)n[nH]6)c5)n4n3)CC2)CC1 ; O=C(Nc1cc(C(F)(F)F)n[nH]1)c1cncc(C#Cc2cnc3ccc(N4CCC(N5CCOCC5)CC4)nn23)c1]

The compound can have the following structure:

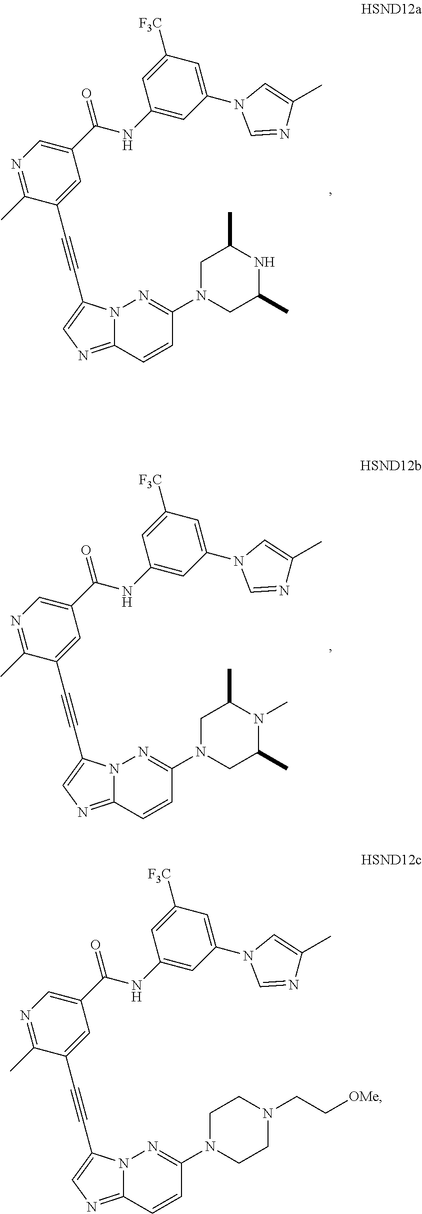

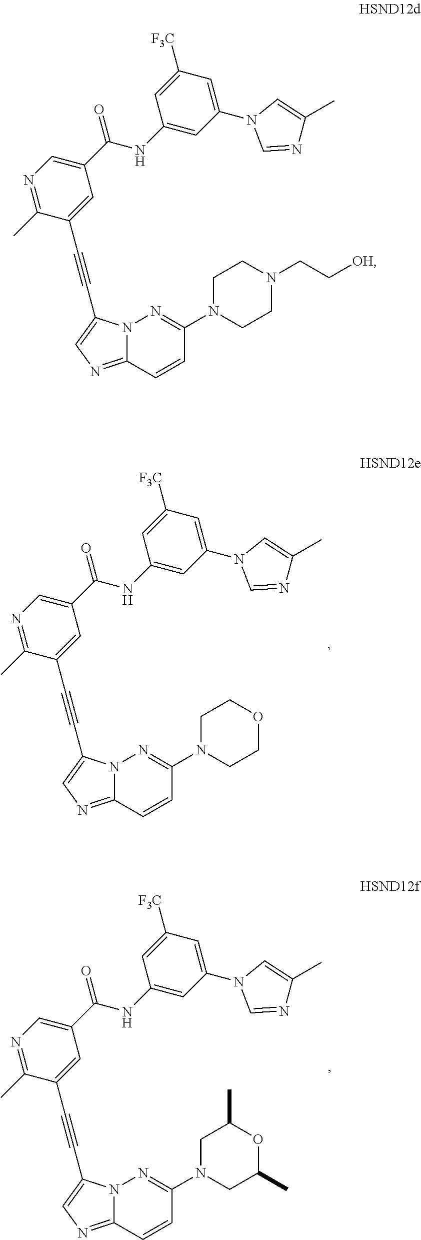

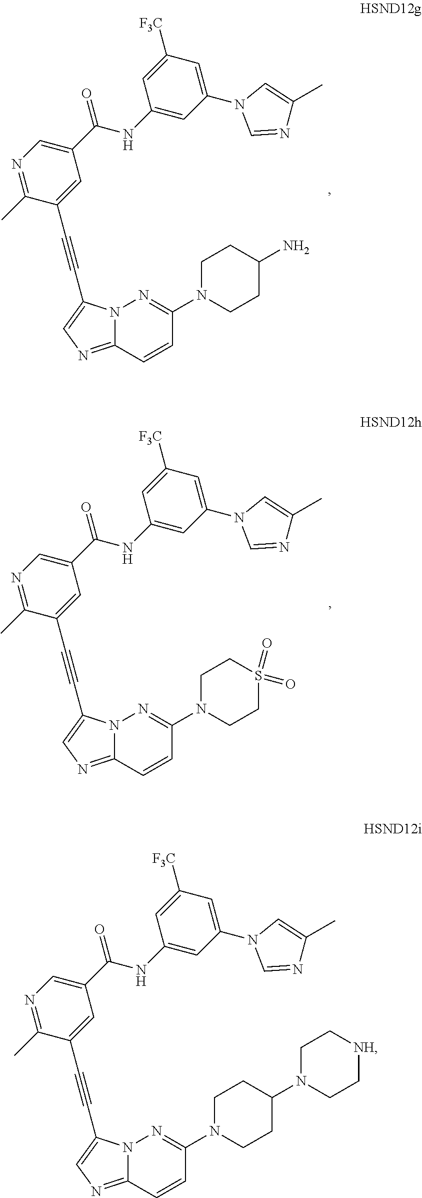

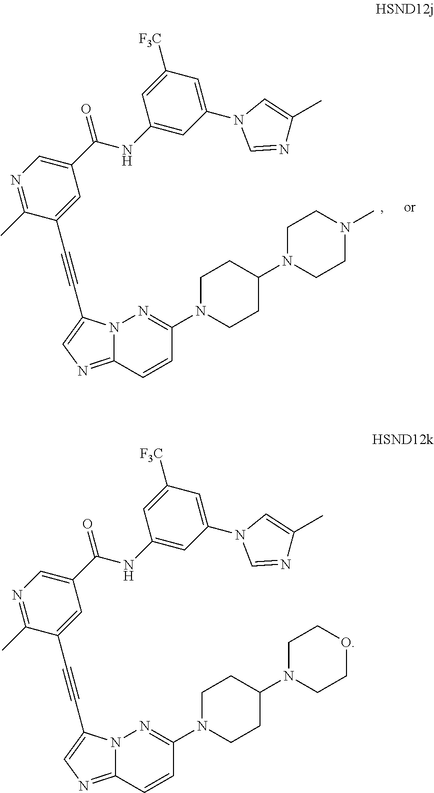

The compound can have the following structure:

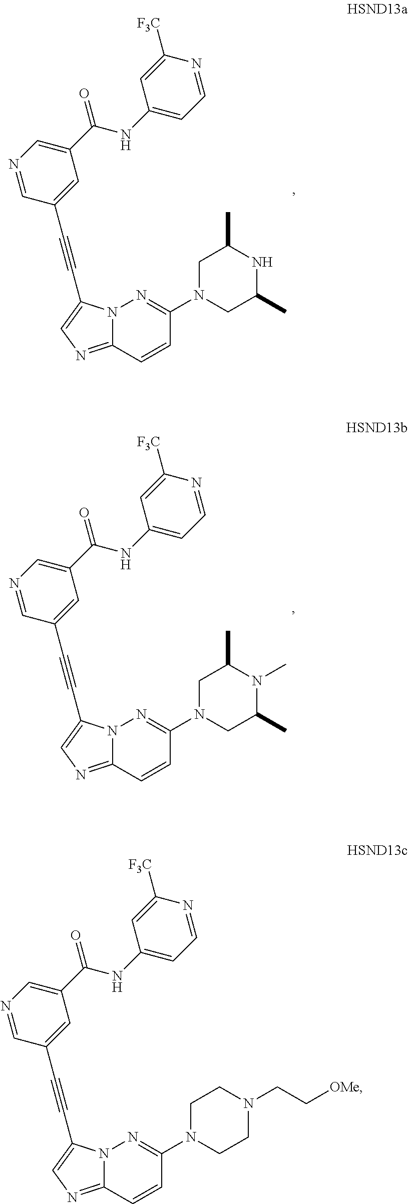 [SMILES: C[C@@H]1CN(c2ccc3ncc(C#Cc4cncc(C(=O)Nc5ccnc(C(F)(F)F)c5)c4)n3n2)C[C@H](C)N1 ; C[C@@H]1CN(c2ccc3ncc(C#Cc4cncc(C(=O)Nc5ccnc(C(F)(F)F)c5)c4)n3n2)C[C@H](C)N1C ; COCCN1CCN(c2ccc3ncc(C#Cc4cncc(C(=O)Nc5ccnc(C(F)(F)F)c5)c4)n3n2)CC1]

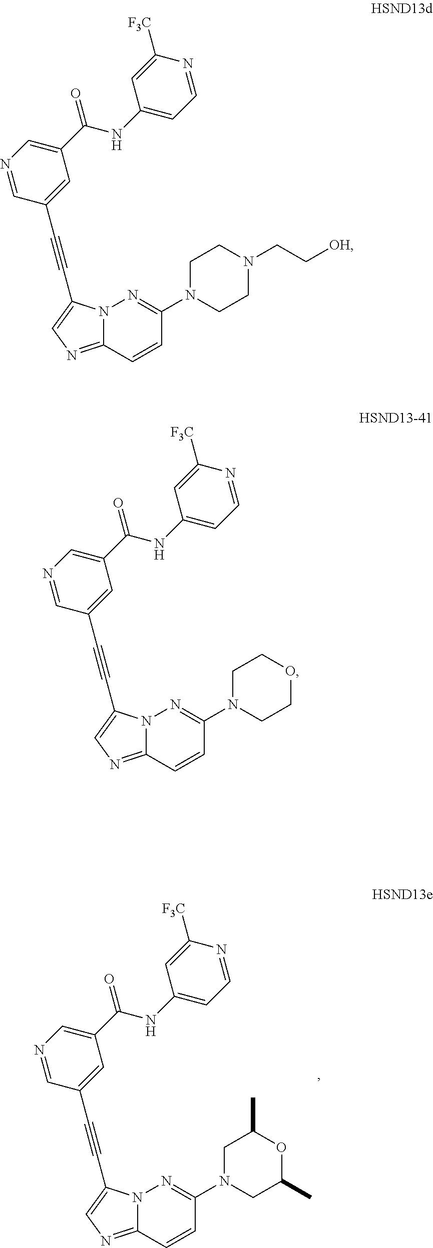 [SMILES: O=C(Nc1ccnc(C(F)(F)F)c1)c1cncc(C#Cc2cnc3ccc(N4CCN(CCO)CC4)nn23)c1 ; O=C(Nc1ccnc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5ccnc(C(F)(F)F)c5)c4)n3n2)C[C@H](C)O1]

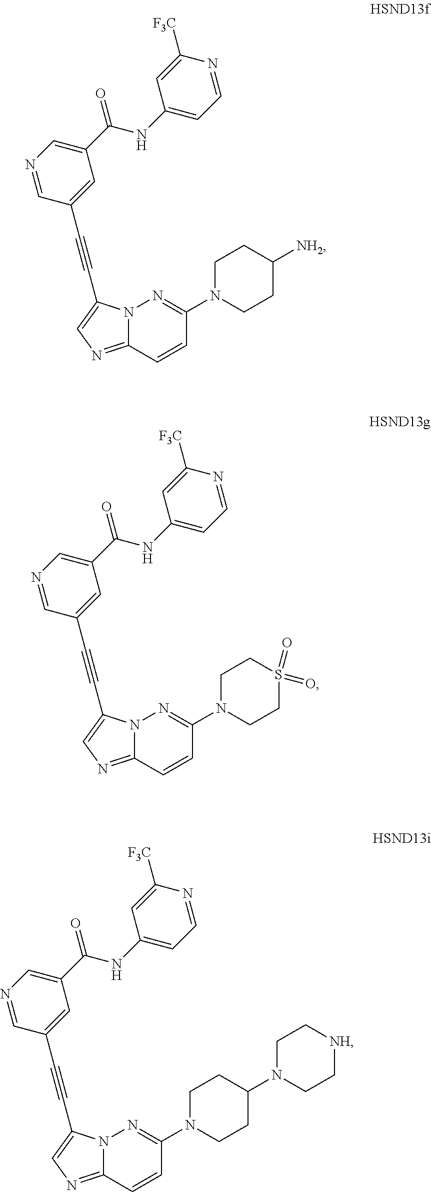 [SMILES: NC1CCN(c2ccc3ncc(C#Cc4cncc(C(=O)Nc5ccnc(C(F)(F)F)c5)c4)n3n2)CC1 ; O=C(Nc1ccnc(C(F)(F)F)c1)c1cncc(C#Cc2cnc3ccc(N4CCS(=O)(=O)CC4)nn23)c1 ; O=C(Nc1ccnc(C(F)(F)F)c1)c1cncc(C#Cc2cnc3ccc(N4CCC(N5CCNCC5)CC4)nn23)c1]

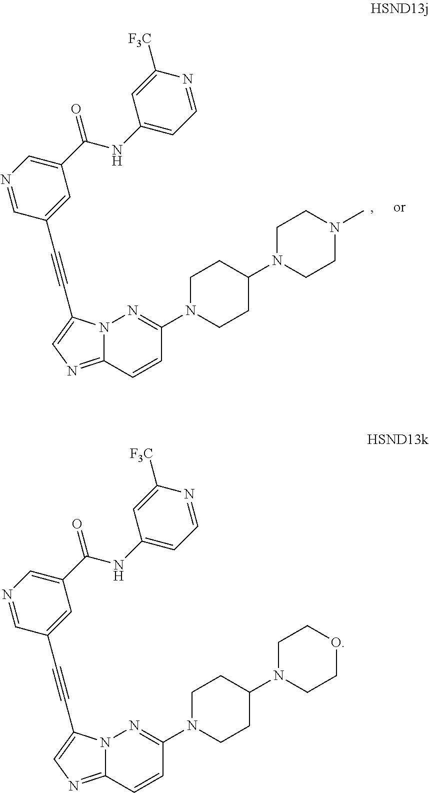 [SMILES: CN1CCN(C2CCN(c3ccc4ncc(C#Cc5cncc(C(=O)Nc6ccnc(C(F)(F)F)c6)c5)n4n3)CC2)CC1 ; O=C(Nc1ccnc(C(F)(F)F)c1)c1cncc(C#Cc2cnc3ccc(N4CCC(N5CCOCC5)CC4)nn23)c1]

The compound can have the following structure:

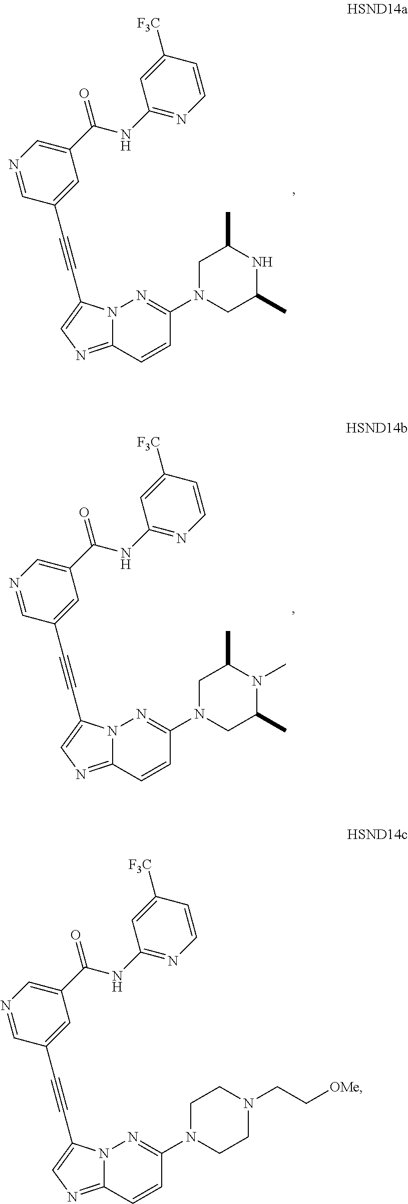 [SMILES: C[C@@H]1CN(c2ccc3ncc(C#Cc4cncc(C(=O)Nc5cc(C(F)(F)F)ccn5)c4)n3n2)C[C@H](C)N1 ; C[C@@H]1CN(c2ccc3ncc(C#Cc4cncc(C(=O)Nc5cc(C(F)(F)F)ccn5)c4)n3n2)C[C@H](C)N1C ; COCCN1CCN(c2ccc3ncc(C#Cc4cncc(C(=O)Nc5cc(C(F)(F)F)ccn5)c4)n3n2)CC1]

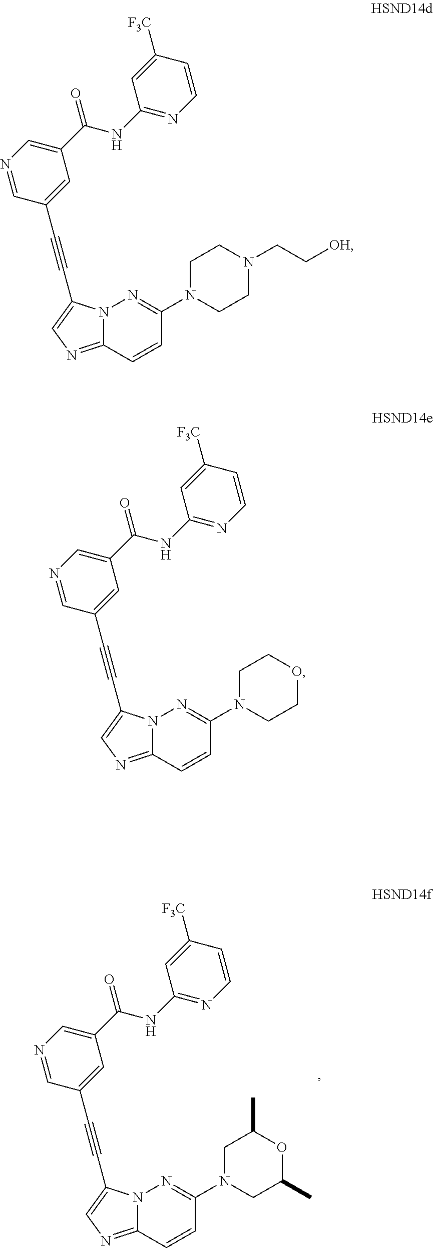 [SMILES: O=C(Nc1cc(C(F)(F)F)ccn1)c1cncc(C#Cc2cnc3ccc(N4CCN(CCO)CC4)nn23)c1 ; O=C(Nc1cc(C(F)(F)F)ccn1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)ccn5)c4)n3n2)C[C@H](C)O1]

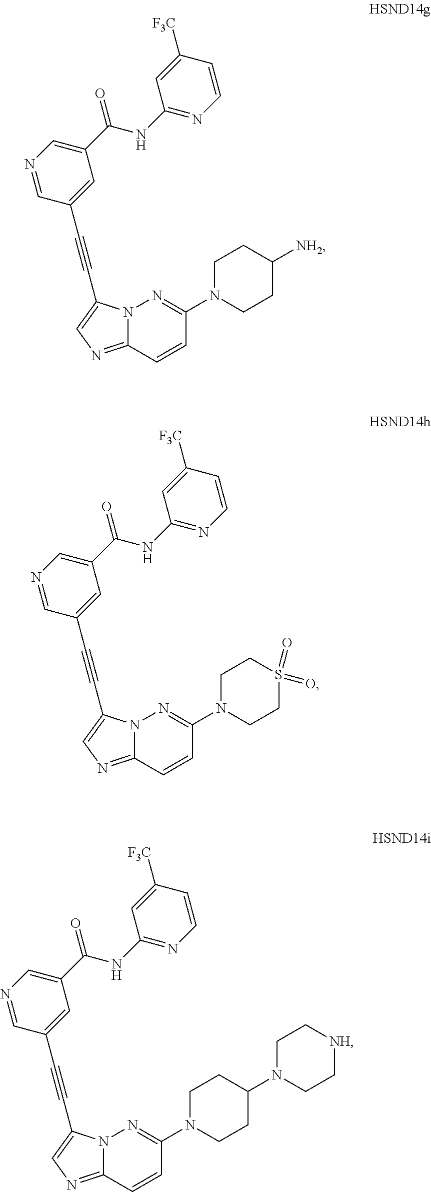 [SMILES: NC1CCN(c2ccc3ncc(C#Cc4cncc(C(=O)Nc5cc(C(F)(F)F)ccn5)c4)n3n2)CC1 ; O=C(Nc1cc(C(F)(F)F)ccn1)c1cncc(C#Cc2cnc3ccc(N4CCS(=O)(=O)CC4)nn23)c1 ; O=C(Nc1cc(C(F)(F)F)ccn1)c1cncc(C#Cc2cnc3ccc(N4CCC(N5CCNCC5)CC4)nn23)c1]

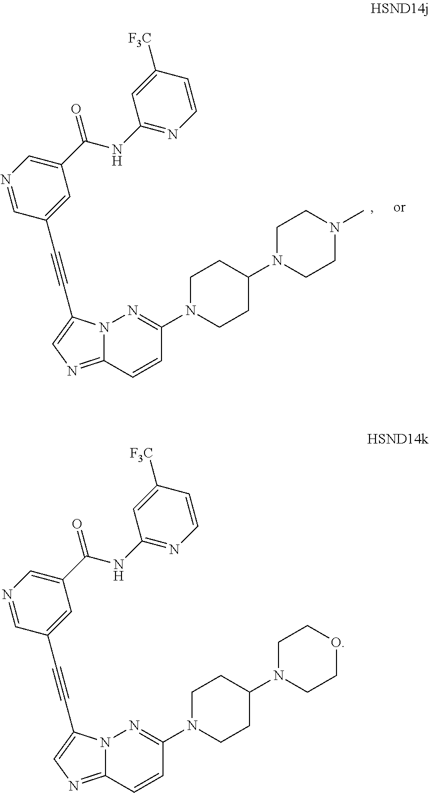 [SMILES: CN1CCN(C2CCN(c3ccc4ncc(C#Cc5cncc(C(=O)Nc6cc(C(F)(F)F)ccn6)c5)n4n3)CC2)CC1 ; O=C(Nc1cc(C(F)(F)F)ccn1)c1cncc(C#Cc2cnc3ccc(N4CCC(N5CCOCC5)CC4)nn23)c1]

The compound can have the following structure:

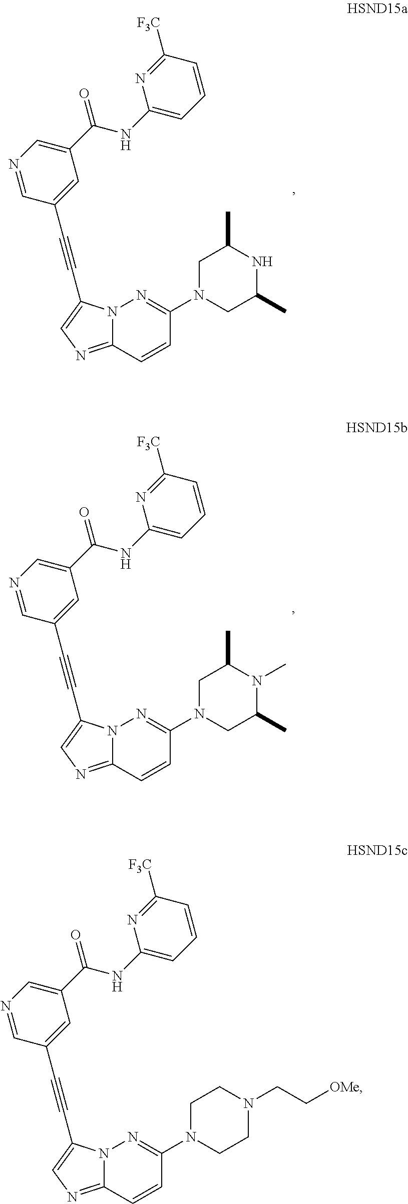 [SMILES: C[C@@H]1CN(c2ccc3ncc(C#Cc4cncc(C(=O)Nc5cccc(C(F)(F)F)n5)c4)n3n2)C[C@H](C)N1 ; C[C@@H]1CN(c2ccc3ncc(C#Cc4cncc(C(=O)Nc5cccc(C(F)(F)F)n5)c4)n3n2)C[C@H](C)N1C ; COCCN1CCN(c2ccc3ncc(C#Cc4cncc(C(=O)Nc5cccc(C(F)(F)F)n5)c4)n3n2)CC1]

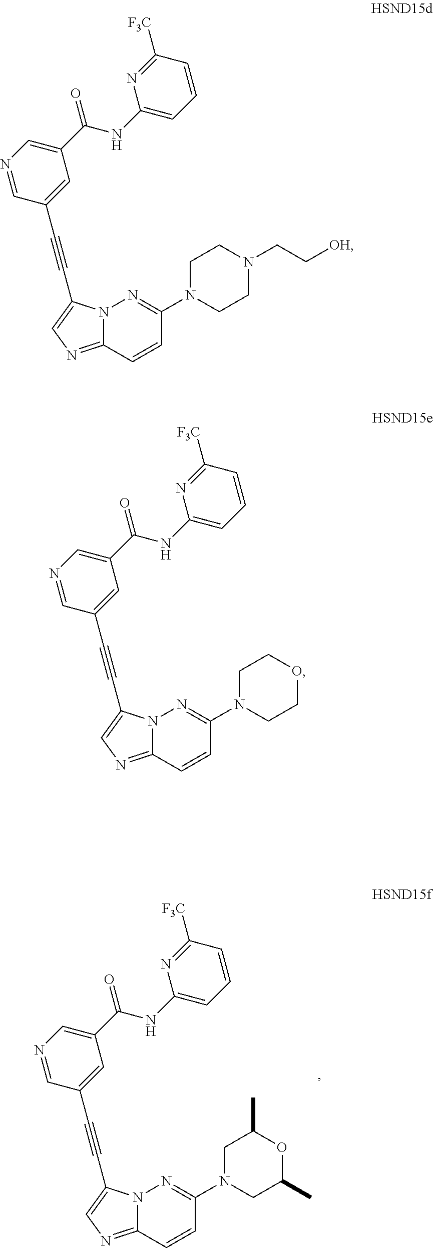 [SMILES: O=C(Nc1cccc(C(F)(F)F)n1)c1cncc(C#Cc2cnc3ccc(N4CCN(CCO)CC4)nn23)c1 ; O=C(Nc1cccc(C(F)(F)F)n1)c1cncc(C#Cc2cnc3ccc(N4CCOCC4)nn23)c1 ; C[C@@H]1CN(c2ccc3ncc(C#Cc4cncc(C(=O)Nc5cccc(C(F)(F)F)n5)c4)n3n2)C[C@H](C)O1]

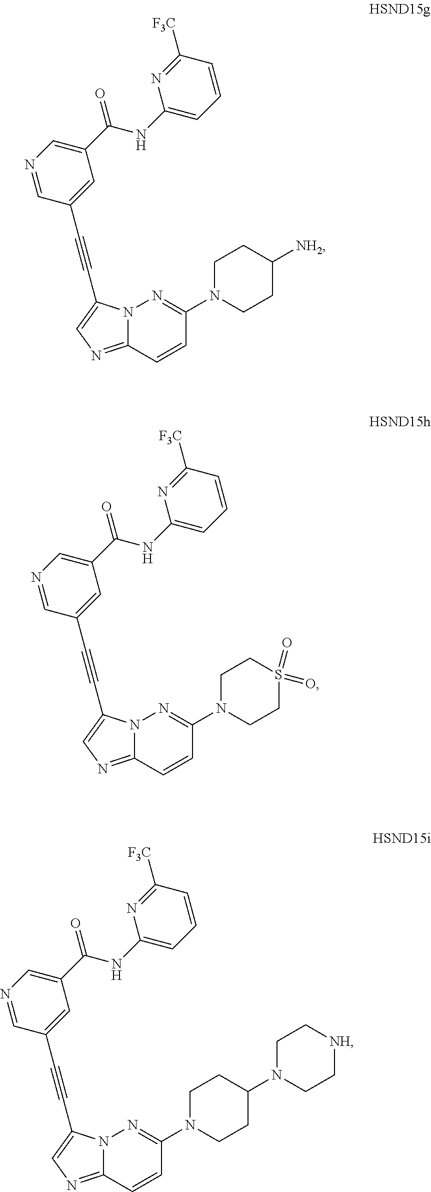 [SMILES: NC1CCN(c2ccc3ncc(C#Cc4cncc(C(=O)Nc5cccc(C(F)(F)F)n5)c4)n3n2)CC1 ; O=C(Nc1cccc(C(F)(F)F)n1)c1cncc(C#Cc2cnc3ccc(N4CCS(=O)(=O)CC4)nn23)c1 ; O=C(Nc1cccc(C(F)(F)F)n1)c1cncc(C#Cc2cnc3ccc(N4CCC(N5CCNCC5)CC4)nn23)c1]

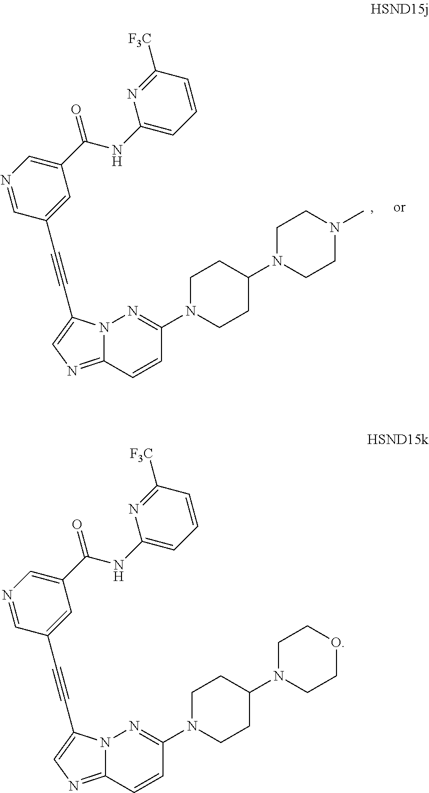 [SMILES: CN1CCN(C2CCN(c3ccc4ncc(C#Cc5cncc(C(=O)Nc6cccc(C(F)(F)F)n6)c5)n4n3)CC2)CC1 ; O=C(Nc1cccc(C(F)(F)F)n1)c1cncc(C#Cc2cnc3ccc(N4CCC(N5CCOCC5)CC4)nn23)c1]

The compound can have the following structure:

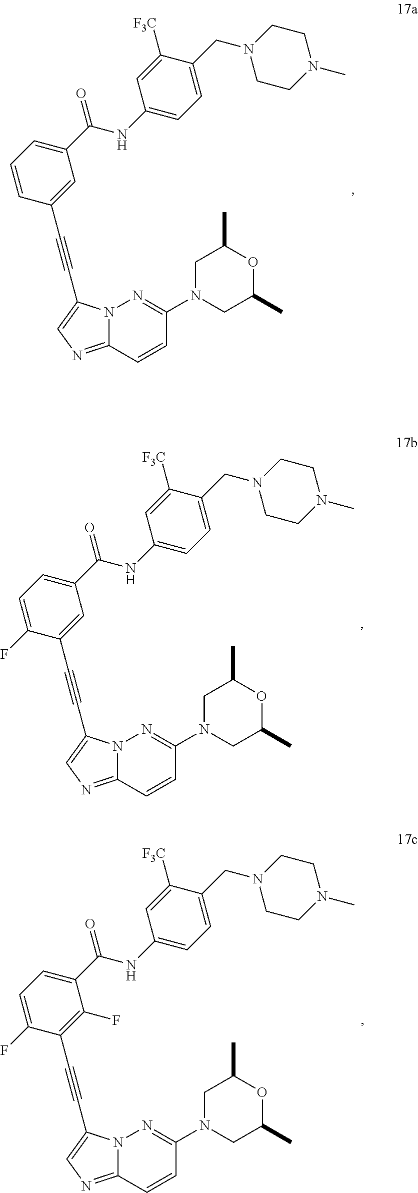 [SMILES: C[C@@H]1CN(c2ccc3ncc(C#Cc4cccc(C(=O)Nc5ccc(CN6CCN(C)CC6)c(C(F)(F)F)c5)c4)n3n2)C[C@H](C)O1 ; C[C@@H]1CN(c2ccc3ncc(C#Cc4cc(C(=O)Nc5ccc(CN6CCN(C)CC6)c(C(F)(F)F)c5)ccc4F)n3n2)C[C@H](C)O1 ; C[C@@H]1CN(c2ccc3ncc(C#Cc4c(F)ccc(C(=O)Nc5ccc(CN6CCN(C)CC6)c(C(F)(F)F)c5)c4F)n3n2)C[C@H](C)O1]

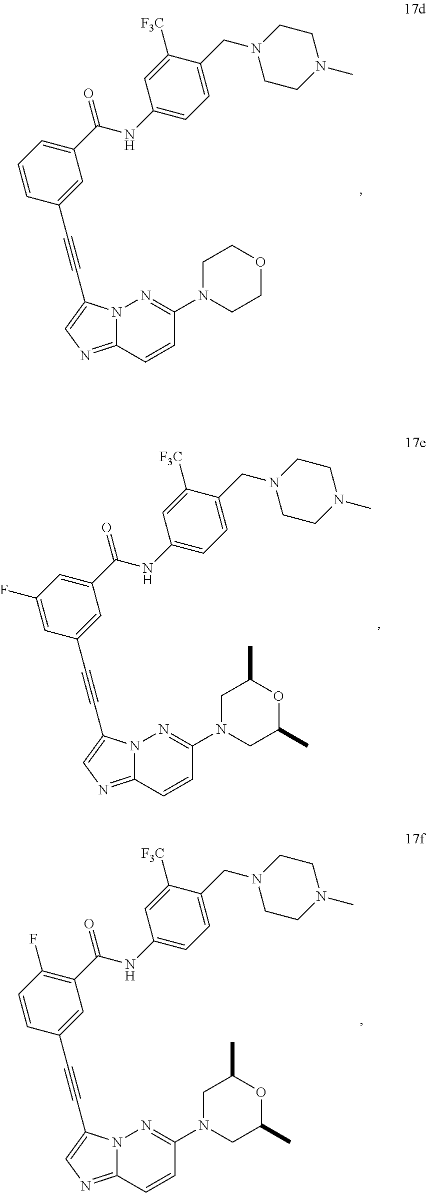 [SMILES: CN1CCN(Cc2ccc(NC(=O)c3cccc(C#Cc4cnc5ccc(N6CCOCC6)nn45)c3)cc2C(F)(F)F)CC1 ; C[C@@H]1CN(c2ccc3ncc(C#Cc4cc(F)cc(C(=O)Nc5ccc(CN6CCN(C)CC6)c(C(F)(F)F)c5)c4)n3n2)C[C@H](C)O1 ; C[C@@H]1CN(c2ccc3ncc(C#Cc4ccc(F)c(C(=O)Nc5ccc(CN6CCN(C)CC6)c(C(F)(F)F)c5)c4)n3n2)C[C@H](C)O1]

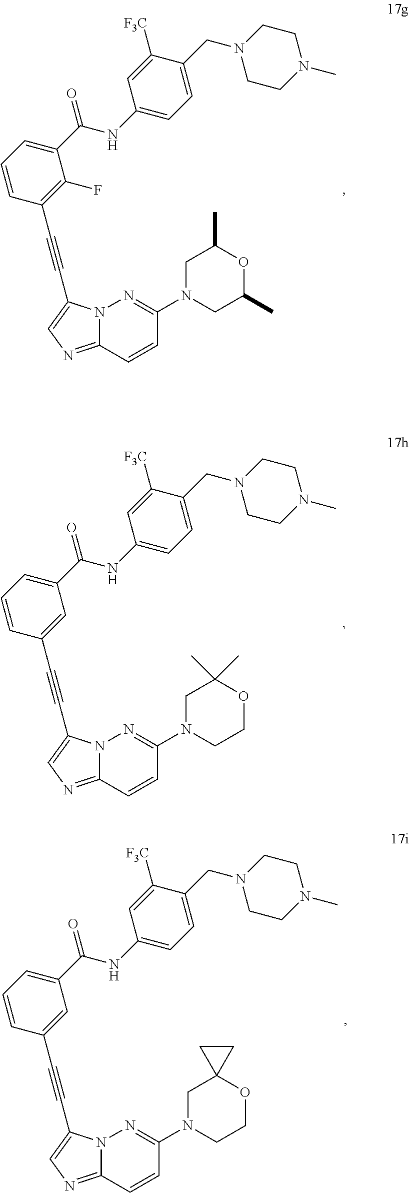 [SMILES: C[C@@H]1CN(c2ccc3ncc(C#Cc4cccc(C(=O)Nc5ccc(CN6CCN(C)CC6)c(C(F)(F)F)c5)c4F)n3n2)C[C@H](C)O1 ; CN1CCN(Cc2ccc(NC(=O)c3cccc(C#Cc4cnc5ccc(N6CCOC(C)(C)C6)nn45)c3)cc2C(F)(F)F)CC1 ; CN1CCN(Cc2ccc(NC(=O)c3cccc(C#Cc4cnc5ccc(N6CCOC7(CC7)C6)nn45)c3)cc2C(F)(F)F)CC1]

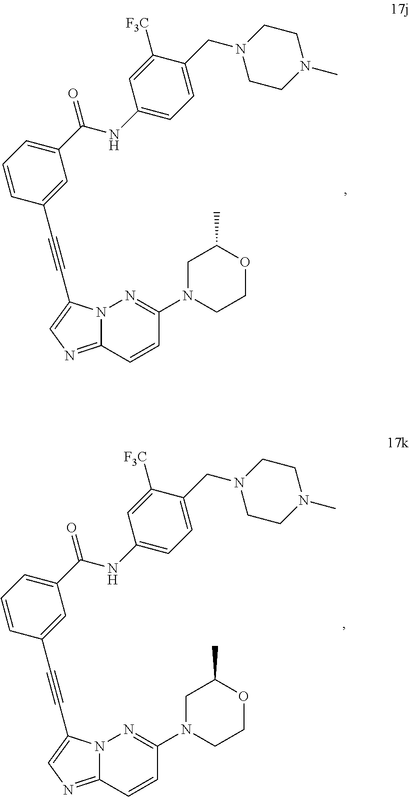 [SMILES: C[C@H]1CN(c2ccc3ncc(C#Cc4cccc(C(=O)Nc5ccc(CN6CCN(C)CC6)c(C(F)(F)F)c5)c4)n3n2)CCO1 ; C[C@@H]1CN(c2ccc3ncc(C#Cc4cccc(C(=O)Nc5ccc(CN6CCN(C)CC6)c(C(F)(F)F)c5)c4)n3n2)CCO1]

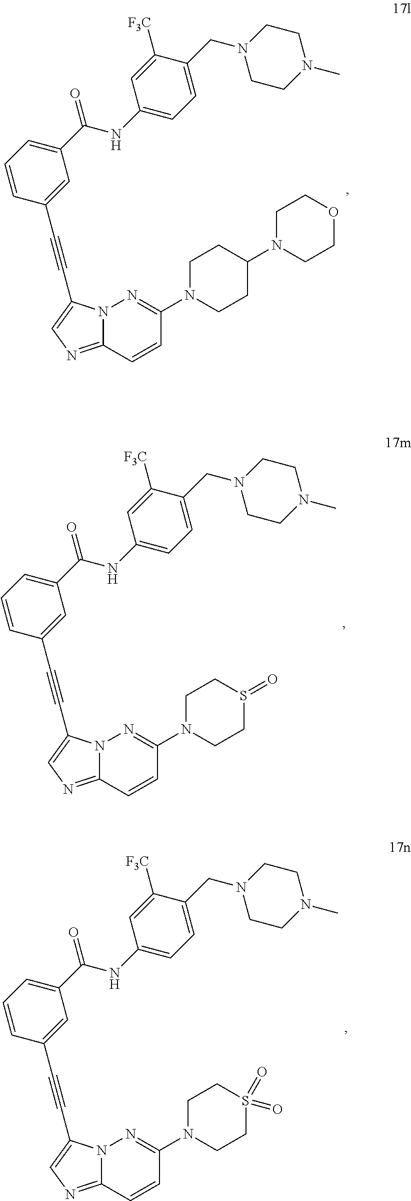 [SMILES: CN1CCN(Cc2ccc(NC(=O)c3cccc(C#Cc4cnc5ccc(N6CCC(N7CCOCC7)CC6)nn45)c3)cc2C(F)(F)F)CC1 ; CN1CCN(Cc2ccc(NC(=O)c3cccc(C#Cc4cnc5ccc(N6CCS(=O)CC6)nn45)c3)cc2C(F)(F)F)CC1 ; CN1CCN(Cc2ccc(NC(=O)c3cccc(C#Cc4cnc5ccc(N6CCS(=O)(=O)CC6)nn45)c3)cc2C(F)(F)F)CC1]

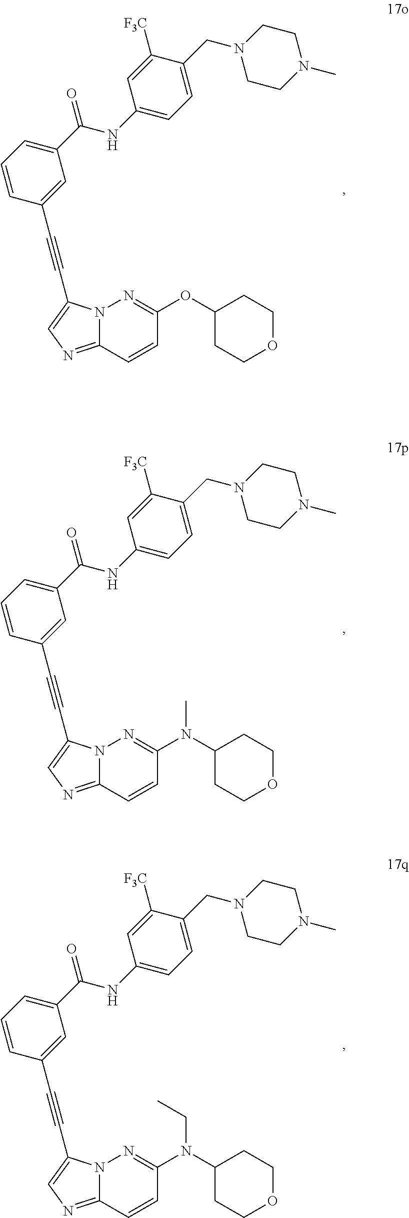 [SMILES: CN1CCN(Cc2ccc(NC(=O)c3cccc(C#Cc4cnc5ccc(OC6CCOCC6)nn45)c3)cc2C(F)(F)F)CC1 ; CN1CCN(Cc2ccc(NC(=O)c3cccc(C#Cc4cnc5ccc(N(C)C6CCOCC6)nn45)c3)cc2C(F)(F)F)CC1 ; CCN(c1ccc2ncc(C#Cc3cccc(C(=O)Nc4ccc(CN5CCN(C)CC5)c(C(F)(F)F)c4)c3)n2n1)C1CCOCC1]

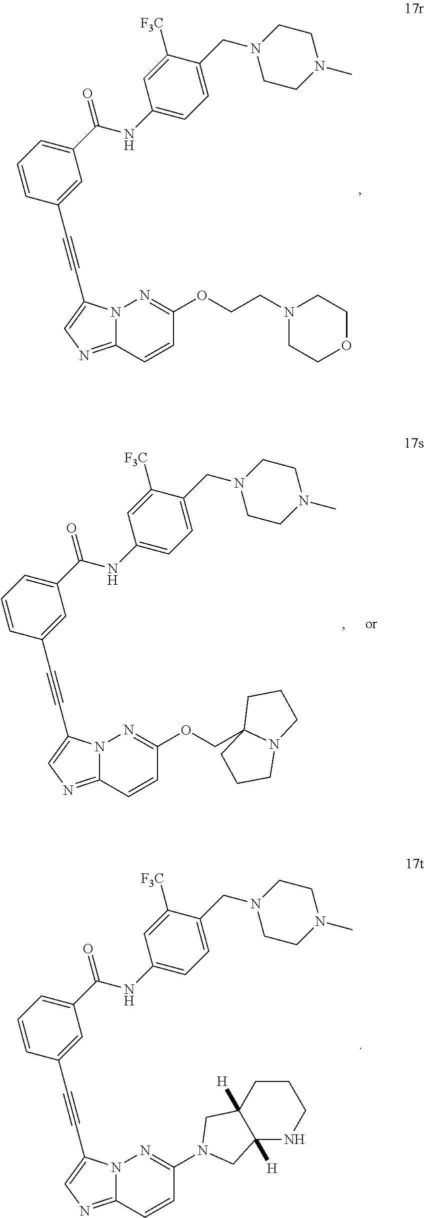 [SMILES: CN1CCN(Cc2ccc(NC(=O)c3cccc(C#Cc4cnc5ccc(OCCN6CCOCC6)nn45)c3)cc2C(F)(F)F)CC1 ; CN1CCN(Cc2ccc(NC(=O)c3cccc(C#Cc4cnc5ccc(OCC67CCCN6CCC7)nn45)c3)cc2C(F)(F)F)CC1 ; CN1CCN(Cc2ccc(NC(=O)c3cccc(C#Cc4cnc5ccc(N6C[C@@H]7CCCN[C@@H]7C6)nn45)c3)cc2C(F)(F)F)CC1]

The compound can have the following structure:

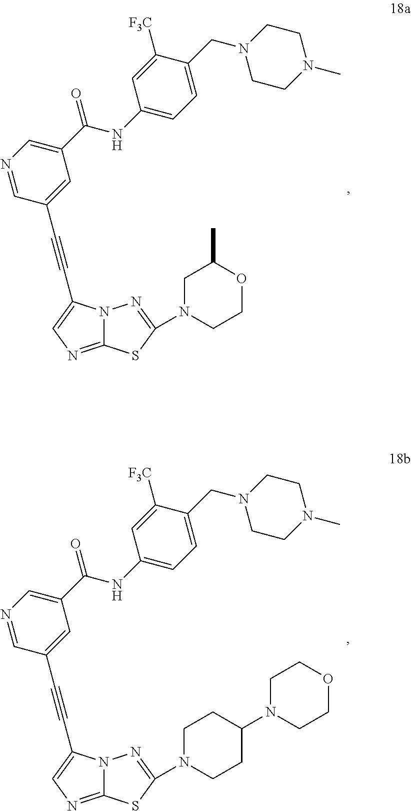 [SMILES: C[C@@H]1CN(c2nn3c(C#Cc4cncc(C(=O)Nc5ccc(CN6CCN(C)CC6)c(C(F)(F)F)c5)c4)cnc3s2)CCO1 ; CN1CCN(Cc2ccc(NC(=O)c3cncc(C#Cc4cnc5sc(N6CCC(N7CCOCC7)CC6)nn45)c3)cc2C(F)(F)F)CC1]

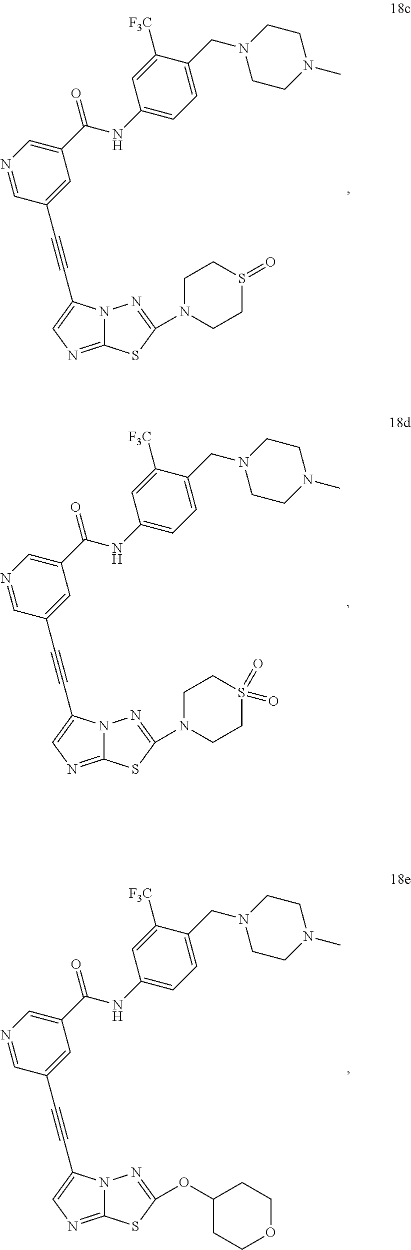 [SMILES: CN1CCN(Cc2ccc(NC(=O)c3cncc(C#Cc4cnc5sc(N6CCS(=O)CC6)nn45)c3)cc2C(F)(F)F)CC1 ; CN1CCN(Cc2ccc(NC(=O)c3cncc(C#Cc4cnc5sc(N6CCS(=O)(=O)CC6)nn45)c3)cc2C(F)(F)F)CC1 ; CN1CCN(Cc2ccc(NC(=O)c3cncc(C#Cc4cnc5sc(OC6CCOCC6)nn45)c3)cc2C(F)(F)F)CC1]

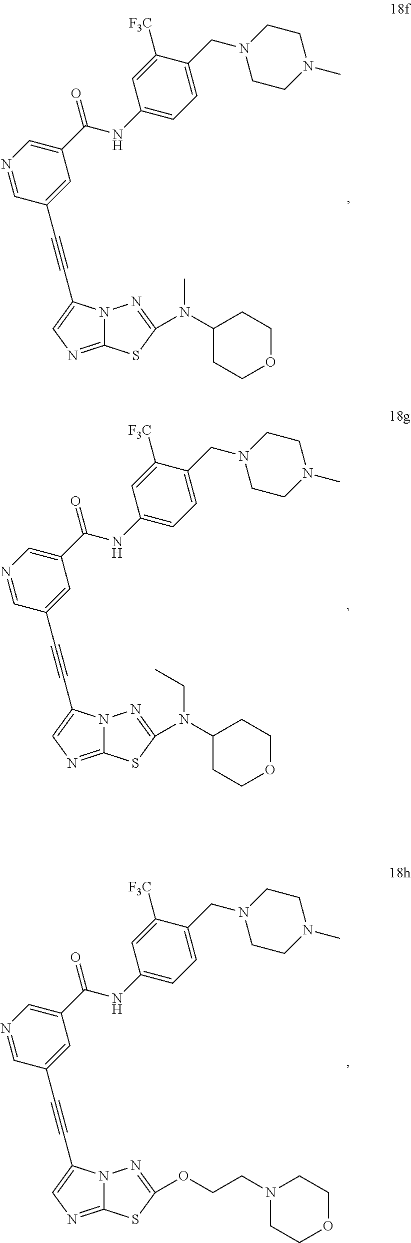 [SMILES: CN1CCN(Cc2ccc(NC(=O)c3cncc(C#Cc4cnc5sc(N(C)C6CCOCC6)nn45)c3)cc2C(F)(F)F)CC1 ; CCN(c1nn2c(C#Cc3cncc(C(=O)Nc4ccc(CN5CCN(C)CC5)c(C(F)(F)F)c4)c3)cnc2s1)C1CCOCC1 ; CN1CCN(Cc2ccc(NC(=O)c3cncc(C#Cc4cnc5sc(OCCN6CCOCC6)nn45)c3)cc2C(F)(F)F)CC1]

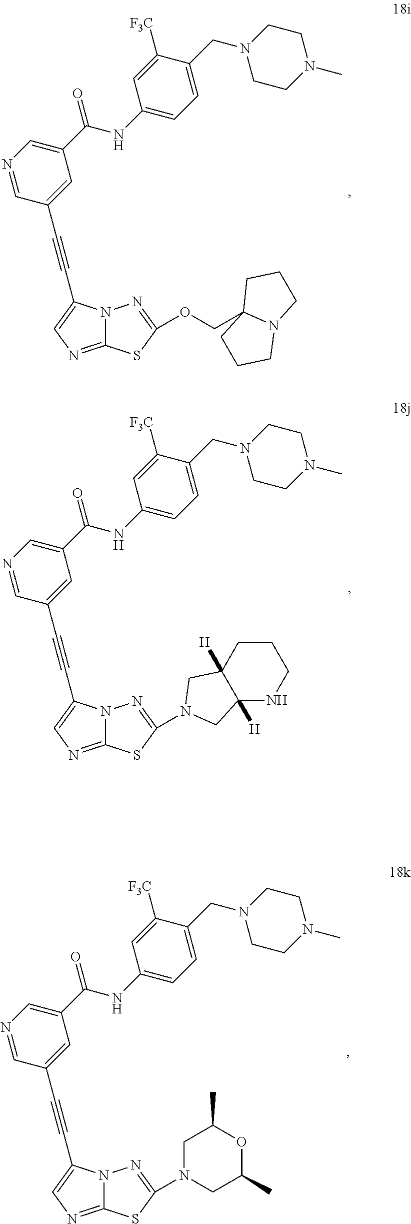 [SMILES: CN1CCN(Cc2ccc(NC(=O)c3cncc(C#Cc4cnc5sc(OCC67CCCN6CCC7)nn45)c3)cc2C(F)(F)F)CC1 ; CN1CCN(Cc2ccc(NC(=O)c3cncc(C#Cc4cnc5sc(N6C[C@@H]7CCCN[C@@H]7C6)nn45)c3)cc2C(F)(F)F)CC1 ; C[C@@H]1CN(c2nn3c(C#Cc4cncc(C(=O)Nc5ccc(CN6CCN(C)CC6)c(C(F)(F)F)c5)c4)cnc3s2)C[C@H](C)O1]

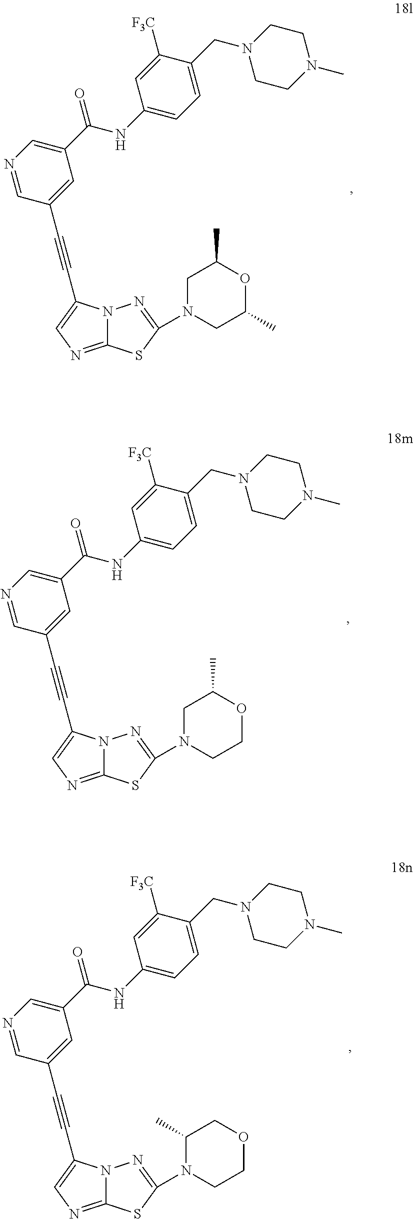 [SMILES: C[C@@H]1CN(c2nn3c(C#Cc4cncc(C(=O)Nc5ccc(CN6CCN(C)CC6)c(C(F)(F)F)c5)c4)cnc3s2)C[C@@H](C)O1 ; C[C@H]1CN(c2nn3c(C#Cc4cncc(C(=O)Nc5ccc(CN6CCN(C)CC6)c(C(F)(F)F)c5)c4)cnc3s2)CCO1 ; C[C@@H]1COCCN1c1nn2c(C#Cc3cncc(C(=O)Nc4ccc(CN5CCN(C)CC5)c(C(F)(F)F)c4)c3)cnc2s1]

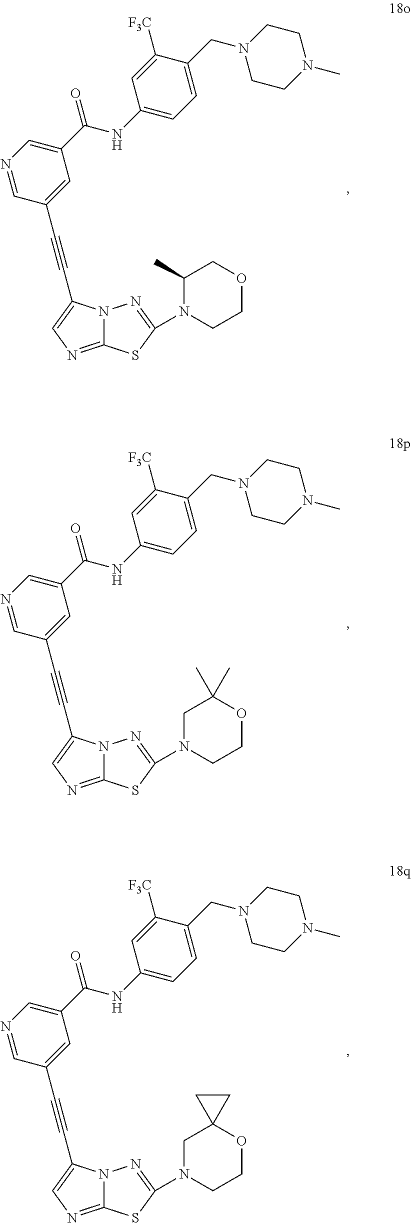 [SMILES: C[C@H]1COCCN1c1nn2c(C#Cc3cncc(C(=O)Nc4ccc(CN5CCN(C)CC5)c(C(F)(F)F)c4)c3)cnc2s1 ; CN1CCN(Cc2ccc(NC(=O)c3cncc(C#Cc4cnc5sc(N6CCOC(C)(C)C6)nn45)c3)cc2C(F)(F)F)CC1 ; CN1CCN(Cc2ccc(NC(=O)c3cncc(C#Cc4cnc5sc(N6CCOC7(CC7)C6)nn45)c3)cc2C(F)(F)F)CC1]

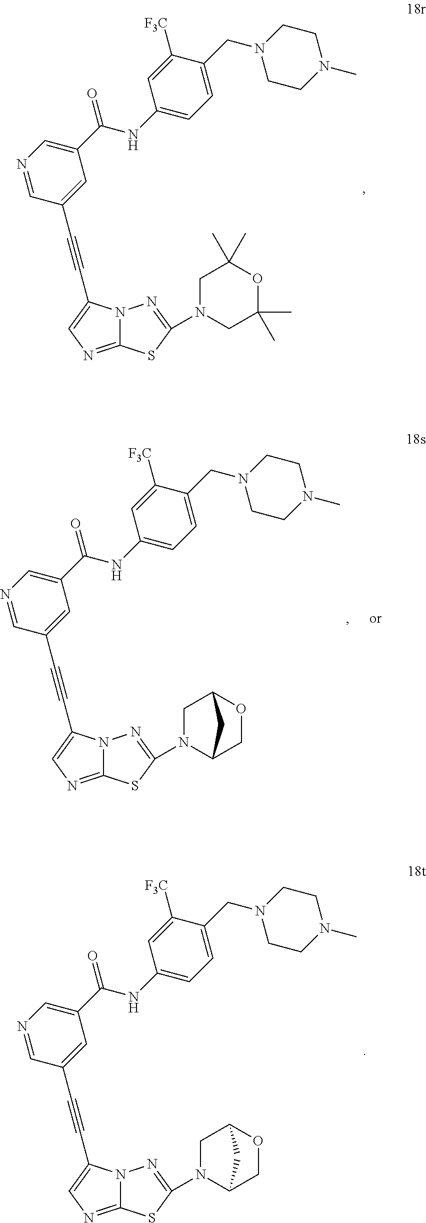 [SMILES: CN1CCN(Cc2ccc(NC(=O)c3cncc(C#Cc4cnc5sc(N6CC(C)(C)OC(C)(C)C6)nn45)c3)cc2C(F)(F)F)CC1 ; CN1CCN(Cc2ccc(NC(=O)c3cncc(C#Cc4cnc5sc(N6C[C@H]7C[C@@H]6CO7)nn45)c3)cc2C(F)(F)F)CC1 ; CN1CCN(Cc2ccc(NC(=O)c3cncc(C#Cc4cnc5sc(N6C[C@@H]7C[C@H]6CO7)nn45)c3)cc2C(F)(F)F)CC1]

The compound can have the following structure:

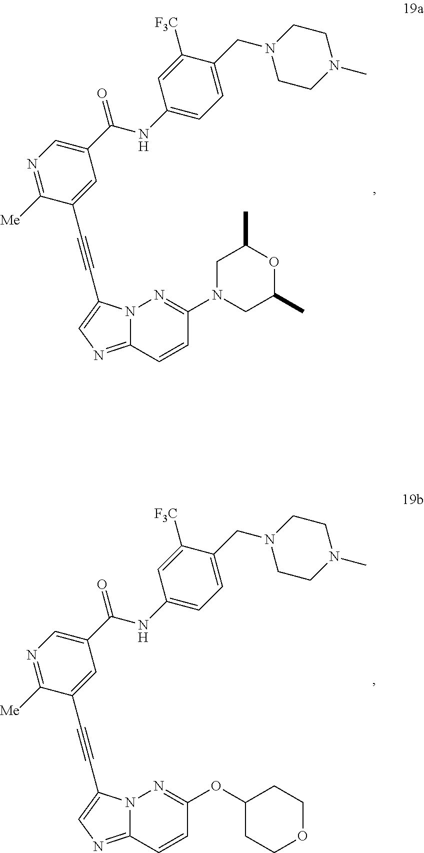 [SMILES: Cc1ncc(C(=O)Nc2ccc(CN3CCN(C)CC3)c(C(F)(F)F)c2)cc1C#Cc1cnc2ccc(N3C[C@@H](C)O[C@@H](C)C3)nn12 ; Cc1ncc(C(=O)Nc2ccc(CN3CCN(C)CC3)c(C(F)(F)F)c2)cc1C#Cc1cnc2ccc(OC3CCOCC3)nn12]

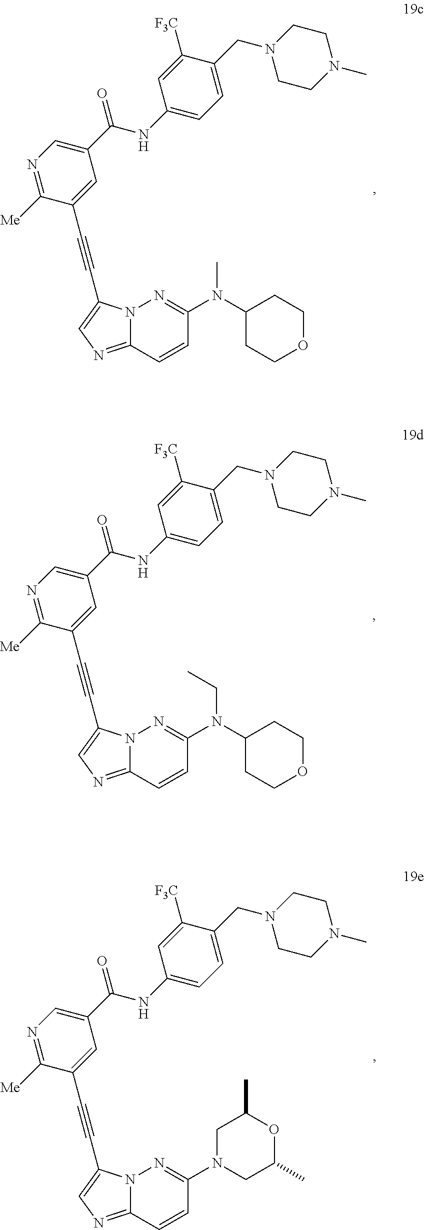 [SMILES: Cc1ncc(C(=O)Nc2ccc(CN3CCN(C)CC3)c(C(F)(F)F)c2)cc1C#Cc1cnc2ccc(N(C)C3CCOCC3)nn12 ; CCN(c1ccc2ncc(C#Cc3cc(C(=O)Nc4ccc(CN5CCN(C)CC5)c(C(F)(F)F)c4)cnc3C)n2n1)C1CCOCC1 ; Cc1ncc(C(=O)Nc2ccc(CN3CCN(C)CC3)c(C(F)(F)F)c2)cc1C#Cc1cnc2ccc(N3C[C@@H](C)O[C@H](C)C3)nn12]

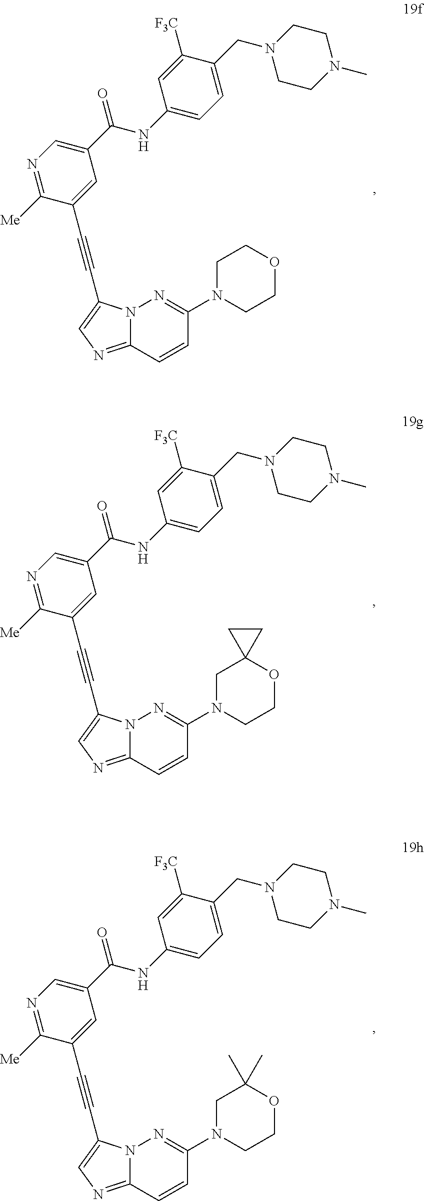 [SMILES: Cc1ncc(C(=O)Nc2ccc(CN3CCN(C)CC3)c(C(F)(F)F)c2)cc1C#Cc1cnc2ccc(N3CCOCC3)nn12 ; Cc1ncc(C(=O)Nc2ccc(CN3CCN(C)CC3)c(C(F)(F)F)c2)cc1C#Cc1cnc2ccc(N3CCOC4(CC4)C3)nn12 ; Cc1ncc(C(=O)Nc2ccc(CN3CCN(C)CC3)c(C(F)(F)F)c2)cc1C#Cc1cnc2ccc(N3CCOC(C)(C)C3)nn12]

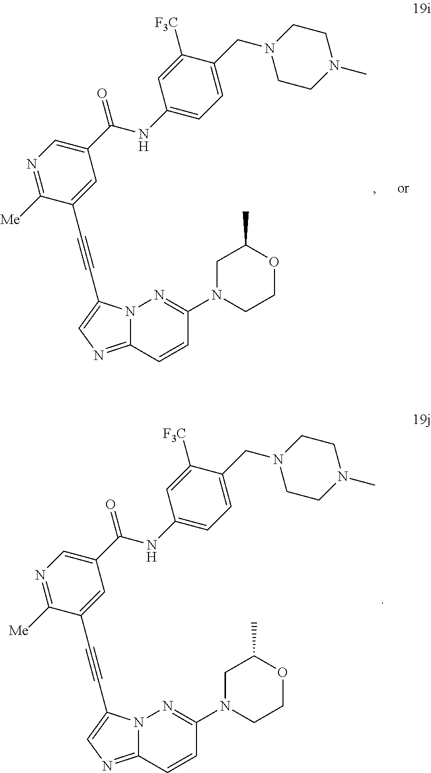 [SMILES: Cc1ncc(C(=O)Nc2ccc(CN3CCN(C)CC3)c(C(F)(F)F)c2)cc1C#Cc1cnc2ccc(N3CCO[C@H](C)C3)nn12 ; Cc1ncc(C(=O)Nc2ccc(CN3CCN(C)CC3)c(C(F)(F)F)c2)cc1C#Cc1cnc2ccc(N3CCO[C@@H](C)C3)nn12]

The compound can have a structure of:

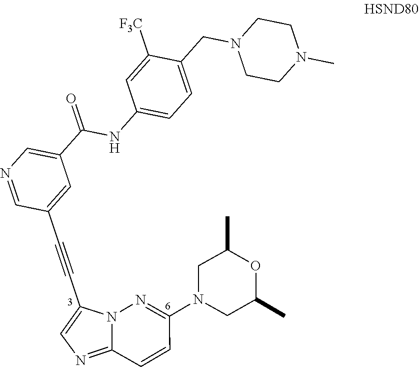 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)O1]

or be a pharmaceutically acceptable salt thereof

The compound can have a structure of:

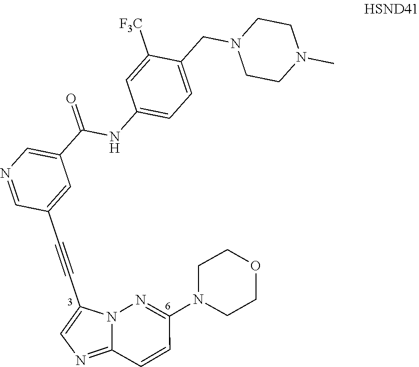 [SMILES: CN1CCN(Cc2ccc(NC(=O)c3cncc(C#Cc4cnc5ccc(N6CCOCC6)nn45)c3)cc2C(F)(F)F)CC1]

or be a pharmaceutically acceptable salt thereof

The compound can have a structure of:

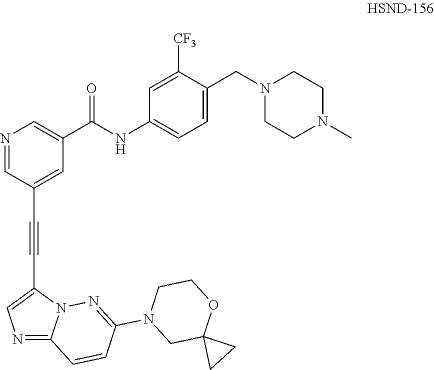 [SMILES: CN1CCN(Cc2ccc(NC(=O)c3cncc(C#Cc4cnc5ccc(N6CCOC7(CC7)C6)nn45)c3)cc2C(F)(F)F)CC1]

or be a pharmaceutically acceptable salt thereof.

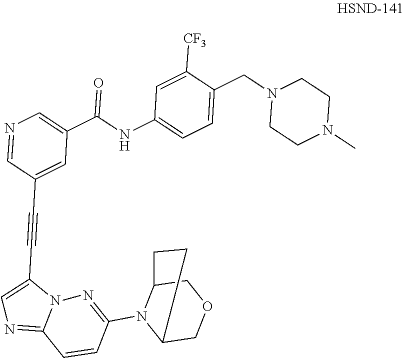 [SMILES: CN1CCN(Cc2ccc(NC(=O)c3cncc(C#Cc4cnc5ccc(N6C7CCC6COC7)nn45)c3)cc2C(F)(F)F)CC1]

or be a pharmaceutically acceptable salt thereof.

The compound can have a structure of:

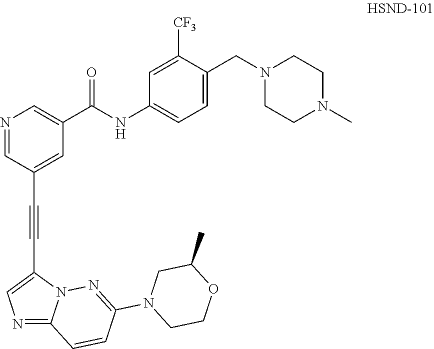 [SMILES: C[C@@H]1CN(c2ccc3ncc(C#Cc4cncc(C(=O)Nc5ccc(CN6CCN(C)CC6)c(C(F)(F)F)c5)c4)n3n2)CCO1]

or be a pharmaceutically acceptable salt thereof.

The compound can have a structure of:

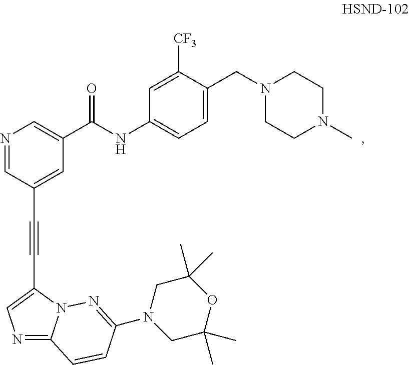 [SMILES: CN1CCN(Cc2ccc(NC(=O)c3cncc(C#Cc4cnc5ccc(N6CC(C)(C)OC(C)(C)C6)nn45)c3)cc2C(F)(F)F)CC1]

or be a pharmaceutically acceptable salt thereof,

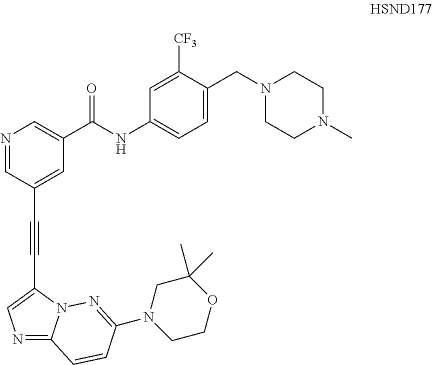 [SMILES: CN1CCN(Cc2ccc(NC(=O)c3cncc(C#Cc4cnc5ccc(N6CCOC(C)(C)C6)nn45)c3)cc2C(F)(F)F)CC1]

or be a pharmaceutically acceptable salt thereof

The compound can have a structure of

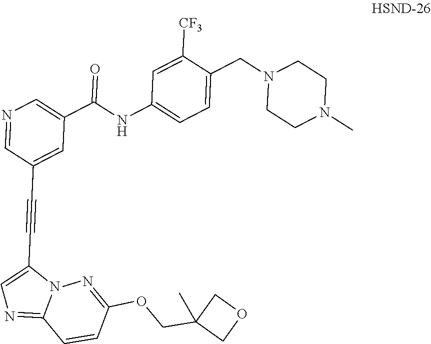 [SMILES: CN1CCN(Cc2ccc(NC(=O)c3cncc(C#Cc4cnc5ccc(OCC6(C)COC6)nn45)c3)cc2C(F)(F)F)CC1]

or be a pharmaceutically acceptable salt thereof

The compound can have a structure of

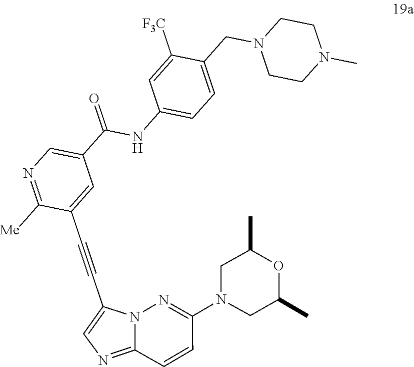 [SMILES: Cc1ncc(C(=O)Nc2ccc(CN3CCN(C)CC3)c(C(F)(F)F)c2)cc1C#Cc1cnc2ccc(N3C[C@@H](C)O[C@@H](C)C3)nn12]

or be a pharmaceutically acceptable salt thereof

The compound can have a structure of:

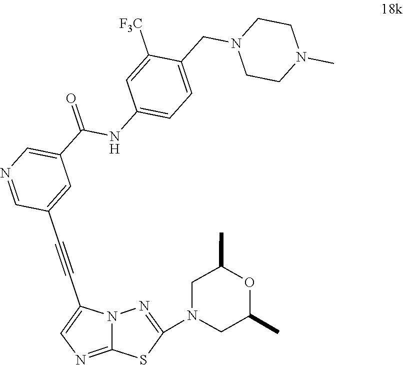 [SMILES: C[C@@H]1CN(c2nn3c(C#Cc4cncc(C(=O)Nc5ccc(CN6CCN(C)CC6)c(C(F)(F)F)c5)c4)cnc3s2)C[C@H](C)O1]

or be a pharmaceutically acceptable salt thereof.

The compound can have a structure of

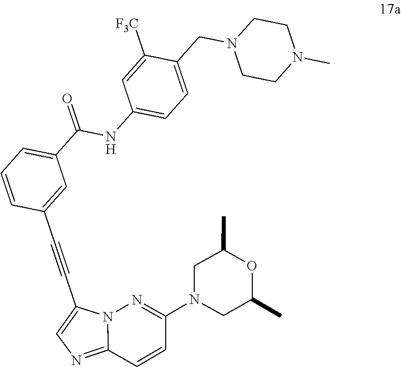 [SMILES: C[C@@H]1CN(c2ccc3ncc(C#Cc4cccc(C(=O)Nc5ccc(CN6CCN(C)CC6)c(C(F)(F)F)c5)c4)n3n2)C[C@H](C)O1]

or be a pharmaceutically acceptable salt thereof.

It is contemplated that the compounds hereof can be provided in prodrug form. The term “prodrug” means an inactive derivative of a parent compound/drug that can hydrolyze, oxidize, or otherwise react under biological conditions (in vitro or in vivo) to provide an active compound, particularly a compound hereof. Prodrugs can be created to overcome one or more barriers to the effective use of the underlying active compound such as instability and/or possible toxicity barriers that exist with the active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. In certain embodiments, a prodrug form of a compound hereof can comprise a protecting group that will, in addition to protecting the compound from oxidation or the like, permit the targeting of specific sites within a subject's body (e.g., a tumor microenvironment). Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

### PROTAC Conjugates

PROTAC conjugates comprising the compounds hereof are also provided. A PROTAC conjugate can have a chemical structure of Formula (X):

A-L′-D (X)

wherein A is a compound described herein (e.g., a kinase inhibitor described herein); L′ is a linker or absent; and D is a ubiquitin pathway protein binding moiety.

While inhibitors of proteins can block or reduce protein activity in a cell, protein degradation in a cell can also reduce activity or remove altogether the target protein. Utilizing a cell's protein degradation pathway can, therefore, provide additional means for reducing or removing protein activity. One of the cell's major degradation pathways is known as the ubiquitin-proteasome system. In this system, a protein is marked for degradation by the proteasome by ubiquitinating the protein. The ubiquitination of the protein can be accomplished by a ubiquitin pathway protein binding moiety that binds to a protein and adds ubiquitin molecules to the protein.

To harness this degradation pathway, PROTACs have been developed. The term “PROTAC” refers to proteolysis-targeting chimera molecules comprising a ubiquitin pathway protein binding moiety, optionally a linker, and targeting moiety. PROTACs bring together the ubiquitin pathway protein binding moiety (e.g., an E3 ubiquitin ligase) with a protein that is to be targeted for degradation. To facilitate a protein for degradation by the proteasome, the PROTAC is comprised of a group that binds to an ubiquitin ligase and a group that binds to the protein targeted for degradation (optionally these groups are connected with a linker). This molecular construct can bring the ubiquitin pathway protein binding moiety in proximity with the protein so that it is ubiquitinated and marked for degradation.

The ubiquitin pathway protein binding moiety is any suitable structure that recognizes and binds to a ubiquitin pathway protein. In general, a ubiquitin pathway protein is any entity or complex that is capable of catalyzing or causing to catalyze the transfer of a ubiquitin or ubiquitin-like modifying polypeptide (e.g., Nedd8, APG12, or ISG15/UCRP) to another protein. In certain embodiments, the ubiquitin pathway protein is a ubiquitin protein ligase or E3 protein. There are at least 100 distinct E3 proteins encoded by the human genome. Winston et al., A Family of Mammalian F-Box Proteins, Current Biology 9(20): 1180-1182 (1999).

In certain embodiments, a ubiquitin pathway protein is a protein that is involved in or a component of a ubiquitin-like pathway, which transfers ubiquitin-like modifying polypeptides (e.g., SUMO, Nedd8, APG12, or ISG15/UCRP). Components of a ubiquitin-like modifying pathway are usually homologs of a ubiquitin pathway. For example, the ubiquitin-like pathway for SUMO includes a homolog of a ubiquitin protein activating enzyme or E1 protein, ubiquitin protein conjugating enzyme or E2 protein and ubiquitin ligase or E3 protein.

In certain embodiments, a ubiquitin pathway protein binding moiety is any suitable ligand to a ubiquitin pathway protein, for example, ubiquitin protein ligase or E3 protein or homologs thereof. In certain embodiments, a ubiquitin pathway protein binding peptide, domain or region of a ligand to a ubiquitin pathway protein. In certain embodiments, a ubiquitin pathway protein binding moiety recognizes and binds to a ubiquitin pathway protein in a regulated manner.

The A component is a compound hereof that binds to a target protein intended to be degraded (e.g., a targeted kinase inhibitor). The term “protein” includes oligopeptides and polypeptide sequences of sufficient length that they can bind to the A component.

Specific A groups are any of the compounds described herein.

The compound and the ubiquitin pathway protein binding moiety can be optionally connected with a linker. The linker can be any suitable linker. The linker can comprise atoms selected from C, N, O, S, Si, and P; C, N, O, S, and P; or C, N, O, and S. The linker can have a backbone that ranges in length, such that there can be as few as two atoms in the backbone of the linker to as many as 100 or more contiguous atoms in the backbone of the linker. The “backbone” of the linker is the shortest chain of contiguous atoms forming a covalently bonded connection between A and D. In some embodiments, a polyvalent linker has a branched backbone, with each branch serving as a section of backbone linker until reaching a terminus.

For example, the linker can have a chain length of at least about 7 atoms. In some embodiments, the linker is at least about 10 atoms in length. In some embodiments, the linker is at least about 14 atoms in length. In some embodiments, the linker is between about 7 and about 31 atoms (such as, about 7 to 31, 7 to about 31, or 7 to 31), between about 7 and about 24 atoms (such as, about 7 to 24, 7 to about 24, or 7 to 24), or between about 7 and about 20 atoms (such as, about 7 to 20, 7 to about 20, or 7 to 20) atoms. In some embodiments, the linker is between about 14 and about 31 atoms (such as, about 14 to 31, 14 to about 31, or 14 to 31), between about 14 and about 24 atoms (such as, about 14 to 24, 14 to about 24, or 14 to 24), or between about 14 and about 20 atoms (such as, about 14 to 20, 14 to about 20, or 14 to 20). In some embodiments, the linker has a chain length of at least 7 atoms, at least 14 atoms, at least 20 atoms, at least 25 atoms, at least 30 atoms, or at least 40 atoms; or from 1 to 15 atoms, 1 to 5 atoms, 5 to 10 atoms, 5 to 20 atoms, 10 to 40 atoms, or 25 to 100 atoms.

The linker can comprise at least one carbon-carbon bond and/or at least one amide bond. The linker can comprise one or more L- or D-configurations, natural or unnatural amino acids, a PEG monomer, a PEG oligomer, a PEG polymer, or a combination of any of the foregoing. For a linker that comprises one or more PEG units, all carbon and oxygen atoms of the PEG units are part of the backbone, unless otherwise specified.

In certain embodiments, the linker is a group comprising one or more covalently connected structural units.

In certain embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, and/or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker is asymmetric. In certain embodiments, the linker is symmetrical.

Alternatively, or in addition to chain length, in some embodiments, the linker can have suitable substituents that affect hydrophobicity or hydrophilicity. Thus, for example, the linker can have a hydrophobic side chain group, such as an alkyl, cycloalkyl, aryl, arylalkyl, or like group, each of which is optionally substituted. If the linker includes one or more amino acids, the linker can contain hydrophobic amino acid side chains, such as one or more amino acid side chains from Phe and Tyr, including substituted variants thereof, and analogs and derivatives of such side chains.

The linker can comprise a spacer (e.g., be conjugated with and/or include a spacer). The spacer can be any suitable spacer. A spacer of the linker can comprise hydrophilic, hydrophobic, amphipathic, non-peptidic, peptidic, and/or aromatic monomers. A length of a spacer can range from 1 to 30 (e.g., 1 to 30 carbon atoms, a PEG with 1-30 units, etc.). Examples of hydrophilic spacers include, but are not limited to, polyethylene glycol polymers and derivatives thereof. Examples of hydrophobic spacers include, but are not limited to, pure or mixed branched hydrocarbons, fluorocarbons, alkane, alkene, and/or alkyne polymers. Examples of amphipathic spacers include, but are not limited to, pure or mixed phospholipids and/or derivatives thereof. Examples of peptidic spacers include, but are not limited to, pure and mixed single, branched, L- or D-configurations, essential, nonessential, natural, and unnatural amino acids and derivatives thereof. Examples of aromatic spacers include, but are not limited to, pure and mixed repeated quinoids.

In some embodiments the linker is formed via click chemistry/click chemistry-derived synthetic methods. Those of skill in the art understand that the terms “click chemistry” and “click chemistry-derived” generally refer to a class of small molecule reactions commonly used in conjugation, allowing the joining of substrates of choice with specific molecules. Click chemistry is not a single specific reaction but describes a way of generating products that follow examples in nature, which also generate substances by joining small modular units. In many applications click reactions join a biomolecule and a reporter molecule. Click chemistry is not limited to biological conditions; the concept of a “click” reaction has been used in pharmacological and various biomimetic applications. However, they have been made notably useful in the detection, localization and qualification of biomolecules.

Click reactions can occur in one pot, typically are not disturbed by water, can generate minimal byproducts, and are “spring-loaded”—characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (e.g., for in vivo systems).

### Salts

The compounds and conjugates can be presented as a pharmaceutically acceptable salt. Examples of acceptable salts include, without limitation, alkali metal (e.g., sodium, potassium, or lithium) or alkaline earth metal (e.g., calcium) salts; however, any salt that is generally non-toxic and effective when administered to the subject being treated is acceptable. Similarly, “pharmaceutically acceptable salt” refers to those salts with counter ions, which can be used in pharmaceuticals. More specifically with respect to the present disclosure, the terms “salts” and “pharmaceutically acceptable salts” as used herein refer to derivatives of the disclosed compounds wherein the parent compound is modified by making acid or base salts thereof.

Such salts can include, without limitation, (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or (2) salts formed when an acidic proton present in the parent compound either is replaced by a metal ion, e.g., an alkali metal ion, an alkaline earth ion, or an aluminum ion, or coordinated with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

Acceptable salts can be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds can be formed from bases that form non-toxic salts. Illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemi-salts of acids and bases, such as hemi-sulphate and hemi-calcium salts, also can be formed.

The compounds and conjugates hereof can be “deuterated,” meaning one or more hydrogen atoms can be replaced with deuterium. As deuterium and hydrogen have nearly the same physical properties, deuterium substitution is the smallest structural change that can be made. Deuteration is well known to those of ordinary skill in the art.

The compounds and conjugates, in some embodiments, can contain one or more asymmetric centers and thus give rise to enantiomers, diastereomers, and other stereoisomeric forms that are defined, in terms of absolute stereochemistry, as (R)- or (S)-. In certain embodiments, the compound or conjugate is of R-configuration. In certain embodiments, the compound or conjugate is of S-configuration. Unless stated otherwise, it is intended that all stereoisomeric forms of the compounds and/or conjugates are contemplated. When the compounds and/or conjugates contain alkene double bonds, and unless specified otherwise, it is intended that both E and Z geometric isomers (e.g., cis or trans) and/or optical isomers are included. In certain embodiments, for example, D and A of a conjugate are arranged in a relative cis orientation. In certain embodiments, D and A of a conjugate are arranged in a relative trans orientation. Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Further, in each of the foregoing and following embodiments, it is to be understood that the formulae include and represent not only all pharmaceutically acceptable salts of the compounds, but also include any and all hydrates and/or solvates of the compound/conjugate formulae or salts thereof. Indeed, hydrates, solvates, and N-oxides of the compounds and conjugates are also contemplated. The term “solvate” means a compound, or a salt thereof, that further includes a stoichiometric or non-stoichiometric amount of solvent bound by non-covalent intermolecular forces. Where the solvent is water, the solvate is a hydrate.

It is to be appreciated that certain functional groups, such as the hydroxy, amino, and like groups form complexes and/or coordination compounds with water and/or various solvents, in the various physical forms of the compounds and conjugates. Accordingly, the above formulae are to be understood to include and represent those various hydrates and/or solvates.

In each of the foregoing and following embodiments, it is also to be understood that the formulae include and represent any and all crystalline forms, partially crystalline forms, and non-crystalline and/or amorphous forms of the compounds.

### Pharmaceutical Compositions

In view of the above, further provided is a pharmaceutical composition comprising any of the compounds or conjugates. In certain embodiments, provided herein is a pharmaceutical composition comprising a compound (e.g., a compound of Formula (I)) or a conjugate (e.g., a conjugate of Formula (X)), and one or more pharmaceutically acceptable carriers or excipients. The term “composition” generally refers to any product comprising more than one ingredient, including the compound or conjugate. The compositions can be prepared from isolated compounds or conjugates or from salts, solutions, hydrates, solvates, and other forms of the compounds and/or conjugates.

In certain embodiments, a compound of the composition comprises a compound of Formula (XX) (or a pharmaceutically acceptable salt thereof):

R₄-L-Z—R₃ (XX)

wherein:
R₄ is a nicotinamide or benzamide, each of the nicotinamide or benzamide optionally substituted;
L is a linker comprising at least one atom;
Z is a bicyclic heterocycle substituted with R₃; and
R₃ comprises one or more ring moieties.

In certain embodiments, a compound of the composition comprises a compound of Formula (I) (or the pharmaceutically acceptable salt thereof):

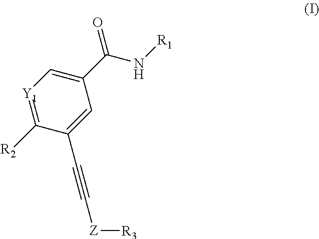

wherein R₁ is an alkyl, a pyridine, an isoxazole, a pyrazole, or aryl (e.g., a phenyl group), each optionally substituted with one or more of a piperazine (e.g., an alkyl piperazine), a pyrazine (e.g., an alkyl pyrazine), an imidazole (e.g., an alkyl imidazole), a cyanide, an amine, a halogen, a trifluoromethyl group, an N-containing heterocycle, and/or an alkyl; Y₁ is N or C; R₂ is H, an alkyl, a heteroalkyl, or a halogen; Z is a bicyclic heterocycle substituted with R₃, wherein the bicyclic heterocycle is imidazo[1,2-b]pyridazine or imidazo[1,2-b]pyrazole; and R₃ is a substituent comprising one or more ring moieties.

R₃ of the compound can further have a structure of Formula (II) or Formula (III) or Formula (IV):

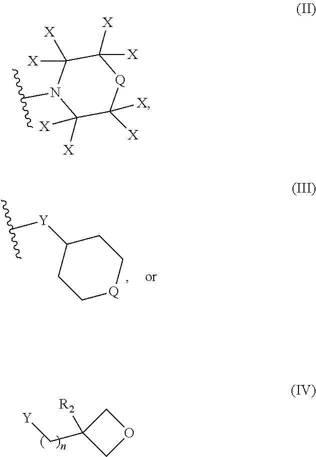

wherein

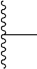

is a point of attachment; each X is independently an alkyl or an H, with the proviso that all X are not H; Q is O, S, N, or C, and is optionally substituted with one or more of an alkyl, a halogen, an O-alkyl, an amine, an —OH group, an alkoxy, a piperazine, a morpholine, an aziridine, a carbocycle or heterocycle, and/or a carbonyl; Y is O or an amine, with the proviso that Y is not NH; and n is 0 or 1.

In certain embodiments, a conjugate of the composition comprises a PROTAC conjugate (or a pharmaceutically acceptable salt thereof) having a chemical structure of Formula (X):

A-L′-D (X)

wherein A is a radical of a compound hereof; L′ is a linker that binds A and D, or absent; and D is a ubiquitin pathway protein binding moiety.

The compositions can be prepared from various amorphous, non-amorphous, partially crystalline, crystalline, and/or other morphological forms of the compounds and/or conjugates, and the compositions can be prepared from various hydrates and/or solvates of the compounds and/or conjugates. Accordingly, such pharmaceutical compositions can include each of, or any combination of, or individual forms of, the various morphological forms and/or solvate or hydrate forms of the compounds and/or conjugates.

The pharmaceutical composition can comprise one or more pharmaceutically acceptable carriers, adjuvants, diluents, excipients, and/or vehicles (e.g., conventional nontoxic pharmaceutically acceptable carriers, adjuvants, and vehicles), and combinations thereof. Any pharmaceutically acceptable carriers, diluents, and excipients as known in the art can be used. Examples include, but are not limited to, an excipient, a color additive, a preservative, and a stabilizer. More specific examples include crystal cellulose, calcium carmellose, sodium carmellose, hydropropylcellulose, hydroxypropylmethylcellulose, ethylcellulose, and magnesium stearate.

Solutions of the compound, conjugate or pharmaceutical composition can be aqueous, optionally mixed with a nontoxic surfactant, and/or can contain carriers or excipients, such as salts, carbohydrates and buffering agents (preferably at a pH of from 3 to 9), but, for some applications, they can be more suitably formulated as a sterile non-aqueous solution or as a dried form to be used in conjunction with a suitable vehicle, such as sterile, pyrogen-free water, or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical composition can further comprise an immunotherapeutic agent, a chemotherapeutic agent, an anti-cancer agent, and/or a hormone therapeutic agent. Alternatively, the compound or conjugate (or pharmaceutical composition comprising the compound or conjugate) can be administered simultaneously or sequentially, in either order, with an immunotherapeutic agent, an anti-cancer agent, a chemotherapeutic agent, and/or a hormone therapeutic agent (or a pharmaceutical composition comprising any of the foregoing).

The immunotherapeutic agent can be any suitable immunotherapeutic drug. Examples of suitable immunotherapeutic drugs include, but are not limited to, a transforming growth factor beta (TGF-β) inhibitor, such as R268712, or PD-L1 inhibitor, such as Keytruda.

The anti-cancer agent can be any suitable anti-cancer drug. Examples of suitable anti-cancer drugs include, but are not limited to, a kinase inhibitor, such as dasatinib.

The chemotherapeutic drug can be any suitable chemotherapeutic drug. Examples of suitable chemotherapeutic drugs include, but are not limited to, an anthracycline, such as doxorubicin, taxane, such as docetaxel, cyclophosphamide, such as Cytoxan, or 5-fluoro-uracil.

The hormone or hormone-related therapeutic agent can be any suitable hormone or hormone-related therapeutic agent. Examples include, but are not limited to, a hormone-production inhibitor, such as Zoladex or letrozole.

The compounds and/or conjugates can be formulated as pharmaceutical compositions and administered to a mammalian host, such as a human patient, in a variety of forms adapted to the chosen route of administration. The pharmaceutical compositions can be formulated, e.g., for a given route of administration, and manufactured in accordance with methods in the art and described, for example, in Remington, The Science and Practice of Pharmacy, 22^{nd} edition (2012).

The composition can be an infusion or an injectable composition, such as a composition that can be injected subcutaneously or intravenously.

The pharmaceutical composition can be administered to a mammalian host, such as a human patient, in a variety of forms adapted to the chosen route of administration. In certain embodiments, the pharmaceutical composition is formulated to be administered subcutaneously.

In certain embodiments, the pharmaceutical composition is formulated to be administered orally.

In certain embodiments, the pharmaceutical composition is formulated to be administered intramuscularly, intravenously, intraarterially, intraperitoneally, or as any other art-recognized route of parenteral administration.

In certain embodiments, the pharmaceutical composition is systemically administered in combination with a pharmaceutically acceptable vehicle. The percentages of the components of the compositions and preparations can vary and can be between about 1 to about 99% weight of the active ingredient(s) (e.g., the compound or conjugate) and a binder, an excipient, a disintegrating agent, a lubricant, and/or a sweetening agent (as are known in the art). The amount of active compound or conjugate in such therapeutically useful compositions is such that an effective dosage level can be obtained (e.g., in the serum or targeted tissue or cell).

Illustrative means of parenteral administration include needle (including microneedle) injectors, needle-free injectors and infusion techniques, as well as any other means of parenteral administration recognized in the art. Parenteral formulations are typically aqueous solutions, which can contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, can readily be accomplished using standard pharmaceutical techniques well-known to those skilled in the art.

The pharmaceutical dosage forms suitable for administration can include sterile aqueous solutions or dispersions or sterile powders comprising the active ingredients that are adapted for the extemporaneous preparation of sterile injectable or infusible solutions or dispersions, optionally encapsulated in liposomes, nanocrystals, or polymeric nanoparticles. In all cases, the ultimate dosage form should be sterile, fluid, and stable under the conditions of manufacture and storage. The liquid carrier or vehicle can be a solvent or liquid dispersion medium comprising, for example and without limitation, water, electrolytes, sugars, ethanol, a polyol (e.g., glycerol, propylene glycol, liquid polyethylene glycols, and the like), vegetable oils, nontoxic glyceryl esters, and/or suitable mixtures thereof. In at least one embodiment, the desired fluidity can be maintained by the formation of liposomes, by the maintenance of the required particle size in the case of dispersions or by the use of surfactants.

Sterile injectable solutions can be prepared by incorporating the pharmaceutical compositions in the required amount of the appropriate solvent with one or more of the other ingredients set forth above, as required, followed by filter sterilization. In the case of sterile powders for the preparation of sterile injectable solutions, vacuum-drying and freeze-drying techniques can be employed, which can yield a powder of the active ingredient plus any additional desired ingredient present in the previously sterile-filtered solutions.

### Methods

Further provided is a method of treating a disease state (e.g., a cancer and/or a solid tumor) in a subject. The method comprises administering to the subject an effective amount of (a) a compound hereof (e.g., a compound of Formula (I) and/or Formula (XX)); (b) conjugate hereof (e.g., a conjugate of Formula (X)); (c) a pharmaceutically acceptable salt, N-oxide, hydrate, solvent, tautomer, or optical isomer of the compound or conjugate; or (d) a pharmaceutical composition comprising one or more of a compound of (a), a conjugate of (b), and/or a pharmaceutically acceptable salt, N-oxide, hydrate, solvent, tautomer, or optical isomer of (c). In certain embodiments, the pharmaceutical composition can further comprise an immunotherapeutic agent, chemotherapeutic agent, anti-cancer drug, or hormone therapeutic agent and a pharmaceutically acceptable carrier or excipient.

Additionally, as desired, the method can further comprise administering an effective amount of (a), (b), (c), or (d) to the subject alone or in further combination with administration of a second pharmaceutical composition comprising an active agent and a second pharmaceutically acceptable carrier or excipient. The active agent can be, for example, an immunotherapeutic agent, chemotherapeutic agent, anti-cancer agent, or hormone therapeutic agent.

As used herein, the term “administering” and its variants include all means of introducing the compound(s) and compositions described herein to the subject, including, without limitation, oral (p.o.), intravenous (i.v.), intramuscular (i.m.), subcutaneous (s.c.), transdermal, via inhalation (e.g., intranasal (i.n.)), buccally, intraocularly, sublingually, vaginally, rectally, and the like.

As used herein, “effective amount” refers to an amount that is sufficient to achieve the desired result or to have an effect on an undesired condition. For example, an effective amount can refer to an amount that is sufficient to achieve the desired therapeutic result or to have an effect on symptoms. The specific effective dose level (e.g., amount) for any subject will depend upon a variety of factors including the disorder/condition being treated and the severity of the disorder/condition (e.g., the type, location, and severity of a fracture, bone injury, or cancer); the specific composition(s), compound(s), and/or conjugate employed (i.e., the potency and/or bioavailability thereof); the age, body weight, general health, sex and diet of the subject; the response of the subject; the time of administration; the route of administration; the rate of excretion of the specific conjugate(s) employed; the duration of the treatment; drugs/active agents used in combination or coincidental with the specific conjugate or compound employed and like factors that are well known in the medical arts. For example, it is well within the skill of the art to start doses of a compound/conjugate/composition at levels lower than those required to achieve the desired therapeutic effect and to gradually increase the dosage until the desired effect is achieved. If desired, the effective amount can be divided into multiple doses for purposes of administration. Consequently, single dose compounds/conjugates/compositions can contain such amounts or submultiples thereof to make up the daily dose. The dosage can be adjusted by the individual physician in the event of any contraindications. Additionally, pharmacogenomic (the effect of genotype on the pharmacokinetic, pharmacodynamic or efficacy profile of the antigen or composition) information about a particular patient can affect the dosage used to achieve an effective amount.

Depending upon the route of administration, a wide range of permissible dosages is contemplated. For example, the effective amount of the compound, conjugate, and/or pharmaceutical composition can range from about 0.1 μg/kg/day, such as 0.5 μg/kg/day, 0.7 μg/kg/day, or 0.01 mg/kg/day up to about 1,000 mg/kg/day. Intravenous doses can be several orders of magnitude lower.

Where the compound, conjugate, and/or first pharmaceutical composition is administered with an active agent or second pharmaceutical composition comprising the active agent, the compound/conjugate/first pharmaceutical composition and the active agent/second pharmaceutical composition can be administered to the subject simultaneously or sequentially, in either order and using any delivery modality. For example, and without limitation, the compound/conjugate/first pharmaceutical composition can be administered intravenously (e.g., as an i.v. solution) and the active agent/second pharmaceutical composition can be administered subcutaneously. In other embodiments, both the compound/conjugate/first pharmaceutical composition and the active agent/second pharmaceutical composition are administered intravenously. The use of a pharmaceutical composition comprising more than one drug/active agent is within the scope of this disclosure.

The method can further comprise administering to the subject a second therapy comprising an effective amount of a chemotherapeutic agent, an immunotherapeutic agent, or a hormone therapeutic agent; or radiation therapy.

The compound, conjugate and/or any other active agents administered therewith (or sequentially therewith) can be administered in therapeutically effective dosages to obtain clinically acceptable results, e.g., reduction or elimination of symptoms or of the tumor. Thus, the conjugate, any active agent(s), and/or any second therapy can be administered concurrently or consecutively in a treatment protocol. The administration of any active agents and/or second therapies can be made according to treatment protocols already known in the art.

Those skilled in the art will appreciate that treatment protocols can be varied according to the needs of the subject. Thus, the combination of conjugates and other compounds (drugs) used in the methods hereof can be administered in variations of the protocols described herein. For example, the conjugates and/or active agents can be administered discontinuously rather than continuously during the treatment cycle.

The method can further comprise the simultaneous or sequential administration, in either order, of an effective amount of an active agent that is a free immunotherapeutic agent, chemotherapeutic agent, anti-cancer drug, or hormone therapeutic agent, or a pharmaceutical composition (e.g., the second pharmaceutical composition) comprising same and a pharmaceutically acceptable carrier or excipient.

The disease state of the subject can be any type of disease or disorder modulated by one or more kinases. In certain embodiments, the disease or disorder is a cancer, disease, inflammatory disease state, or neurological disease modulated by one or more kinases. The disease or disorder can be modulated by one or more protein kinases selected from the group consisting of AB1, AB12, AFK, ALK, AMPK group, ATM, ATR, Aurora A, Aurora B, AXL, BCKDK, BLK, BMPR1B, BMX, Brk, BRSK1, BTK, CaM-KIalpha, CaM-Kllalpha, CaMKK group, CaM-KIV, CaM-KKalpha, CaM-KKbeta, CCDPK, CCRK, CDKI, CDK11, CDK2, CDK4, CDK5, CDK6, CDK7, CDK9, CDK group, CDPK, Chak1, CHK1, CHK2, CK1 alpha, CK1 delta, CK1 epsilon, CK1 group, CK2 alpha, CK2 beta, CK2 group, CLKJCSFIR, Csk, DAPK1, DAPK2, DAPK3, DAPK group, DCAMKL1, DMPK group, DNA-PK, DYRK1A, DYRK1B, DYRK2, DYRK3, eEF2K, Eg 3 kinase, EGFR, EIF2AK2, EphA2, EphA3, EphA4, EphA8, EphBl, EphB2, EphB3, EphB5, ErbB2, FAK, Fer, Fes, FGFR1, FGFR3, FGFR4, FGFR group, Fgr, FLT1, FLT3, FLT4, Fyn, GRK-1, GRK-2, GRK-3, GRK-4, GRK-5, GRK-6, GRK group, GSK-3alpha, GSK-3beta, GSK-3 group, HCK, HIPK2, HIPK3, HRI, ICK, IGF1R, IKK-alpha, IKK-beta, IKK-epsilon ILK, InsR, IPL1, IRAKI, IRAK4, ITK, JAK1, JAK2, JAK3, JAK group, JNK group, KDR, KIS, Kit, KSR1, Lck, LIMK1, LIMK2, LKB1, LOK, Lyn, MAP2K1, MAP2K2, MAP2K3, MAP2K4, MAP2K6, MAP2K7, MAPK2 group, MAP3K1, MAP3K11, MAP3K14, MAP3K5, MAP3K7, MAP3K8, MAPK3 group, MAP4K1, MAP4K2, MAP4K4, MAPK1, MAPK10, MAPK11, MAPK12, MAPK13, MAPK14, MAPK3, MAPK4, MAPK6, MAPK7, MAPK8, MAPK9, MAPK group, MAPKAPK2, MARK group, Mer, Met, MHCK, MLCK group, Mnkl,Mnk2, MOS, MRCKa, MSTI, MST3, mTOR, NDR1, NDR2, NEK1, NEK2, NEK6, NEK9, NEK group, NLK, NuaKl, p37 kinase, p38 group, p70S6K, p70R6Kb, P70S6K group, PAKI, PAK2, PAK3, PAK5, PAK6, PAK group, PASK, P-CIP2, PCTAIRE1, PDGFR alpha, PDGFR beta, PDGFR group, PDHK1, PDHK2, PDHK3, PDHK4, PDK-1, PDK-2, PDK group, PHK group, PIK3CA, PIK3CB, PIK3CD, PIK3CG, Pim-1, PKA alpha, Pka group, PKB beta, PKB group, PKC alpha, PKC beta, PKC delta, PKC epsilon, PKC eta, PKC gamma, PKC iota, PKC theta, PKC zeta, PKC group, PKD1, PKD2, PKD3, PKGl/cGK-I, PKG2/cGK-IL, PKG2/cGK group, PKN1, PLK1, PLK2, PLK3, PRP4, PYK2, RAF1, Ret, ROCK1, ROCK2, Ron, RPL10, RSK-1, RSK-2, RSK-3, RSK-5, SDK1, SGK group, SIK, Sky, Src, Sr c group, STLK3, Syk, TBK1, Tec, TESKI, TESK2, TGFbRI, TGFbR2, Tiel, Tie2, Titin kinase, TNK2, TRKA, TRKB, tropomyosin kinase, TSSK3, TXK, Tyk2, TYK2, I KK 1, Wee I, Wnkl, WNK I, Yes, and ZAP70. In certain embodiments, the disease state is modulated by MERTK and AXL, which are both inhibited by administration of an effective amount of a compound, conjugate, composition, or prodrug hereof.

In certain embodiments, the disease state is a cancer, such as acute myeloid leukemia, chronic myeloid leukemia, ovarian cancer, cervical cancer, pancreatic cancer, breast cancer, brain cancer, cervical cancer, pancreatic cancer, breast cancer (e.g., metastatic breast cancer), brain cancer, skin cancer, lung cancer, prostate cancer, lymphoma, leukemia, colon cancer, head cancer, neck cancer, thyroid cancer, kidney cancer, liver cancer, or stomach cancer. In certain embodiments, the cancer is a solid tumor cancer (e.g., breast cancer, colon cancer, lung cancer, etc.).

In certain embodiments, the method can further comprise imaging a population of cancer cells in the subject (e.g., following administration of the compound, conjugate, a first pharmaceutical composition comprising the compound or conjugate, or a first pharmaceutical composition comprising the compound or conjugate and an immunotherapeutic agent, a chemotherapeutic agent, an anti-cancer agent, and/or a hormone therapeutic agent). The population of cancer cells in the subject can be a solid tumor. Imaging can be performed through any now known or hereinafter developed imaging techniques relevant to the medical arts. In certain embodiments, the imaging can be performed through a hybrid scanning, utilizing a functional imaging modality such as a single photon emission computer tomography (SPECT) or PET in combination with computed tomography (CT) and/or magnetic resonance imaging (MRI) techniques, and combinations thereof. Ultrasound imaging can also be used.

When the population of cancer cells in the subject is imaged (either in connection with the method or separate therefrom), the method can further comprise diagnosing whether the subject has cancer.

When the subject has been treated for cancer and the bone in the subject is imaged, the method can further comprise assessing or monitoring the efficacy of treatment. For example, the compounds, conjugates and/or pharmaceutical compositions can be used to monitor tumor or lesion growth and proliferation quantitatively in vivo. In certain embodiments, a method of monitoring a progression of a cancer (e.g., a myeloid cancer) in a subject is provided, comprising administering to a subject a compound, conjugate, a pharmaceutically acceptable salt thereof, or a pharmaceutical composition comprising the compound or conjugate or a pharmaceutical acceptable salt thereof. Such method can further comprise imaging the cancer of the subject. In certain embodiments, a reduction in the size of a tumor microenvironment (TME), a population of cancer cells, and/or solid tumor in the subject as compared to the size of such TME, population and/or solid tumor prior to or earlier in treatment is indicative of an effective therapy or treatment.

The TME, population of cancer cells, and/or solid tumor of a subject can be imaged periodically over the course of a therapeutic treatment, and a practitioner can compare the images and/or otherwise quantify lesion or cancer growth to determine therapeutic efficacy (e.g., if there is a differential killing effect of the cancer cells over the course of the therapeutic treatment, or a relative increase in lesion size or cancer growth). Accordingly, a method is provided for determining the likelihood of success of a therapeutic treatment in a subject. It will be appreciated that such information can be utilized by one of skill in the art to affect dosing of the compound, conjugate or pharmaceutical composition depending on if positive results are detected (i.e., the treatment is effective or if the TME, cancer cell population and/or solid tumor is not decreasing in size, then perhaps adjusting the dosage or dosing regimen).

A method of suppressing T cell response in a TME of a subject is also provided, the method comprising administering to the subject an effective amount of. (a) a compound hereof (e.g., a compound of Formula (I) and/or Formula (XX)); (b) conjugate hereof (e.g., a conjugate of Formula (X)); (c) a pharmaceutically acceptable salt, N-oxide, hydrate, solvent, tautomer, or optical isomer of the compound or conjugate; or (d) a pharmaceutical composition comprising one or more of a compound of (a), a conjugate of (b), and/or a pharmaceutically acceptable salt, N-oxide, hydrate, solvent, tautomer, or optical isomer of (c). The subject can have cancer. The subject can have a solid tumor cancer. Because the compounds (e.g., compounds hereof comprising a morpholine substituent), conjugates, and pharmaceutical compositions hereof can inhibit at least MERTK and AXL, which suppresses an innate inflammatory immune response in the TME (and, for example, the recruitment of myeloid suppressor macrophages to the TME), administration of an effective amount of the same to a subject can suppress pro-inflammatory T cell response in the TME and thus leverage the subject's own immune system to further facilitate and anti-cancer environment.

The compounds, conjugates, and pharmaceutical compositions can be administered in unit dosage forms and/or compositions.

For methods described herein, the compound(s), conjugate(s), and compositions can be administered in a single dose, or via a combination of multiple dosages, which can be administered by any suitable means, contemporaneously, simultaneously, sequentially, or separately. Where the dosages are administered in separate dosage forms, the number of dosages administered per day for each compound, conjugate, or composition can be the same or different. The compound, conjugate and/or composition dosages can be administered via the same or different routes of administration. The compounds, conjugates, or compositions can be administered according to simultaneous or alternating regimens, at the same or different times during the course of the therapy, concurrently in divided or single forms.

Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease state or disorder.

The compound/conjugate/composition can be administered more than once, such as daily (1-3 or more times per day; q.d. (once a day), b.i.d. (twice a day), t.i.d. (three times a day)), weekly (including 1-3 or more times on a given day), bi-weekly (including 1-3 or more times on a given day), monthly (including 1-3 or more times on a given day), or bimonthly (including 1-3 or more times on a given day). In each case it is understood that the effective amounts described herein correspond to the instance of administration, or alternatively to the total daily, weekly, month, or quarterly dose, as determined by the dosing protocol.

### Certain Definitions

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range. The term “substantially” can allow for a degree of variability in a value or range, for example, within 90%, within 95%, 99%, 99.5%, 99.9%, 99.99%, or at least about 99.999% or more of a stated value or of a stated limit of a range.

The terms “a,” “an,” or “the” are used to include one or more than one unless the context clearly dictates otherwise. The term “or” is used to refer to a nonexclusive “or” unless otherwise indicated.

In addition, it is to be understood that the phraseology or terminology employed herein, and not otherwise defined, is for the purpose of description only and not of limitation. Any use of section headings is intended to aid reading of the document and is not to be interpreted as limiting.

Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups.

Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms(C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “patient” and “subject” are used interchangeably and include a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale. The patient to be treated is preferably a mammal, in particular a human being.

While the concepts of the present disclosure are illustrated and described in detail in the figures and descriptions herein, results in the figures and their description are to be considered as exemplary and not restrictive in character; it being understood that only the illustrative embodiments are shown and described and that all changes and modifications that come within the spirit of the disclosure are desired to be protected. Indeed, the numerous specific details provided are set forth to provide a thorough understanding of the present disclosure.

Any use of section headings is intended to aid reading of the document and is not to be interpreted as limiting. Further, information that is relevant to a section heading may occur within or outside of that particular section.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Various techniques and mechanisms of the present disclosure will sometimes describe a connection or link between two components. Words such as attached, linked, coupled, connected, tethered and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

### EXAMPLES

The following examples serve to illustrate the present disclosure. The examples are not intended to limit the scope of the claimed invention in any way.

### Example 1

Effect of Substituents on the Imidazole[1,2-b]pyridazine Moiety

The nature of the substituent on the imidazo[1,2-b]pyridazine moiety at position-6 was evaluated in in vitro studies performed at Reaction Biology Corporation (Malvern, PA). The results indicated the specific substituent dramatically affected the inhibition of p70S6K. For example, at 25 nM, both ponatinib and compound HSN748 (comprising a linear substituent at position-6) inhibited p70S6K at only 38% and 55% respectively ([ATP]=10 uM), whereas compounds HSND77, HSND79 and HSND80 (6-ring-substituted analogs) inhibited p70S6K at 95%, 95% and 97%, respectively, under the same conditions.

At 50 nM compound and 10 uM ATP, compounds HSND77, HSND79 and HSND80 hereof also inhibited MNK2 better than compound HSN748 (92% (compound HSND77), 94% (compound HSND79) and 96% (compound HSND80), versus 89% (compound HSN748 comprising a linear substituent at position-6). This data supports that the modification of the imidazo[1,2-b]pyridazine moiety positively affects the inhibition of p70S6K by the nicotinamide class of kinase inhibitors.

### Example 2

### 6-Position Ring Substituted Compounds Inhibit TAM Kinases, AXL and MERTK

The affinity of several compounds hereof having a substituent on the imidazo[1,2-b]pyridazine moiety at position-6, wherein the substituent comprised one or more ring moieties was evaluated with respect to two TAM kinases: AXL and MERTK. Perhaps more specifically, the equilibrium dissociation constant between each of compounds HSND141, compound HSND149, HSND150, HSND151, and HSND80 and AXL and MERTK was evaluated at Eurofins DiscoverX using KINOMEscan™, which is a competition binding assay that quantitively measured the ability of each tested compound to displace an immobilized, active-site directed ligand. The assay uses DNA-tagged kinase, an immobilized ligand, and the test compound. Quantitative polymerase chain reaction (PCR) of the DNT tag was then used to measure the ability of the tested compound to displace the immobilized ligand.

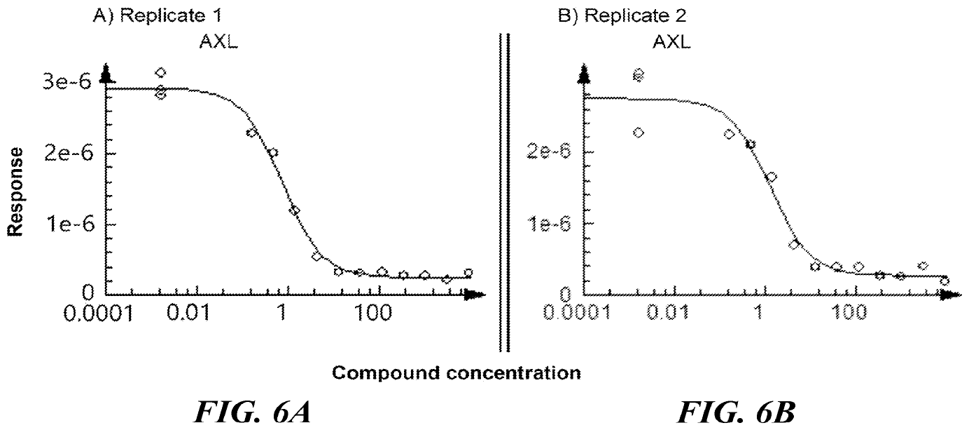

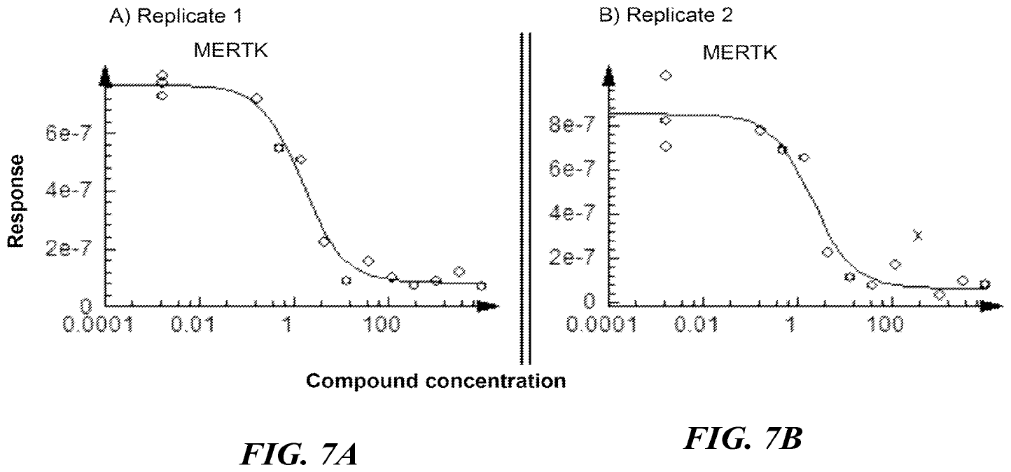

FIGS. 6A-7B shows graphical results from such evaluations where HSND80 was the compound tested and Table 1 shows the measured K_{D} in nM for all tested compounds (with K_{D} values obtained using DiscoverX (The Eurofins Discovery)).

TABLE 1
Kd in nM. Kd was obtained at DiscoverX Eurofins

| Compound | AXL | MERTK |
|---|---|---|
| HSND141 | 1.2 | 0.22 |
| HSND149 | 3.7 | 0.2 |
| HSND150 | 2.8 | <0.17 |
| HSND151 | 3.3 | <0.17 |
| HSND80 | 1.1 | <0.17 |

In sum, each of compounds HSND141, compound HSND149, HSND150, HSND151, and HSND80 exhibited a high affinity for/strong binding with both AXL and MERTK, which are notably not targeted by the ponatinib or HSN748 compounds. HSND80, in particular, potently bound to MERTK.

### Example 3

### Compound HSND80 and Analogs Thereof Inhibits NCI-60 Cell Lines Better than Ponatinib and Parent Compounds Having Linear Substituent Groups

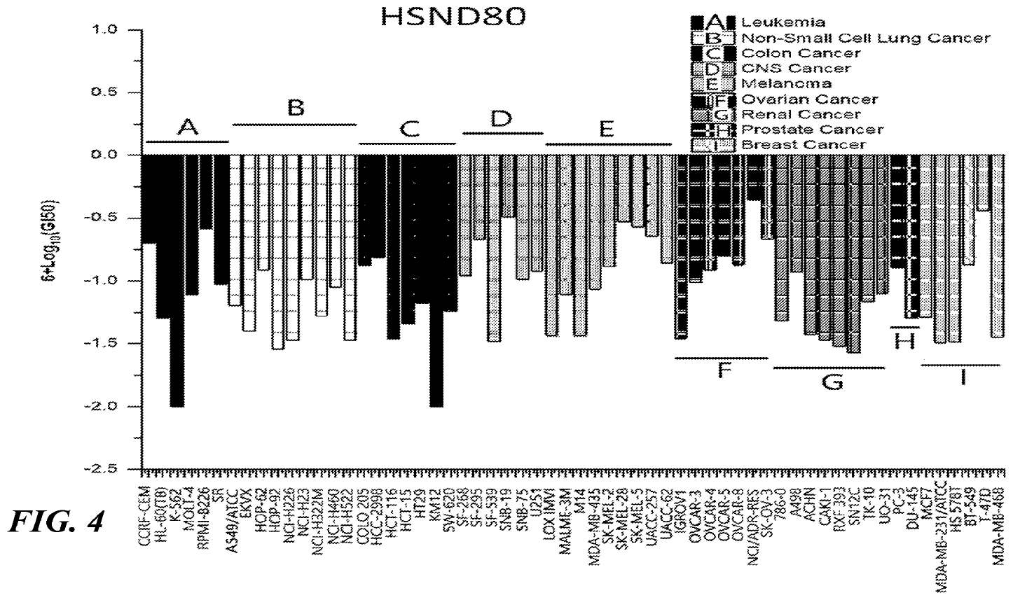

The degree of inhibition of various kinase-driven cancers by 6-linear substituted ponatinib analogs as compared to the compounds hereof was evaluated in an NCI-60 human tumor cell line screen. At 200 nM, compound HSND80 and analogs thereof completely inhibited (100%) the growth of Caki-1 (renal cancer) or MDA-MB-231 (breast cancer) (see FIG. 4). The IC₅₀ for the inhibition of MDA-MB-231 by compound HSND80 (72 hours) was 8.6 nM. Compound HSND80 and analogs also potently inhibited multiple myeloma cell lines (FIG. 4).

Interestingly, other 6-substituted compounds, such as compounds HSND37, HSND39 and HSND40 were only moderate inhibitors of MDA-MB-231 with GI50 values (NCI-60 screening) of 413 nM (compound HSND37), 571 nM (compound HSND39) and 889 nM (compound HSND40). Under similar conditions compound HSND41 (a ring analog comprising unsubstituted morpholine) inhibited MDA-MB-231 with GI50 value of 35 nM.

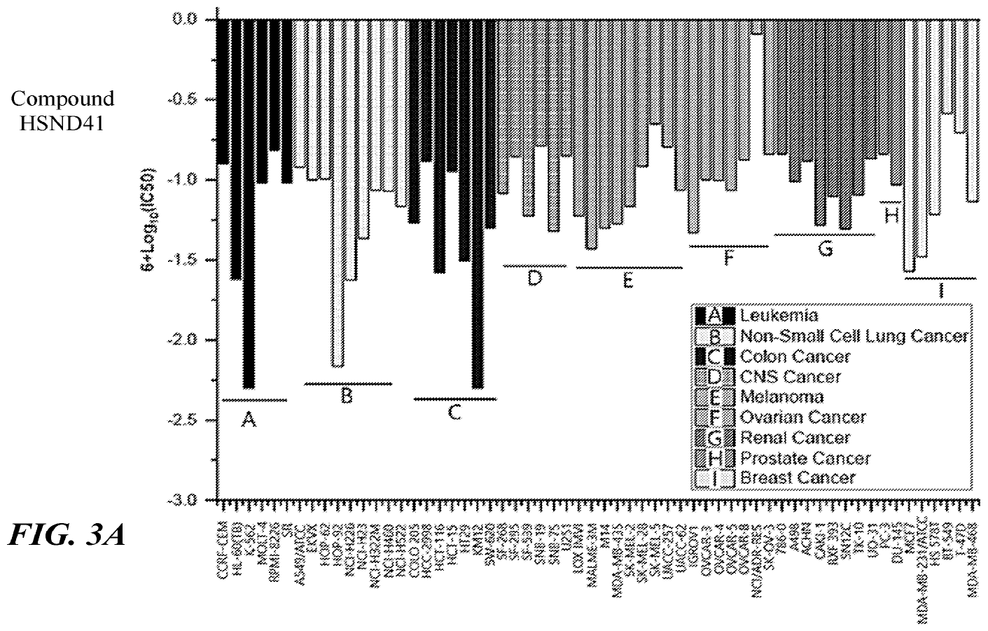

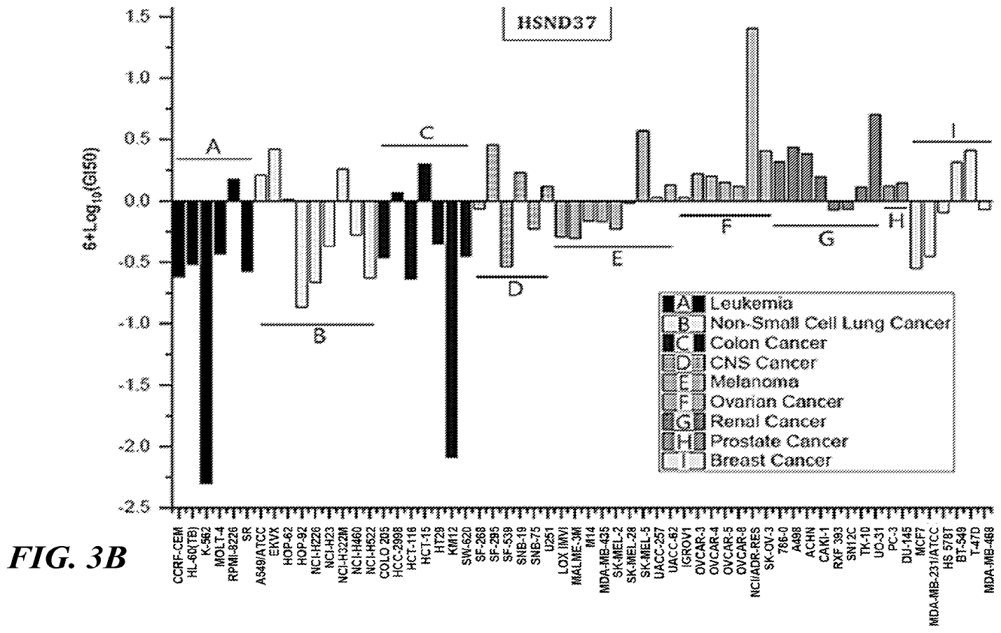

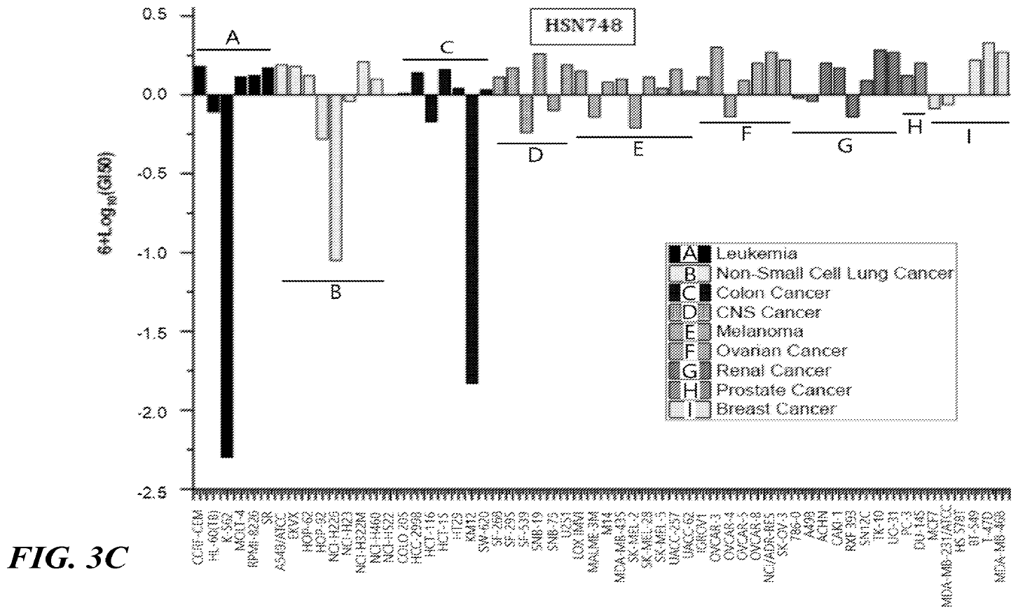

This supports that the nature of the 6-substitution on the imidazo[1,2-b]pyridazine moiety is vital for anticancer activities (compare compounds HSN748, HSND37 and HSND41, FIGS. 3A-3C, respectively).

### Example 4

### Bioavailability Studies

The bioavailability and Cₘₐₓ of the ring-6-substituted imidazo[1,2-b]pyridazine compounds were also assessed.

Briefly, compounds were dissolved in 10% dimethylsulfoxide (DMSO)/90% (20% HP-β-CD in water (w/w)). Male CD1 Mice (n=3) were dosed orally with 10 mg/Kg of each compound (HSND80, HSND41, HSND101, HSND102, HSND141, and HSND156). Blood was sampled at 0.25 hours, 0.5 hour, 1 hour, 2 hours, 4 hours, 8 hours, and 24 hours post dose, and the concentration of compound in blood at each specific time point was determined via liquid chromatography-mass spectrometry (LC-MS) (high-performance liquid chromatography (HPLC) instrument: Shimadzu; MS instrument: AB API 5500 LC/MS/MS; Column: HALO C18 90 A 2.7 μm (30*2.1 mm); Mobile phase A: 5% Acetonitrile in Water (0.1% Formic acid); Mobile phase B: 95% Acetonitrile in Water (0.1% Formic acid)).

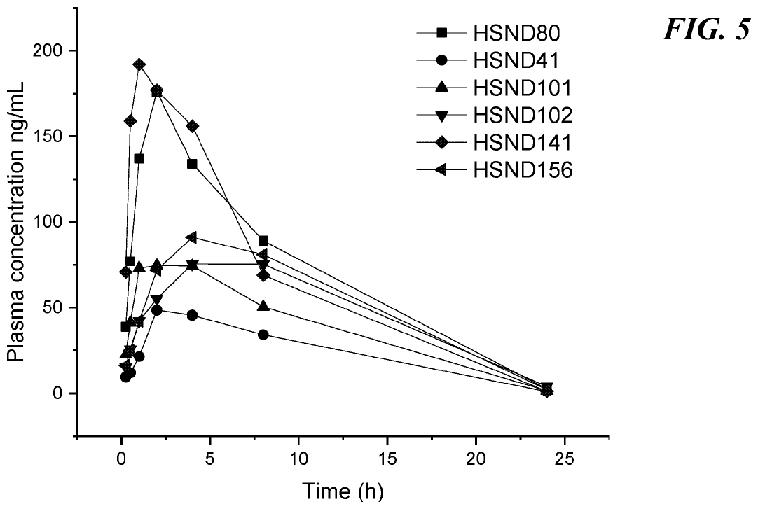

The data shown in FIG. 5 supports all compounds tested were bioavailable, with the dimethylmorpholine compound HSND80 exhibiting more bioavailablity than unsubstituted morpholine (compound HSND41).

### Example 5

### Efficacy Studies—4T1 In Vivo Tumor Reduction by HSND80

0.5×10⁶ 4T1 cells were injected subcutaneously (SQ) into the right flank of Female Balb-C mice. Each group consisted of 5 tumor-bearing mice. 7 days after tumor implantation, mice were dosed orally QD with vehicle control (5% DMSO, 40% PEG300, 5% tween 80, 50% water) or HSND80 (15 mg/Kg). Mice were dosed 5 days on and 2 days off (drug holiday). Tumors were measured every other day (L, W, H) to give tumor volume in mm³.

At 15 mg/kg, HSND80 reduced the growth of 4T1 (breast) syngeneic tumor by ˜60% (data not shown).

### Example 6

### Efficacy Studies—CT-26 In Vivo Tumor Reduction by HSND80 and HSND100

Each BALB/c female mouse was inoculated subcutaneously at the right flank with CT-26 C2 tumor cells (0.3×10⁶/mouse) in 0.1 mL PBS for tumor development. Treatments for the efficacy study were started on day 10 after tumor inoculation when the average tumor size reached approximately 71 mm³. Each group consisted of 6 tumor-bearing mice. Vehicle control was 25 mM citrate buffer (pH 2.5). HSND80 was dosed orally QD at 15 mg/Kg for 4 days and 10 mg/Kg afterwards. HSND100 was dosed at 15 mg/Kg. Mice were dosed 5 days on and 2 days off (drug holiday). Tumors were measured every other day (L, W, H) to give tumor volume in mm³.

On day 17 (from start of dosing), HSND80 effected 45% tumor reduction (compared to vehicle control) and HSND100 effected 36% tumor reduction.

### Example 7

### Compound Synthesis and Characterization

### General Procedure for the Synthesis of Substituted Imidazo[1,2-b]Pyridazine Substrates:

In a sealed tube, 3-Bromo-6-chloroimidazo[1,2-b]pyridazine (500 mg) and the appropriate amine (4 eq.) in n-propanol (1 mL) or amine as solvent was refluxed at 150° C. overnight. After completion, the reaction was extracted with ethyl acetate and washed with brine. The organic layer was collected, dried over sodium sulfate, and concentrated under reduced pressure. Crude was purified via silica gel column chromatography to yield the desired product.

### General Procedure for Sonogashira Coupling:

Substituted imidazo[1,2-b]pyridazine substrate (1 mmol), alkyne substrate (1.1 mmol), PdCl₂(PPh₃)₂ (3 mol %), XPhos (2 mol %), Cs₂CO₃ (3 equiv) and CuI (1 mol %) were deoxygenated using argon gas. An anhydrous N, N-dimethylformamide (DMF) (5 mL) and N, N-diisopropylethylamine (DIPEA) (2.5 mL) solution was then added to the deoxygenated solution under inert conditions. The reaction mixture was allowed to stir at 60° C. overnight.

After completion, the reaction mixture was concentrated and extracted with ethyl acetate. The organic layer was washed with brine solution (20 mL). The organic layer was dried over anhydrous sodium sulfate, filtered, and concentrated in vacuo, and then purified via silica gel column chromatography to yield the desired product.

Specific compounds/products and their substrates are described below, along with data confirming the structure of the synthesized compounds.

### (R)-3-bromo-6-((tetrahydrofuran-3-yl)oxy)imidazo[1,2-b]pyridazine

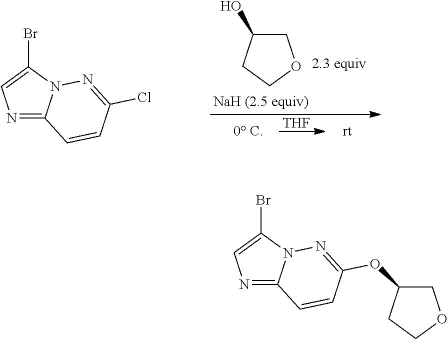 [SMILES: Clc1ccc2ncc(Br)n2n1 ; O[C@@H]1CCOC1 ; Brc1cnc2ccc(O[C@@H]3CCOC3)nn12 ; "THF"]

In accordance with the general procedure described above, a reaction mixture of 3-bromo-6-chloroimidazo[1,2-b]pyridazine (500 mg) and (R)-tetrahydrofuran-3-ol (2.3 equiv) in dry tetrahydrofuran (THF) (20 mL) was cooled to 0° C. followed by addition of sodium hydride (NaH) (2.5 equiv). The reaction mixture was stirred at room temperature overnight. After completion, the reaction was extracted with ethyl acetate and washed with brine. The organic layer was collected and dried over sodium sulfate and concentrated under reduced pressure to get the desired product. Off-white solid, 84% yield.

### (R)-N-(4-((4-Methylpiperazin-1-yl)methyl)-3-(trifluoromethyl)phenyl)-5-((6-((tetrahydrofuran-3-yl)oxy)imidazo[1,2-b]pyridazin-3-yl)ethynyl)nicotinamide (Compound HSND-28)

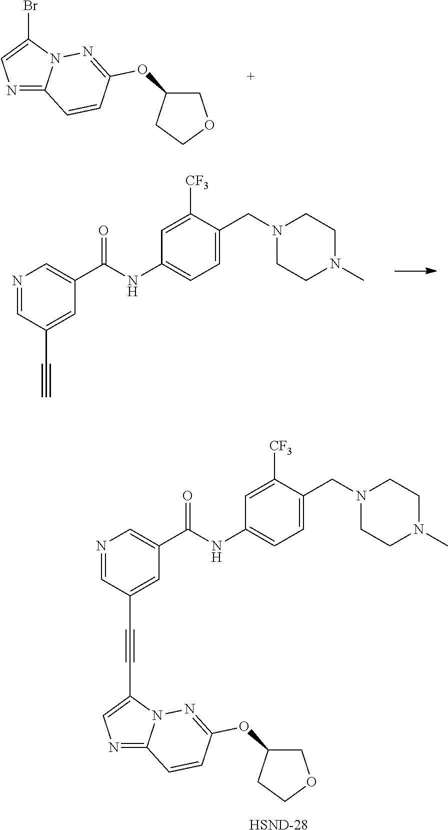

Off-white solid, Yield: 58%. H NMR (500 MHz, DMSO-d₆) δ10.79 (s, 1H), 9.10 (s, 1H), 8.96 (d, J=1.9 Hz, 1H), 8.53-8.45 (m, 1H), 8.19 (s, 1H), 8.12 (d, J=9.6 Hz, 1H), 8.04 (d, J=15.3 Hz, 2H), 7.71 (d, J=8.5 Hz, 1H), 7.03 (d, J=9.6 Hz, 1H), 5.61-5.53 (m, 1H), 4.04-3.89 (m, 2H), 3.86 (q, J=7.7 Hz, 1H), 3.75 (td, J=8.3, 4.9 Hz, 1H), 3.56 (s, 2H), 2.53-2.29 (m, 9H), 2.22 (s, 3H), 2.17-2.07 (m, 1H); ¹³C NMR (126 MHz, DMSO-d₆) δ 163.8, 160.0, 153.8, 148.7, 138.4, 138.3, 137.8, 137.4, 132.8, 131.8, 130.3, 128.7, 127.8 (q, J=30.2 Hz), 125.8 (q, J=274.6 Hz), 123.9, 119.2, 117.6, 114.4, 111.9, 94.9, 81.2, 78.6, 72.8, 66.8, 57.7, 54.9, 52.6, 45.6, 32.9.

### (S)-3-bromo-6-((tetrahydrofuran-3-yl)oxy)imidazo[1,2-b]pyridazine

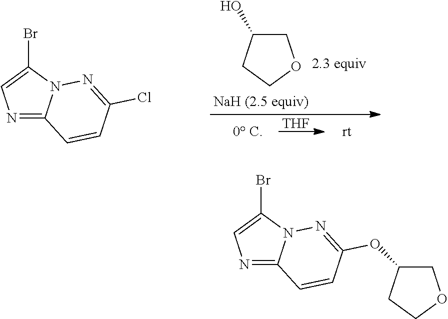 [SMILES: Clc1ccc2ncc(Br)n2n1 ; O[C@H]1CCOC1 ; Brc1cnc2ccc(O[C@H]3CCOC3)nn12 ; "THF"]

In accordance with the general procedure described above, a reaction mixture of 3-bromo-6-chloroimidazo[1,2-b]pyridazine (500 mg) and (S)-tetrahydrofuran-3-ol (2.3 equiv) in dry THF (20 mL) was cooled to 0° C. and NaH (2.5 equiv) was then added thereto. The reaction mixture was stirred at room temperature overnight. After completion, the reaction was extracted with ethyl acetate and washed with brine. The organic layer was collected and dried over sodium sulfate and concentrated under reduced pressure to get the desired product. Off-white solid, 77% yield.

¹H NMR (500 MHz, Chloroform-d) δ 7.74 (d, J=9.6 Hz, 1H), 7.57 (s, 1H), 6.69 (d, J=9.6 Hz, 1H), 5.58-5.51 (m, 111), 4.13-4.06 (m, 1H), 4.03-3.92 (m, 2H), 3.92-3.82 (m, 1H), 2.34 (dtd, J=14.5, 8.0, 6.5 Hz, 1H), 2.23-2.14 (m, 1H); ¹³C NMR (126 MHz, CDCl₃) δ 159.4, 137.7, 132.7, 127.7, 112.1, 100.8, 78.0, 73.1, 67.1, 32.8.

### (S)-N-(4-((4-methylpiperazin-1-yl)methyl)-3-(trifluoromethyl)phenyl)-5-((6-((tetrahydrofuran-3-yl)oxy)imidazo[1,2-b]pyridazin-3-yl)ethynyl)nicotinamide (Compound HSND-29)

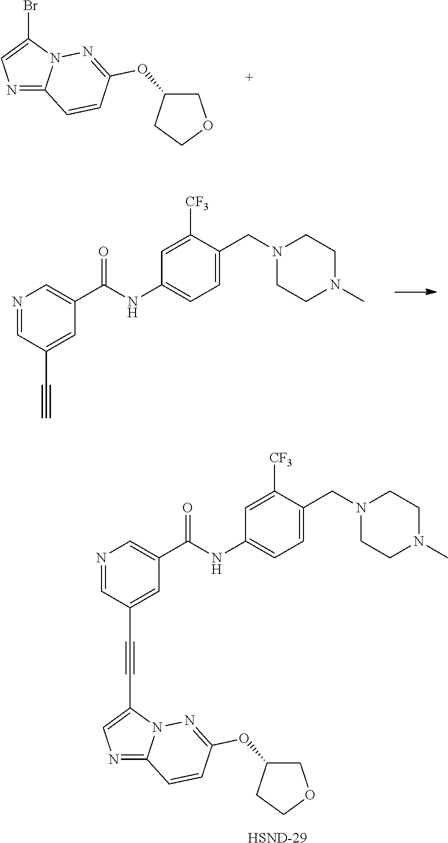 [SMILES: Brc1cnc2ccc(O[C@H]3CCOC3)nn12 ; C#Cc1cncc(C(=O)Nc2ccc(CN3CCN(C)CC3)c(C(F)(F)F)c2)c1 ; CN1CCN(Cc2ccc(NC(=O)c3cncc(C#Cc4cnc5ccc(O[C@H]6CCOC6)nn45)c3)cc2C(F)(F)F)CC1 ; "HSND-29"]

Off-white solid, Yield: 59%. ¹H NMR (500 MHz, DMSO-d₆) δ 10.81 (s, 1H), 9.10 (d, J=2.2 Hz, 1H), 8.95 (d, J=2.0 Hz, 1H), 8.49 (t. J=2.1 Hz. 1H), 8.19 (d, J=2.2 Hz, 1H), 8.12 (d, J=9.7 Hz, 1H), 8.07-8.00 (m, 2H), 7.71 (d, J=8.5 Hz, 1H), 7.03 (d, J=9.7 Hz, 1H), 5.59-5.54 (m, 1H), 4.01-3.97 (m, 111), 3.95-3.90 (m, 1H), 3.85 (t, J=7.7 Hz, 1H), 3.78-3.70 (m, 1H), 3.56 (s, 2H), 2.44-2.30 (m, 9H), 2.19 (s, 3H), 2.16-2.08 (m, 1H); ¹³C NMR (126 MHz, DMSO-d₆) δ 163.8, 160.0, 153.8, 148.7, 138.4, 138.3, 137.8, 137.4, 132.9, 131.8, 130.3, 128.7, 128.0 (q, J=30.2 Hz), 125.8 (q, J=274.6 Hz), 123.9, 119.2, 117.6, 114.4, 111.9, 94.9, 81.2, 78.6, 72.8, 66.8, 57.8, 55.0, 52.8, 45.8, 32.9.

### 3-Bromo-6-((tetrahydro-2H-pyran-4-yl)oxy)imidazo[1,2-b]pyridazine

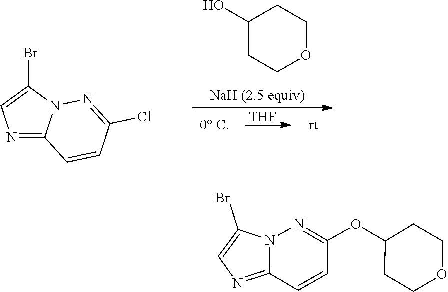 [SMILES: Clc1ccc2ncc(Br)n2n1 ; OC1CCOCC1 ; Brc1cnc2ccc(OC3CCOCC3)nn12 ; "THF"]

In accordance with the general procedure described above, a reaction mixture of 3-bromo-6-chloroimidazo[1,2-b]pyridazine (500 mg) and tetrahydro-2H-pyran-4-ol (2.3 equiv) in dry THF (20 mL) was cooled to 0° C., and NaH (2.5 equiv) was added thereto. The reaction mixture was then stirred at room temperature overnight. After completion, the reaction was extracted with ethyl acetate and washed with brine. The organic layer was collected and dried over sodium sulfate and concentrated under reduced pressure to result in the desired product. Off-white solid, 82% yield.

¹H NMR (500 MHz, Chloroform-d) δ 7.77 (d, J=9.6 Hz, 1H), 7.59 (s, 1H), 6.71 (d, J=9.6 Hz, 1H), 5.27 (dt, J=8.5, 4.3 Hz, 1H), 4.01 (dt, J=11.8, 4.5 Hz, 2H), 3.73-3.54 (m, 2H), 2.29-2.11 (m, 3H), 1.95-1.77 (m, 2H); ¹³C NMR (126 MHz, CDCl₃) δ 159.2, 137.8, 132.6, 127.6, 112.4, 100.6, 72.2, 65.3, 31.5.

### N-(4-((4-methylpiperazin-1-yl)methyl)-3-(trifluoromethyl)phenyl)-5-((6-((tetrahydro-2H-pyran-4-yl)oxy)imidazo[1,2-b]pyridazin-3-yl)ethynyl)nicotinamide (Compound HSND-34)

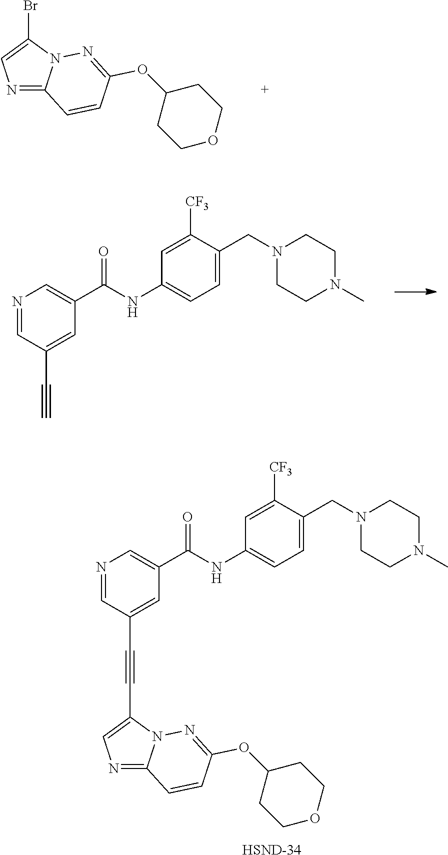 [SMILES: Brc1cnc2ccc(OC3CCOCC3)nn12 ; C#Cc1cncc(C(=O)Nc2ccc(CN3CCN(C)CC3)c(C(F)(F)F)c2)c1 ; CN1CCN(Cc2ccc(NC(=O)c3cncc(C#Cc4cnc5ccc(OC6CCOCC6)nn45)c3)cc2C(F)(F)F)CC1 ; "HSND-34"]

¹H NMR (500 MHz, Methanol-d₄) δ 9.05 (d, J=2.1 Hz, 1H), 8.84 (d, J=2.0 Hz, 1H), 8.42 (t, J=2.1 Hz, 1H), 8.13 (d, J=2.2 Hz, 1H), 7.97-7.86 (m, 3H), 7.76 (d, J=8.5 Hz, 1H), 6.96 (d, J=9.6 Hz, 1H), 5.36-5.25 (m, 1H), 3.99 (dt, J=11.7, 4.5 Hz, 2H), 3.71-3.62 (m, 4H), 2.73 (s, 5H), 2.59 (s, 4H), 2.45 (s, 3H), 2.28-2.20 (m, 2H), 1.92-1.84 (m, 2H); ¹³C NMR (126 MHz, Methanol-d₄) δ 163.9, 159.8, 152.9, 147.1, 138.1, 137.6, 137.5, 136.1, 132.7, 131.2, 130.5, 128.9 (q, J=30.2 Hz), 127.0, 125.3 (q, J=273.4 Hz), 123.5, 120.0, 117.7, 114.6, 112.3, 93.8, 80.2, 72.7, 64.9, 57.3, 54.3, 51.5, 43.9, 31.3.

### 3-Bromo-6-isopropoxyimidazo[1,2-b]pyridazine

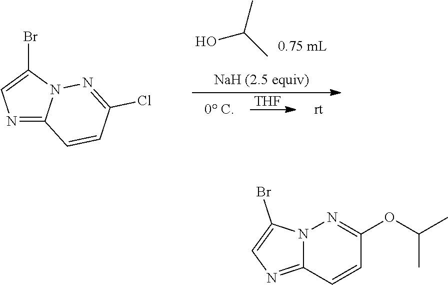

In accordance with the general procedure described above, a reaction mixture of 3-bromo-6-chloroimidazo[1,2-b]pyridazine (500 mg) and isopropanol (0.75 equiv) in dry THF (20 mL) was cooled to 0° C., followed by the addition of NaH (2.5 equiv). The reaction mixture was then stirred at room temperature overnight. After completion, the reaction was extracted with ethyl acetate and washed with brine. The organic layer was collected and dried over sodium sulfate and concentrated under reduced pressure to result in the desired product.

Off-white solid, ¹H NMR (500 MHz, Chloroform-d) δ 7.73 (dd, J=9.6, 1.4 Hz, 1H), 7.57 (d, J=1.3 Hz, 1H), 6.65 (dd, J=9.6, 1.4 Hz, 1H), 5.40-5.27 (m, 1H), 1.44 (dd, J=6.1, 1.4 Hz, 6H); ¹³C NMR (126 MHz, CDCl₃) δ 159.5, 137.7, 132.4, 127.3, 112.7, 100.6, 70.8, 21.6.

### 5-((6-Isopropoxyimidazo[1,2-b]pyridazin-3-yl)ethynyl)-N-(4-((4-methylpiperazin-1-yl)methyl)-3-(trifluoromethyl)phenyl)nicotinamide (Compound HSND-35)

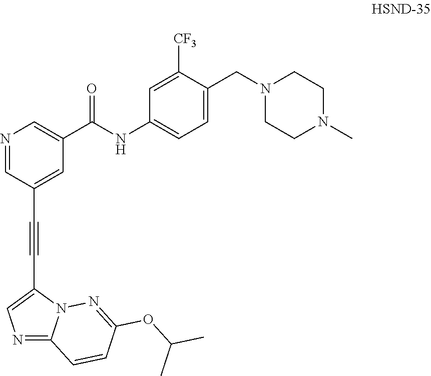 [SMILES: CC(C)Oc1ccc2ncc(C#Cc3cncc(C(=O)Nc4ccc(CN5CCN(C)CC5)c(C(F)(F)F)c4)c3)n2n1]

¹H NMR (500 MHz, Methanol-d₄) δ 9.06 (s, 1H), 8.87 (s, 1H), 8.45 (dq, J=4.0, 1.8 Hz, 1H), 8.13 (d, J=2.1 Hz, 1H), 7.97-7.86 (m, 3H), 7.77 (dd, J=8.6, 2.2 Hz, 1H), 6.91 (dd, J=9.6, 3.0 Hz, 1H), 5.42-5.27 (m, 1H), 2.58 (s, 1011), 2.36 (s, 3H), 1.47 (d, J=6.1 Hz, 6H); ¹³C NMR (126 MHz, Methanol-d₄) δ 164.0, 160.1, 153.1, 147.2, 137.5, 137.4, 136.2, 132.9, 131.2, 130.6, 128.6 (q, J=31.5 Hz), 126.7, 125.3 (q, J=273.4 Hz), 123.5, 120.1, 117.7, 114.9, 93.65, 80.2, 71.1, 57.4, 54.4, 51.9, 44.2, 20.5.

### 5-((6-Aminoimidazo[1,2-b]pyridazin-3-yl)ethynyl)-N-(4-((4-methylpiperazin-1-yl)methyl)-3-(trifluoromethyl)phenyl)nicotinamide (Compound HSND-37)

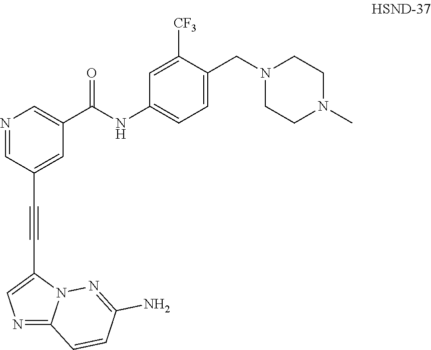 [SMILES: CN1CCN(Cc2ccc(NC(=O)c3cncc(C#Cc4cnc5ccc(N)nn45)c3)cc2C(F)(F)F)CC1]

¹H NMR (500 MHz, Methanol-d₄) δ 9.02 (d, J=2.2 Hz, 1H), 8.89-8.82 (m, 1H), 8.49-8.43 (m. 1H), 8.11 (d, J=2.4 Hz, 1H), 7.93 (dd, J=8.5, 2.3 Hz, 1H), 7.78-7.70 (m, 2H), 7.67 (dd, J=9.6, 2.9 Hz, 1H), 6.80 (d, J=9.6 Hz, 1H), 3.64 (s, 2H), 2.52 (s, 8H), 2.30 (s, 3H); ¹³C NMR (126 MHz, Methanol-d₄) δ 164.0, 156.0, 153.0, 147.0, 137.5, 137.4, 137.2, 135.1, 132.9, 131.2, 130.4, 128.8 (q, J=30.2 Hz), 125.3 (q, J=273.4 Hz), 125.0, 123.5, 120.3, 117.6, 114.0, 111.3, 93.1, 80.9, 57.4, 54.5, 52.2, 44.5.

### 2-((3-Bromoimidazo[1,2-b]pyridazin-6-yl)amino)ethan-1-ol

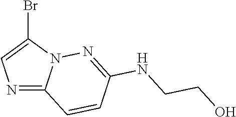 [SMILES: OCCNc1ccc2ncc(Br)n2n1]

¹H NMR (500 MHz, DMSO-d₆) δ 7.66 (d, J=9.7 Hz, 1H), 7.45 (s, 1H), 7.14 (t, J=5.4 Hz, 1H), 6.74 (d, J=9.7 Hz, 1H), 4.76 (s, 1H), 3.61 (q, J=5.7 Hz, 2H), 3.34-3.31 (m, 2H); ¹³C NMR (126 MHz, D DMSO-d₆) S 154.7, 137.0, 130.7, 125.7, 113.5, 99.5, 59.4, 44.0.

### 5-((6-((2-Hydroxyethyl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND-38)

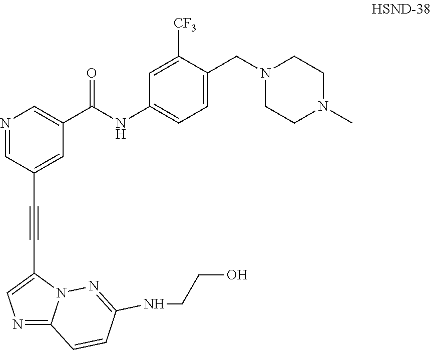 [SMILES: CN1CCN(Cc2ccc(NC(=O)c3cncc(C#Cc4cnc5ccc(NCCO)nn45)c3)cc2C(F)(F)F)CC1]

¹H NMR (500 MHz, Methanol-d₄) δ 8.98 (d, J=2.1 Hz, 1H), 8.78 (d, J=2.0 Hz, 1H), 8.38 (t, J=2.1 Hz, 1H), 8.08 (d, J=2.2 Hz, 1H), 7.90 (dd, J=8.4, 2.3 Hz, 1H), 7.71 (d, J=8.5 Hz, 1H), 7.66 (s, 1H), 7.55 (d, J=9.7 Hz, 1H). 6.73 (d, J=9.7 Hz, 1H), 3.83 (t, J=5.7 Hz, 2H), 3.61 (s, 2H), 3.54 (t, J=5.7 Hz, 2H), 2.49 (s, 8H), 2.27 (s, 3H); ¹³C NMR (126 MHz, Methanol-d₄) δ 163.9, 154.7, 152.9, 146.9, 137.4, 137.1, 137.0, 134.7, 132.9, 131.1, 130.2, 128.7 (q, J=30.2 Hz), 125.3 (q, J=273.4 Hz), 124.2, 123.4, 123.1, 120.3, 117.6, 114.6, 111.7, 93.2, 81.1, 60.0, 57.4, 54.6, 52.2, 44.6, 43.7.

### N¹-(3-Bromoimidazo[1,2-b]pyridazin-6-yl)-N²,N²-dimethylethane-1,2-diamine

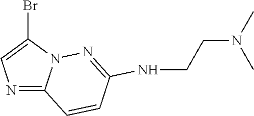 [SMILES: CN(C)CCNc1ccc2ncc(Br)n2n1]

¹H NMR (500 MHz, Chloroform-d) δ 7.45 (dd, J=9.6, 1.8 Hz, 1H), 7.39 (d, J=1.8 Hz, 1H), 6.41 (dd, J=9, 6, 1.8 Hz, 1H), 5.40 (t, J=4.9 Hz, 1H), 3.43-3.36 (m, 2H), 2.49 (dt, J=6.2, 3.0 Hz, 2H); ¹³C NMR (126 MHz, Chloroform-d) δ 153.8, 137.0, 131.0, 125.5, 112.1, 99.9, 57.2, 45.1, 38.8.

### 5-((6-((2-(Dimethylamino)ethyl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND 39)

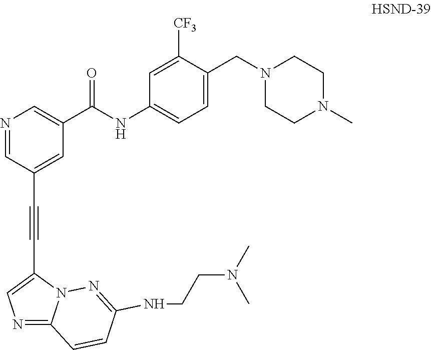 [SMILES: CN(C)CCNc1ccc2ncc(C#Cc3cncc(C(=O)Nc4ccc(CN5CCN(C)CC5)c(C(F)(F)F)c4)c3)n2n1]

¹H NMR (500 MHz, Methanol-d₄) δ 9.03 (d, J=2.2 Hz, 1H), 8.87-8.80 (m, 1H), 8.49-8.39 (m, 1H), 8.12 (d, J=2.2 Hz, 1H), 7.93 (dd, J=8.4, 2.3 Hz, 1H), 7.76 (d, J=8.5 Hz, 1H), 7.71 (s, 1H), 7.60 (d, J=9.7 Hz, 1H), 6.74 (d, J=9.7 Hz, 1H), 3.64 (s, 2H), 3.56 (t, J=6.9 Hz, 2H), 2.66 (t, J=6.9 Hz, 2H), 2.60-2.40 (m, 8H), 2.31 (s, 6H), 2.28 (s. 3H); ¹³C NMR (126 MHz, Methanol-d₄) δ 164.0, 154.5, 152.9, 147.0, 137.5, 137.3, 137.1, 134.7, 133.0, 131.2, 130.4, 128.8 (q, J=30.2 Hz), 125.3 (q, J=273.4), 124.3, 123.5, 120.3, 117.6, 114.6, 111.7, 92.9, 81.1, 57.5, 57.3, 54.6, 52.3, 44.6, 44.2, 38.5.

### N¹-(3-Bromoimidazo[1,2-b]pyridazin-6-yl)-N¹,N²,N²-trimethylethane-1,2-diamine

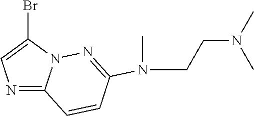 [SMILES: CN(C)CCN(C)c1ccc2ncc(Br)n2n1]

¹H NMR (500 MHz, Methanol-d₄) δ 7.77 (dd, J=10.0, 1.9 Hz, 1H), 7.53 (s, 1H), 7.12 (dd, J=10.0, 1.8 Hz, 1H), 4.05 (t, J=6.4 Hz, 2H), 3.50 (t, J=6.4 Hz, 2H), 3.21 (s, 3H), 3.05 (s, 6H); ¹³C NMR (126 MHz, Methanol-d₄) δ 155.2, 136.7, 130.7, 125.4, 110.0, 99.9, 54.7, 45.1, 43.1, 36.0.

### 5-((6-((2-(Dimethylamino)ethyl)(methyl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND-40)

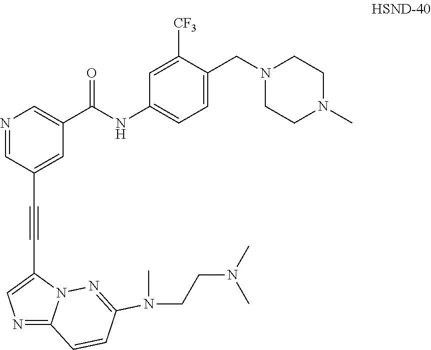 [SMILES: CN(C)CCN(C)c1ccc2ncc(C#Cc3cncc(C(=O)Nc4ccc(CN5CCN(C)CC5)c(C(F)(F)F)c4)c3)n2n1]

¹H NMR (500 MHz, Methanol-d₄) δ 8.99 (d, J=2.2 Hz, 1H), 8.77 (d, J=2.0 Hz, 1H), 8.37 (t, J=2.3 Hz, 1H), 8.10 (d, J=2.2 Hz, 1H), 7.92 (dd, J=8.4, 2.4 Hz, 1H), 7.77-7.70 (m, 2H), 7.68 (d, J=10.0 Hz, 1H), 7.03 (d, J=9.9 Hz, 1H), 3.72 (t, J=7.4 Hz, 2H), 3.62 (s, 2H), 3.13 (d, J=1.1 Hz, 3H), 2.61 (t, J=7.4 Hz, 2H), 2.58-2.37 (m, 8H), 2.31 (s, 6H), 2.27 (s, 3H); ¹³C NMR (126 MHz, Methanol-d₄) δ 163.8, 154.6, 152.9, 146.9, 137.5, 137.2, 136.7, 135.3, 133.0, 131.2, 130.3, 128.8 (q, J=30.2 Hz), 125.3 (q, J=273.4 Hz), 124.6, 123.4, 120.2, 117.6, 111.5, 111.0, 93.1, 81.2, 57.5, 55.3, 54.6, 52.3, 44.5, 35.8.

### 4-(3-Bromoimidazo[1,2-b]pyridazin-6-yl)morpholine

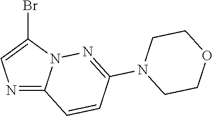 [SMILES: Brc1cnc2ccc(N3CCOCC3)nn12]

¹H NMR (500 MHz, Chloroform-d) δ 7.67 (dd, J=9.9, 1.8 Hz, 1H), 7.53 (s, 1H), 6.79 (d, J=9.9 Hz, 1H), 3.84 (t, J=4.9 Hz, 4H), 3.53 (t, J=4.9 Hz, 4H); ¹³C NMR (125 MHz, Chloroform-d) δ 155.2, 137.0, 132.3, 126.1, 109.2, 100.3, 66.4, 46.3.

### N-(4-((4-Methylpiperazin-1-yl)methyl)-3-(trifluoromethyl)phenyl)-5-((6-morpholinoimidazo[1,2-b]pyridazin-3-yl)ethynyl)nicotinamide (Compound HSND-41)

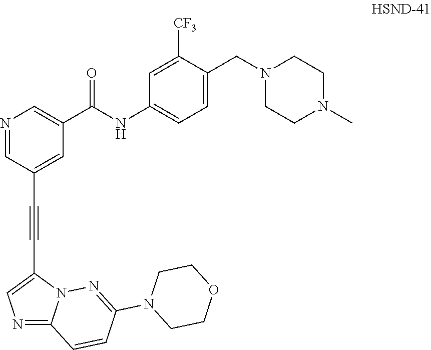 [SMILES: CN1CCN(Cc2ccc(NC(=O)c3cncc(C#Cc4cnc5ccc(N6CCOCC6)nn45)c3)cc2C(F)(F)F)CC1]

¹H NMR (500 MHz, Methanol-d₄) δ 8.94 (d, J=2.2 Hz, 1H), 8.71 (d, J=2.0 Hz, 1H), 8.31 (t, J=2.1 Hz, 1H), 8.06 (d, J=2.2 Hz, 1H), 7.88 (dd, J=8.4, 2.3 Hz, 1H), 7.75-7.64 (m, 3H), 7.10 (d, J=9.9 Hz, 1H), 3.81-3.75 (m, 4H), 3.60 (s, 2H), 3.52 (t, J=4.8 Hz, 4H), 2.52 (s, 8H), 2.32 (s, 3H); ¹³C NMR (125 MHz, Methanol-d₄) δ 163.7, 155.6, 152.9, 146.9, 137.4, 137.2, 135.8, 132.8, 131.1, 130.1, 128.7 (q, J=30.2 Hz), 125.3 (q, J=273.4 Hz), 124.9, 123.4, 120.1, 117.5 (q, J=6.3 Hz), 111.7, 93.5, 80.9, 66.0, 57.4, 54.5, 52.0, 45.7, 44.4.

### (R)-2-((3-Bromoimidazo[1,2-b]pyridazin-6-yl)amino)butan-1-ol

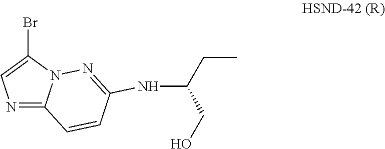 [SMILES: CC[C@H](CO)Nc1ccc2ncc(Br)n2n1]

¹H NMR (500 MHz, Methanol-d₄) δ 7.53 (d, J=9.7 Hz, 1H), 7.37 (s, 1H), 6.73 (d, J=9.7 Hz, 1H), 3.94 (dq, J=8.1, 5.3 Hz, 1H), 3.74-3.60 (m, 2H), 1.84-1.72 (m, 1H), 1.67-1.55 (m, 1H), 1.01 (t, J=7.5 Hz, 3H); ¹³C NMR (126 MHz, Methanol-d₄) δ 154.5, 136.8, 129.4, 124.1, 113.7, 99.8, 62.4, 54.3, 23.5, 9.6.

### (R)-5-((6-((1-Hydroxybutan-2-yl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND-42)

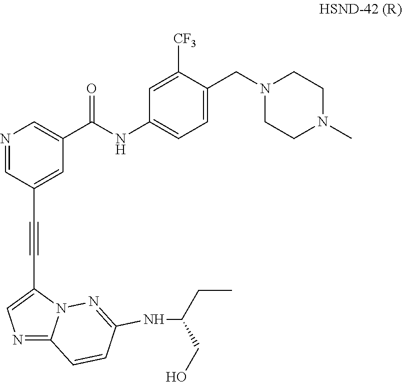 [SMILES: CC[C@H](CO)Nc1ccc2ncc(C#Cc3cncc(C(=O)Nc4ccc(CN5CCN(C)CC5)c(C(F)(F)F)c4)c3)n2n1]

¹H NMR (500 MHz, Methanol-d₄) δ 9.01 (s, 1H), 8.83 (s, 1H), 8.50-8.36 (m, 1H), 8.09 (s, 1H), 7.91 (d, J=8.6 Hz, 1H), 7.74 (d, J=8.5 Hz, 1H), 7.69 (s, 1H), 7.57 (dd, J=9.7, 1.7 Hz, 1H), 6.79 (dd, J=9.7, 1.7 Hz, 1H), 4.04-3.93 (m, 1H), 3.72 (d, J=5.4 Hz, 2H), 3.64 (s, 2H), 2.51 (s, 98H), 2.28 (s, 2H), 1.82-1.69 (m, 1H), 1.67-1.55 (m, 1H), 1.00 (t, J=7.4 Hz, 2H); ¹³C NMR (126 MHz, Methanol-d₄) δ 164.1, 154.8, 152.9, 147.0, 137.4, 137.3, 137.0, 134.5, 133.0, 131.1, 130.5, 128.8 (q, J=31.5 Hz), 125.3 q, J=274.6 Hz, 124.2, 123.4, 120.3, 117.5, 114.8, 111.6, 93.1, 81.2, 62.9, 57.4, 54.6, 52.3, 44.6, 23.6, 9.6.

### (S)-2-((3-Bromoimidazo[1,2-b]pyridazin-6-yl)amino)butan-1-ol

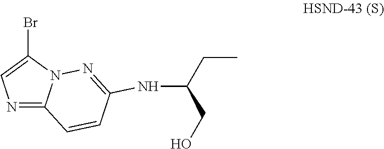 [SMILES: CC[C@@H](CO)Nc1ccc2ncc(Br)n2n1]

¹H NMR (500 MHz, Methanol-d₄) δ 7.53 (d, J=9.7 Hz, 1H), 7.38 (s, 1H), 6.73 (d, J=9.7 Hz, 1H), 3.97-3.88 (m, 1H), 3.73-3.65 (m, 2H), 1.85-1.73 (m, 1H), 1.69-1.54 (m, 1H), 1.01 (t, J=7.5 Hz, 3H); ¹³C NMR (126 MHz, Methanol-d₄) δ 154.5, 136.8, 129.4, 129.4, 124.1, 124.0, 113.7, 99.8, 62.4, 54.3, 54.2, 23.5, 23.5, 9.6, 9.5.

### (S)-5-((6-((1-Hydroxybutan-2-yl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND-43)

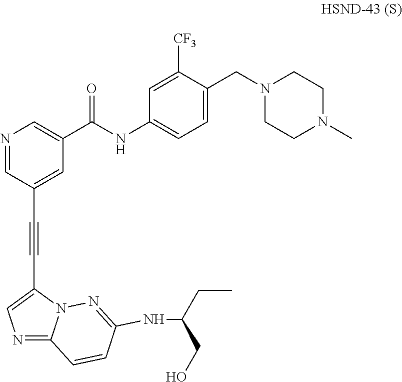 [SMILES: CC[C@@H](CO)Nc1ccc2ncc(C#Cc3cncc(C(=O)Nc4ccc(CN5CCN(C)CC5)c(C(F)(F)F)c4)c3)n2n1]

¹H NMR (500 MHz, Methanol-d₄) δ 9.00 (d, J=2.2 Hz, 1H), 8.82 (d, J=2.0 Hz, 1H), 8.49 (t, J=2.1 Hz, 1H), 8.10 (d, J=2.3 Hz, 1H), 7.93 (dd, J=8.5, 2.3 Hz, 1H), 7.73 (d, J=8.5 Hz, 1H), 7.68 (s, 1H), 7.57 (d, J=9.6 Hz. 1H), 6.80 (d, J=9.6 Hz, 1H), 4.07-3.95 (m, 1H), 3.74-3.62 (m, 4H), 3.06-2.92 (m, 4H), 2.72-2.57 (m, 7H), 1.79-1.70 (m, 1H), 1.66-1.54 (m, 1H), 0.98 (t, J=7.4 Hz, 3H); ¹³C NMR (126 MHz, Methanol-d₄) δ 164.1, 154.8, 153.0, 147.0, 137.7, 137.4, 134.5, 132.2, 131.2, 130.4, 128.8 (q, J=28.9 Hz), 125.3 (q, J=274.6 Hz), 124.2, 123.5, 120.3, 117.7, 114.9, 111.6, 93.0, 81.2, 63.0, 57.0, 54.5, 54.0, 50.7, 43.1, 23.6, 9.6.

### 5-((6-((2-Methoxyethyl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND-44)

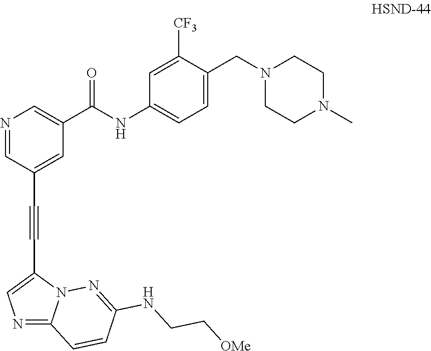 [SMILES: COCCNc1ccc2ncc(C#Cc3cncc(C(=O)Nc4ccc(CN5CCN(C)CC5)c(C(F)(F)F)c4)c3)n2n1]

¹H NMR (500 MHz, DMSO-d₆) δ 10.79 (s, 1H), 9.08 (d, J=2.2 Hz, 1H), 8.91 (d, J=2.0 Hz, 1H), 8.45 (t, J=2.1 Hz, 1H), 8.20 (d, J=2.2 Hz, 1H), 8.03 (dd, J=8.4, 2.2 Hz, 1H), 7.81 (s, 1H), 7.74 (dd, J=22.8, 9.1 Hz, 2H), 7.22 (t, J=5.4 Hz, 1H), 6.77 (d, J=9.7 Hz, 1H), 3.42 (t, J=6.2 Hz, 2H), 3.18 (s, 3H), 2.40 (s, 8H), 2.19 (s, 3H), 1.92-1.81 (m, 2H); ¹³C NMR (126 MHz, DMSO-d₆) δ 163.8, 154.7, 153.7, 148.4, 138.3, 137.5, 137.2, 136.1, 132.9, 131.8, 130.3, 128.0 (q, J=30.2 Hz), 125.8, 123.9, 123.6 (q, J=274.6 Hz), 119.6, 117.6, 114.8, 111.0, 94.2, 82.5, 70.2, 58.3, 57.8, 55.0, 52.8, 45.8, 38.6, 28.7.

### 5-((6-((3-Hydroxypropyl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND-45)

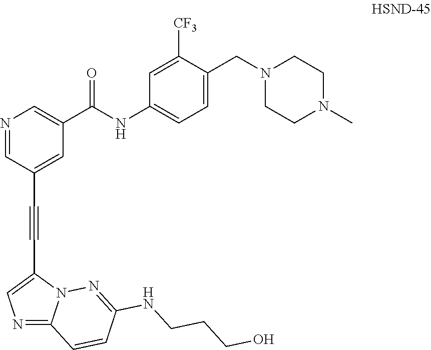 [SMILES: CN1CCN(Cc2ccc(NC(=O)c3cncc(C#Cc4cnc5ccc(NCCCO)nn45)c3)cc2C(F)(F)F)CC1]

¹H NMR (500 MHz, Methanol-d₄) δ 9.03 (d, J=2.2 Hz, 1H), 8.87 (d, J=2.0 Hz, 1H), 8.46 (t, J=2.1 Hz, 1H), 8.13 (d, J=2.3 Hz, 1H), 7.94 (dd, J=8.5, 2.3 Hz, 1H), 7.79-7.66 (m, 2H), 7.60 (d, J=9.7 Hz, 1H), 6.75 (d, J=9.6 Hz, 1H), 3.70 (t, J=6.2 Hz, 2H), 3.65 (d, J=1.6 Hz, 2H), 3.52 (t, J=6.8 Hz, 2H), 2.51 (s, 8H), 2.29 (s, 3H), 1.96-1.88 (m, 2H); ¹³C NMR (126 MHz, Methanol-d₄) δ 164.1, 154.9, 153.0, 147.0, 137.5, 137.2, 134.6, 133.0, 131.2, 130.4, 128.8 (q, J=30.2 Hz), 125.3 (q, J=274.6 Hz), 124.3, 123.5, 120.3, 117.6, 114.7, 93.2, 81.0, 59.1, 57.5, 54.6, 52.3, 44.6, 37.9, 31.5.

### 3-Bromo-6-(piperazin-1-yl)imidazo[1,2-b]pyridazine

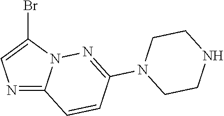 [SMILES: Brc1cnc2ccc(N3CCNCC3)nn12]

¹H NMR (500 MHz, Methanol-d₄) δ 7.69 (d, J=10.0 Hz, 1H), 7.49 (s, 1H), 7.15 (d, J=10.0 Hz, 1H), 3.65-3.56 (m, 4H), 3.03-2.95 (m, 4H); ¹³C NMR (125 MHz, Methanol-d₄) δ 155.5, 136.8, 130.7, 124.8, 111.0, 100.1, 45.8, 44.4.

### N-(4-((4-Methylpiperazin-1-yl)methyl)-3-(trifluoromethyl)phenyl)-5-((6-(piperazin-1-yl)imidazo[1,2-b]pyridazin-3-yl)ethynyl)nicotinamide (Compound HSND-76)

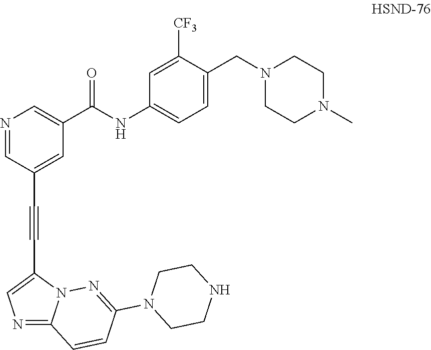 [SMILES: CN1CCN(Cc2ccc(NC(=O)c3cncc(C#Cc4cnc5ccc(N6CCNCC6)nn45)c3)cc2C(F)(F)F)CC1]

¹H NMR (500 MHz, Methanol-d₄) δ 9.15-8.93 (m, 1H), 8.93-8.78 (m, 1H), 8.52-8.37 (m, 1H), 8.14 (q, J=2.8 Hz, 1H), 7.96-7.89 (m, 1H), 7.86-7.73 (m, 3H), 7.28-7.20 (m, 1H), 3.68-3.53 (m, 6H), 3.02-2.91 (m, 4H), 2.52 (s, 8H), 2.29 (q, J=4.0 Hz, 3H); ¹³C NMR (125 MHz, Methanol-d₄) δ 164.1, 155.8, 153.0, 147.1, 137.5, 137.4, 137.1, 135.7, 133.0, 131.2, 130.5, 128.6 (q, J=31.2 Hz), 125.3 (q, J=270 Hz), 124.8, 123.5, 120.2, 117.6, 112.3, 111.9, 93.3, 80.8, 57.4, 54.5, 52.2, 46.0, 44.6, 44.5.

### 3-Bromo-6-(4-methylpiperazin-1-yl)imidazo[1,2-b]pyridazine

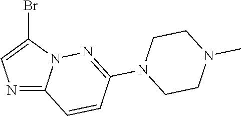 [SMILES: CN1CCN(c2ccc3ncc(Br)n3n2)CC1]

¹H NMR (500 MHz, Methanol-d₄) δ 7.69 (d, J=10.0 Hz, 1H), 7.49 (s, 1H), 7.15 (d, J=10.0 Hz, 1H), 3.64-3.57 (m, 4H), 2.63-2.55 (m, 4H), 2.35 (s, 3H). ¹³C NMR (125 MHz, Methanol-d₄) δ 155.4, 136.7, 130.7, 124.8, 110.9, 100.1, 54.0, 45.1, 44.7.

### 5-((6-(4-Methylpiperazin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-77)

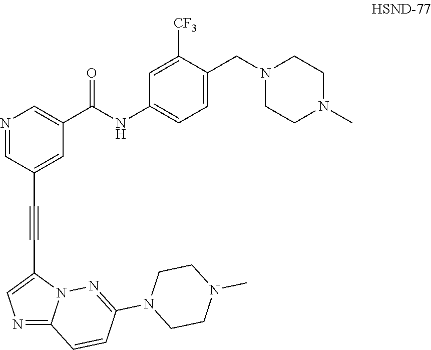 [SMILES: CN1CCN(Cc2ccc(NC(=O)c3cncc(C#Cc4cnc5ccc(N6CCN(C)CC6)nn45)c3)cc2C(F)(F)F)CC1]

¹H NMR (500 MHz, Methanol-d₄) δ 9.01 (s, 1H), 8.81 (s, 1H), 8.45-8.38 (m, 1H), 8.12 (d, J=2.2 Hz, 1H), 7.92 (dd, J=8.5, 2.3 Hz, 1H), 7.83-7.71 (m, 3H), 7.22 (dd, J=10.0, 3.2 Hz, 1H), 3.69-3.61 (m, 6H), 2.73-2.49 (m, 12H), 2.38 (s. 3H), 2.34 (s, 3H); ¹³C NMR (125 MHz, Methanol-d₄) δ 164.0, 155.5, 153.0, 147.0, 137.5, 137.3, 135.7, 132.8, 131.2, 130.4, 128.8 (q, J=30 Hz), 125.3 (q, J=272.5 Hz), 124.9, 123.5, 120.2, 117.6, 112.2, 93.4, 80.9, 57.3, 54.4, 54.0, 51.9, 45.0, 44.7, 44.2.

### 3-Bromo-6-(piperidin-1-yl)imidazo[1,2-b]pyridazine

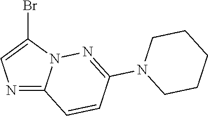 [SMILES: Brc1cnc2ccc(N3CCCCC3)nn12]

¹H NMR (500 MHz, Methanol-d₄) δ 7.60 (d, J=10.0 Hz, 1H), 7.43 (s, 1H), 7.06 (d, J=10.0 Hz, 1H), 3.53 (dd, J=6.1, 3.9 Hz, 4H), 1.64 (dt, J=5.4, 2.8 Hz, 6H); ¹³C NMR (125 MHz, Methanol-d₄) δ 155.3, 136.5, 130.3, 124.5, 111.1, 99.9, 46.6, 25.0, 24.1.

### N-(4-((4-Methylpiperazin-1-yl)methyl)-3-(trifluoromethyl)phenyl)-5-((6-(piperidin-1-yl)imidazo[1,2-b]pyridazin-3-yl)ethynyl)nicotinamide (Compound HSND-78)

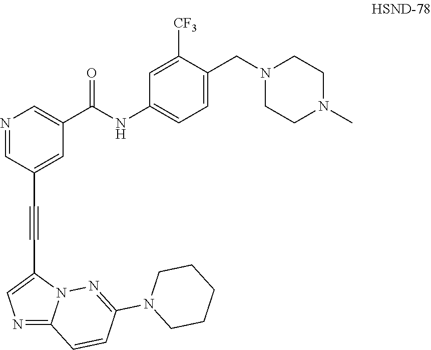 [SMILES: CN1CCN(Cc2ccc(NC(=O)c3cncc(C#Cc4cnc5ccc(N6CCCCC6)nn45)c3)cc2C(F)(F)F)CC1]

¹H NMR (500 MHz, Methanol-d₄) δ 9.04 (s, 1H), 8.84 (s, 1H), 8.47-8.34 (m, 1H), 8.12 (d, J=2.2 Hz, 1H), 7.94 (dd, J=8.5, 2.3 Hz, 1H), 7.81-7.64 (m, 3H), 7.22 (d, J=8.4 Hz, 1H), 3.67 (s, 2H), 3.64-3.59 (m, 4H). 2.60 (d, J=40.4 Hz. 8H), 2.39 (s, 3H), 1.76-1.67 (m, 6H).

### 4-(3-Bromoimidazo[1,2-b]pyridazin-6-yl)thiomorpholine

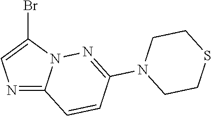 [SMILES: Brc1cnc2ccc(N3CCSCC3)nn12]

¹H NMR (500 MHz, Methanol-d₄) δ 7.67 (d, J=10.0 Hz, 1H), 7.47 (s, 1H), 7.11 (d, J=10.0 Hz, 1H), 3.99-3.92 (m, 4H), 2.73-2.61 (m, 4H). ¹³C NMR (125 MHz, Methanol-d₄) δ 154.6, 136.5, 130.6, 124.9, 111.0, 100.1, 48.4, 25.4.

### N-(4-((4-Methylpiperazin-1-yl)methyl)-3-(trifluoromethyl)phenyl)-5-((6-thiomorpholinoimidazo[1,2-b]pyridazin-3-yl)ethynyl)nicotinamide (Compound HSND-79)

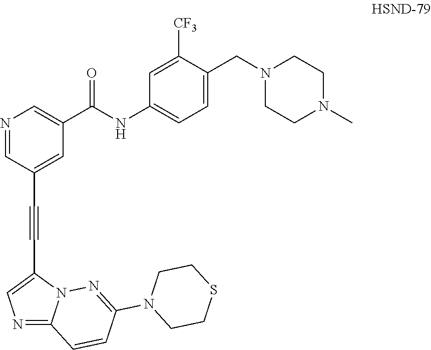 [SMILES: CN1CCN(Cc2ccc(NC(=O)c3cncc(C#Cc4cnc5ccc(N6CCSCC6)nn45)c3)cc2C(F)(F)F)CC1]

¹H NMR (500 MHz, DMSO-d₆) δ 10.81 (s, 1H), 9.08 (d, J=2.1 Hz, 1H), 8.90 (s, 1H), 8.51-8.41 (m, 1H), 8.19 (d, J=2.2 Hz, 1H), 8.03 (dd, J=8.5, 2.2 Hz, 1H), 7.96-7.88 (m, 2H), 7.70 (d, J=8.5 Hz, 1H), 7.30 (d, J=9.9 Hz, 1H), 3.96-3.90 (m, 4H), 3.55 (s, 2H), 2.74-2.66 (m, 4H), 2.40 (s, 8H), 2.20 (s, 3H).; ¹³C NMR (125 MHz, DMSO-d₆) δ 163.8, 154.9, 153.8, 148.5, 138.3, 137.3, 137.2, 132.8, 131.7, 130.3, 128.0 (q, J=28.7 Hz), 126.6, 125.8 (J=272.5 Hz), 123.9, 119.4, 117.7, 112.7, 111.2, 94.5, 82.0, 57.7, 54.9, 52.7, 48.6, 45.7, 25.8.

### (2S,6R)-4-(3-Bromoimidazo[1,2-b]pyridazin-6-yl)-2,6-dimethylmorpholine

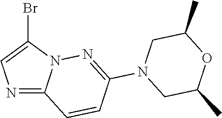 [SMILES: C[C@@H]1CN(c2ccc3ncc(Br)n3n2)C[C@H](C)O1]

¹H NMR (500 MHz, Chloroform-d) δ 7.66 (d, J=9.9 Hz, 1H), 7.52 (s, 1H), 6.80 (d, J=9.9 Hz, 1H), 4.01-3.89 (m, 2H), 3.81-3.65 (m, 2H), 2.65 (dd, J=12.8, 10.6 Hz, 2H), 1.28 (d, J=6.3 Hz, 6H). ¹³C NMR (125 MHz, Chloroform-d) δ 154.8, 136.9, 132.1, 126.0, 109.4, 100.3, 71.3, 51.3, 18.9.

### 5-((6-((2S,6R)-2,6-Dimethylmorpholino)imidazo[1,2-b]pyridazin-3-yl)ethynyl)-N-(4-((4-methylpiperazin-1-yl)methyl)-3-(trifluoromethyl)phenyl)nicotinamide (Compound HSND-80)

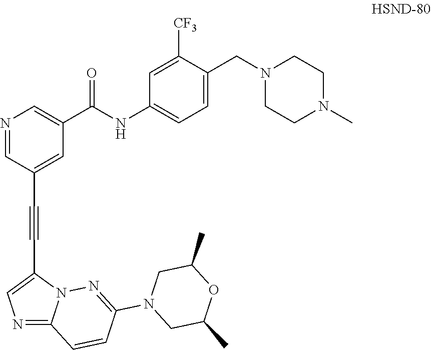 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)O1]

¹H NMR (500 MHz, Methanol-d₄) δ 8.97 (s, 1H), 8.73 (s, 1H), 8.32 (s, 1H), 8.10 (s, 1H), 7.90 (d, J=8.5 Hz, 1H), 7.77-7.54 (m, 3H), 7.15 (d, J=8.4 Hz, 1H), 4.05 (d, J=12.7 Hz, 2H), 3.77-3.66 (m, 2H), 3.64 (s, 2H), 2.79-2.45 (m, 11H), 2.40 (s, 3H), 1.20 (d, J=6.2 Hz, 6H); ¹³C NMR (125 MHz, Methanol-d₄) δ 163.7, 155.2, 152.8, 146.8, 137.5, 137.2, 132.7, 131.2, 131.2, 130.3, 128.8 (q, J=30 Hz), 127.5, 125.3 (q, J=272.5 Hz), 125.0, 123.4, 120.3, 117.6, 111.9, 93.7, 81.2, 71.2, 57.3, 54.4, 51.7, 50.8, 44.1, 17.7.

### N-(4-chloro-3-(trifluoromethyl)phenyl)-5-ethynylnicotinamide

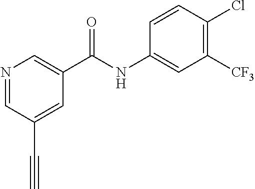 [SMILES: C#Cc1cncc(C(=O)Nc2ccc(Cl)c(C(F)(F)F)c2)c1]

¹H NMR (500 MHz, Methanol-d₄) δ 9.06 (d, J=2.2 Hz, 1H), 8.81 (d, J=2.0 Hz, 1H), 8.42 (t, J=2.1 Hz, 1H), 8.26 (d, J=2.6 Hz, 1H), 7.97 (dd, J=8.8, 2.6 Hz, 1H), 7.60 (d, J=8.8 Hz, 1H), 3.93 (s, 1H). ¹³C NMR (125 MHz, Methanol-d₄) δ 164.1, 154.1, 147.5, 138.4, 137.7, 131.7, 130.2, 128.1 (J=31.5 Hz), 126.4, 124.7, 123.8 (J=270 Hz), 119.8, 119.2, 82.5, 78.6.

### N-(4-Chloro-3-(trifluoromethyl)phenyl)-5-((6-morpholinoimidazo[1,2-b]pyridazin-3-yl)ethynyl)nicotinamide (Compound HSND-91)

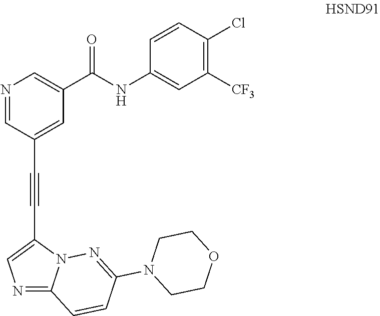 [SMILES: O=C(Nc1ccc(Cl)c(C(F)(F)F)c1)c1cncc(C#Cc2cnc3ccc(N4CCOCC4)nn23)c1]

¹H NMR (500 MHz, DMSO-d₆) δ 10.88 (s, 1H), 9.09 (s, 1H), 8.96 (s, 1H), 8.48 (q, J=1.8 Hz, 1H), 8.33 (t, J=1.9 Hz, 1H), 8.15-7.91 (m, 4H), 7.74 (d, J=8.8 Hz, 1H), 7.32 (d, J=8.1 Hz, 1H), 3.79-3.69 (m, 4H), 3.60-3.48 (m, 4H); ¹³C NMR (125 MHz, DMSO-d₆) δ 164.0, 155.9, 154.0, 148.6, 138.7, 137.4, 132.6, 130.1, 127.3, 126.7, 125.4, 125.2, 124.2 (q, J=270 Hz), 119.4, 112.2, 94.5, 82.1, 66.2, 46.0.

### 5-Ethynyl-N-(3-(trifluoromethyl)phenyl)nicotinamide

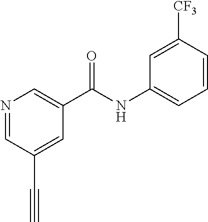 [SMILES: C#Cc1cncc(C(=O)Nc2cccc(C(F)(F)F)c2)c1]

¹H NMR (500 MHz, Methanol-d₄) δ 9.04 (d, J=2.1 Hz, 1H), 8.78 (d, J=1.9 Hz, 1H), 8.40 (t, J=2.1 Hz, 1H), 8.14 (d, J=2.0 Hz, 1H), 7.97-7.87 (m, 1H), 7.53 (t, J=8.0 Hz, 1H), 7.45-7.38 (m, 1H), 3.91 (s, 1H); ¹³C NMR (125 MHz, Methanol-d₄) δ 164.1, 154.1, 147.5, 139.0, 138.4, 130.9 (q, J=31.2 Hz), 130.4, 129.3, 125.1 (q, J=270 Hz), 123.6, 120.7, 119.7, 116.9, 82.5, 78.6.

### 5-((6-Morpholinoimidazo[1,2-b]pyridazin-3-yl)ethynyl)-N-(3-(trifluoromethyl)phenyl)nicotinamide (Compound HSND-92)

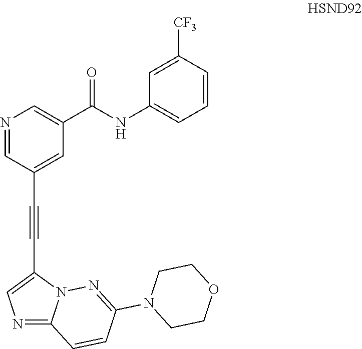 [SMILES: O=C(Nc1cccc(C(F)(F)F)c1)c1cncc(C#Cc2cnc3ccc(N4CCOCC4)nn23)c1]

¹H NMR (500 MHz, DMSO-d₆) δ 10.80 (s, 1H), 9.09 (d, J=2.1 Hz, 1H), 8.99-8.92 (m, 1H), 8.49 (t, J=2.1 Hz, 1H), 8.23 (d, J=2.0 Hz, 1H), 8.09-7.91 (m, 3H), 7.69-7.59 (m, 1H), 7.50-7.44 (m, 1H), 7.32 (d, J=9.9 Hz, 1H), 3.74 (t, J=4.9 Hz, 4H), 3.58-3.50 (m, 4H); ¹³C NMR (125 MHz, DMSO-d₆) δ 164.0, 155.9, 153.9, 148.6, 140.0, 137.4, 130.5, 130.3, 130.0 (q, J=31.2 Hz), 126.6, 125.6 (q, J=273.4 Hz), 124.2, 120.9, 119.4, 116.8, 112.3, 94.5, 82.0, 66.2, 46.0.

### 3-Bromo-6-((3S,5R)-3,5-dimethylpiperazin-1-yl)imidazo[1,2-b]pyridazine

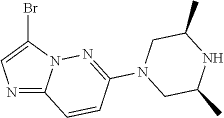 [SMILES: C[C@@H]1CN(c2ccc3ncc(Br)n3n2)C[C@H](C)N1]

¹H NMR (500 MHz, Chloroform-d) δ 7.64 (d, J=9.9 Hz, 1H), 7.50 (s, 1H), 6.82 (d, J=9.9 Hz, 1H), 4.09-3.98 (m, 2H), 2.98 (ddt, J=12.9, 6.4, 3.3 Hz, 2H), 2.53 (dd, J=12.7, 10.6 Hz, 2H), 2.00 (s, 2H), 1.16 (d, J=6.3 Hz, 6H); ¹³C NMR (125 MHz, Chloroform-d) δ 154.9, 136.8, 132.0, 125.9, 109.8, 100.2, 52.5, 50.4, 19.6.

### 5-((6-((3S,5R)-3,5-Dimethylpiperazin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-96)

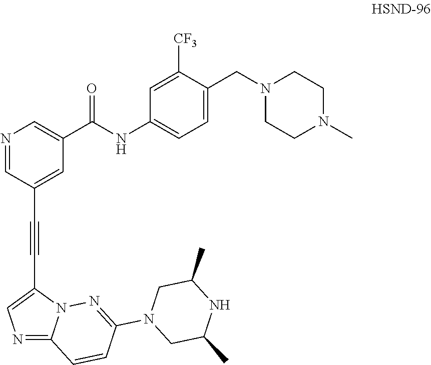 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)N1]

¹H NMR (500 MHz, Methanol-d₄) δ 9.03 (d, J=2.2 Hz, 1H), 8.83 (d, J=2.0 Hz, 1H), 8.42 (t, J=2.1 Hz, 1H), 8.12 (d, J=2.3 Hz, 1H), 7.93 (dd, J=8.5, 2.3 Hz, 1H), 7.81-7.71 (m, 3H), 7.25 (d, J=10.0 Hz, 1H), 4.24-4.16 (m, 2H), 3.65 (d, J=1.6 Hz, 2H), 3.01-2.91 (m, 10H), 2.62-2.42 (m, 2H), 2.29 (s, 3H), 1.17 (d, J=6.4 Hz, 6H); ¹³C NMR (125 MHz, Methanol-d₄) δ 164.0, 155.3, 152.9, 147.0, 137.5, 137.3, 135.5, 133.0, 131.2, 130.5, 128.8 (q, J=30 Hz), 125.3 (q, J=272.5 Hz), 124.8, 123.4, 120.3, 117.6, 112.4, 93.4, 80.9, 57.5, 54.6, 52.3, 51.5, 50.0, 44.6, 17.7.

### 3-Bromo-6-(4-(2-methoxyethyl)piperazin-1-yl)imidazo[1,2-b]pyridazine

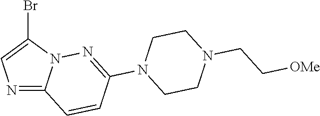 [SMILES: COCCN1CCN(c2ccc3ncc(Br)n3n2)CC1]

¹H NMR (500 MHz, Methanol-d₄) δ 7.66 (d, J=9.9 Hz, 1H), 7.47 (s, 1H), 7.10 (d, J=10.0 Hz, 1H), 3.61-3.51 (m, 6H), 3.34 (s, 3H), 2.66-2.58 (m, 6H).; ¹³C NMR (125 MHz, Methanol-d₄) δ 155.3, 136.7, 130.6, 124.8, 110.9, 100.1, 69.6, 57.6, 57.2, 52.6, 45.1.

### 5-((6-(4-(2-Methoxyethyl)piperazin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-97)

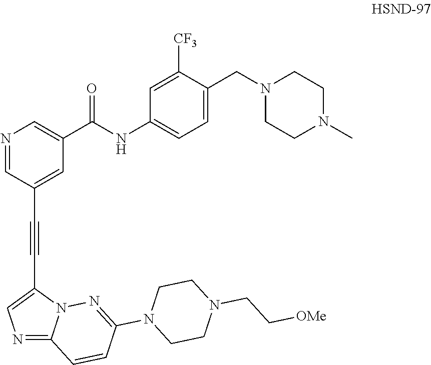 [SMILES: COCCN1CCN(c2ccc3ncc(C#Cc4cncc(C(=O)Nc5ccc(CN6CCN(C)CC6)c(C(F)(F)F)c5)c4)n3n2)CC1]

¹H NMR (500 MHz, Methanol-d₄) δ 8.96 (d, J=2.2 Hz, 1H), 8.73 (d, J=2.0 Hz, 1H), 8.35 (t, J=2.1 Hz, 1H), 8.07 (d, J=2.2 Hz, 1H), 7.89 (dd, J=8.4, 2.3 Hz, 1H), 7.74-7.61 (m, 3H), 7.12 (d, J=10.0 Hz, 1H), 3.59 (dd, J=9.3, 3.9 Hz, 6H), 3.51 (t, J=5.5 Hz, 2H), 3.31 (s, 3H), 2.66-2.41 (m, 14H), 2.28 (s, 3H). ¹³C NMR (125 MHz, Methanol-d₄) δ 163.7, 155.4, 152.9, 146.9, 137.4, 137.2, 136.9, 135.6, 132.9, 131.1, 130.2, 128.7 (q, J=30 Hz), 128.5, 125.3 (q, J=272.5 Hz), 124.8, 123.3, 123.1, 120.1, 117.5, 112.0, 111.8, 93.5, 81.0, 69.6, 69.5, 57.6, 57.4, 57.2, 57.2, 54.6, 54.5, 52.7, 52.6, 52.2, 52.2, 45.1, 44.6.

### 3-Bromo-6-((3S,5R)-3,4,5-trimethylpiperazin-1-yl)imidazo[1,2-b]pyridazine

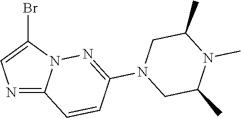 [SMILES: C[C@@H]1CN(c2ccc3ncc(Br)n3n2)C[C@H](C)N1C]

¹H NMR (500 MHz, Chloroform-d) δ 7.66 (d, J=9.8 Hz, 1H). 7.52 (s, 1H), 6.82 (dd, J=9.9, 2.6 Hz, 1H), 4.14-3.84 (m, 2H), 2.84 (t, J=11.9 Hz, 2H), 2.35 (d, J=13.4 Hz, 5H), 1.22 (d, J=6.3 Hz, 6H). ¹³C NMR (125 MHz, Chloroform-d) δ 154.5, 136.8, 132.1, 126.0, 109.5, 100.3, 57.4, 52.6, 37.6, 17.9.

### N-(4-((4-Methylpiperazin-1-yl)methyl)-3-(trifluoromethyl)phenyl)-5-((6-((3S,5R)-3,4,5-trimethylpiperazin-1-yl)imidazo[1,2-b]pyridazin-3-yl)ethynyl)nicotinamide (Compound HSND-99)

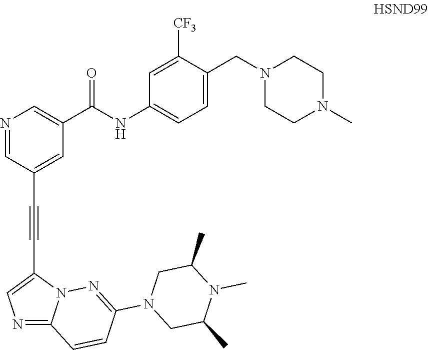 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)N1C]

¹H NMR (500 MHz, Methanol-d₄) δ 9.03 (s, 1H), 8.84 (s, 1H), 8.44-8.40 (m, 1H), 8.12 (t, J=2.1 Hz, 1H), 7.93 (dt, J=8.6, 2.1 Hz, 1H), 7.85-7.69 (m, 4H), 7.25 (dd, J=9.9, 1.4 Hz, 1H), 4.18-4.12 (m, 2H), 3.65 (s, 2H), 2.88-2.74 (m, 2H), 2.61-2.38 (m, 10H), 2.34-2.31 (m, 3H), 2.31-2.28 (m, 3H), 1.20 (dd, J=6.2, 1.8 Hz, 6H); ¹³C NMR (125 MHz, Methanol-d₄) δ 164.0, 155.0, 152.8, 147.0, 137.5, 137.3, 135.5, 133.0, 131.2, 130.5, 128.8 (q, J=30 Hz), 125.3 (q, J=272.5 Hz), 124.9, 123.4, 120.3, 117.6, 112.2, 93.5, 80.9, 57.4, 57.3, 54.6, 52.2, 51.7, 44.5, 36.4, 16.3.

### (2S,6S)-4-(3-Bromoimidazo[1,2-b]pyridazin-6-yl)-2,6-dimethylmorpholine

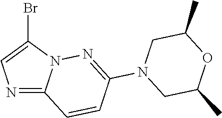 [SMILES: C[C@@H]1CN(c2ccc3ncc(Br)n3n2)C[C@H](C)O1]

¹H NMR (500 MHz, Chloroform-d) δ 7.68 (d, J=9.9 Hz, 1H), 7.53 (s, 1H), 6.79 (d, J=9.9 Hz, 1H), 4.27-4.06 (m, 2H), 3.65 (dd, J=12.8, 3.4 Hz, 2H), 3.27 (dd, J=12.8, 6.3 Hz, 2H), 1.30 (d, J=6.4 Hz, 6H); ¹³C NMR (125 MHz, Chloroform-d) δ 155.4, 136.8, 132.0, 126.0, 109.4, 100.3, 66.0, 50.7, 17.9.

### 5-((6-((2R,6R)-2,6-Dimethylmorpholino)imidazo[1,2-b]pyridazin-3-yl)ethynyl)-N-(4-((4-methylpiperazin-1-yl)methyl)-3-(trifluoromethyl)phenyl)nicotinamide (Compound HSND-100)

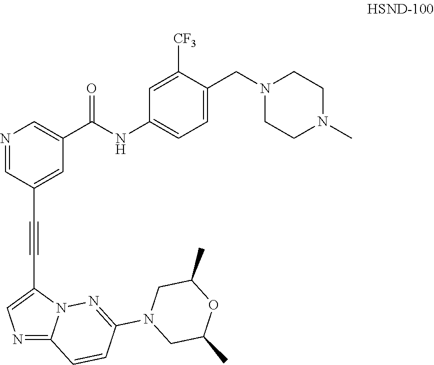 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)O1]

¹H NMR (500 MHz, Methanol-d₄) δ 9.08 (s, 1H), 8.89 (s, 1H), 8.46 (s, 1H), 8.16 (d, J=2.2 Hz, 1H), 8.00-7.72 (m, 4H), 7.26 (d, J=6.9 Hz, 1H), 4.21-4.11 (m, 2H), 3.73 (dd, J=13.0, 3.3 Hz, 2H), 3.68 (s, 2H), 3.41-3.34 (m, 2H), 2.80-2.50 (m, 8H), 2.42 (s, 3H), 1.27 (d, J=6.4 Hz, 6H). ¹³C NMR (125 MHz, Methanol-d₄) δ 164.1, 155.9, 153.0, 147.1, 137.6, 137.4, 132.8, 131.2, 128.8, 128.6, 125.3, 125.1, 123.5, 123.2, 117.7, 112.0, 93.4, 66.0, 57.3, 54.3, 51.7, 50.1, 44.0, 29.3, 16.6.

### (S)-4-(3-Bromoimidazo[1,2-b]pyridazin-6-yl)-2-methylmorpholine

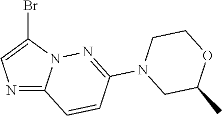 [SMILES: C[C@H]1CN(c2ccc3ncc(Br)n3n2)CCO1]

¹H NMR (500 MHz, Methanol-d₄) δ 7.71 (d, J=10.0 Hz, 1H), 7.50 (s, 1H), 7.17 (d, J=10.0 Hz, 1H), 4.13-3.95 (m, 3H), 3.76-3.65 (m, 2H), 3.09-2.97 (m, 1H), 2.74-2.61 (m, 1H), 1.24 (d, J=6.3 Hz, 3H); ¹³C NMR (125 MHz, Methanol-d₄) δ 155.5, 136.9, 130.7, 124.9, 110.8, 100.2, 71.4, 65.8, 51.6, 45.1, 17.6.

### (R)-5-((6-(2-Methylmorpholino)imidazo[1,2-b]pyridazin-3-yl)ethynyl)-N-(4-((4-methylpiperazin-1-yl)methyl)-3-(trifluoromethyl)phenyl)nicotinamide (Compound HSND-101)

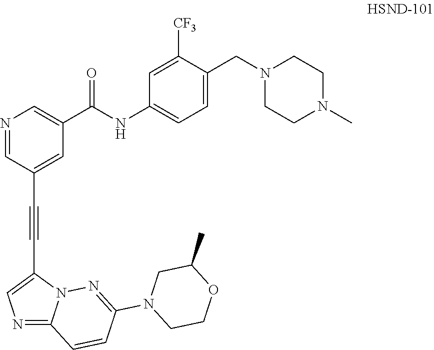 [SMILES: C[C@@H]1CN(c2ccc3ncc(C#Cc4cncc(C(=O)Nc5ccc(CN6CCN(C)CC6)c(C(F)(F)F)c5)c4)n3n2)CCO1]

¹H NMR (500 MHz, Methanol-d₄) δ 9.03 (s, 1H), 8.84 (s, 1H), 8.43 (t, J=2.0 Hz, 1H), 8.13 (d, J=2.2 Hz, 1H), 7.94 (dd, J=8.5, 2.2 Hz, 1H), 7.88-7.71 (m, 3H), 7.23 (d, J=9.6 Hz, 1H), 4.15-4.04 (m, 2H), 4.03-3.98 (m, 1H), 3.78-3.67 (m, 2H), 3.67 (s, 2H), 3.10-3.02 (m, 1H), 2.76-2.46 (m, 10H), 2.40 (s, 3H), 1.23 (d, J=6.2 Hz, 3H); ¹³C NMR (125 MHz, Methanol-d₄) δ 164.0, 155.6, 153.0, 147.0, 137.5, 137.4, 132.8, 131.2, 130.5, 128.9 (q, J=30 Hz), 125.3 (q, J=271.2 Hz), 125.0, 120.2, 117.6, 112.0, 93.4, 80.9, 71.4, 65.8, 57.3, 54.4, 51.8, 51.6, 45.0, 44.1, 17.6.

### 4-(3-Bromoimidazo[1,2-b]pyridazin-6-yl)-2,2,6,6-tetramethylmorpholine

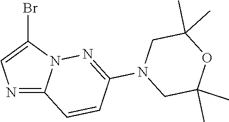 [SMILES: CC1(C)CN(c2ccc3ncc(Br)n3n2)CC(C)(C)O1]

¹H NMR (500 MHz, Chloroform-d) δ 7.68 (dd, J=9.9, 1.3 Hz, 1H), 7.52 (s, 1H), 6.77 (d, J=9.9 Hz, 1H), 3.42 (s, 4H), 1.31 (s, 12H). ¹³C NMR (125 MHz, Chloroform-d) δ 155.1, 136.8, 132.0, 126.1, 109.2, 100.2, 71.6, 54.5, 28.6.

### N-(4-((4-Methylpiperazin-1-yl)methyl)-3-(trifluoromethyl)phenyl)-5-((6-(2,2,6,6-tetramethylmorpholino)imidazo[1,2-b]pyridazin-3-yl)ethynyl)nicotinamide (Compound HSND-102)

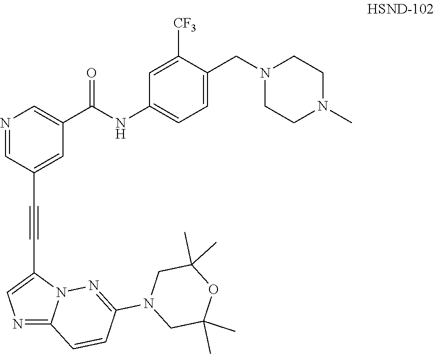 [SMILES: CN1CCN(Cc2ccc(NC(=O)c3cncc(C#Cc4cnc5ccc(N6CC(C)(C)OC(C)(C)C6)nn45)c3)cc2C(F)(F)F)CC1]

¹H NMR (500 MHz, Methanol-d₄) δ 9.03 (d, J=2.1 Hz, 1H), 8.83 (d, J=2.0 Hz, 1H), 8.47-8.39 (m, 1H), 8.12 (d, J=2.2 Hz, 1H), 7.94 (dd, J=8.4, 2.3 Hz, 1H), 7.84-7.70 (m, 3H), 7.22 (d, J=9.9 Hz, 1H), 3.71-3.62 (m, 2H), 3.54 (s, 4H), 2.57 (s, 8H), 2.36 (d, J=1.5 Hz, 3H), 1.29 (d, J=1.5 Hz, 12H); ¹³C NMR (125 MHz, Methanol-d₄) δ 164.0, 155.5, 152.9, 146.9, 137.5, 137.4, 135.5, 132.9, 131.2, 130.5, 128.8 (q, J=30 Hz), 125.3 (q, J=271.2 Hz), 125.0, 123.4, 123.2, 120.3, 117.6, 111.7, 93.4, 81.0, 71.7, 57.4, 54.5, 53.7, 52.0, 44.3, 27.5.

### 3-Bromo-6-(3,5-dimethylpiperidin-1-yl)imidazo[1,2-b]pyridazine

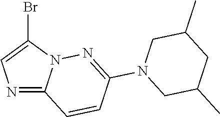 [SMILES: CC1CC(C)CN(c2ccc3ncc(Br)n3n2)C1]

¹H NMR (500 MHz, Chloroform-d) δ 7.71-7.53 (m, 1H), 7.56-7.43 (m, 1H), 6.91-6.73 (m, 1H), 4.14 (dt, J=12.4, 5.4 Hz, 2H), 2.41 (q, J=12.2, 11.2 Hz, 2H), 1.90-1.82 (m, 1H), 1.81-1.68 (m, 2H), 1.04-0.89 (m, 6H), 0.81 (q, J=12.0 Hz, 1H). ¹³C NMR (125 MHz, Chloroform-d) δ 154.9, 136.7, 131.7, 125.7, 110.2, 100.1, 53.4, 42.3, 30.7, 19.2.

### 5-((6-(3,5-Dimethylpiperidin-1-yl)imidazo[1,2-b]pyridazin-3-yl)ethynyl)-N-(4-((4-methylpiperazin-1-yl)methyl)-3-(trifluoromethyl)phenyl)nicotinamide

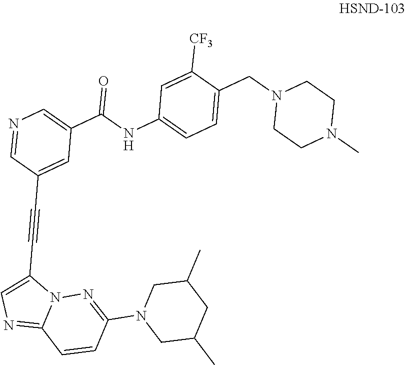 [SMILES: CC1CC(C)CN(c2ccc3ncc(C#Cc4cncc(C(=O)Nc5ccc(CN6CCN(C)CC6)c(C(F)(F)F)c5)c4)n3n2)C1]

¹H NMR (500 MHz, Methanol-d₄) δ 9.11 (s, 1H), 8.92 (s, 1H), 8.43 (s, 1H), 8.13 (d, J=2.2 Hz, 1H), 7.95 (d, J=8.4 Hz, 1H), 7.85-7.55 (m, 3H), 7.32 (s, 1H), 4.29 (d, J=12.8 Hz, 2H), 3.68 (s, 2H), 2.75-2.51 (m, 8H), 2.47 (t, J=12.2 Hz, 1H), 2.41 (s, 2H), 1.86 (d, J=12.9 Hz, 1H), 1.74 (s, 2H), 0.96 (d, J=6.5 Hz, 6H), 0.88-0.82 (m, 1H).

### N-(3-(4-Methyl-1H-imidazol-1-yl)-5-(trifluoromethyl)phenyl)-5-((6-morpholinoimidazo[1,2-b]pyridazin-3-yl)ethynyl)nicotinamide (Compound HSND-105)

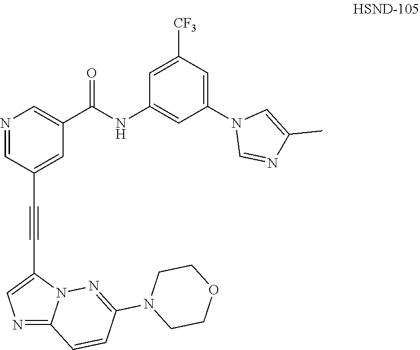 [SMILES: Cc1cn(-c2cc(NC(=O)c3cncc(C#Cc4cnc5ccc(N6CCOCC6)nn45)c3)cc(C(F)(F)F)c2)cn1]

¹H NMR (500 MHz, Methanol-d₄) δ 9.06 (s, 1H), 8.85 (s, 1H), 8.45 (s, 1H), 8.23 (d, J=2.5 Hz, 1H), 8.17 (s, 1H), 8.06 (s, 1H), 7.85-7.74 (m, 2H), 7.64 (d, J=4.4 Hz, 1H), 7.38 (s, 1H), 7.22 (d, J=9.8 Hz, 1H), 3.83 (t, J=4.9 Hz, 4H), 3.59 (t, J=4.9 Hz, 4H), 2.26 (s, 3H).

### 5-((6-((2S,6R)-2,6-Dimethylmorpholino)imidazo[1,2-b]pyridazin-3-yl)ethynyl)-N-(3-(trifluoromethyl)phenyl)nicotinamide (Compound HSND-106)

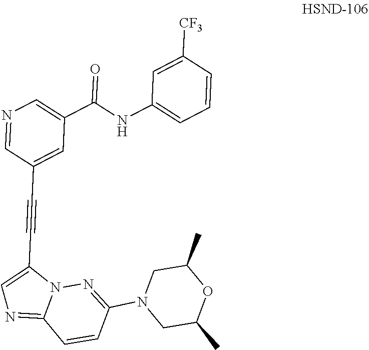 [SMILES: C[C@@H]1CN(c2ccc3ncc(C#Cc4cncc(C(=O)Nc5cccc(C(F)(F)F)c5)c4)n3n2)C[C@H](C)O1]

¹H NMR (500 MHz, Methanol-d₄) δ 9.03 (d, J=2.1 Hz, 1H), 8.84 (d, J=2.0 Hz, 1H), 8.43 (t, J=2.1 Hz, 1H), 8.17 (s, 1H), 7.98-7.92 (m, 1H), 7.83-7.71 (m, 2H), 7.55 (t, J=8.0 Hz, 1H), 7.45 (d, J=7.8 Hz, 1H), 7.23 (d, J=9.9 Hz, 1H), 4.12 (dt, J=12.4, 1.6 Hz, 2H), 3.85-3.68 (m, 2H), 2.73-2.54 (m, 2H), 1.24 (d, J=6.3 Hz, 6H); ¹³C NMR (125 MHz, Methanol-d₄) δ 164.1, 155.4, 152.9, 147.0, 139.1, 137.4, 135.6, 130.9 (q, J=31.2 Hz), 130.5, 129.3, 125.1 (q, J=270 Hz), 124.9, 123.6, 120.6, 120.3, 116.8, 112.1, 93.4, 80.9, 71.3, 50.8, 26.9, 17.7.

### 3-((6-((2S,6R)-2,6-Dimethylmorpholino)imidazo[1,2-b]pyridazin-3-yl)ethynyl)-N-(4-((4-methylpiperazin-1-yl)methyl)-3-(trifluoromethyl)phenyl)benzamide (Compound HSND-107)

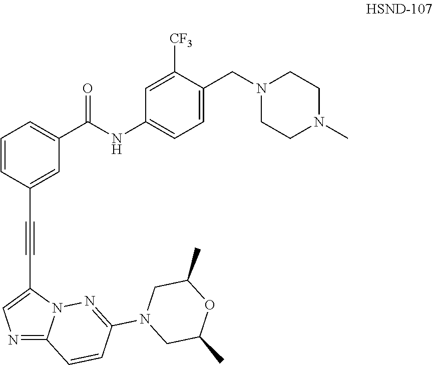 [SMILES: C[C@@H]1CN(c2ccc3ncc(C#Cc4cccc(C(=O)Nc5ccc(CN6CCN(C)CC6)c(C(F)(F)F)c5)c4)n3n2)C[C@H](C)O1]

¹H NMR (500 MHz, DMSO-d₆) δ 10.63 (s, 1H), 8.21-8.13 (m, 2H), 8.05 (dd, J=8.4, 2.2 Hz, 1H), 7.99-7.92 (m, 2H), 7.89 (s, 1H), 7.76 (dt, J=7.7, 1.4 Hz, 1H), 7.69 (d, J=8.5 Hz, 1H), 7.62 (t, J=7.8 Hz, 1H), 7.31 (d, J=10.0 Hz, 1H), 4.14 (dd, J=13.1, 2.3 Hz, 2H), 3.71-3.62 (m, 2H), 2.57 (dd, J=12.9, 10.6 Hz, 2H), 2.37 (s, 8H), 2.14 (s, 3H), 1.13 (d, J=6.2 Hz, 7H).

¹³C NMR (126 MHz, DMSO-d₆) δ 165.30, 155.40, 138.58, 137.38, 136.72, 135.55, 134.11, 132.68, 131.73, 130.34, 129.78, 128.63, 128.01 (q, J=30.2 Hz), 126.55, 125.89 (q, J=274.6 Hz), 123.91, 122.84, 117.67, 112.19, 111.66, 97.75, 79.01, 71.20, 57.92, 55.20, 53.16, 51.04, 46.19, 19.11.

### 5-((6-((3R,5S)-3,5-Dimethylpiperidin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-108)

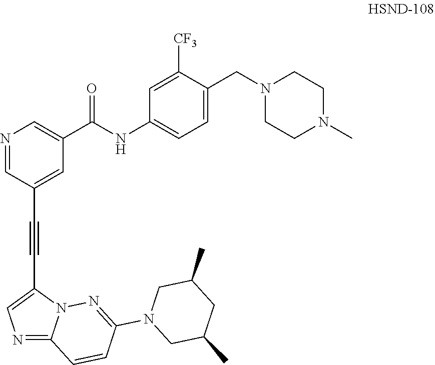 [SMILES: C[C@@H]1C[C@H](C)CN(c2ccc3ncc(C#Cc4cncc(C(=O)Nc5ccc(CN6CCN(C)CC6)c(C(F)(F)F)c5)c4)n3n2)C1]

¹H NMR (500 MHz, DMSO-d₆) δ 10.79 (s, 1H), 9.07 (d, J=2.2 Hz, 1H), 8.87 (d, J=2.0 Hz, 1H), 8.44 (t, J=2.2 Hz, 1H), 8.17 (d, J=2.2 Hz, 1H), 8.03 (dd, J=8.5, 2.2 Hz, 1H), 7.90-7.85 (m, 2H), 7.70 (d, J=8.5 Hz, 1H), 7.32 (d, J=10.0 Hz, 1H), 4.27-4.20 (m, 2H), 3.54 (s, 2H), 2.37 (dt, J=34.7, 16.7 Hz, 1OH), 2.13 (s, 3H), 1.74 (dd, J=12.7, 3.8 Hz, 1H), 1.64 (dqd, J=15.4, 7.3, 5.0, 4.0 Hz. 2H), 0.87 (d, J=6.5 Hz, 6H), 0.77 (q, J=12.0 Hz. 1H); ¹³C NMR (126 MHz, DMSO-d₆) δ 163.79, 155.21, 153.48, 148.45, 138.29, 137.32, 137.15, 136.81, 132.98, 131.76, 130.35, 128.04 (q, J=28.9 Hz), 126.32, 125.83 (q, J=273.4 Hz), 123.89, 119.61, 117.65, 112.78, 111.16, 94.63, 82.36, 57.91, 55.20, 53.16, 52.92, 46.18, 42.27, 30.60, 19.40.

### 5-((6-(4-Isopropylpiperazin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-113)

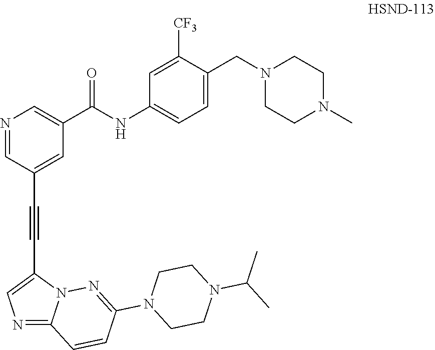 [SMILES: CC(C)N1CCN(c2ccc3ncc(C#Cc4cncc(C(=O)Nc5ccc(CN6CCN(C)CC6)c(C(F)(F)F)c5)c4)n3n2)CC1]

¹H NMR (500 MHz, DMSO-d₆) δ 10.76 (s, 1H), 9.07 (d, J=2.2 Hz, 1H), 8.92 (d, J=2.0 Hz, 1H), 8.46 (t, J=2.2 Hz, 1H), 8.19 (d, J=2.4 Hz, 1H), 8.02 (dd, J=8.5, 2.2 Hz, 1H), 7.92-7.86 (m, 2H), 7.71 (d, J=8.5 Hz, 1H), 7.31 (d, J=10.0 Hz, 1H), 4.25 (dt, J=13.6, 3.7 Hz, 2H), 3.55 (s, 2H), 3.00-2.81 (m, 2H), 2.40-2.21 (m, 11H), 2.21-2.05 (m, 9H), 1.85 (dd, J=13.2, 3.6 Hz, 2H), 1.48-1.30 (m, 2H); ¹³C NMR (126 MHz, DMSO-d₆) δ 163.85, 155.53, 153.72, 148.55, 138.29, 137.39, 137.11, 133.02, 131.79, 130.38, 128.06 (q, J=28.9 Hz), 126.38, 125.84 (q, J=275.9 Hz), 123.91, 119.53, 117.66, 112.77, 111.19, 94.54, 82.25, 61.67, 57.90, 55.20, 53.17, 46.19, 45.21, 41.90, 28.11.

### 5-((6-(4-(Dimethylamino)piperidin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-115)

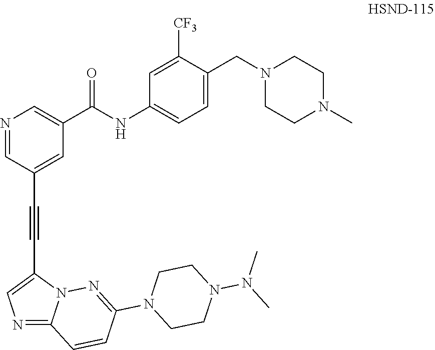 [SMILES: CN1CCN(Cc2ccc(NC(=O)c3cncc(C#Cc4cnc5ccc(N6CCN(N(C)C)CC6)nn45)c3)cc2C(F)(F)F)CC1]

¹H NMR (500 MHz, DMSO-d₆) δ 10.75 (s, 1H), 9.07 (s, 1H), 8.91 (s, 1H), 8.46 (s, 1H), 8.18 (s, 1H), 8.01 (d, J=8.6 Hz, 1H), 7.89 (d, J=9.6 Hz, 2H), 7.70 (d, J=8.5 Hz, 1H), 7.31 (d, J=10.0 Hz, 1H), 4.29-4.21 (m, 2H), 3.54 (s, 2H), 2.99-2.90 (m, 2H), 2.48 (s, 2H), 2.41-2.23 (m, 9H), 2.15-2.11 (m, 9H), 1.87-1.81 (m, 2H), 1.48-1.36 (m, 2H); ¹³C NMR (126 MHz, DMSO-d₆) δ 163.84, 155.53, 153.71, 148.53, 138.28, 137.38, 137.10, 133.01, 131.77, 130.36, 128.06 (q, J=28.9 Hz), 126.37, 125.83 (q, J=274.6 Hz), 123.90, 119.53, 117.64, 112.75, 111.19, 94.53, 82.25, 61.67, 57.90, 55.20, 53.16, 41.90, 28.11.

### 5-((6-(4-Hydroxypiperidin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-116)

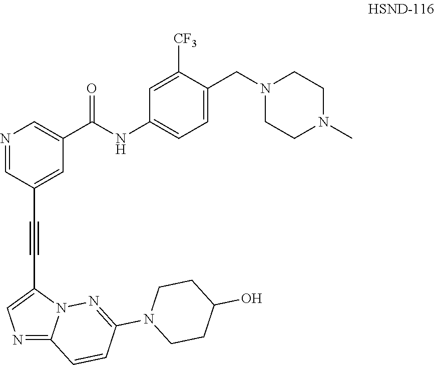 [SMILES: CN1CCN(Cc2ccc(NC(=O)c3cncc(C#Cc4cnc5ccc(N6CCC(O)CC6)nn45)c3)cc2C(F)(F)F)CC1]

¹H NMR (500 MHz, DMSO-d₆) δ 10.76 (s, 1H), 9.08 (s, 1H), 8.92 (s, 1H), 8.47 (d, J=2.5 Hz, 1H), 8.19 (s, 1H), 8.02 (d, J=8.6 Hz, 1H), 7.90 (d, J=9.5 Hz, 2H), 7.72 (d, J=8.6 Hz, 1H), 7.32 (d, J=10.0 Hz, 1H), 4.73 (s, 1H), 4.02-3.94 (m, 2H), 3.77-3.69 (m, 1H), 3.27-3.19 (m, 2H), 2.16 (s, 3H), 1.89-1.76 (m, 2H), 1.52-1.38 (m, 2H); ¹³C NMR (126 MHz, DMSO-d₆) δ 163.85, 155.56, 153.87, 148.57, 138.29, 137.32, 137.21, 133.00, 131.83, 130.38, 128.02 (q, J=22.8 Hz), 126.39, 125.84 (q, J=274.6 Hz), 123.97, 119.54, 117.70, 112.79, 111.16, 94.42, 82.15, 66.12, 57.87, 55.11, 53.04, 46.04, 43.76, 34.01.

### 3-bromo-6-(4-methyl-1,4-diazepan-1-yl)imidazo[1,2-b]pyridazine

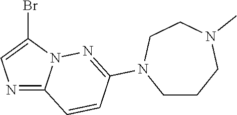 [SMILES: CN1CCCN(c2ccc3ncc(Br)n3n2)CC1]

¹H NMR (500 MHz, Chloroform-d) δ 7.54 (d, J=9.8 Hz, 1H), 7.40 (s, 1H), 6.63 (d, J=9.9 Hz, 1H), 3.83-3.67 (m, 3H), 3.66-3.52 (m, 2H), 2.84-2.59 (m, 2H), 2.54-2.41 (m, 2H), 2.31 (s, 2H), 2.10-1.75 (m, 3H); ¹³C NMR (126 MHz, CDCl₃) δ 153.97, 136.51, 131.45, 125.74, 108.51, 99.84, 57.82, 57.04, 47.37, 47.25, 46.75, 27.41.

### 5-((6-(4-Methyl-1,4-diazepan-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-117)

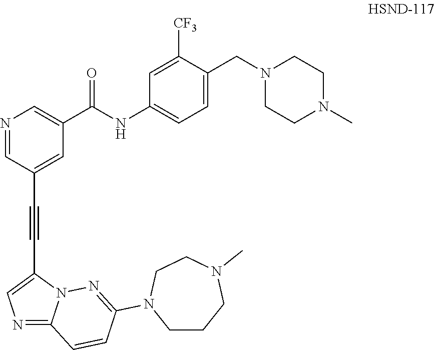 [SMILES: CN1CCN(Cc2ccc(NC(=O)c3cncc(C#Cc4cnc5ccc(N6CCCN(C)CC6)nn45)c3)cc2C(F)(F)F)CC1]

¹H NMR (500 MHz, DMSO-d₆) δ 10.76 (s, 1H), 9.07 (d, J=2.1 Hz, 1H), 8.90 (d, J=2.0 Hz, 1H), 8.44 (t, J=2.1 Hz, 1H), 8.18 (d, J=2.2 Hz, 1H), 8.02 (dd, J=8.5, 2.2 Hz, 1H), 7.89 (d, J=10.1 Hz, 2H), 7.72 (d, J=8.5 Hz, 1H), 7.16 (d, J=10.0 Hz, 1H), 3.75 (t, J=5.0 Hz, 2H), 3.69 (t, J=6.2 Hz, 2H), 2.66 (t, J=5.0 Hz, 2H), 2.48-2.46 (m, 2H), 2.45-2.26 (m, 8H), 2.24 (s, 3H), 2.14 (s, 3H), 1.94-1.88 (m, 2H); ¹³C NMR (126 MHz, DMSO-d₆) δ 163.88, 154.64, 153.66, 148.45, 138,28, 137.30, 136.70, 133.04, 131.83, 130.42, 128.07 (q, J=28.9 Hz), 126.29, 125.84 (q, J=274.8 Hz), 123,93, 119.65, 117,65, 111,57, 111.00, 94.47, 82.44, 57.90, 57.44, 56.76, 55.20, 53.16, 47.53, 47.36, 46.49, 46.19, 27.22.

### (S)-3-Bromo-6-(3-methoxypyrrolidin-1-yl)imidazo[1,2-b]pyridazine

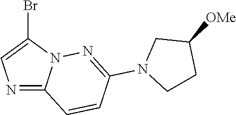 [SMILES: CO[C@H]1CCN(c2ccc3ncc(Br)n3n2)C1]

¹H NMR (500 MHz, DMSO-d₆) δ 7.81 (dd, J=9.9, 0.7 Hz, 1H), 7.53 (s, 1H), 6.86 (dd, J=9.8, 0.8 Hz, 1H), 4.11-4.05 (m, 1H), 3.59-3.49 (m, 3H), 3.44 (td, J=9.7, 7.3 Hz, 1H), 3.26 (s, 3H), 2.13-1.99 (m, 2H); ¹³C NMR (126 MHz, DMSO) δ 153.49, 136.85, 131.36, 126.22, 110.97, 99.28, 79.39, 56.34, 52.33, 45.43, 30.49.

### (S)-5-((6-(3-Methoxypyrrolidin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-138)

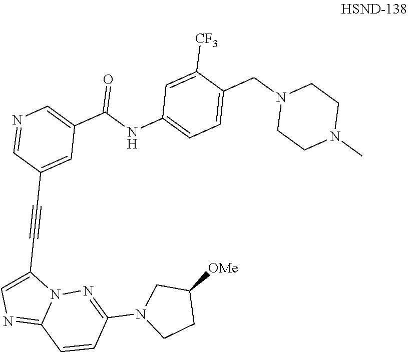 [SMILES: CO[C@H]1CCN(c2ccc3ncc(C#Cc4cncc(C(=O)Nc5ccc(CN6CCN(C)CC6)c(C(F)(F)F)c5)c4)n3n2)C1]

¹H NMR (500 MHz, DMSO-d₆) δ 10.75 (s, 1H), 9.07 (d, J=2.1 Hz, 1H), 8.93 (d, J=2.0 Hz, 1H), 8.47 (t, J=2.1 Hz, 1H), 8.19 (d, J=2.2 Hz, 1H), 8.02 (dd, J=8.5, 2.2 Hz, 1H), 7.95-7.85 (m, 2H), 7.72 (d, J=8.5 Hz, 1H), 6.97 (d, J=9.8 Hz, 1H), 4.13-4.06 (m, 1H), 3.65-3.54 (m, 5H), 3.54-3.45 (m, 1H), 3.26 (s, 3H), 2.46-2.25 (m, 12H), 2.16 (s, 3H), 2.13-2.01 (m, 2H); ¹³C NMR (126 MHz, DMSO-d₆) δ 163.89, 153.83, 153.61, 148.51, 138.29, 137.38, 137.29, 136.77, 133.01, 131.84, 130.38, 128.07 (q, J=30.4 Hz), 126.24, 125.84 (q, J=273.4 Hz), 123.96, 119.63, 117.66, 112.41, 110.87, 94.38, 82.44, 79.40, 57.89, 56.31, 55.16, 53.09, 52.36, 46.12, 45.39, 30.43.

### N-(4-((4-Methylpiperazin-1-yl)methyl)-3-(trifluoromethyl)phenyl)-5-((6-((2,2,6,6-tetramethyltetrahydro-2H-pyran-4-yl)oxy)imidazo[1,2-b]pyridazin-3-yl)ethynyl)nicotinamide (Compound HSND-140)

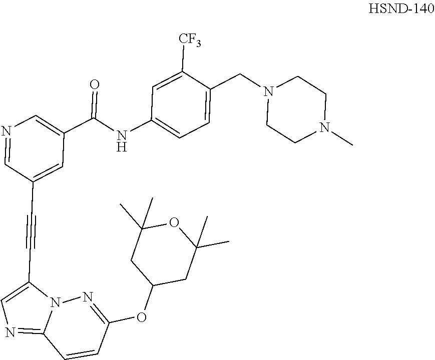 [SMILES: CN1CCN(Cc2ccc(NC(=O)c3cncc(C#Cc4cnc5ccc(OC6CC(C)(C)OC(C)(C)C6)nn45)c3)cc2C(F)(F)F)CC1]

¹H NMR (500 MHz, DMSO-d₆) δ 10.72 (s, 1H), 9.08 (d, J=2.0 Hz, 1H), 8.90 (d, J=1.9 Hz, 1H), 8.48 (d, J=2.4 Hz, 1H), 8.15 (d, J=2.2 Hz, 1H), 8.11 (d, J=9.6 Hz, 1H), 8.07-7.97 (m, 2H), 7.70 (d, J=8.5 Hz, 1H), 6.98 (d, J=9.6 Hz, 1H), 5.50 (tt, J=11.3, 4.1 Hz. 1H), 3.56 (s, 2H), 2.46-2.34 (m, 8H), 2.21 (d, J=12.3 Hz, 5H), 1.38 (t, J=11.7 Hz, 2H), 1.22 (s, 6H), 1.12 (s, 6H); ¹³C NMR (126 MHz, DMSO-d₆) δ 163.65, 159.76, 153.73, 148.80, 138.41, 138.29, 137.94, 137.58, 132.86, 131.84, 130.20, 128.76, 127.87 (q, J=30.4 Hz), 125.81 (q, J=273.4 Hz), 124.73, 123.85, 119.13, 117.62, 114.60, 111.82, 94.60, 81.19, 73.12, 70.85, 57.77, 54.91, 52.67, 52.39, 45.66, 42.03, 33.83, 28.51.

### 5-((6-(3-Oxa-8-azabicyclo[3.2.1]octan-8-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-141)

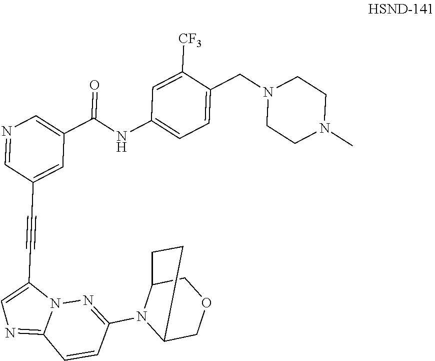 [SMILES: CN1CCN(Cc2ccc(NC(=O)c3cncc(C#Cc4cnc5ccc(N6C7CCC6COC7)nn45)c3)cc2C(F)(F)F)CC1]

¹H NMR (500 MHz, DMSO-d₆) δ 10.75 (s, 1H), 9.07 (d, J=2.1 Hz, 1H), 8.92 (d, J=2.0 Hz, 1H), 8.46 (t, J=2.1 Hz, 1H), 8.18 (d, J=2.2 Hz, 1H), 8.02 (dd, J=8.6, 2.2 Hz, 1H), 7.96 (d, J=9.7 Hz, 1H), 7.92 (s, 1H), 7.71 (d, J=8.5 Hz, 1H), 7.22 (d, J=9.9 Hz, 1H), 4.53 (d, J=4.0 Hz, 2H), 3.73 (d, J=11.0 Hz, 2H). 3.62-3.48 (m, 4H), 2.48 (p, J=1.9 Hz, 2H), 2.36 (s, 10H), 2.14 (s, 3H), 2.03-1.92 (m, 4H); ¹³C NMR (126 MHz, DMSO-d₆) δ 163.85, 153,86, 153,71, 148.56, 138.28, 137.56, 137.34, 137.21, 133.00, 131.80, 130.34, 128.06 (q, J=30.4 Hz), 126.86, 125.84 (q, J=273.4 Hz), 123.92, 119.49, 117.65, 113.57, 111.22, 94.50, 82.04, 70.30, 57.89, 56.34, 55.16, 53.11, 46.12, 26.91.

### 5-(3-Bromoimidazo[1,2-b]pyridazin-6-yl)-2-oxa-5-azabicyclo[2.2.2]octane

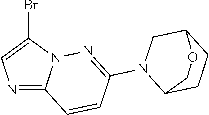 [SMILES: Brc1cnc2ccc(N3CC4CCC3CO4)nn12]

¹H NMR (500 MHz, Methanol-d₄) δ 7.69 (dd, J=11.5, 9.2 Hz, 1H), 7.48-7.42 (m, 1H), 7.00-6.93 (m, 1H), 4.47 (d, J=8.6 Hz, 1H), 4.20-4.12 (m, 1H), 4.11-4.04 (m, 2H), 3.91 (td, J=9.5, 8.3, 5.4 Hz, 1H), 3.66 (t, J=10.8 Hz, 1H), 3.33-3.27 (m, 1H), 2.24-2.06 (m, 2H), 2.06-1.95 (m, 1H), 1.82 (dp, J=12.2, 7.6, 6.0 Hz, 1H); ¹³C NMR (126 MHz, Methanol-d₄) S 153,68, 136.70, 130.26, 124.87, 109.76, 99.80, 69.76, 65.68, 51.58, 44.65, 25.39, 23.92.

### (R)-5-((6-(3-Methoxypyrrolidin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-145)

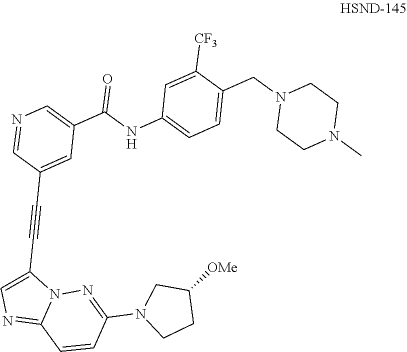 [SMILES: CO[C@@H]1CCN(c2ccc3ncc(C#Cc4cncc(C(=O)Nc5ccc(CN6CCN(C)CC6)c(C(F)(F)F)c5)c4)n3n2)C1]

¹H NMR (500 MHz, DMSO-d₆) δ 10.76 (s, 1H), 9.07 (t, J=1.7 Hz, 1H), 8.92 (d, J=1.8 Hz, 1H), 8.47 (q, J=1.9 Hz, 1H), 8.19 (d, J=2.1 Hz, 1H), 8.02 (d, J=8.5 Hz, 1H), 7.93-7.86 (m, 2H), 7.71 (d, J=8.5 Hz, 1H), 6.96 (d, J=9.8 Hz, 1H), 4.09 (p, J=3.4 Hz, 1H), 3.63-3.55 (m, 5H), 3.49 (q, J=8.8 Hz, 1H), 3.25 (s, 3H), 2.38 (s, 8H), 2.17 (s, 3H), 2.13-1.99 (m, 3H); ¹³C NMR (126 MHz, DMSO-d₆) δ 163.87, 153.82, 153.59, 148.50, 138.30, 137.37, 137.28, 136.75, 132.97, 131.82, 130.35, 127.83 (q, J=30.2 Hz), 126.22, 125.84 (q, J=274.6 Hz), 123.95, 119.63, 117.65, 112.39, 110.87, 94.38, 82.44, 79.40, 57.87, 56.31, 55.11, 53.01, 52.35, 46.02, 45.38, 30.43.

### 6-(3-Bromoimidazo[1,2-b]pyridazin-6-yl)-2-oxa-6-azaspiro[3.3]heptane

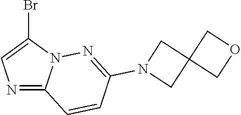 [SMILES: Brc1cnc2ccc(N3CC4(COC4)C3)nn12]

¹H NMR (500 MHz, DMSO-d₆) δ 7.83 (d, J=9.6 Hz, 1H), 7.56 (s. 1H), 6.67 (dd, J=9.7, 1.0 Hz, 1H), 4.77-4.64 (m, 4H), 4.28-4.16 (m, 4H); ¹³C NMR (126 MHz, DMSO) δ 156.45, 137,15, 131.75, 126,47, 109,97, 99.55, 80.23, 60.54.

### 5-((6-(2-Oxa-6-azaspiro[3.3]heptan-6-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-149)

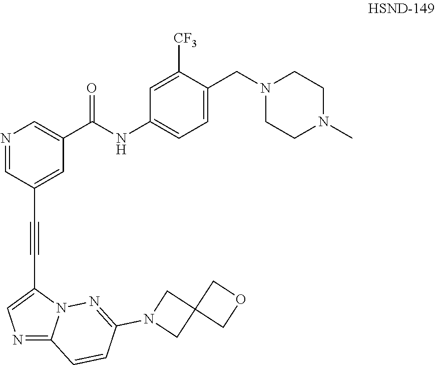 [SMILES: CN1CCN(Cc2ccc(NC(=O)c3cncc(C#Cc4cnc5ccc(N6CC7(COC7)C6)nn45)c3)cc2C(F)(F)F)CC1]

¹H NMR (500 MHz, Methanol-d₄) δ 9.05 (d, J=2.1 Hz, 1H), 8.87 (d, J=1.9 Hz, 1H), 8.46 (t, J=2.1 Hz, 1H), 8.15 (d, J=2.2 Hz, 1H), 7.95 (dd, J=8.4, 2.3 Hz, 1H), 7.81-7.76 (m, 2H), 7.74 (d, J=9.7 Hz, 1H), 6.74 (d, J=9.7 Hz, 1H), 4.86 (s, 4H), 4.33 (s. 4H), 3.68 (d, J=1.7 Hz, 2H), 2.76-2.45 (m, 9H), 2.40 (s, 3H); ¹³C NMR (126 MHz, Methanol-d₄) δ 164.14, 156,22, 153.12, 147.09, 137.60, 137.39, 135.53, 132.89, 131.29, 130.53, 128.92 (q, J=30.4 Hz), 125.38 (q, J=273.4 Hz), 124.93, 123.57, 120.32, 117.73, 110.89, 93.36, 80.77, 59.92, 57.38, 54.44, 51.86, 44.18, 39.09.

### (1R,4R)-5-(3-Bromoimidazo[1,2-b]pyridazin-6-yl)-2-oxa-5-azabicyclo[2.2.1]heptane

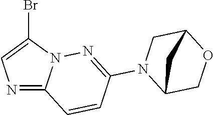 [SMILES: Brc1cnc2ccc(N3C[C@H]4C[C@@H]3CO4)nn12]

¹H NMR (500 MHz, Methanol-d₄) δ 7.66 (d, J=9.8 Hz, 1H), 7.45 (s, 1H), 6.86 (d, J=9.8 Hz, 1H), 4.90-4.86 (m, 1H), 4.73-4.69 (m, 1H), 3.88 (s, 2H), 3.59 (dd, J=10.0, 1.6 Hz, 1H), 3.48-3.42 (m, 1H), 2.06-1.96 (m, 2H); ¹³C NMR (126 MHz, MeOD) δ 153,36, 136,77, 130.30, 124.87, 111.06, 99.79, 76.28, 72.73, 57.29, 56.28, 35.97.

### 5-((6-((1R,4R)-2-Oxa-5-azabicyclo[2.2.1]heptan-5-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-150)

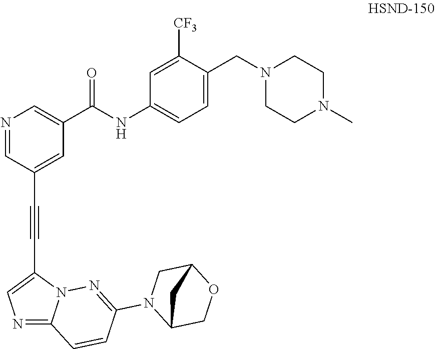 [SMILES: CN1CCN(Cc2ccc(NC(=O)c3cncc(C#Cc4cnc5ccc(N6C[C@H]7C[C@@H]6CO7)nn45)c3)cc2C(F)(F)F)CC1]

¹H NMR (500 MHz, Methanol-d₄) δ 9.00 (d, J=2.2 Hz, 1H), 8.80 (d, J=2.0 Hz, 1H), 8.40 (t, J=2.1 Hz, 1H), 8.10 (d, J=2.3 Hz, 1H), 7.91 (dd, J=8.5, 2.3 Hz, 1H), 7.77-7.68 (m, 3H), 6.92 (d, J=9.8 Hz, 1H), 4.94-4.91 (m, 1H), 4.73-4.66 (m, 1H), 3.93-3.89 (m, 2H), 3.65-3.58 (m, 3H), 3.52-3.46 (m, 1H), 2.51 (s, 8H), 2.28 (s, 3H), 2.06-1.96 (m, 2H); ¹³C NMR (126 MHz, Methanol-d₄) δ 164.00, 153.49, 153.03, 146.97, 137.47, 137.33, 137.04, 135.36, 133.04, 131.21, 130.41, 128.83 (q, J=30.2 Hz), 125.37 (q, J=273.4 Hz), 124.90, 123.49, 120.30, 117.63, 112.26, 111.61, 93.34, 81.04, 76.27, 72.82, 57.49, 57.38, 56.29, 54.59, 52.29, 44.59, 36.04.

### (1S,4S)-5-(3-Bromoimidazo[1,2-b]pyridazin-6-yl)-2-oxa-5-azabicyclo[2.2.1]heptane

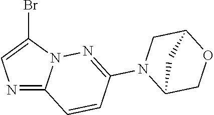 [SMILES: Brc1cnc2ccc(N3C[C@@H]4C[C@H]3CO4)nn12]

¹H NMR (500 MHz, Methanol-d₄) δ 7.71-7.63 (m, 1H), 7.48-7.43 (m, 1H), 6.91-6.84 (m, 1H), 4.89 (d, J=5.3 Hz, 2H), 4.72 (s, 1H), 3.91-3.86 (m, 2H), 3.63-3.56 (m, 1H), 3.49-3.43 (m, 1H), 2.07-1.95 (m, 2H); ¹³C NMR (126 MHz, MeOD) δ 153.38, 136.78, 130.30, 124.88, 111.07, 99.80, 76.29, 72.74, 57.30, 56.28, 35.97.

### 5-((6-((1S,4S)-2-Oxa-5-azabicyclo[2.2.1]heptan-5-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-151)

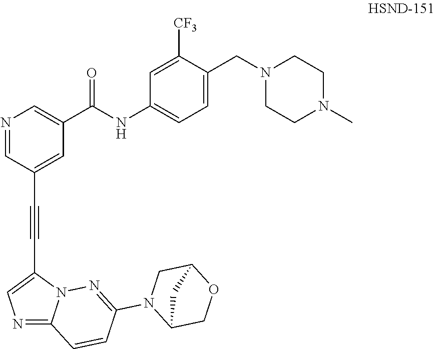 [SMILES: CN1CCN(Cc2ccc(NC(=O)c3cncc(C#Cc4cnc5ccc(N6C[C@@H]7C[C@H]6CO7)nn45)c3)cc2C(F)(F)F)CC1]

¹H NMR (500 MHz, Methanol-d₄) δ 9.00 (d, J=2.1 Hz, 1H), 8.81 (d, J=2.0 Hz, 1H), 8.40 (t, J=2.1 Hz, 1H), 8.10 (d, J=2.2 Hz, 1H), 7.91 (dd, J=8.5, 2.3 Hz, 1H), 7.80-7.64 (m, 3H), 6.93 (d, J=9.8 Hz, 1H), 4.96-4.90 (m, 1H), 4.75-4.66 (m, 1H), 3.91 (s, 2H), 3.64-3.57 (m, 3H), 3.49 (d, J=10.0 Hz, 1H), 2.77-2.32 (m, 8H), 2.30 (s, 3H), 2.07-1.96 (m, 2H); ¹³C NMR (126 MHz, Methanol-d₄) δ 164.01, 153.49, 153.03, 146.98, 137.49, 137.33, 137.05, 135.36, 133.00, 131.21, 130.42, 128.84 (q, J=30.4 Hz), 125.37 (q, J=273.4 Hz), 124.90, 123.49, 120.30, 117.64, 112.26, 111.62, 93.34, 81.04, 76.27, 72.82, 57.47, 57.38, 56.29, 54.56, 52.22, 44.52, 36.04.

### 4-(1-(3-Bromoimidazo[1,2-b]pyridazin-6-yl)piperidin-4-yl)morpholine

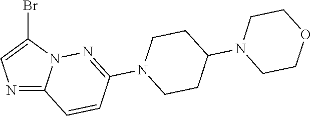 [SMILES: Brc1cnc2ccc(N3CCC(N4CCOCC4)CC3)nn12]

¹H NMR (500 MHz, Methanol-d₄) δ 7.68 (dd, J=10.1, 2.9 Hz, 1H), 7.48 (s, 1H), 7.21-7.13 (m, 1H), 4.38-4.30 (m, 2H), 3.72-3.67 (m, 4H), 3.01-2.91 (m, 2H), 2.63-2.56 (m, 4H), 2.46 (dtq, J=10.6, 7.0, 3.8 Hz, 1H), 2.05-1.98 (m, 2H), 1.62-1.46 (m, 2H).

¹³C NMR (126 MHz, MeOD) δ 155.24, 136.65, 130.53, 124.76, 111.28, 100.09, 66.50, 61.87, 49.60, 45.04, 27.33.

### N-(4-((4-Methylpiperazin-1-yl)methyl)-3-(trifluoromethyl)phenyl)-5-((6-(4-morpholinopiperidin-1-yl)imidazo[1,2-b]pyridazin-3-yl)ethynyl)nicotinamide (Compound HSND-152)

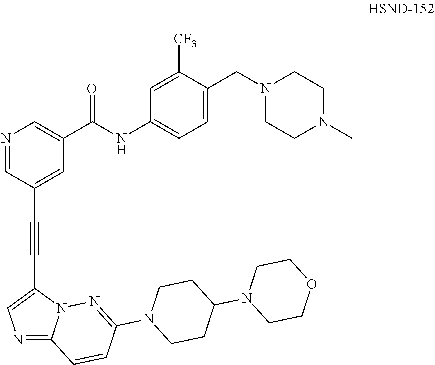 [SMILES: CN1CCN(Cc2ccc(NC(=O)c3cncc(C#Cc4cnc5ccc(N6CCC(N7CCOCC7)CC6)nn45)c3)cc2C(F)(F)F)CC1]

¹H NMR (500 MHz, Methanol-d₄) δ 8.99 (d, J=2.1 Hz, 1H), 8.80-8.74 (m, 1H), 8.41-8.36 (m, 1H), 8.09 (d, J=2.3 Hz, 1H), 7.90 (dd, J=8.4, 2.2 Hz, 1H), 7.79-7.70 (m, 2H), 7.71-7.64 (m, 1H), 7.18 (dd, J=10.6, 3.2 Hz, 1H), 4.33 (d, J=13.2 Hz, 2H), 3.68-3.61 (m, 6H), 2.96 (t, J=12.8 Hz, 2H). 2.72-2.33 (m, 14H), 2.28 (s, 3H), 2.03-1.97 (m, 2H), 1.58-1.47 (m, 2H); ¹³C NMR (126 MHz, Methanol-d₄) δ 163.89, 155.32, 152.90, 146.93, 137.48, 137.41, 136.90, 135.48, 133.03, 131.18, 130.38, 128.80 (q, J=30.4 Hz), 125.37 (q, J=274.6 Hz), 124.76, 123.41, 120.26, 117.56, 112.45, 111.81, 93.52, 81.14, 66.47, 61.80, 57.49, 54.61, 52.30, 49.61, 45.01, 44.61, 27.42.

### 3-Bromo-6-(4-(4-methylpiperazin-1-yl)piperidin-1-yl)imidazo[1,2-b]pyridazine

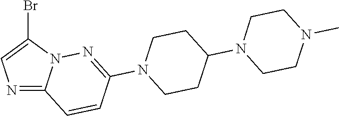 [SMILES: CN1CCN(C2CCN(c3ccc4ncc(Br)n4n3)CC2)CC1]

¹H NMR (500 MHz, Methanol-d₄) δ 7.66 (d, J=10.0 Hz, 1H), 7.47 (s, 1H), 7.15 (d, J=10.0 Hz, 1H), 4.37-4.29 (m, 2H), 2.98-2.89 (m, 2H), 2.76-2.37 (m, 9H), 2.27 (s, 3H), 2.03-1.95 (m, 2H), 1.54 (qd, J=12.4, 4.1 Hz, 2H).

¹³C NMR (126 MHz, MeOD) δ 155.20, 136.64, 130.54, 124.76, 111.25, 100.09, 61.53, 54.50, 48.35, 45.13, 44.59, 27.42.

### N-(4-((4-Methylpiperazin-1-yl)methyl)-3-(trifluoromethyl)phenyl)-5-((6-(4-(4-methylpiperazin-1-yl)piperidin-1-yl)imidazo[1,2-b]pyridazin-3-yl)ethynyl)nicotinamide (Compound HSND-153)

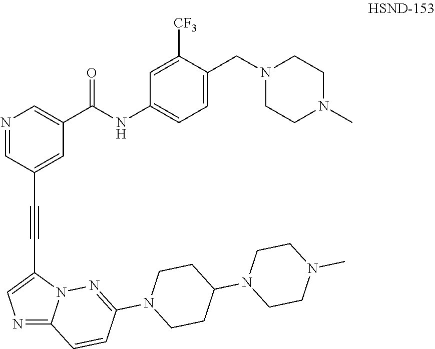 [SMILES: CN1CCN(Cc2ccc(NC(=O)c3cncc(C#Cc4cnc5ccc(N6CCC(N7CCN(C)CC7)CC6)nn45)c3)cc2C(F)(F)F)CC1]

¹H NMR (500 MHz, Methanol-d₄) δ 9.01 (s, 1H), 8.83 (s, 1H), 8.43 (d, J=2.3 Hz, 1H), 8.12 (d, J=2.3 Hz, 1H), 7.92 (dd, J=8.5, 2.3 Hz, 1H), 7.79-7.68 (m, 3H), 7.22 (dd, J=10.3, 2.9 Hz, 1H), 4.37 (d, J=13.2 Hz, 2H), 3.64 (s, 2H), 2.98 (t, J=12.8 Hz, 2H), 2.91-2.34 (m, 17H), 2.27 (d, J=15.0 Hz, 6H), 2.02 (d, J=12.4 Hz, 2H), 1.56 (qd, J=12.1, 3.9 Hz, 2H).

¹³C NMR (126 MHz, Methanol-d₄) δ 164.01, 155.36, 152.99, 147.01, 137.51, 137.41, 136.93, 135.52, 133.05, 131.22, 130.49, 128.84 (q, J=30.4 Hz), 125.39 (q, J=273.4 Hz), 124.78, 123.46, 123.22, 120.31, 117.60, 112.51, 111.83, 93.41, 81.03, 61.51, 57.51, 54.62, 54.47, 52.33, 48.37, 45.12, 44.63, 44.56, 27.49.

### 7-(3-Bromoimidazo[1,2-b]pyridazin-6-yl)-4-oxa-7-azaspiro[2.5]octane

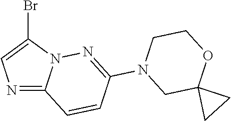 [SMILES: Brc1cnc2ccc(N3CCOC4(CC4)C3)nn12]

¹H NMR (500 MHz, Methanol-d₄) δ 7.71 (d, J=10.0 Hz, 1H), 7.51 (s, 1H), 7.16 (d, J=10.0 Hz, 1H), 3.95-3.82 (m, 2H), 3.74-3.64 (m, 2H), 3.57 (s, 2H), 0.81 (t, J=3.5 Hz, 2H), 0.74-0.66 (m, 2H).

### 5-((6-(4-Oxa-7-azaspiro[2.5]octan-7-yl)imidazo[1,2-b]pyrida

¹³C NMR (126 MHz, MeOD) δ 155.65, 130.71, 124.92, 110.95, 65.16, 58.24, 50.97, 45.39, 10.57.zin-3-yl)ethynyl)-N-(4-((4-methylpiperazin-1-yl)methyl)-3-(trifluoromethyl)phenyl)nicotinamide (Compound HSND-156)

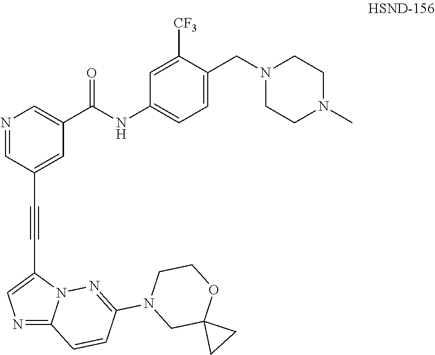 [SMILES: CN1CCN(Cc2ccc(NC(=O)c3cncc(C#Cc4cnc5ccc(N6CCOC7(CC7)C6)nn45)c3)cc2C(F)(F)F)CC1]

¹H NMR (500 MHz, DMSO-d₆) δ 10.75 (s, 1H), 9.07 (d, J=2.1 Hz, 1H), 8.93 (d, J=2.0 Hz, 1H), 8.46 (t, J=2.1 Hz, 1H), 8.18 (d, J=2.2 Hz, 1H), 8.03 (dd, J=8.5, 2.2 Hz, 1H), 8.00-7.92 (m, 2H), 7.72 (d, J=8.5 Hz, 1H), 7.31 (d, J=9.9 Hz, 1H), 3.79 (dd, J=5.7, 4.0 Hz, 2H), 3.64 (dd, J=5.8, 4.0 Hz, 2H), 3.56 (d, J=2.8 Hz, 4H), 2.39-2.35 (m, 8H), 2.15 (s, 3H), 0.75-0.70 (m, 2H), 0.70-0.64 (m, 2H); ¹³C NMR (126 MHz, DMSO-d₆) δ 163.87, 155.94, 153.78, 148.58, 138.28, 137.61, 137.39, 133.03, 131.84, 130.38, 128.07 (q, J=30.4 Hz), 126.55, 125.84 (274.6 Hz), 123.93, 119.47, 117.63, 112.66, 111.26, 94.63, 82.07, 65.43, 58.64, 57.90, 55.17, 53.13, 51.21, 46.16, 45.61, 11.51.

### (2S,6R)-4-(3-Bromo-2-methylimidazo[1,2-b]pyridazin-6-yl)-2,6-dimethylmorpholine

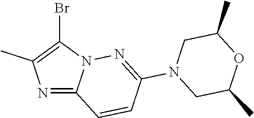

¹H NMR (500 MHz, Methanol-d₄) δ 7.62 (d, J=9.9 Hz, 1H), 7.12 (d, J=9.9 Hz, 1H), 4.07 (dd, J=13.5, 2.0 Hz, 2H), 3.74 (dqd, J=12.5, 6.2, 2.3 Hz, 2H), 2.58 (dd, J=12.9, 10.6 Hz, 2H), 2.36 (s, 3H), 1.24 (d, J=6.2 Hz, 6H); ¹³C NMR (126 MHz, MeOD) δ 155.11, 139.14, 136.09, 124.04, 110.41, 98.27, 71.36, 51.05, 17.68, 12.05.

### 5-((6-((2S,6R)-2,6-Dimethylmorpholino)-2-methylimidazo[1,2-b]pyridazin-3-yl)ethynyl)-N-(4-((4-methylpiperazin-1-yl)methyl)-3-(trifluoromethyl)phenyl)nicotinamide (Compound HSND-157)

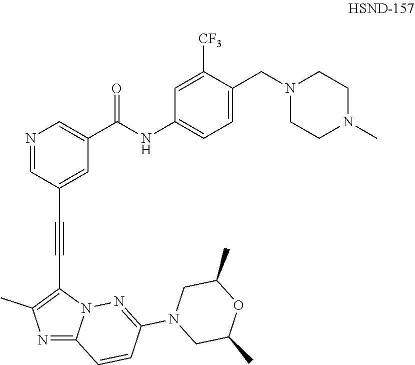

¹H NMR (500 MHz, Methanol-d₄) δ 9.01 (d, J=2.1 Hz, 1H), 8.83 (d, J=2.0 Hz, 1H), 8.42 (t, J=2.1 Hz, 1H), 8.13 (d, J=2.3 Hz, 1H), 7.93 (dd, J=8.4, 2.3 Hz, 1H), 7.77 (d, J=8.5 Hz, 1H), 7.66 (d, J=9.9 Hz, 1H), 7.17 (d, J=9.9 Hz, 1H), 4.12-4.06 (m, 2H), 3.80-3.70 (m, 2H), 3.66 (s, 2H). 2.71-2.40 (m, 13H), 2.32 (s, 3H), 1.23 (d, J=6.2 Hz, 6H); ¹³C NMR (126 MHz, Methanol-d₄) δ 164.04, 155.18, 152.75, 146.85, 146.21, 137.54, 137.15, 136.29, 133.00, 131,25, 130,51, 128.89 (q, J=30.4 Hz), 125.38 (q, J=273.4 Hz), 124.07, 123.50, 120.53, 117.66, 111.71, 109.58, 95.31, 81.29, 71.34, 57.47, 54.55, 52.19, 50.96, 44.49, 17.72, 12.81.

### 6-(3-Bromoimidazo[1,2-b]pyridazin-6-yl)-2-oxa-6-azaspiro[3.4]octane

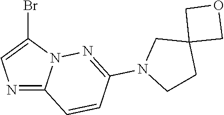 [SMILES: Brc1cnc2ccc(N3CCC4(COC4)C3)nn12]

¹H NMR (500 MHz, Methanol-d₄) δ 7.67 (d, J=9.8 Hz, 1H), 7.45 (s, 1H), 6.89 (d, J=9.8 Hz, 1H), 4.74 (d, J=6.2 Hz, 2H), 4.68 (d, J=6.2 Hz, 2H), 3.81 (s, 2H), 3.58 (t, J=7.0 Hz, 2H), 2.37 (t, J=7.0 Hz, 2H); ¹³C NMR (126 MHz, MeOD) δ 153.31, 136.66, 130.10, 124.57, 110.74, 99.65, 80.70, 55.28, 45.50, 45.22, 34.55.

### 5-((6-(2-Oxa-6-azaspiro[3.4]octan-6-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-158)

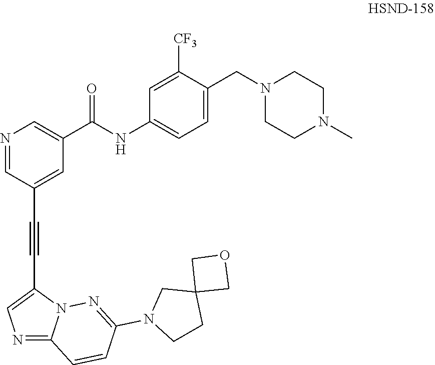 [SMILES: CN1CCN(Cc2ccc(NC(=O)c3cncc(C#Cc4cnc5ccc(N6CCC7(COC7)C6)nn45)c3)cc2C(F)(F)F)CC1]

¹H NMR (500 MHz, DMSO-d₆) δ 10.80 (s, 1H), 9.09 (d, J=2.2 Hz, 1H), 8.94 (d, J=2.0 Hz, 1H), 8.49 (t, J=2.2 Hz, 1H), 8.21 (d, J=2.2 Hz, 1H), 8.04 (dd, J=8.5, 2.2 Hz, 1H), 7.93-7.85 (m, 2H), 7.72 (d, J=8.5 Hz, 1H), 6.95 (d, J=9.9 Hz, 1H), 4.61 (d, J=6.1 Hz, 2H), 4.51 (d, J=6.1 Hz, 2H), 3.77 (s, 2H), 3.57 (s, 2H), 3.52 (t, J=6.9 Hz, 2H), 2.42-2.26 (m, 8H), 2.27 (t, J=6.9 Hz, 2H), 2.22 (s, 3H); ¹³C NMR (126 MHz, DMSO-d₆) δ 163.89, 153.88, 153.51, 148.55, 138.35, 137.36, 136.85, 132.89, 131.85, 130.36, 127.85 (q, J=30.2 Hz), 126.24, 125.84 (q, J=273.4 Hz), 123.98, 119.61, 117.74, 112.36, 110.87, 94.39, 82.40, 80.14, 57.80, 55.64, 54.94, 52.75, 46.11, 45.74, 45.24, 34.92.

### 6-(3-Bromoimidazo[1,2-b]pyridazin-6-yl)-1-oxa-6-azaspiro[3.4]octane

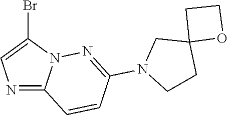 [SMILES: Brc1cnc2ccc(N3CCC4(CCO4)C3)nn12]

¹H NMR (500 MHz, Methanol-d₄) δ 7.65 (dd, J=9.8, 1.2 Hz, 1H), 7.44 (d, J=1.2 Hz, 1H), 6.84 (dd, J=9.9, 1.2 Hz, 1H), 4.62-4.53 (m, 2H), 3.95 (d, J=11.9 Hz, 1H), 3.70-3.63 (m, 1H), 3.60 (d, J=12.0 Hz, 1H), 3.54 (td, J=10.0, 6.6 Hz, 1H), 2.91-2.82 (m, 1H), 2.82-2.72 (m, 1H), 2.54-2.45 (m, 1H), 2.25-2.13 (m, 1H); ¹³C NMR (126 MHz, MeOD) δ 153.19, 136.60, 130.03, 124.52, 110.52, 99.62, 90.91, 65.24, 57.78, 44.77, 37.14, 27.95.

### 5-((6-(1-Oxa-6-azaspiro[3.4]octan-6-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-159)

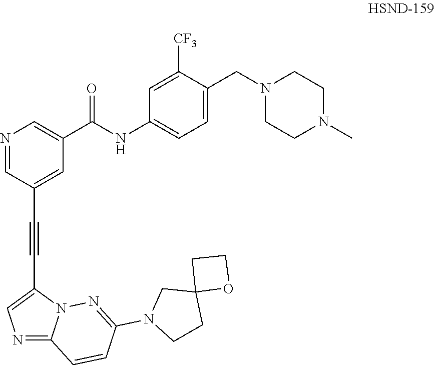 [SMILES: CN1CCN(Cc2ccc(NC(=O)c3cncc(C#Cc4cnc5ccc(N6CCC7(CCO7)C6)nn45)c3)cc2C(F)(F)F)CC1]

¹H NMR (500 MHz, DMSO-d₆) δ 10.77 (s, 1H), 9.08 (d, J=2.1 Hz, 1H), 8.94 (d, J=2.0 Hz, 1H), 8.49 (t, J=2.1 Hz, 1H), 8.19 (d, J=2.2 Hz, 1H), 8.03 (dd, J=8.5, 2.2 Hz, 1H), 7.93-7.86 (m, 2H), 7.71 (d, J=8.6 Hz, 1H), 6.93 (d, J=9.9 Hz, 1H), 4.45-4.36 (m, 2H), 3.87 (dd, J=11.9, 1.4 Hz, 1H), 3.67-3.57 (m, 2H), 3.56 (s, 2H), 3.52-3.43 (m, 1H), 2.81-2.72 (m, 1H), 2.68-2.59 (m, 1H), 2.46 (s, 8H), 2.18 (s, 3H), 2.16-2.09 (m, 1H); ¹³C NMR (126 MHz, DMSO-d₆) δ 163.87, 153.83, 153.47, 148.54, 138.33, 137.38, 136.82, 132.92, 131.81, 130.33, 128.07 (q, J=30.2 Hz), 126.30, 125.84 (q, J=274.6 Hz), 123.94, 119.60, 117.70, 112.16, 110.90, 94.41, 90.05, 82.41, 64.89, 58.21, 57.84, 55.04, 52.90, 45.90, 45.42, 40.34, 37.56, 28.76.

### 1-(3-Bromoimidazo[1,2-b]pyridazin-6-yl)-4-methylpiperidin-4-ol

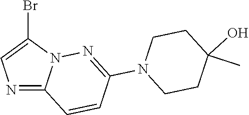 [SMILES: CC1(O)CCN(c2ccc3ncc(Br)n3n2)CC1]

¹H NMR (500 MHz, Methanol-d₄) δ 7.66 (d, J=10.0 Hz, 1H), 7.46 (s, 1H), 7.17 (d, J=10.0 Hz, 1H), 3.94-3.86 (m, 2H), 3.46 (dt, J=13.7, 7.1 Hz, 2H), 1.68 (dd, J=7.1, 4.4 Hz, 4H), 1.26 (s, 3H); ¹³C NMR (126 MHz, MeOD) δ 155.30, 136.61, 130.39, 124.63, 111.28, 100.03, 67.07, 42.18, 37.28, 28.49.

### 5-((6-(4-Hydroxy-4-methylpiperidin-1-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-168)

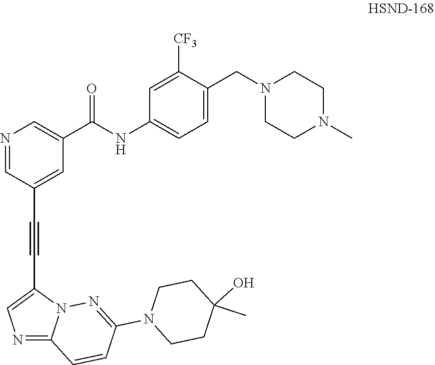 [SMILES: CN1CCN(Cc2ccc(NC(=O)c3cncc(C#Cc4cnc5ccc(N6CCC(C)(O)CC6)nn45)c3)cc2C(F)(F)F)CC1]

¹H NMR (500 MHz, DMSO-d₆) δ 10.77 (s, 1H), 9.08 (d, J=2.2 Hz, 1H), 8.92 (d, J=2.0 Hz, 1H), 8.46 (t, J=2.1 Hz, 1H), 8.19 (d, J=2.2 Hz, 1H), 8.03 (dd, J=8.4, 2.2 Hz, 1H), 7.90 (d, J=10.1 Hz, 2H), 7.72 (d, J=8.5 Hz, 1H), 7.32 (d, J=10.0 Hz, 1H), 4.39 (s. 1H), 3.91-3.83 (m, 2H), 3.57 (s, 2H), 3.42-3.37 (m, 2H), 2.20 (s, 3H), 1.60-1.50 (m, 4H), 1.14 (s, 3H); ¹³C NMR (126 MHz, DMSO-d₆) δ 163.88, 155.52, 153.82, 148.56, 138.31, 137.33, 137.11, 132.95, 131.86, 130.39, 128.13 (q, J=28.6 Hz), 126.34, 125.84 (q, J=274.6 Hz), 123.97, 123.66, 119.56, 117.67, 112.80, 111.12, 94.45, 82.24, 66.52, 57.83, 55.04, 52.89, 45.90, 42.63, 38.07, 30.13.

### N-(4-((4-Methylpiperazin-1-yl)methyl)-3-(trifluoromethyl)phenyl)-5-((6-((tetrahydro-1H-pyrrolizin-7a(5H)-yl)methoxy)imidazo[1,2-b]pyridazin-3-yl)ethynyl)nicotinamide (Compound HSND174)

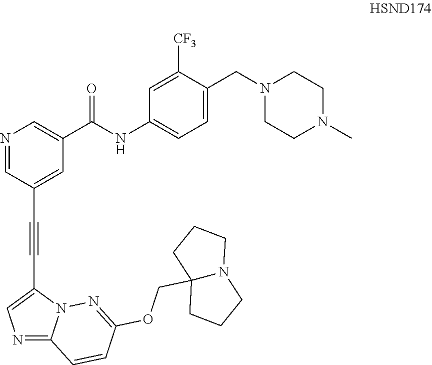 [SMILES: CN1CCN(Cc2ccc(NC(=O)c3cncc(C#Cc4cnc5ccc(OCC67CCCN6CCC7)nn45)c3)cc2C(F)(F)F)CC1]

¹H NMR (500 MHz, Methanol-d₄) δ 9.07 (s, 1H), 8.92 (s, 1H), 8.52 (td, J=2.1, 1.0 Hz, 1H), 8.14 (d, J=2.3 Hz, 1H), 8.00-7.88 (m, 3H), 7.78 (d, J=8.5 Hz, 1H), 7.02 (dd, J=9.6, 1.0 Hz, 1H), 4.27 (s, 2H), 3.66 (s, 2H), 3.10-3.02 (m, 2H), 2.74-2.67 (m, 3H), 2.67-2.36 (m, 8H), 2.29 (s, 3H), 2.10-2.00 (m, 2H). 1.96-1.88 (m, 3H), 1.88-1.80 (m, 2H), 1.79-1.71 (m, 2H).

### 5-((6-(Ethyl(tetrahydro-2H-pyran-4-yl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND175)

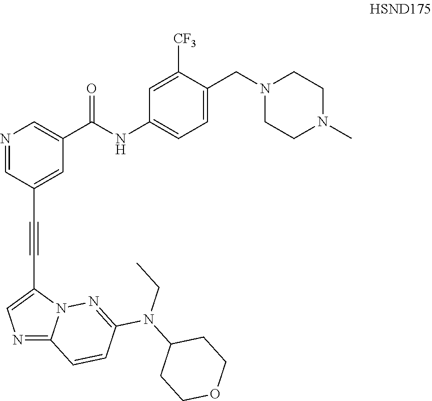 [SMILES: CCN(c1ccc2ncc(C#Cc3cncc(C(=O)Nc4ccc(CN5CCN(C)CC5)c(C(F)(F)F)c4)c3)n2n1)C1CCOCC1]

¹H NMR (500 MHz, Methanol-d₄) δ 9.05-9.01 (m, 1H), 8.85 (s, 1H), 8.44 (t, J=2.0 Hz, 1H), 8.10 (d, J=2.3 Hz, 1H), 7.94 (dd, J=8.4, 2.3 Hz, 1H), 7.75 (dd, J=15.4, 9.2 Hz, 3H), 7.12 (d, J=9.9 Hz, 1H), 4.36 (tt, J=11.8, 3.9 Hz, 1H), 4.08 (dd, J=11.4, 4.4 Hz, 2H), 3.65 (s, 2H), 3.61-3.52 (m, 4H), 2.52 (s, 8H), 2.30 (s, 3H), 2.21 (tt, J=12.2, 6.1 Hz, 3H), 1.79 (ddd, J=12.4, 4.0, 1.8 Hz, 2H), 1.29 (t, J=7.0 Hz, 3H).

### 3-Bromo-N-methyl-N-(tetrahydro-2H-pyran-4-yl)imidazo[1,2-b]pyridazin-6-amine

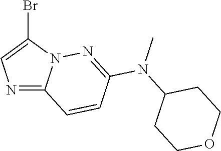 [SMILES: CN(c1ccc2ncc(Br)n2n1)C1CCOCC1]

¹H NMR (500 MHz, Methanol-d₄) δ 7.66 (dd, J=10.0, 1.1 Hz, 1H), 7.45 (d, J=1.0 Hz, 1H), 7.10 (dd, J=10.0, 1.2 Hz, 1H), 4.50-4.40 (m, 1H), 4.08-4.01 (m, 2H), 3.56 (td, J=11.9, 1.9 Hz, 2H), 3.00 (s, 3H), 1.98-1.86 (m, 2H), 1.74-1.67 (m, 2H).

¹³C NMR (126 MHz, MeOD) δ 154.81, 136.54, 130.25, 124.64, 110.63, 99.84, 67.18, 53.27, 29.53, 29.16.

### 5-((6-(Methyl(tetrahydro-2H-pyran-4-yl)amino)imidazo[1,2-b]pyridazin-3-yl)ethynyl)-N-(4-((4-methylpiperazin-1-yl)methyl)-3-(trifluoromethyl)phenyl)nicotinamide (Compound HSND176)

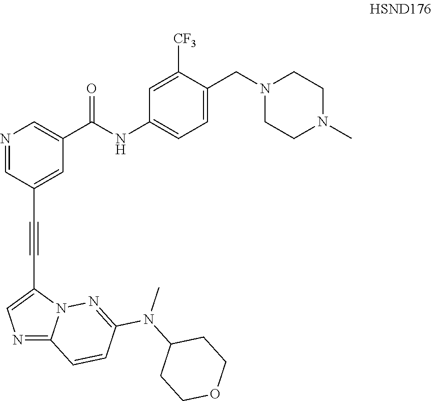 [SMILES: CN1CCN(Cc2ccc(NC(=O)c3cncc(C#Cc4cnc5ccc(N(C)C6CCOCC6)nn45)c3)cc2C(F)(F)F)CC1]

¹H NMR (500 MHz, DMSO-d₆) δ 10.74 (s, 1H), 9.07 (d, J=2.1 Hz, 1H), 8.89 (d, J=1.9 Hz, 1H), 8.44 (t, J=2.0 Hz, 1H), 8.18 (d, J=2.2 Hz, 1H), 8.03 (dd, J=8.5, 2.2 Hz, 1H), 7.92-7.86 (m. 2H), 7.69 (d, J=8.5 Hz, 1H), 7.23 (d, J=9.9 Hz, 1H), 4.44-4.34 (m, 1H), 3.92 (dd, J=11.2, 4.4 Hz, 2H), 3.44 (td, J=11.8, 1.9 Hz, 3H), 2.96 (s, 3H), 2.47-2.28 (m, 8H), 2.17 (s, 3H), 1.91-1.78 (m, 2H), 1.67-1.60 (m, 2H); ¹³C NMR (126 MHz, DMSO-d₆) δ 163.78, 154.99, 153.59, 148.41, 138.29, 137.31, 137.11, 136.74, 132.91, 131.76, 130.31, 128.06 (q, J=30.4 Hz), 126.25, 125.83 (q, J=273.4 Hz), 123.92, 119.65, 117.69, 112.26, 111.05, 94.51, 82.36, 67.14, 57.84, 55.04, 53.46, 52.90, 45.90, 30.45, 29.93.

### 5-((6-(2,2-Dimethylmorpholino)imidazo[1,2-b]pyridazin-3-yl)ethynyl)-N-(4-((4-methylpiperazin-1-yl)methyl)-3-(trifluoromethyl)phenyl)nicotinamide (Compound HSND177)

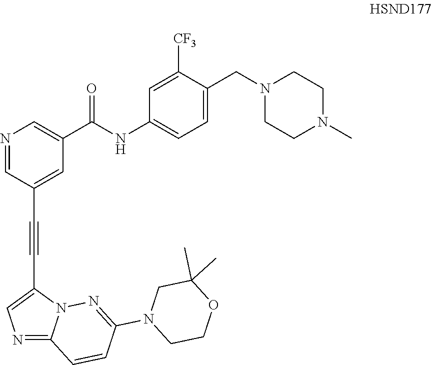 [SMILES: CN1CCN(Cc2ccc(NC(=O)c3cncc(C#Cc4cnc5ccc(N6CCOC(C)(C)C6)nn45)c3)cc2C(F)(F)F)CC1]

¹H NMR (500 MHz, DMSO-d₆) δ 10.74 (s, 1H), 9.07 (d, J=2.1 Hz, 1H), 8.92 (d, J=2.0 Hz, 1H), 8.46 (t, J=2.1 Hz, 1H), 8.17 (d, J=2.3 Hz, 1H), 8.02 (dd, J=8.4, 2.2 Hz, 1H), 7.97-7.90 (m, 2H), 7.70 (d, J=8.5 Hz, 1H), 7.30 (d, J=10.0 Hz, 1H), 3.75 (t, J=5.0 Hz, 2H), 3.54 (d, J=10.2 Hz, 4H), 3.40 (s, 2H), 2.45-2.20 (m, 8H), 2.13 (s, 3H), 1.20 (s, 6H); ¹³C NMR (126 MHz, DMSO-d₆) δ 163.83, 155.92, 153.74, 148.55, 138.27, 137.50, 137.35, 137.24, 133.01, 131.79, 130.35, 128.06 (q, J=30.4 Hz),126.55, 125.83 (q, J=274.8 Hz), 123.88, 119.49, 117.63, 112.35, 111.22, 94.59, 82.13, 71.23, 60.01, 57.91, 55.20, 54.85, 53.17, 46.19, 45.39, 24.64.

### N-(4-((4-Methylpiperazin-1-yl)methyl)-3-(trifluoromethyl)phenyl)-5-((6-(1-oxidothiomorpholino)imidazo[1,2-b]pyridazin-3-yl)ethynyl)nicotinamide (Compound HSND178)

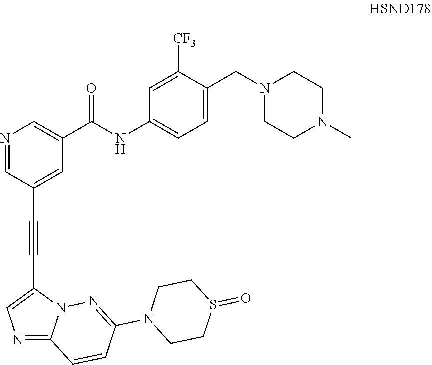 [SMILES: CN1CCN(Cc2ccc(NC(=O)c3cncc(C#Cc4cnc5ccc(N6CCS(=O)CC6)nn45)c3)cc2C(F)(F)F)CC1]

¹H NMR (500 MHz, DMSO-d₆) δ 9.09 (d, J=2.1 Hz, 1H), 8.91 (d, J=2.0 Hz, 1H), 8.47 (t, J=2.1 Hz, 1H), 8.18 (d, J=2.2 Hz, 1H), 8.03-7.94 (m, 3H), 7.69 (d, J=8.5 Hz, 1H), 7.40 (d, J=10.0 Hz, 1H), 4.21-4.13 (m, 2H), 4.03-3.94 (m, 2H), 3.55 (s, 2H), 3.06-2.97 (m, 2H), 2.81-2.74 (m, 2H), 2.45-2.20 (m, 8H), 2.14 (s, 3H); ¹³C NMR (126 MHz, DMSO-d₆) δ 163.93, 154.72, 153.61, 148.67, 139.30, 137.41, 137.35, 132.55, 131.74, 130.95, 128.00 (q, J=30.4 Hz), 126.85, 125.92 (q, J=274.6 Hz), 124.23, 119.38, 117.92, 112.54, 111.34, 94.74, 81.79, 57.95, 55.21, 53.18, 46.21, 43.88, 37.72.

### 5-((6-(2,2-Dimethylmorpholino)imidazo[1,2-b]pyridazin-3-yl)ethynyl)-N-(4-((4-methylpiperazin-1-yl)methyl)-3-(trifluoromethyl)phenyl)nicotinamide (Compound HSND177)

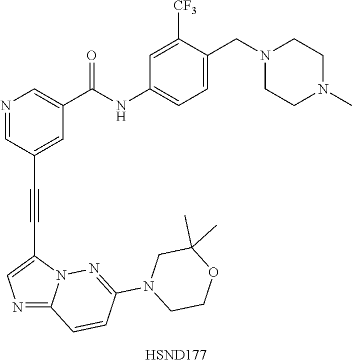

¹H NMR (500 MHz, DMSO-d₆) δ 10.74 (s, 1H), 9.07 (d, J=2.1 Hz, 1H), 8.92 (d, J=2.0 Hz, 1H), 8.46 (t, J=2.1 Hz, 1H), 8.17 (d, J=2.3 Hz, 1H), 8.02 (dd, J=8.4, 2.2 Hz, 1H), 7.97-7.90 (m, 2H), 7.70 (d, J=8.5 Hz, 1H), 7.30 (d, J=10.0 Hz, 1H), 3.75 (t, J=5.0 Hz, 2H), 3.54 (d, J=10.2 Hz, 4H), 3.40 (s, 2H), 2.45-2.20 (m, 8H), 2.13 (s, 3H), 1.20 (s, 6H); ¹³C NMR (126 MHz, DMSO-d₆) δ 163.83, 155,92, 153,74, 148.55, 138,27, 137,50, 137.35, 137,24, 133,01, 131,79, 130.35, 128.06 (q, J=30.4 Hz),126.55, 125.83 (q, J=274.8 Hz), 123.88, 119.49, 117.63, 112.35, 111.22, 94.59, 82.13, 71.23, 60.01, 57.91, 55.20, 54.85, 53.17, 46.19, 45.39, 24.64.

### N-(4-((4-Methylpiperazin-1-yl)methyl)-3-(trifluoromethyl)phenyl)-5-((6-(1-oxidothiomorpholino)imidazo[1,2-b]pyridazin-3-yl)ethynyl)nicotinamide (Compound HSND178)

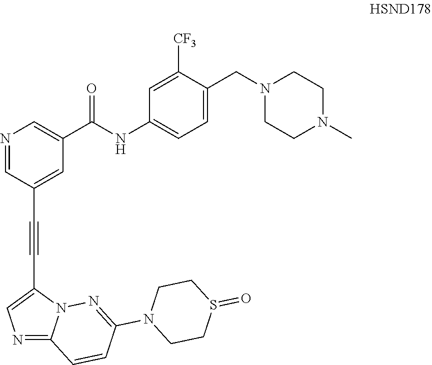 [SMILES: CN1CCN(Cc2ccc(NC(=O)c3cncc(C#Cc4cnc5ccc(N6CCS(=O)CC6)nn45)c3)cc2C(F)(F)F)CC1]

¹H NMR (500 MHz, DMSO-d₆) δ 9.09 (d, J=2.1 Hz, 1H), 8.91 (d, J=2.0 Hz, 1H), 8.47 (t, J=2.1 Hz, 1H), 8.18 (d, J=2.2 Hz, 1H), 8.03-7.94 (m, 3H), 7.69 (d, J=8.5 Hz, 1H), 7.40 (d, J=10.0 Hz, 1H), 4.21-4.13 (m, 2H), 4.03-3.94 (m, 2H), 3.55 (s, 2H), 3.06-2.97 (m, 2H), 2.81-2.74 (m, 2H), 2.45-2.20 (m, 8H), 2.14 (s, 3H); ¹³C NMR (126 MHz, DMSO-d₆) δ 163.93, 154.72, 153.61, 148.67, 139.30, 137.41, 137.35, 132.55, 131.74, 130.95, 128.00 (q, J=30.4 Hz), 126.85, 125.92 (q, J=274.6 Hz), 124.23, 119.38, 117.92, 112.54, 111.34, 94.74, 81.79, 57.95, 55.21, 53.18, 46.21, 43.88, 37.72.

### 9-(3-Bromoimidazo[1,2-b]pyridazin-6-yl)-3-oxa-9-azaspiro[5.5]undecane

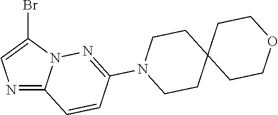 [SMILES: Brc1cnc2ccc(N3CCC4(CCOCC4)CC3)nn12]

¹H NMR (500 MHz, Methanol-d₄) δ 7.65 (d, J=10.0 Hz, 1H), 7.46 (s, 1H), 7.13 (d, J=10.0 Hz, 1H), 3.69-3.66 (m, 4H), 3.60-3.46 (m, 4H), 1.73-1.61 (m, 4H), 1.55 (t, J=5.4 Hz, 4H); ¹³C NMR (126 MHz, Methanol-d₄) δ 155.50, 136.64, 130.44, 124.61, 111.19, 100.03, 63.02, 41.19, 35.85, 34.86, 29.00.

### 5-((6-(3-Oxa-9-azaspiro[5.5]undecan-9-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-182)

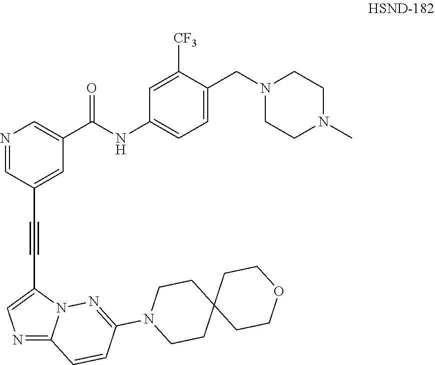 [SMILES: CN1CCN(Cc2ccc(NC(=O)c3cncc(C#Cc4cnc5ccc(N6CCC7(CCOCC7)CC6)nn45)c3)cc2C(F)(F)F)CC1]

¹H NMR (500 MHz, Methanol-d₄) δ 8.97 (d, J=2.2 Hz, 1H), 8.75 (d, J=2.0 Hz, 1H), 8.37 (t, J=2.1 Hz, 1H), 8.09 (d, J=2.3 Hz, 1H), 7.91 (dd, J=8.5, 2.3 Hz, 1H), 7.73 (d, J=8.6 Hz, 2H), 7.66 (d, J=10.0 Hz, 1H), 7.15 (d, J=10.0 Hz, 1H), 3.66-3.61 (m, 6H), 3.60-3.55 (m, 4H), 2.67-2.38 (m, 8H), 2.31 (s, 3H), 1.70-1.59 (m, 4H), 1.51 (t, J=5.3 Hz, 4H); ¹³C NMR (126 MHz, Methanol-d₄) δ 163.89, 155.56, 152.87, 146.89, 137.49, 137.36, 136.90, 135.40, 132.98, 131.20, 130.35, 128.83 (q, J=31.5 Hz), 125.37 (q, J=274.6 Hz), 124.61, 123.43, 120.27, 117.57, 112.34, 111.77, 93.49, 81.23, 62.98, 57.46, 54.56, 52.17, 44.48, 41.16, 35.82, 34.88, 28.97.

### 8-(3-Bromoimidazo[1,2-b]pyridazin-6-yl)-2-oxa-8-azaspiro[4.5]decane

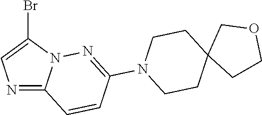 [SMILES: Brc1cnc2ccc(N3CCC4(CCOC4)CC3)nn12]

¹H NMR (500 MHz, Methanol-d₄) δ 7.65 (dd, J=10.0, 1.3 Hz, 1H), 7.46 (s, 1H), 7.13 (dd, J=10.0, 1.4 Hz, 1H), 3.87 (td, J=7.2, 1.3 Hz, 2H), 3.69-3.61 (m, 2H), 3.59-3.56 (m, 2H), 3.55-3.49 (m, 2H), 1.83 (td, J=7.2, 1.3 Hz, 2H), 1.67 (t, J=5.7 Hz, 4H); ¹³C NMR (126 MHz, Methanol-d₄) δ 155.37, 136.64, 130.51, 124.68, 111.26, 100.06, 77.30, 66.89, 43.67, 41.66, 36.50, 33.85.

### 5-((6-(2-Oxa-8-azaspiro[4.5]decan-8-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-183)

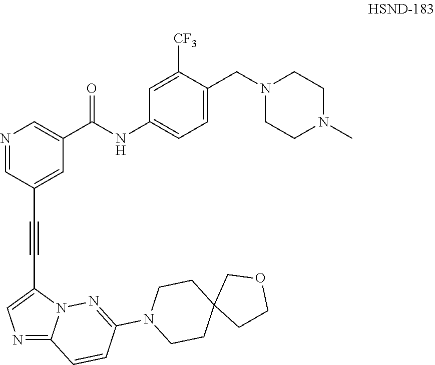 [SMILES: CN1CCN(Cc2ccc(NC(=O)c3cncc(C#Cc4cnc5ccc(N6CCC7(CCOC7)CC6)nn45)c3)cc2C(F)(F)F)CC1]

¹H NMR (500 MHz, Methanol-d₄) δ 9.04 (s, 1H), 8.84 (s, 1H), 8.41 (s, 1H), 8.12 (d, J=2.2 Hz, 1H), 8.01-7.90 (m, 1H), 7.76 (d, J=8.4 Hz, 1H), 7.24 (s, 1H), 3.86 (t, J=7.1 Hz, 2H), 3.75-3.65 (m, 4H), 3.62-3.53 (m, 4H), 2.73-2.47 (m, 8H), 2.38 (s, 3H), 1.84 (t, J=7.2 Hz, 2H), 1.71 (t, J=5.7 Hz, 4H).

### 7-(3-Bromoimidazo[1,2-b]pyridazin-6-yl)-2-oxa-7-azaspiro[3.5]nonane

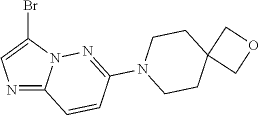 [SMILES: Brc1cnc2ccc(N3CCC4(CC3)COC4)nn12]

¹H NMR (500 MHz, Methanol-d₄) δ 7.66 (d, J=9.5 Hz, 1H), 7.47 (s, 1H), 7.14 (d, J=10.0 Hz, 1H), 4.49 (s, 4H), 3.54 (t, J=5.7 Hz, 4H), 1.96 (t. J=5.7 Hz, 4H); ¹³C NMR (126 MHz, Methanol-d₄) δ 155,33, 136,65, 130,55, 124,75, 111,27, 100,07, 81.37, 42,82, 38.67, 33.65.

### 5-((6-(2-Oxa-7-azaspiro[3.5]nonan-7-yl)imidazo[1,2-b]pyridazin-3-yl)ethynyl)-N-(4-((4-methylpiperazin-1-yl)methyl)-3-(trifluoromethyl)phenyl)nicotinamide (Compound HSND-184)

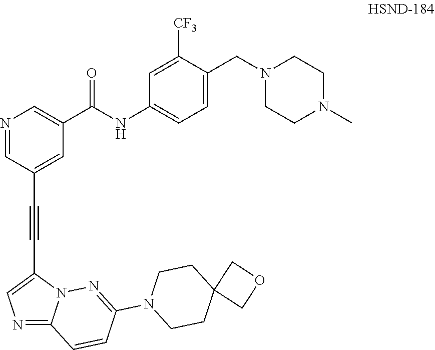 [SMILES: CN1CCN(Cc2ccc(NC(=O)c3cncc(C#Cc4cnc5ccc(N6CCC7(CC6)COC7)nn45)c3)cc2C(F)(F)F)CC1]

¹H NMR (500 MHz, Methanol-d₄) δ 9.03 (s, 1H), 8.83 (s, 1H), 8.42 (d, J=2.4 Hz, 1H), 8.13 (d, J=2.4 Hz, 1H), 7.94 (dd, J=8.4, 2.3 Hz, 1H), 7.86-7.65 (m, 3H), 7.22 (dd, J=9.9, 2.1 Hz, 1H), 4.55-4.44 (m, 4H), 3.58 (t, J=5.8 Hz, 4H), 1.98 (t, J=5.7 Hz, 4H).

### 4-(2-((3-Bromoimidazo[1,2-b]pyridazin-6-yl)oxy)ethyl)morpholine

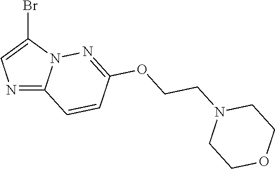 [SMILES: Brc1cnc2ccc(OCCN3CCOCC3)nn12]

¹H NMR (500 MHz, DMSO-d₆) δ 8.09-7.95 (m, 1H), 7.81-7.64 (m, 1H), 7.10-6.81 (m, 1H), 4.55-4.39 (m, 2H), 3.64-3.46 (m, 4H), 2.82-2.66 (m, 2H), 2.57-2.42 (m, 4H); ¹³C NMR (126 MHz, DMSO-d₆) δ 160.55, 138.05, 132.86, 128.57, 112.88, 100.66, 66.69, 64.93, 56.93, 54.02.

### N-(4-((4-Methylpiperazin-1-yl)methyl)-3-(trifluoromethyl)phenyl)-5-((6-(2-morpholinoethoxy)imidazo[1,2-b]pyridazin-3-yl)ethynyl)nicotinamide (Compound HSND185)

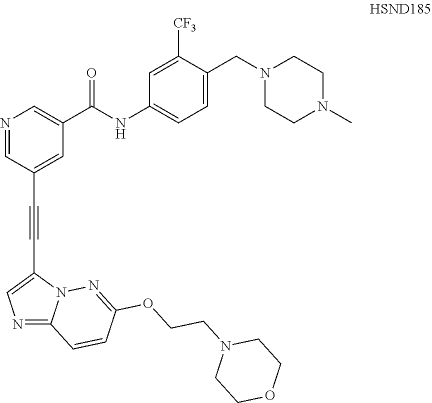 [SMILES: CN1CCN(Cc2ccc(NC(=O)c3cncc(C#Cc4cnc5ccc(OCCN6CCOCC6)nn45)c3)cc2C(F)(F)F)CC1]

¹H NMR (500 MHz, DMSO-d₆) δ 10.74 (s, 1H), 9.07 (s, 1H), 8.88 (s, 1H), 8.44 (s, 1H), 8.17 (s, 1H), 8.09-7.93 (m, 3H), 7.79-7.64 (m, 1H), 7.46-7.30 (m, 1H), 4.45-4.30 (m, 2H), 4.18-4.04 (m, 2H), 3.80-3.66 (m, 1H), 3.55 (s, 2H), 3.34-3.27 (m, 1H), 3.24-3.03 (m, 3H), 2.51-2.46 (m, 2H), 2.43-2.22 (m, 8H), 2.13 (s, 3H).

### (2S,6R)-4-(5-Iodoimidazo[2,1-b][1,3,4]thiadiazol-2-yl)-2,6-dimethylmorpholine

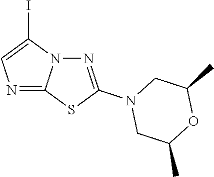 [SMILES: C[C@@H]1CN(c2nn3c(I)cnc3s2)C[C@H](C)O1]

¹H NMR (500 MHz, DMSO-d₆) δ 7.09 (s, 1H), 3.74-3.66 (m, 2H), 3.65-3.55 (m, 2H), 2.88-2.67 (m, 2H), 1.14 (d, J=6.2 Hz, 6H); ¹³C NMR (126 MHz, DMSO-d₆) δ 164.76, 140.45, 136.62, 70.91, 62.98, 53.11, 18.83.

### 5-((2-((2S,6R)-2,6-Dimethylmorpholino)imidazo[2,1-b][1,3,4]thiadiazol-5-yl)ethynyl)-N-(4-((4-methylpiperazin-1-yl)methyl)-3-(trifluoromethyl)phenyl)nicotinamide (Compound HSND186)

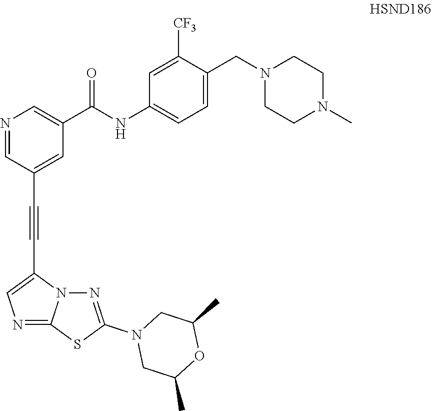 [SMILES: C[C@@H]1CN(c2nn3c(C#Cc4cncc(C(=O)Nc5ccc(CN6CCN(C)CC6)c(C(F)(F)F)c5)c4)cnc3s2)C[C@H](C)O1]

¹H NMR (500 MHz, DMSO-d₆) δ 10.74 (s, 1H), 9.08 (s, 1H), 8.92 (s, 1H), 8.49 (s, 1H), 8.18 (s, 1H), 8.06-7.95 (m, 1H), 7.71 (d, J=8.5 Hz, 1H), 7.56 (s, 1H), 3.76-3.63 (m, 4H), 3.55 (s, 2H), 2.83 (t, J=11.8 Hz, 2H), 2.45-2.20 (m, 8H), 2.14 (s, 3H), 1.15 (d, J=6.0 Hz, 6H).

### 1-((3-Bromoimidazo[1,2-b]pyridazin-6-yl)oxy)-2-methylpropan-2-ol

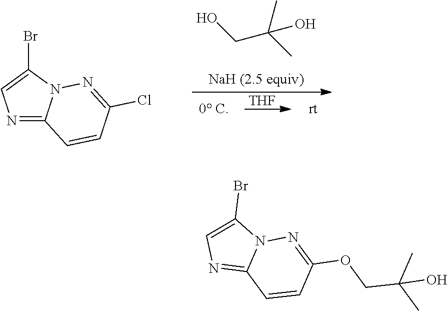

A reaction mixture of 3-bromo-6-chloroimidazo[1,2-b]pyridazine (500 mg) and 2-methylpropane-1,2-diol (2.3 requiv) in dry THF (20 mL) was cooled to 0° C. followed by the addition of NaH (2.5 equiv). The reaction mixture was then stirred at room temperature overnight. After completion, the reaction was extracted with ethyl acetate and washed with brine. The organic layer was collected and dried over sodium sulfate and concentrated under reduced pressure to get the desired product. Off-white solid, 93% yield. Off-white solid, Yield: 67%.

¹H NMR (500 MHz, Methanol-d₄) δ 7.86 (dd, J=9.4, 1.5 Hz, 1H), 7.60 (s, OH), 6.96 (dd, J=9.8, 1.7 Hz, 1H), 4.23 (s, 2H), 1.16 (s, 6H); ¹³C NMR (126 MHz, Methanol-d₄) δ 160.8, 137.9, 131.5, 126.8, 112.9, 100.8, 74.8, 68.9, 24.6.

### 5-((6-(2-Hydroxy-2-methylpropoxy)imidazo[1,2-b]pyridazin-3-yl)ethynyl)-N-(4-((4-methylpiperazin-1-yl)methyl)-3-(trifluoromethyl)phenyl)nicotinamide (HSND-25)

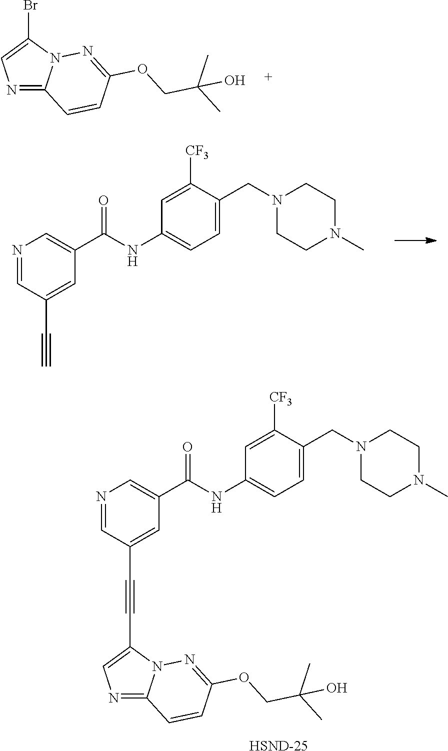

Off-white solid, Yield: 70%.

¹H NMR (500 MHz, Methanol-d₄) δ 9.13-8.98 (m, 1H), 8.90-8.80 (m, 1H), 8.51-8.41 (m, 1H), 8.17-8.07 (m, 1H), 8.00-7.84 (m, 3H), 7.76 (t, J=8.4 Hz, 1H), 7.02 (dd, J=12.2, 8.7 Hz, 1H), 4.29 (s, 2H), 3.66 (s, 2H), 2.56 (s, 8H), 2.35 (s, 3H), 1.35 (s, 7H); ¹³C NMR (126 MHz, Methanol-d₄) δ 163.9, 160.9, 153.1, 147.3, 138.1, 137.5, 136.4, 132.9, 131.2, 130.4, 128.8 (q, J=27.7 Hz), 126.8, 125.3 (q, J=274.6 Hz), 123.4, 119.9, 117.6, 114.2, 112.4, 93.7, 80.1, 74.9, 68.9, 57.4, 54.5, 52.0, 44.3, 25.1.

### 3-Bromo-6-((3-methyloxetan-3-yl)methoxy)imidazo[1,2-b]pyridazine

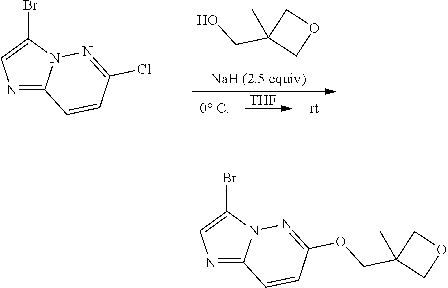

A reaction mixture of 3-bromo-6-chloroimidazo[1,2-b]pyridazine (500 mg) and (3-methyloxetan-3-yl)methanol (2.3 equiv) in dry THF (20 mL) was cooled to 0° C. followed by the addition of NaH (2.5 equiv). The reaction mixture was stirred at room temperature overnight. After completion, the reaction was extracted with ethyl acetate and washed with brine. The organic layer was collected and dried over sodium sulfate and concentrated under reduced pressure to get the desired product. Off-white solid, 90% yield.

¹H NMR (500 MHz, Methanol-d₄) δ 7.89 (dd, J=9.7, 0.6 Hz, 1H), 7.62 (s, 1H), 6.98 (dd, J=9.6, 0.6 Hz, 1H), 4.70 (d, J=6.0 Hz, 2H), 4.52 (s, 2H). 4.47 (d, J=6.0 Hz, 2H), 1.46 (s, 3H); ¹³C NMR (126 MHz, Methanol-d₄) δ 160.8, 137.9, 131.6, 126.9, 112.8, 100.8, 79.4, 71.6, 66.6, 39.1, 19.9.

### 5-((6-((3-Methyloxetan-3-yl)methoxy)imidazo[1,2-b]pyridazin-3-yl)ethynyl)-N-(4-((4-methylpiperazin-1-yl)methyl)-3-(trifluoromethyl)phenyl)nicotinamide (HSND-26)

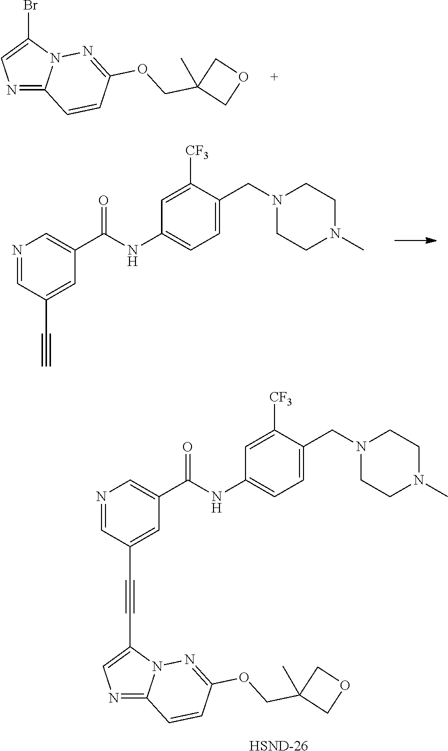

Off-white solid, Yield: 66%. H NMR (500 MHz, DMSO-d₆) δ10.79 (s, 1H), 9.10 (s, 1H), 8.97 (s, 1H), 8.52 (t, J=2.1 Hz, 1H), 8.18 (d, J=2.3 Hz, 1H), 8.13 (d, J=9.6 Hz, 1H), 8.09-8.00 (m, 2H), 7.71 (d, J=8.5 Hz, 1H), 7.07 (d, J=9.6 Hz, 1H), 4.58 (d, J=5.9 Hz, 2H), 4.52 (s, 2H), 4.29 (d, J=5.9 Hz, 2H), 3.55 (s, 2H), 2.38 (bs, 8H), 2.17 (s, 3H), 1.37 (s, 3H); ¹³C NMR (126 MHz, DMSO-d₆) δ 163.79, 160.9, 153.8, 148.8, 138.2, 138.0, 137.5, 132.9, 131.8, 130.3, 128.6, 128.0 (q, J=28.9 Hz), 125.8 (q, J=275.9 Hz), 123.9, 119.1, 117.6, 117.6, 114.2, 111.9, 95.0, 81.3, 79.1, 72.2, 57.8, 55.0, 52.9, 46.0, 39.2, 21.3.

### 3-Bromo-6-(oxetan-3-ylmethoxy)imidazo[1,2-b]pyridazine

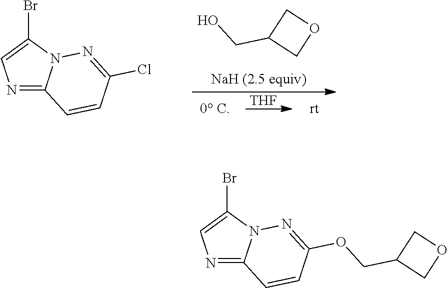 [SMILES: Clc1ccc2ncc(Br)n2n1 ; OCC1COC1 ; Brc1cnc2ccc(OCC3COC3)nn12 ; "THF"]

A reaction mixture of 3-bromo-6-chloroimidazo[1,2-b]pyridazine (500 mg) and oxetan-3-ylmethanol (2.3 equiv) in dry THF (20 mL) was cooled to 0° C. followed by addition of NaH (2.5 equiv). The reaction mixture was stirred at room temperature overnight. After completion, the reaction was extracted with ethyl acetate and washed with brine. The organic layer was collected and dried over sodium sulfate and concentrated under reduced pressure to get the desired product. Off-white solid, 93% yield.

¹H NMR (500 MHz, Methanol-d₄) δ 7.87 (d, J=9.7 Hz, 1H), 7.62 (s, 1H), 6.93 (d, J=9.7 Hz, 1H), 4.89 (dd, J=8.0, 6.2 Hz, 2H), 4.69-4.61 (m, 4H), 3.58-3.48 (m, 1H); ¹³C NMR (126 MHz, Methanol-d₄) δ 160.6, 137.9, 131.5, 126.9, 112.8, 100.8, 74.1, 68.2, 34.1.

### N-(4-((4-Methylpiperazin-1-yl)methyl)-3-(trifluoromethyl)phenyl)-5-((6-(oxetan-3-ylmethoxy)imidazo[1,2-b]pyridazin-3-yl)ethynyl)nicotinamide (HSND-27)

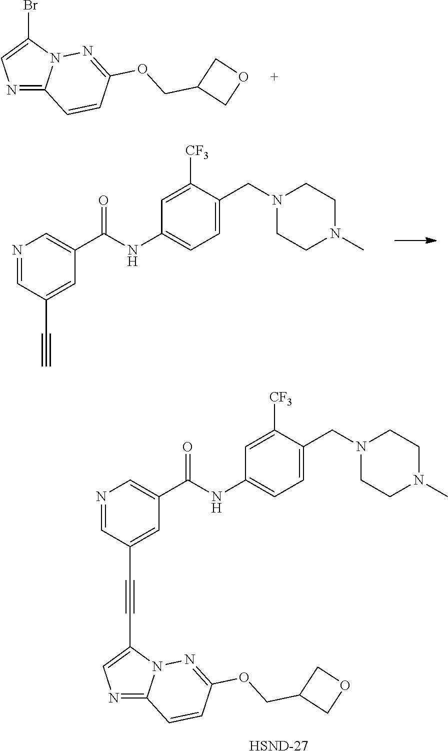

Off-white solid, Yield. 61%. ¹H NMR (500 MHz, DMSO-d₆) δ 10.76 (s, 1H), 9.09 (d, J=2.1 Hz, 1H), 8.98 (d, J=2.0 Hz, 1H), 8.51 (t, J=2.1 Hz, 1H), 8.18 (d, J=2.2 Hz, 1H), 8.13 (d, J=9.7 Hz, 1H), 8.07 (s, 1H), 8.02 (dd, J=8.5, 2.2 Hz, 1H), 7.72 (d, J=8.5 Hz, 1H), 7.05 (d, J=9.7 Hz, 1H), 4.70 (dd, J=8.0, 6.1 Hz, 2H), 4.64 (d, J=6.5 Hz, 2H), 4.53 (t, J=6.1 Hz, 2H), 3.56 (s, 2H), 3.51-3.42 (m, 1H), 2.37-2.32 (m, 8H), 2.14 (s, 3H); ¹³C NMR (126 MHz, DMSO-d₆) δ 163.8, 160.7, 153.9, 148.8, 138.5, 138.2, 138.0, 137.5, 133.0, 131.8, 130.4, 128.6, 127.8, 125.8 (q, J=275.9 Hz), 123.9, 119.2, 117.6, 117.6, 114.2, 111.8, 94.9, 81.3, 73.6, 73.6, 69.0, 57.9, 55.2, 53.1, 46.2, 34.1.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A compound having a structure of Formula (XX):
R₄-L-Z—R₃ (XX)
or a pharmaceutically acceptable salt thereof, wherein:
R₄ is a nicotinamide or benzamide, each of the nicotinamide or benzamide optionally substituted;
L is a linker comprising at least one atom;
Z is a bicyclic heterocycle substituted with R₃; and
R₃ comprises one or more ring moieties.

2. The compound of claim 1 having a structure of Formula (I): 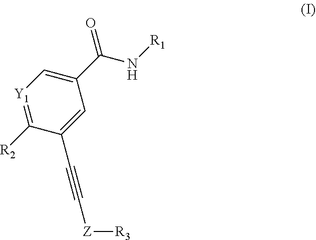
or a pharmaceutically acceptable salt thereof, wherein:
R₁ is a pyridine, an alkyl, an isoxazole, a pyrazole, or a phenyl group, each optionally substituted with one or more of a trifluoromethyl group, a piperazine (e.g., an alkyl piperazine), a pyrazine (e.g., an alkyl pyrazine), an imidazole (e.g., an alkyl imidazole), a cyanide, an amine, a halogen, an N-containing heterocycle, and/or an alkyl;
Y₁ is N or C;
R₂ is H, an alkyl, a heteroalkyl, or a halogen;
Z is imidazo[1,2-b]pyridazine, imidazo[1,2-b]pyrazole, or imidazo[2,1-b][1,3,4]thiadiazole; and
R₃ has a structure of Formula (II), Formula (III), or Formula (IV): 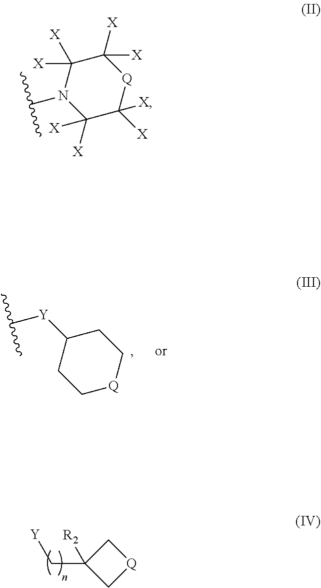
wherein: 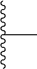
is a point of attachment;
each X is independently an alkyl or an H, with the proviso that all X are not H;
Q is O, S, N, or C, and is optionally substituted with one or more of an alkyl, a halogen, an O-alkyl, an amine, an —OH group, an alkoxy, a piperazine, a morpholine, an aziridine, a carbocycle or heterocycle, and/or a carbonyl;
Y is O or an amine, with the proviso that Y is not NH and
n is 0 or 1.

3. The compound of claim 1 or claim 2, wherein R₃ is a substituted morpholine.

4. The compound of claim 1, wherein the linker is an alkyne.

5. The compound of any one of claims 1, 2, or 4, wherein R₃ is an azetidine, optionally substituted with one or more of an alkyl, a halogen, an amine, a O-alkyl, and/or an —OH group.

6. The compound of any one of claims 1, 2, or 4, wherein R₃ is a pyrolidine, optionally substituted with an oxetane.

7. The compound of claim 1, wherein R₃ is or comprises

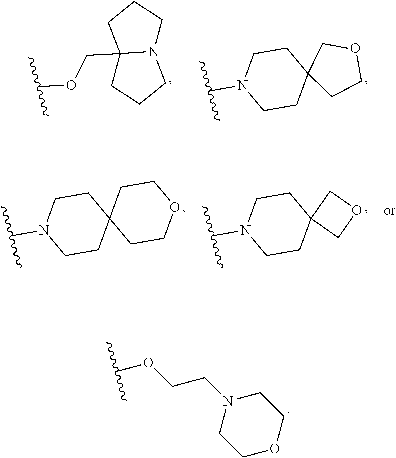

8. The compound of any one of claims 1, 2, or 4, wherein Z has a structure of: 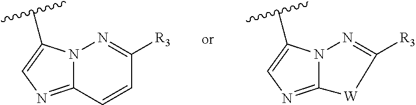
wherein 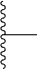
is a point of attachment, and W is C or S.

9. The compound of claim 1 or claim 2 having a structure of Formula (V):

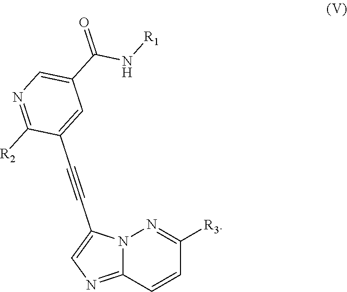

10. The compound of claim 2, wherein R₃ has a structure of Formula (III) and Y is a methylamine (—NMe) or an ethylamine (—NEt).

11. The compound of claim 2, wherein Q is O.

12. The compound of claim 2, wherein R₃ has the structure of Formula (II) and at least two X are linked together to form a bicyclic heterocycle.

13. The compound of any one of claims 1, 2, or 5-9, wherein R₃ comprises morpholine.

14. The compound of any one of claims 1, 2, or 11, wherein R₃ comprises unsubstituted morpholine.

15. The compound of claim 1, wherein R₃ comprises morpholine substituted with at least two methyl groups.

16. The compound of any one of claims 1, 2, 10-12, or 15, wherein R₃ comprises a 6-membered heterocycle.

17. The compound of any one of claims 1, 2,10-12, or 15, wherein R₃ comprises a 4-6-membered heterocycle.

18. The compound of any one of claims 1, 2, 10-12, or 15, wherein R₃ comprises an oxygen-containing heterocycle.

19. The compound of any one of claims 1, 2, or 9 having a structure:

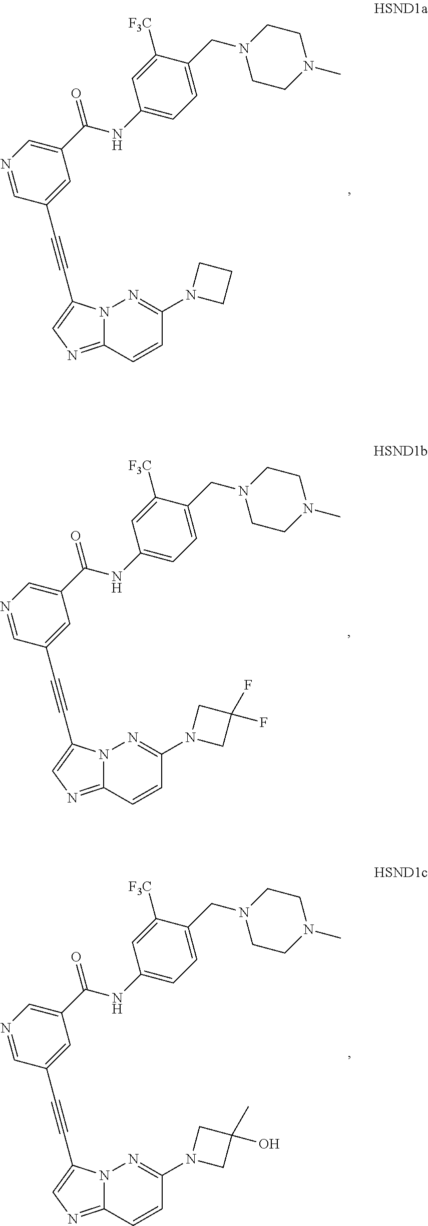 [SMILES: CN1CCN(Cc2ccc(NC(=O)c3cncc(C#Cc4cnc5ccc(N6CCC6)nn45)c3)cc2C(F)(F)F)CC1 ; CN1CCN(Cc2ccc(NC(=O)c3cncc(C#Cc4cnc5ccc(N6CC(F)(F)C6)nn45)c3)cc2C(F)(F)F)CC1 ; CN1CCN(Cc2ccc(NC(=O)c3cncc(C#Cc4cnc5ccc(N6CC(C)(O)C6)nn45)c3)cc2C(F)(F)F)CC1]

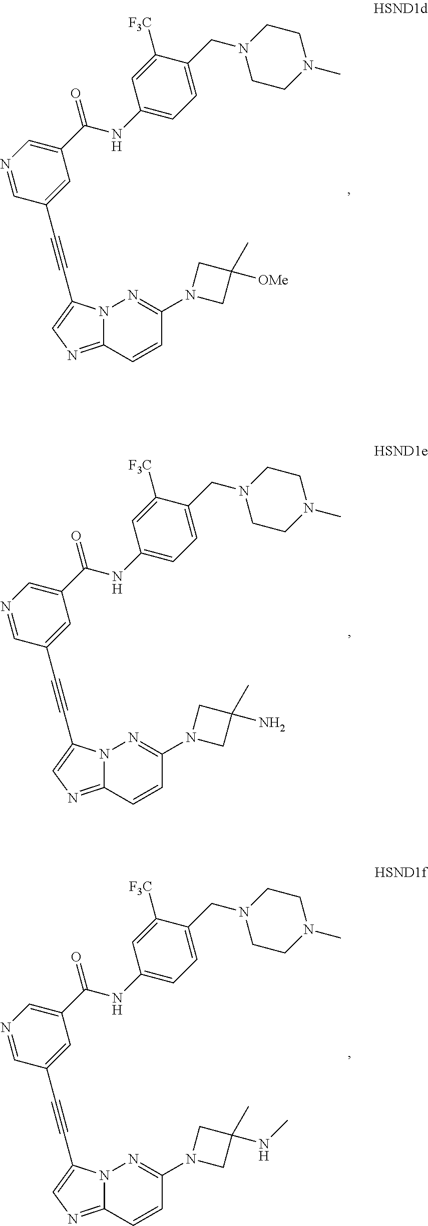 [SMILES: COC1(C)CN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CC(C)(N)C6)nn45)c3)cc2C(F)(F)F)CC1 ; CNC1(C)CN(c2ccc3ncc(C#Cc4cncc(C(=O)Nc5ccc(CN6CCN(C)CC6)c(C(F)(F)F)c5)c4)n3n2)C1]

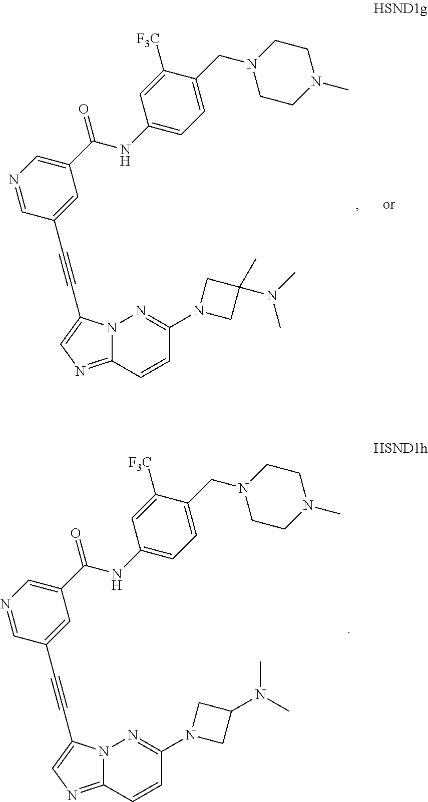 [SMILES: CN1CCN(Cc2ccc(NC(=O)c3cncc(C#Cc4cnc5ccc(N6CC(C)(N(C)C)C6)nn45)c3)cc2C(F)(F)F)CC1 ; CN1CCN(Cc2ccc(NC(=O)c3cncc(C#Cc4cnc5ccc(N6CC(N(C)C)C6)nn45)c3)cc2C(F)(F)F)CC1]

20. The compound of any one of claims 1, 2, or 9 having a structure:

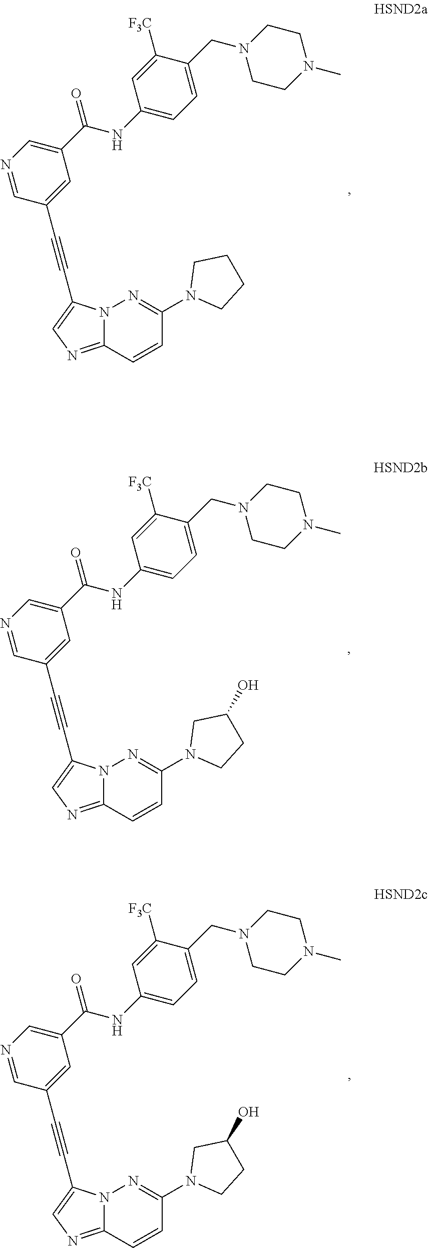 [SMILES: CN1CCN(Cc2ccc(NC(=O)c3cncc(C#Cc4cnc5ccc(N6CCCC6)nn45)c3)cc2C(F)(F)F)CC1 ; CN1CCN(Cc2ccc(NC(=O)c3cncc(C#Cc4cnc5ccc(N6CC[C@@H](O)C6)nn45)c3)cc2C(F)(F)F)CC1 ; CN1CCN(Cc2ccc(NC(=O)c3cncc(C#Cc4cnc5ccc(N6CC[C@H](O)C6)nn45)c3)cc2C(F)(F)F)CC1]

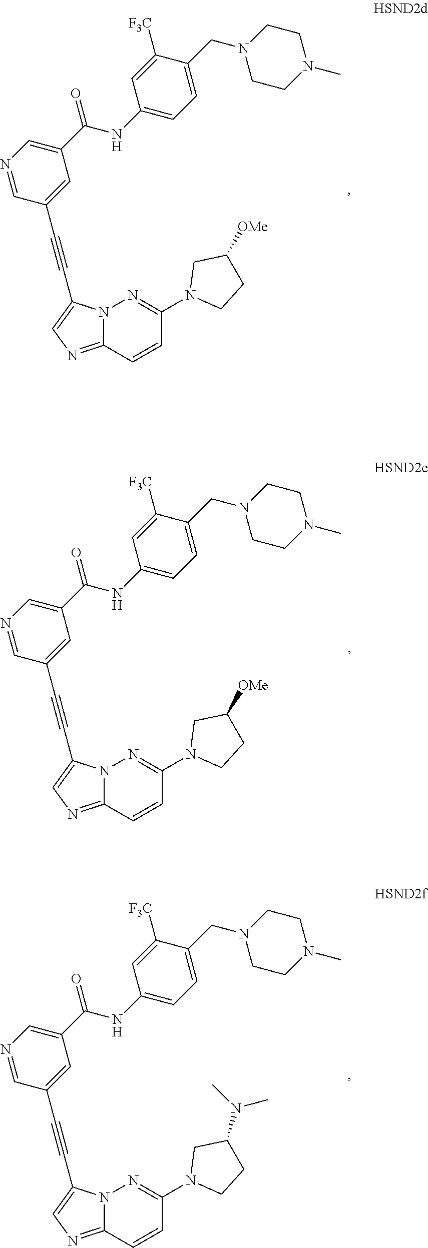 [SMILES: CO[C@@H]1CCN(c2ccc3ncc(C#Cc4cncc(C(=O)Nc5ccc(CN6CCN(C)CC6)c(C(F)(F)F)c5)c4)n3n2)C1 ; CO[C@H]1CCN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CC[C@@H](N(C)C)C6)nn45)c3)cc2C(F)(F)F)CC1]

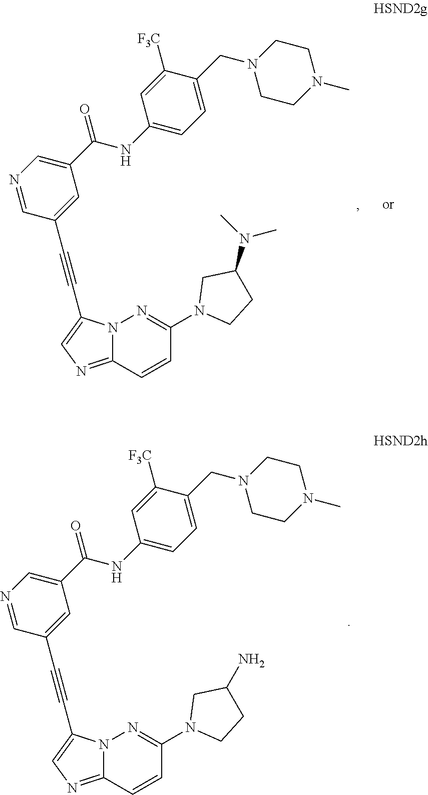 [SMILES: CN1CCN(Cc2ccc(NC(=O)c3cncc(C#Cc4cnc5ccc(N6CC[C@H](N(C)C)C6)nn45)c3)cc2C(F)(F)F)CC1 ; CN1CCN(Cc2ccc(NC(=O)c3cncc(C#Cc4cnc5ccc(N6CCC(N)C6)nn45)c3)cc2C(F)(F)F)CC1]

21. The compound of any one of claims 1, 2, or 9 having a structure:

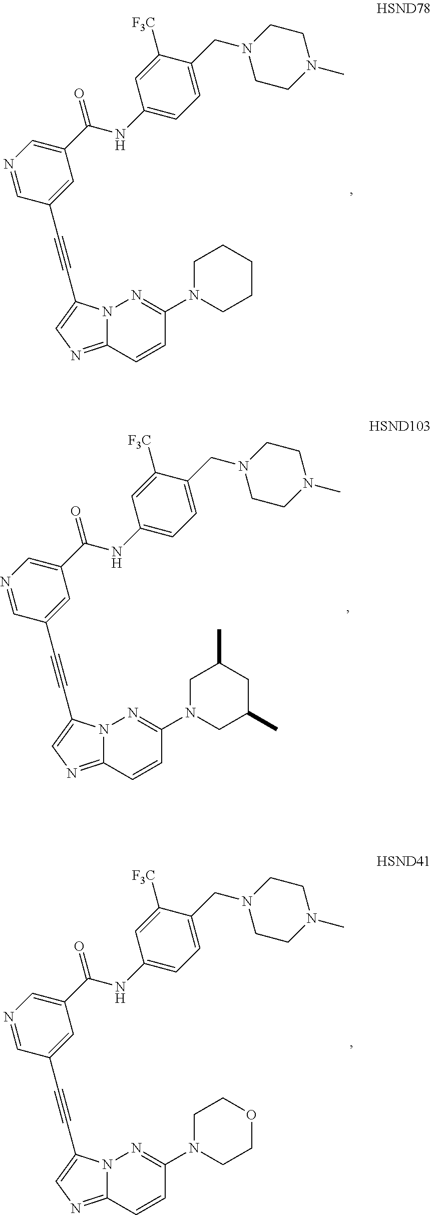 [SMILES: CN1CCN(Cc2ccc(NC(=O)c3cncc(C#Cc4cnc5ccc(N6CCCCC6)nn45)c3)cc2C(F)(F)F)CC1 ; C[C@@H]1C[C@H](C)CN(c2ccc3ncc(C#Cc4cncc(C(=O)Nc5ccc(CN6CCN(C)CC6)c(C(F)(F)F)c5)c4)n3n2)C1 ; CN1CCN(Cc2ccc(NC(=O)c3cncc(C#Cc4cnc5ccc(N6CCOCC6)nn45)c3)cc2C(F)(F)F)CC1]

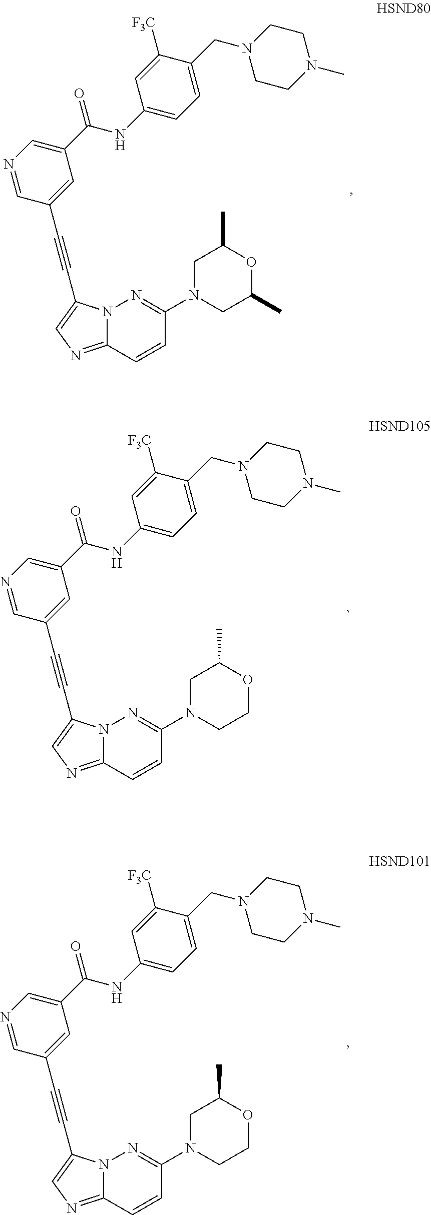 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)O1 ; C[C@H]1CN(c2ccc3ncc(C#Cc4cncc(C(=O)Nc5ccc(CN6CCN(C)CC6)c(C(F)(F)F)c5)c4)n3n2)CCO1 ; C[C@@H]1CN(c2ccc3ncc(C#Cc4cncc(C(=O)Nc5ccc(CN6CCN(C)CC6)c(C(F)(F)F)c5)c4)n3n2)CCO1]

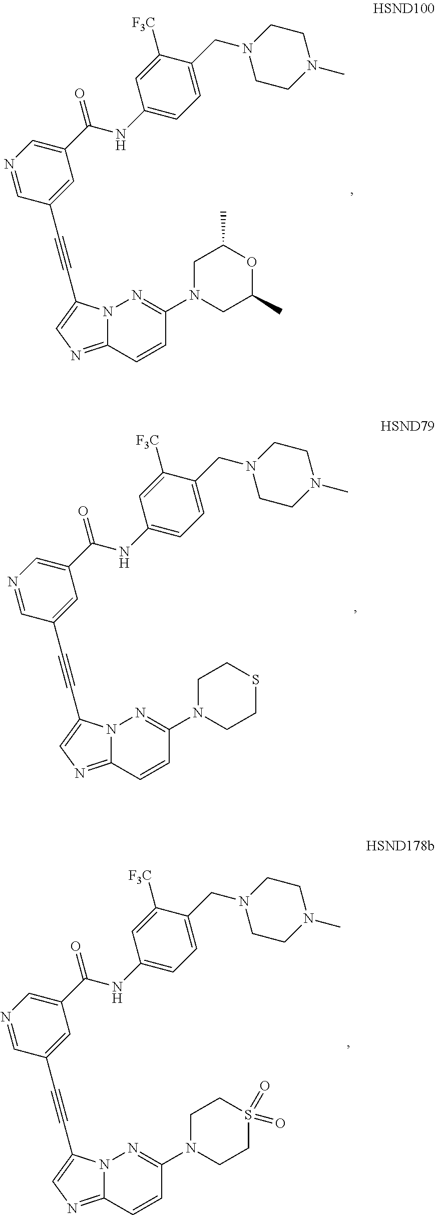 [SMILES: C[C@H]1CN(c2ccc3ncc(C#Cc4cncc(C(=O)Nc5ccc(CN6CCN(C)CC6)c(C(F)(F)F)c5)c4)n3n2)C[C@H](C)O1 ; CN1CCN(Cc2ccc(NC(=O)c3cncc(C#Cc4cnc5ccc(N6CCSCC6)nn45)c3)cc2C(F)(F)F)CC1 ; CN1CCN(Cc2ccc(NC(=O)c3cncc(C#Cc4cnc5ccc(N6CCS(=O)(=O)CC6)nn45)c3)cc2C(F)(F)F)CC1]

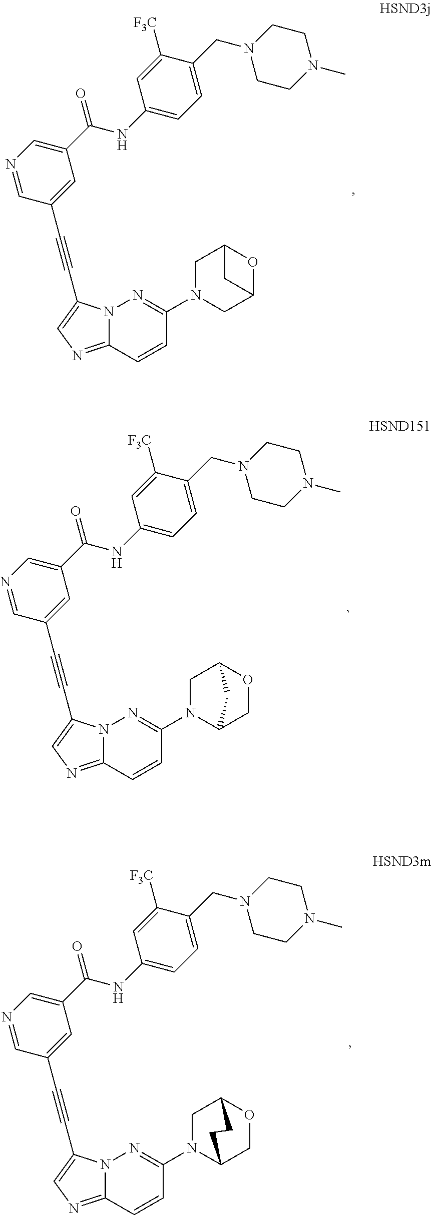

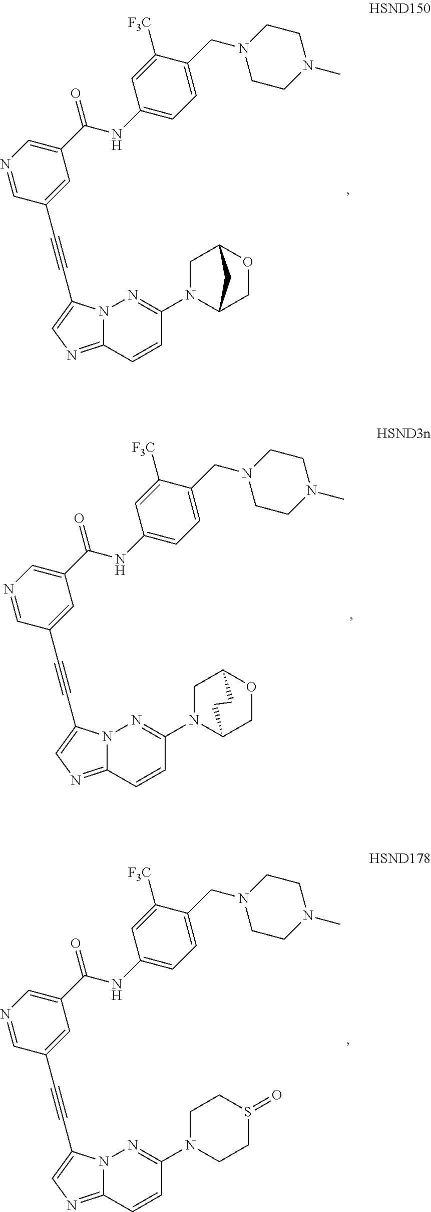 [SMILES: CN1CCN(Cc2ccc(NC(=O)c3cncc(C#Cc4cnc5ccc(N6C[C@H]7C[C@@H]6CO7)nn45)c3)cc2C(F)(F)F)CC1 ; CN1CCN(Cc2ccc(NC(=O)c3cncc(C#Cc4cnc5ccc(N6C[C@@H]7CC[C@H]6CO7)nn45)c3)cc2C(F)(F)F)CC1 ; CN1CCN(Cc2ccc(NC(=O)c3cncc(C#Cc4cnc5ccc(N6CCS(=O)CC6)nn45)c3)cc2C(F)(F)F)CC1]

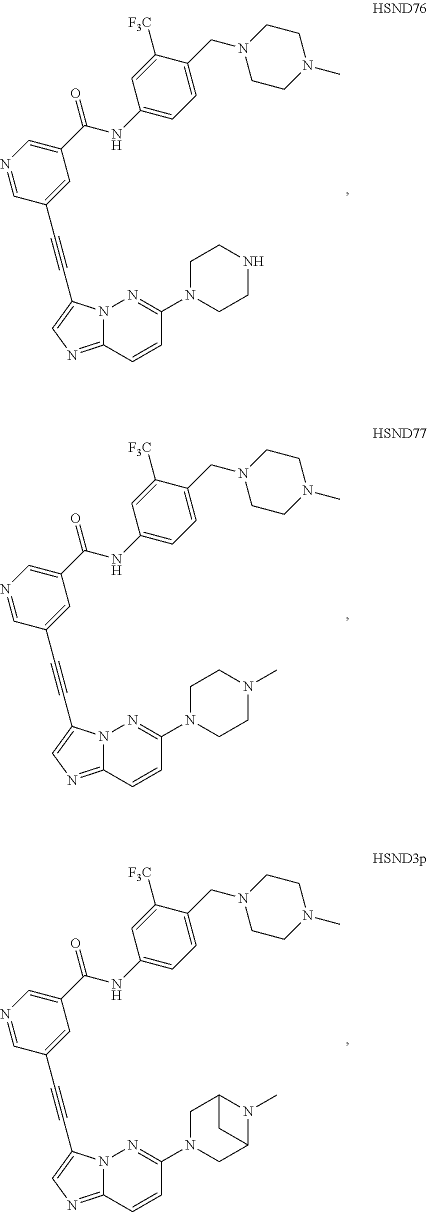 [SMILES: CN1CCN(Cc2ccc(NC(=O)c3cncc(C#Cc4cnc5ccc(N6CCNCC6)nn45)c3)cc2C(F)(F)F)CC1 ; CN1CCN(Cc2ccc(NC(=O)c3cncc(C#Cc4cnc5ccc(N6CCN(C)CC6)nn45)c3)cc2C(F)(F)F)CC1 ; CN1CCN(Cc2ccc(NC(=O)c3cncc(C#Cc4cnc5ccc(N6CC7CC(C6)N7C)nn45)c3)cc2C(F)(F)F)CC1]

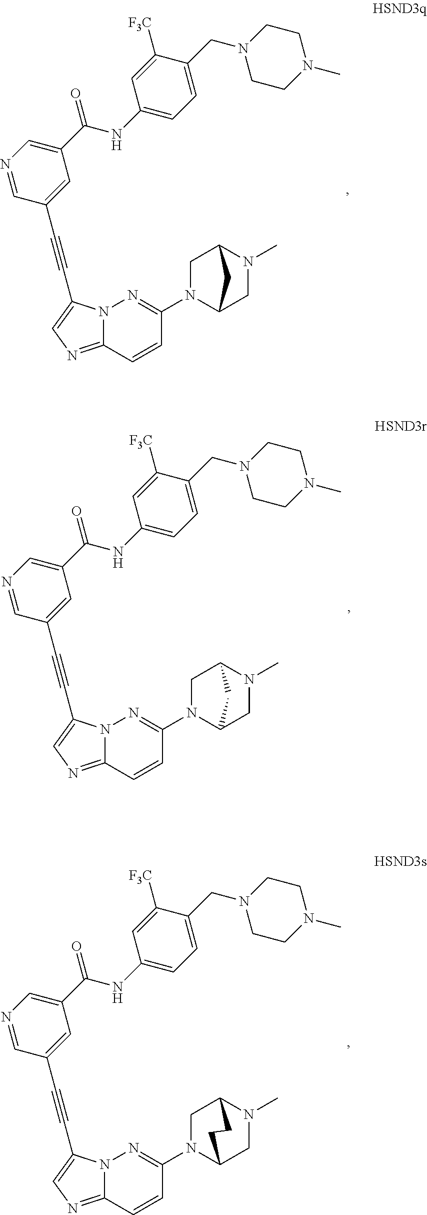

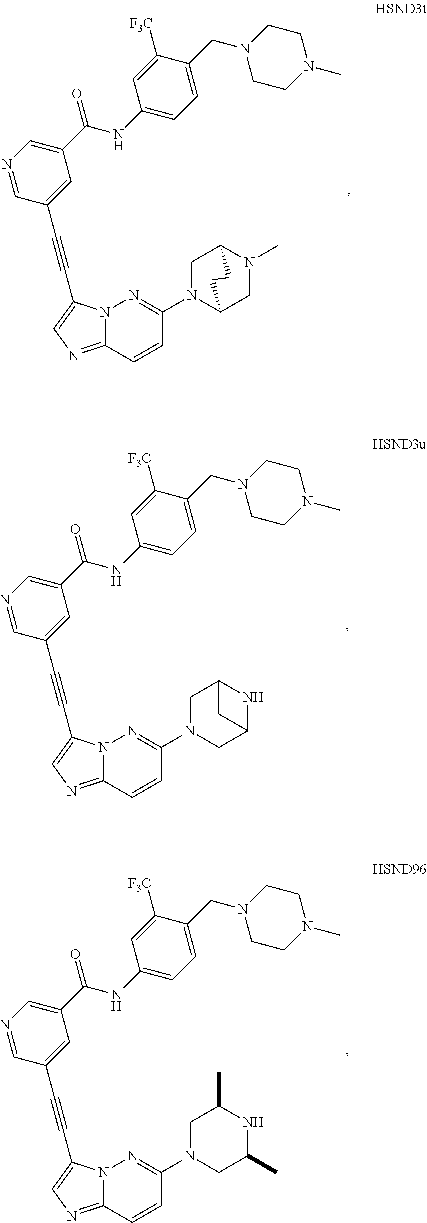 [SMILES: CN1CCN(Cc2ccc(NC(=O)c3cncc(C#Cc4cnc5ccc(N6C[C@@H]7CC[C@H]6CN7C)nn45)c3)cc2C(F)(F)F)CC1 ; CN1CCN(Cc2ccc(NC(=O)c3cncc(C#Cc4cnc5ccc(N6CC7CC(C6)N7)nn45)c3)cc2C(F)(F)F)CC1 ; C[C@@H]1CN(c2ccc3ncc(C#Cc4cncc(C(=O)Nc5ccc(CN6CCN(C)CC6)c(C(F)(F)F)c5)c4)n3n2)C[C@H](C)N1]

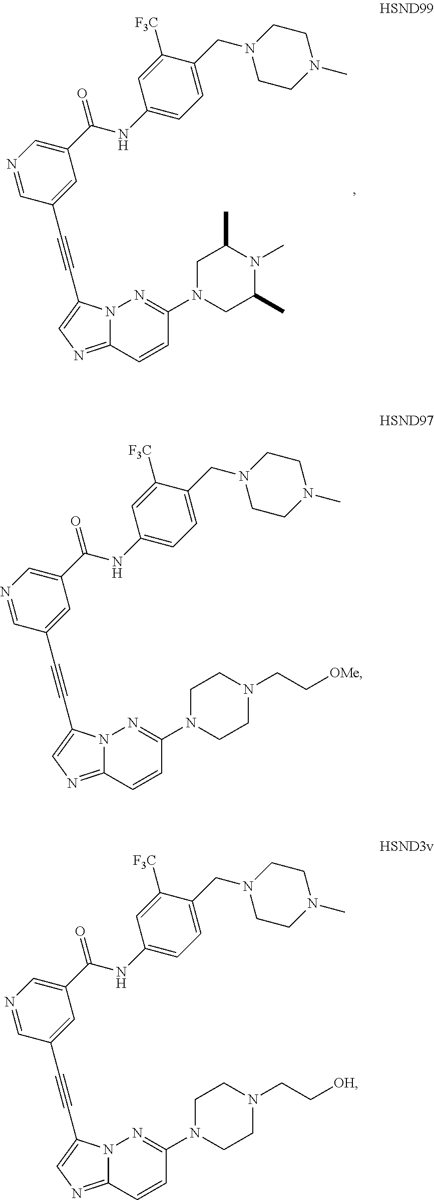 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)N1C ; COCCN1CCN(c2ccc3ncc(C#Cc4cncc(C(=O)Nc5ccc(CN6CCN(C)CC6)c(C(F)(F)F)c5)c4)n3n2)CC1 ; CN1CCN(Cc2ccc(NC(=O)c3cncc(C#Cc4cnc5ccc(N6CCN(CCO)CC6)nn45)c3)cc2C(F)(F)F)CC1]

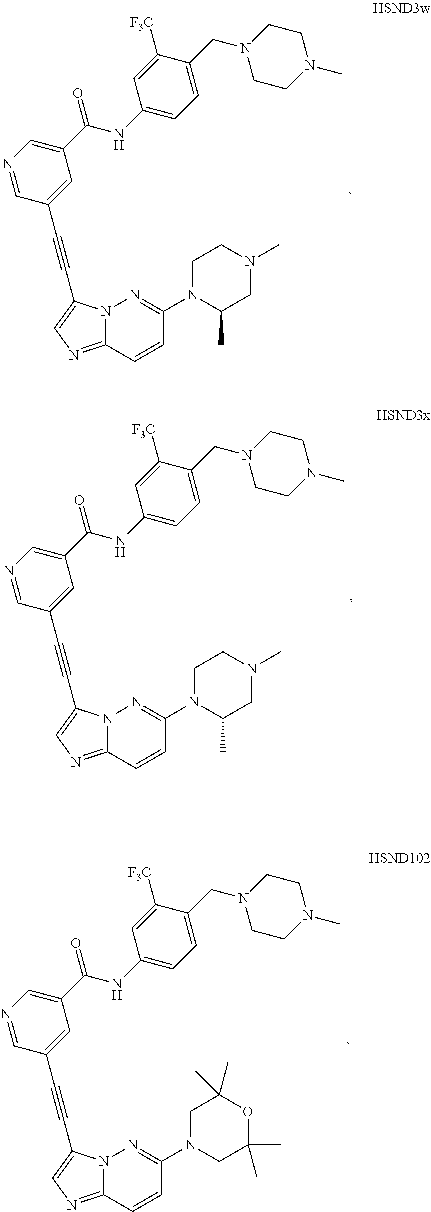 [SMILES: C[C@@H]1CN(C)CCN1c1ccc2ncc(C#Cc3cncc(C(=O)Nc4ccc(CN5CCN(C)CC5)c(C(F)(F)F)c4)c3)n2n1 ; C[C@H]1CN(C)CCN1c1ccc2ncc(C#Cc3cncc(C(=O)Nc4ccc(CN5CCN(C)CC5)c(C(F)(F)F)c4)c3)n2n1 ; CN1CCN(Cc2ccc(NC(=O)c3cncc(C#Cc4cnc5ccc(N6CC(C)(C)OC(C)(C)C6)nn45)c3)cc2C(F)(F)F)CC1]

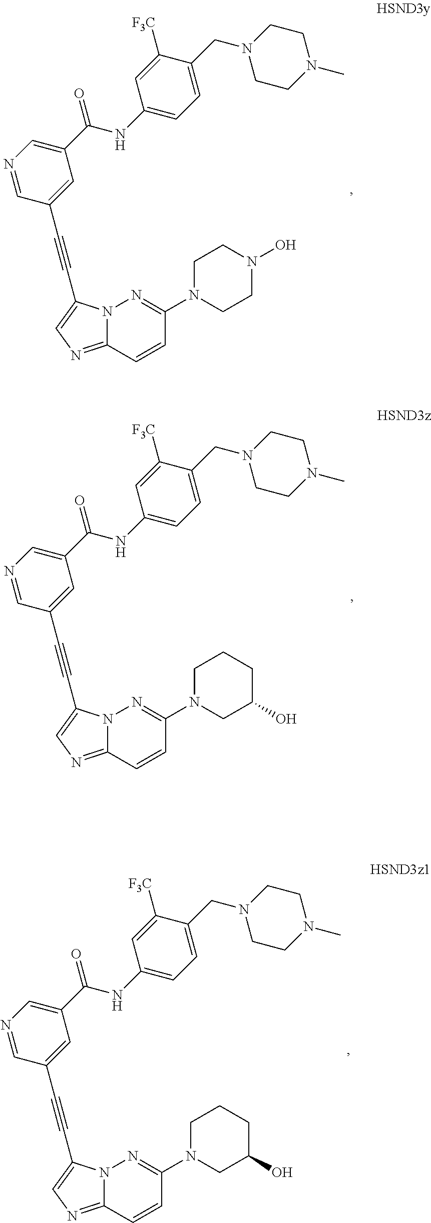 [SMILES: CN1CCN(Cc2ccc(NC(=O)c3cncc(C#Cc4cnc5ccc(N6CCN(O)CC6)nn45)c3)cc2C(F)(F)F)CC1 ; CN1CCN(Cc2ccc(NC(=O)c3cncc(C#Cc4cnc5ccc(N6CCC[C@H](O)C6)nn45)c3)cc2C(F)(F)F)CC1 ; CN1CCN(Cc2ccc(NC(=O)c3cncc(C#Cc4cnc5ccc(N6CCC[C@@H](O)C6)nn45)c3)cc2C(F)(F)F)CC1]

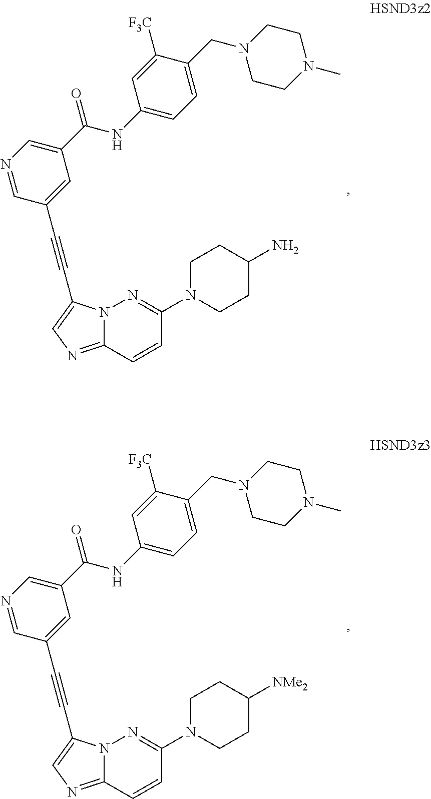 [SMILES: CN1CCN(Cc2ccc(NC(=O)c3cncc(C#Cc4cnc5ccc(N6CCC(N)CC6)nn45)c3)cc2C(F)(F)F)CC1 ; CN1CCN(Cc2ccc(NC(=O)c3cncc(C#Cc4cnc5ccc(N6CCC(N(C)C)CC6)nn45)c3)cc2C(F)(F)F)CC1]

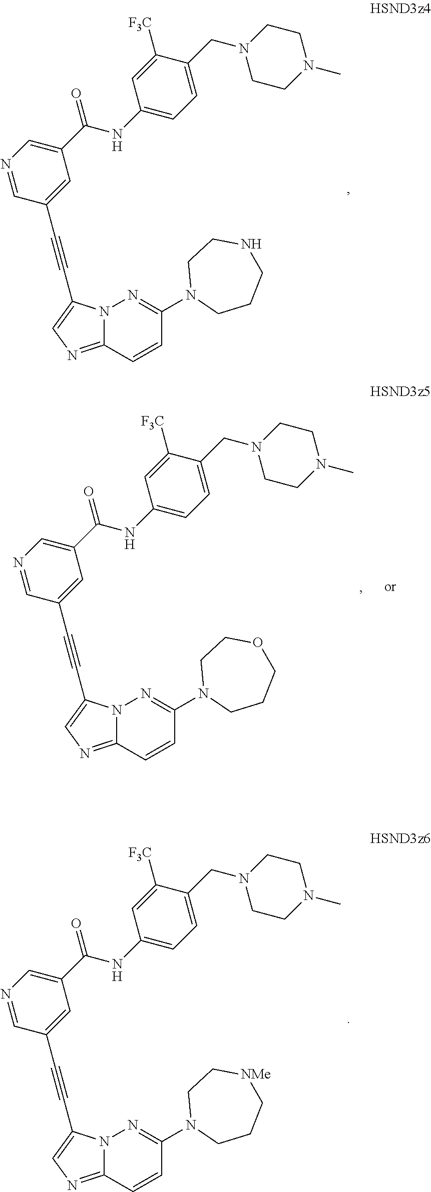 [SMILES: CN1CCN(Cc2ccc(NC(=O)c3cncc(C#Cc4cnc5ccc(N6CCCNCC6)nn45)c3)cc2C(F)(F)F)CC1 ; CN1CCN(Cc2ccc(NC(=O)c3cncc(C#Cc4cnc5ccc(N6CCCOCC6)nn45)c3)cc2C(F)(F)F)CC1 ; CN1CCN(Cc2ccc(NC(=O)c3cncc(C#Cc4cnc5ccc(N6CCCN(C)CC6)nn45)c3)cc2C(F)(F)F)CC1]

22. The compound of any one of claims 1, 2, or 9 having a structure:

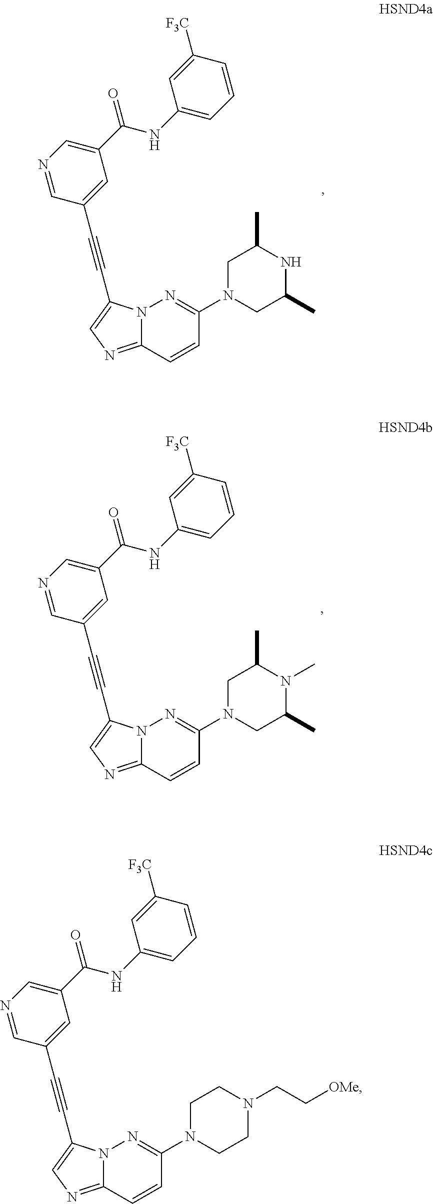 [SMILES: C[C@@H]1CN(c2ccc3ncc(C#Cc4cncc(C(=O)Nc5cccc(C(F)(F)F)c5)c4)n3n2)C[C@H](C)N1 ; C[C@@H]1CN(c2ccc3ncc(C#Cc4cncc(C(=O)Nc5cccc(C(F)(F)F)c5)c4)n3n2)C[C@H](C)N1C ; COCCN1CCN(c2ccc3ncc(C#Cc4cncc(C(=O)Nc5cccc(C(F)(F)F)c5)c4)n3n2)CC1]

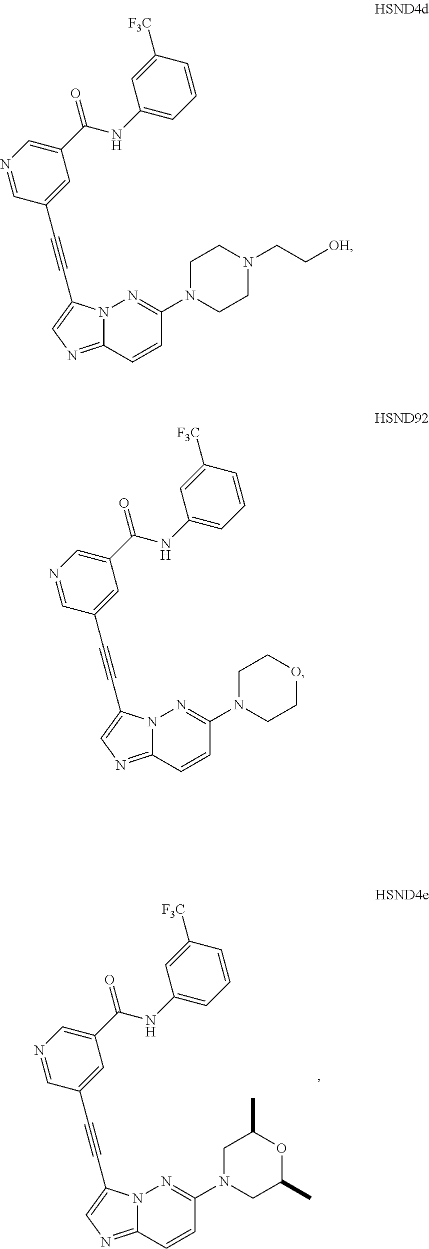 [SMILES: O=C(Nc1cccc(C(F)(F)F)c1)c1cncc(C#Cc2cnc3ccc(N4CCN(CCO)CC4)nn23)c1 ; O=C(Nc1cccc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5cccc(C(F)(F)F)c5)c4)n3n2)C[C@H](C)O1]

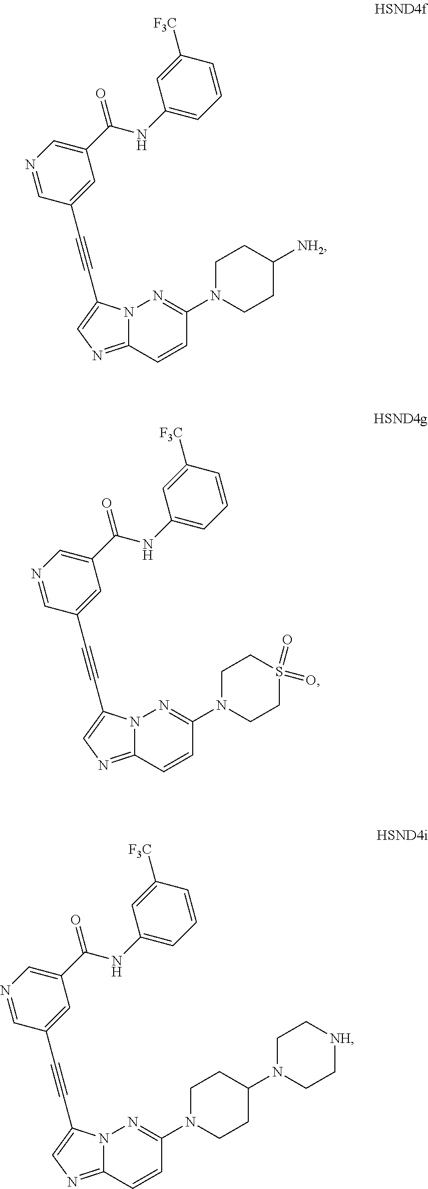 [SMILES: NC1CCN(c2ccc3ncc(C#Cc4cncc(C(=O)Nc5cccc(C(F)(F)F)c5)c4)n3n2)CC1 ; O=C(Nc1cccc(C(F)(F)F)c1)c1cncc(C#Cc2cnc3ccc(N4CCS(=O)(=O)CC4)nn23)c1 ; O=C(Nc1cccc(C(F)(F)F)c1)c1cncc(C#Cc2cnc3ccc(N4CCC(N5CCNCC5)CC4)nn23)c1]

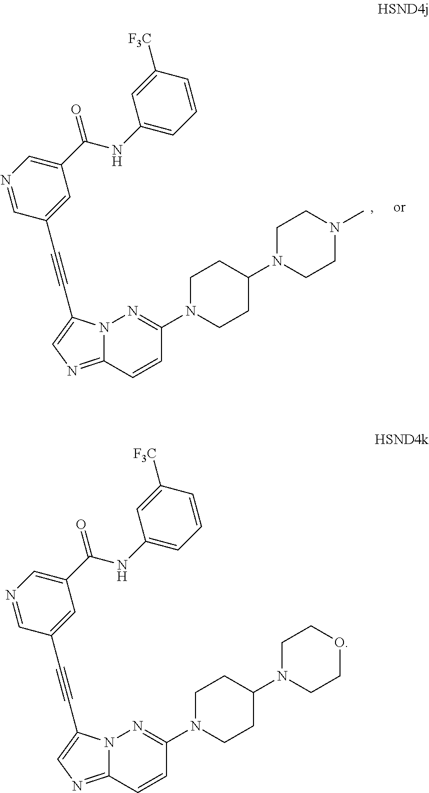 [SMILES: CN1CCN(C2CCN(c3ccc4ncc(C#Cc5cncc(C(=O)Nc6cccc(C(F)(F)F)c6)c5)n4n3)CC2)CC1 ; O=C(Nc1cccc(C(F)(F)F)c1)c1cncc(C#Cc2cnc3ccc(N4CCC(N5CCOCC5)CC4)nn23)c1]

23. The compound of any one of claims 1, 2, or 9 having a structure:

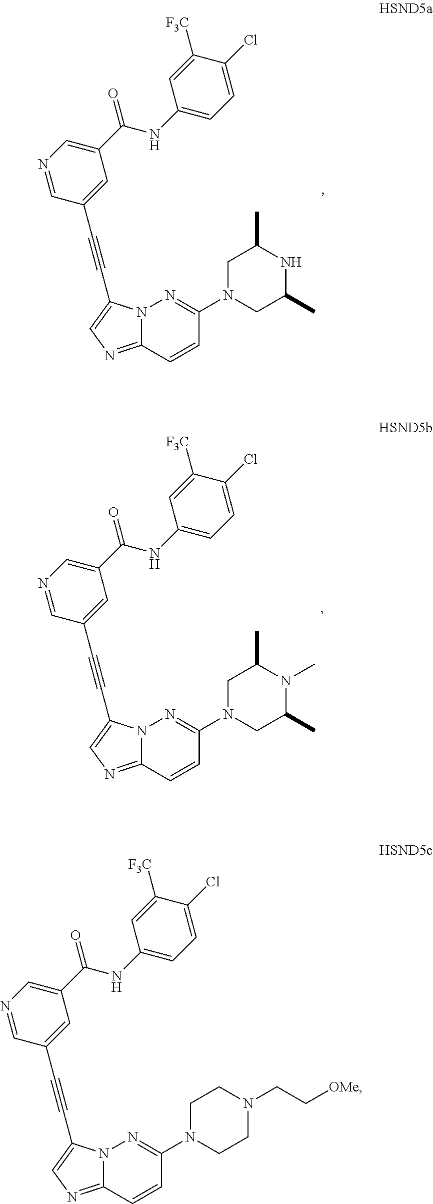 [SMILES: C[C@@H]1CN(c2ccc3ncc(C#Cc4cncc(C(=O)Nc5ccc(Cl)c(C(F)(F)F)c5)c4)n3n2)C[C@H](C)N1 ; C[C@@H]1CN(c2ccc3ncc(C#Cc4cncc(C(=O)Nc5ccc(Cl)c(C(F)(F)F)c5)c4)n3n2)C[C@H](C)N1C ; COCCN1CCN(c2ccc3ncc(C#Cc4cncc(C(=O)Nc5ccc(Cl)c(C(F)(F)F)c5)c4)n3n2)CC1]

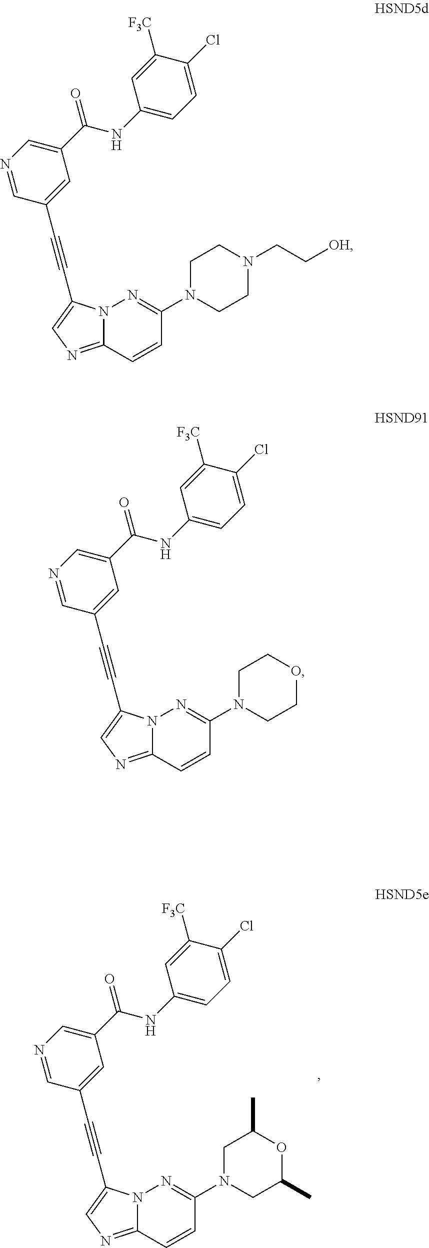 [SMILES: O=C(Nc1ccc(Cl)c(C(F)(F)F)c1)c1cncc(C#Cc2cnc3ccc(N4CCN(CCO)CC4)nn23)c1 ; O=C(Nc1ccc(Cl)c(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5ccc(Cl)c(C(F)(F)F)c5)c4)n3n2)C[C@H](C)O1]

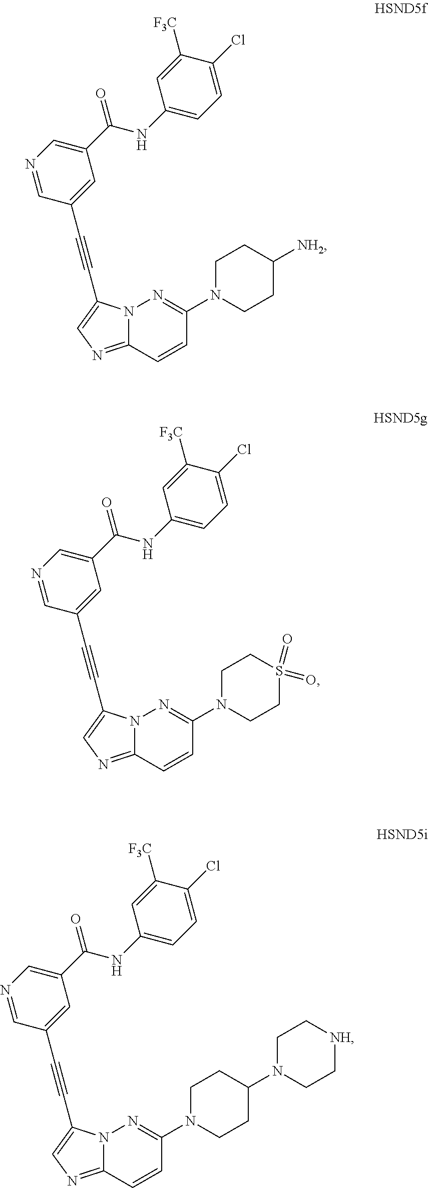 [SMILES: NC1CCN(c2ccc3ncc(C#Cc4cncc(C(=O)Nc5ccc(Cl)c(C(F)(F)F)c5)c4)n3n2)CC1 ; O=C(Nc1ccc(Cl)c(C(F)(F)F)c1)c1cncc(C#Cc2cnc3ccc(N4CCS(=O)(=O)CC4)nn23)c1 ; O=C(Nc1ccc(Cl)c(C(F)(F)F)c1)c1cncc(C#Cc2cnc3ccc(N4CCC(N5CCNCC5)CC4)nn23)c1]

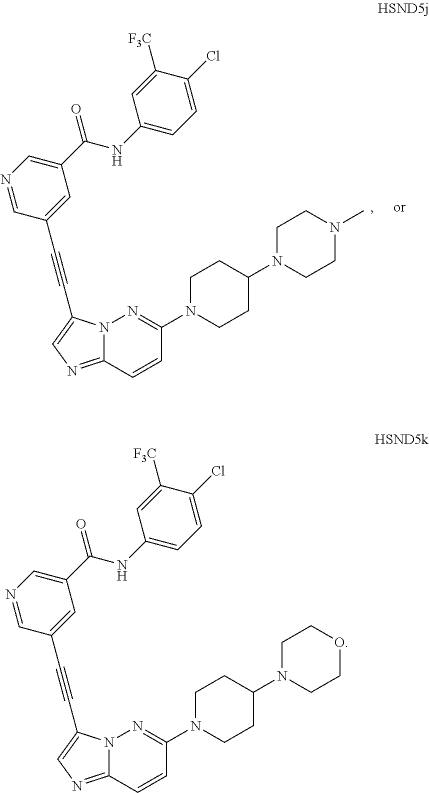 [SMILES: CN1CCN(C2CCN(c3ccc4ncc(C#Cc5cncc(C(=O)Nc6ccc(Cl)c(C(F)(F)F)c6)c5)n4n3)CC2)CC1 ; O=C(Nc1ccc(Cl)c(C(F)(F)F)c1)c1cncc(C#Cc2cnc3ccc(N4CCC(N5CCOCC5)CC4)nn23)c1]

24. The compound of any one of claims 1, 2, or 9 having a structure:

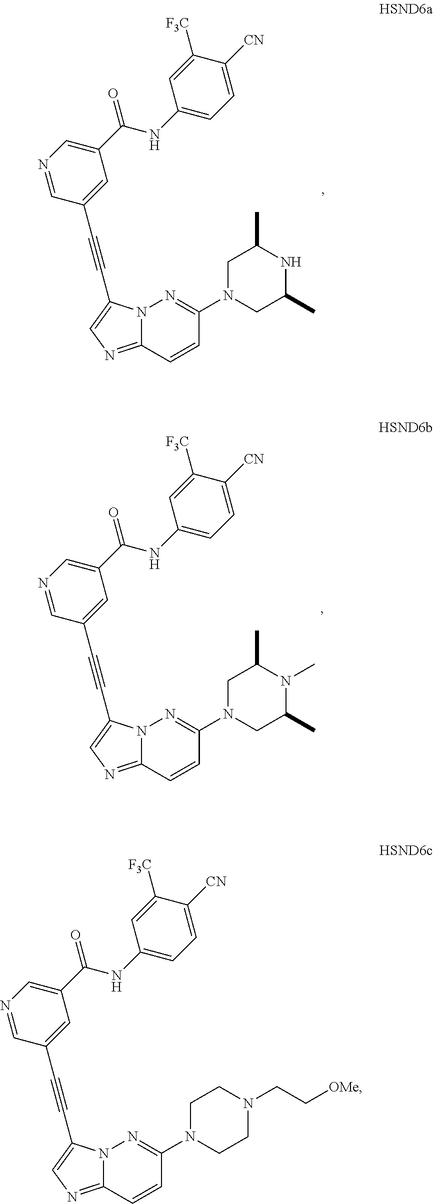 [SMILES: C[C@@H]1CN(c2ccc3ncc(C#Cc4cncc(C(=O)Nc5ccc(C#N)c(C(F)(F)F)c5)c4)n3n2)C[C@H](C)N1 ; C[C@@H]1CN(c2ccc3ncc(C#Cc4cncc(C(=O)Nc5ccc(C#N)c(C(F)(F)F)c5)c4)n3n2)C[C@H](C)N1C ; COCCN1CCN(c2ccc3ncc(C#Cc4cncc(C(=O)Nc5ccc(C#N)c(C(F)(F)F)c5)c4)n3n2)CC1]

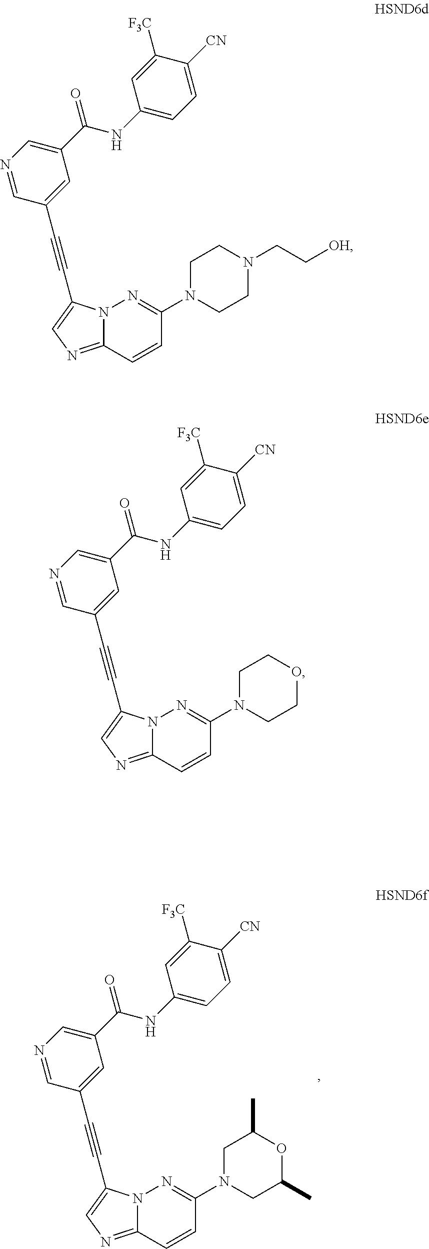 [SMILES: N#Cc1ccc(NC(=O)c2cncc(C#Cc3cnc4ccc(N5CCN(CCO)CC5)nn34)c2)cc1C(F)(F)F ; N#Cc1ccc(NC(=O)c2cncc(C#Cc3cnc4ccc(N5CCOCC5)nn34)c2)cc1C(F)(F)F ; C[C@@H]1CN(c2ccc3ncc(C#Cc4cncc(C(=O)Nc5ccc(C#N)c(C(F)(F)F)c5)c4)n3n2)C[C@H](C)O1]

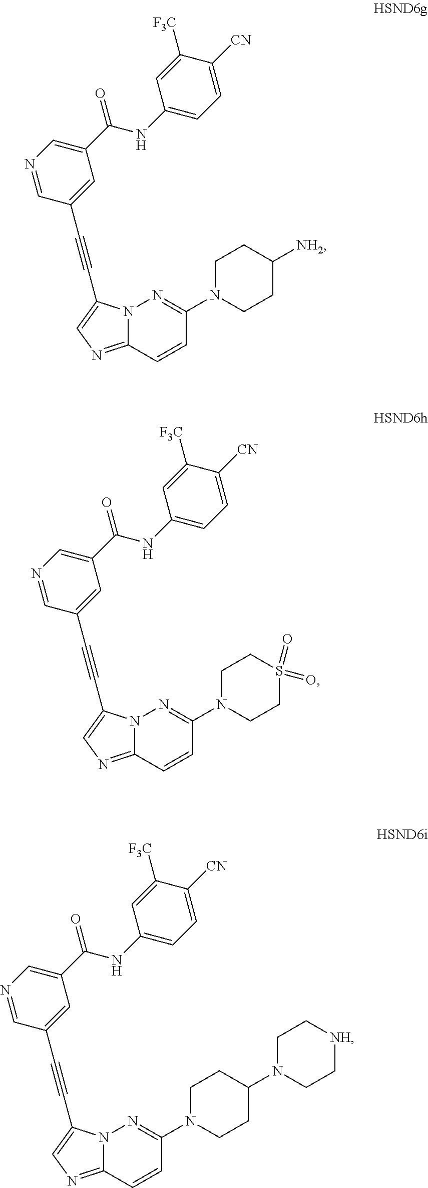 [SMILES: N#Cc1ccc(NC(=O)c2cncc(C#Cc3cnc4ccc(N5CCC(N)CC5)nn34)c2)cc1C(F)(F)F ; N#Cc1ccc(NC(=O)c2cncc(C#Cc3cnc4ccc(N5CCS(=O)(=O)CC5)nn34)c2)cc1C(F)(F)F ; N#Cc1ccc(NC(=O)c2cncc(C#Cc3cnc4ccc(N5CCC(N6CCNCC6)CC5)nn34)c2)cc1C(F)(F)F]

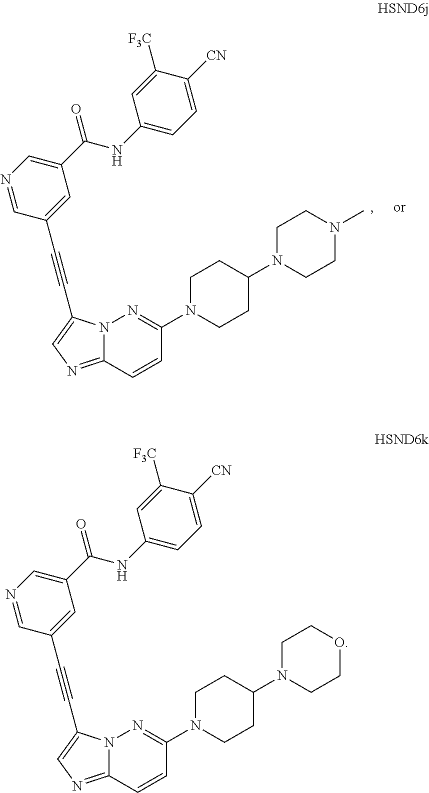 [SMILES: CN1CCN(C2CCN(c3ccc4ncc(C#Cc5cncc(C(=O)Nc6ccc(C#N)c(C(F)(F)F)c6)c5)n4n3)CC2)CC1 ; N#Cc1ccc(NC(=O)c2cncc(C#Cc3cnc4ccc(N5CCC(N6CCOCC6)CC5)nn34)c2)cc1C(F)(F)F]

25. The compound of any one of claims 1, 2, or 9 having a structure:

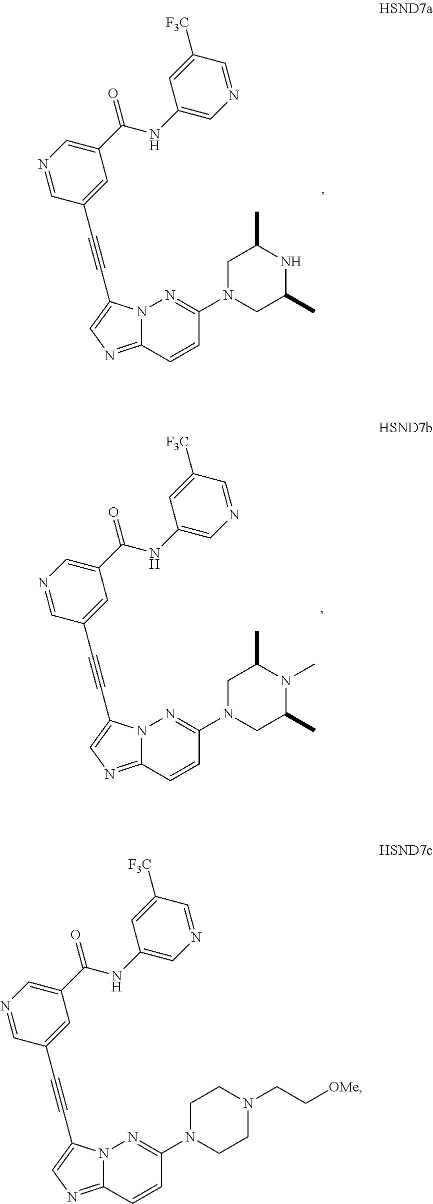 [SMILES: C[C@@H]1CN(c2ccc3ncc(C#Cc4cncc(C(=O)Nc5cncc(C(F)(F)F)c5)c4)n3n2)C[C@H](C)N1 ; C[C@@H]1CN(c2ccc3ncc(C#Cc4cncc(C(=O)Nc5cncc(C(F)(F)F)c5)c4)n3n2)C[C@H](C)N1C ; COCCN1CCN(c2ccc3ncc(C#Cc4cncc(C(=O)Nc5cncc(C(F)(F)F)c5)c4)n3n2)CC1]

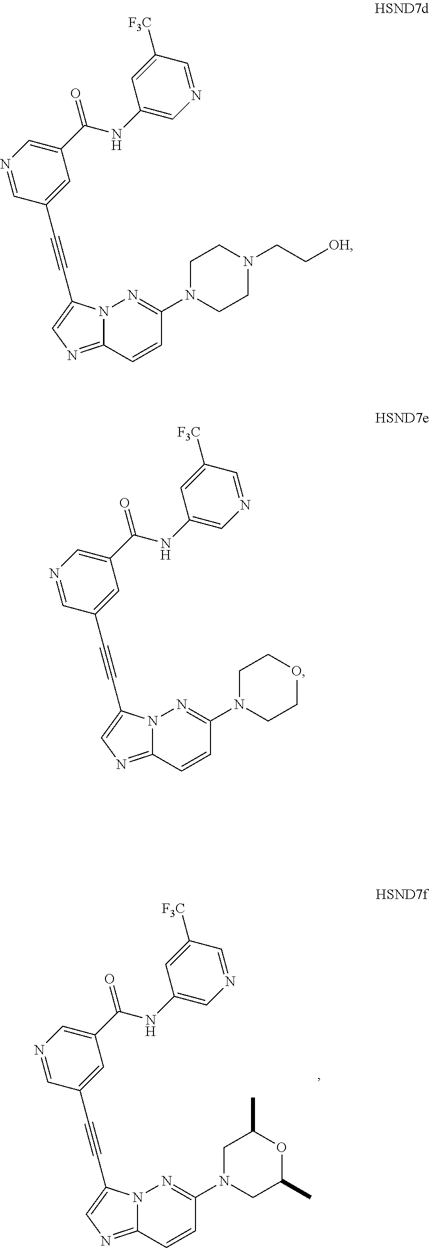 [SMILES: O=C(Nc1cncc(C(F)(F)F)c1)c1cncc(C#Cc2cnc3ccc(N4CCN(CCO)CC4)nn23)c1 ; O=C(Nc1cncc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5cncc(C(F)(F)F)c5)c4)n3n2)C[C@H](C)O1]

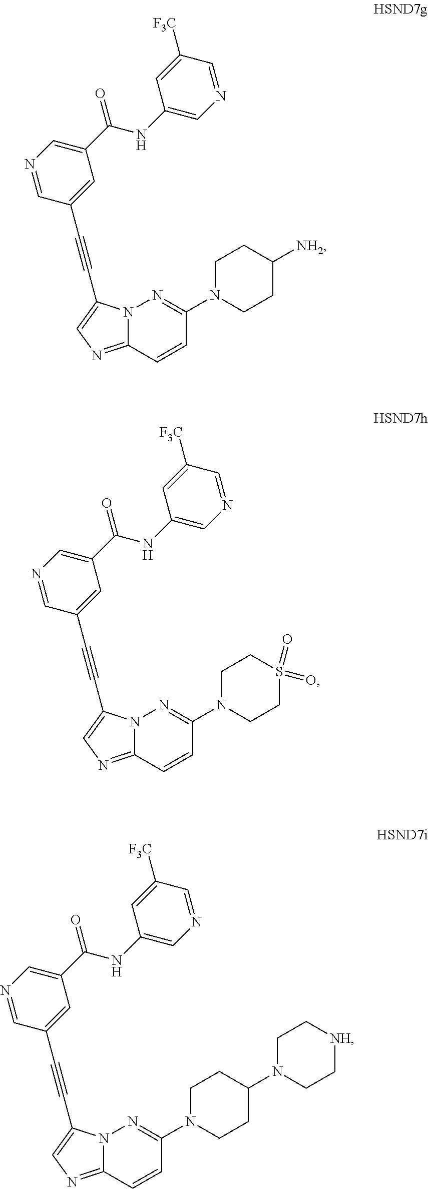 [SMILES: NC1CCN(c2ccc3ncc(C#Cc4cncc(C(=O)Nc5cncc(C(F)(F)F)c5)c4)n3n2)CC1 ; O=C(Nc1cncc(C(F)(F)F)c1)c1cncc(C#Cc2cnc3ccc(N4CCS(=O)(=O)CC4)nn23)c1 ; O=C(Nc1cncc(C(F)(F)F)c1)c1cncc(C#Cc2cnc3ccc(N4CCC(N5CCNCC5)CC4)nn23)c1]

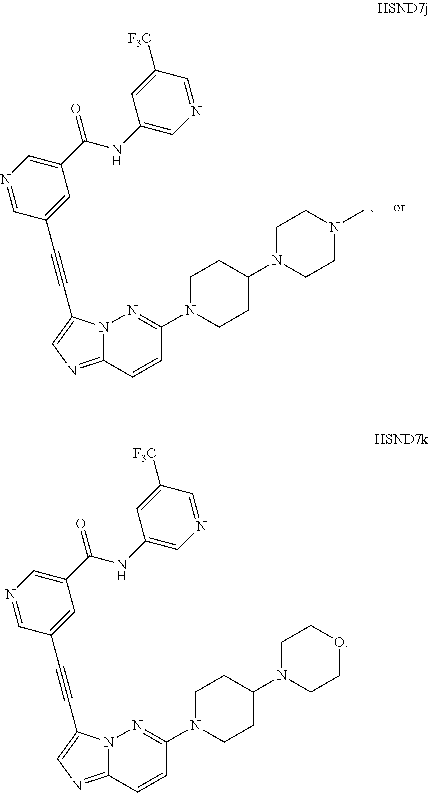 [SMILES: CN1CCN(C2CCN(c3ccc4ncc(C#Cc5cncc(C(=O)Nc6cncc(C(F)(F)F)c6)c5)n4n3)CC2)CC1 ; O=C(Nc1cncc(C(F)(F)F)c1)c1cncc(C#Cc2cnc3ccc(N4CCC(N5CCOCC5)CC4)nn23)c1]

26. The compound of any one of claims 1, 2, or 9 having a structure:

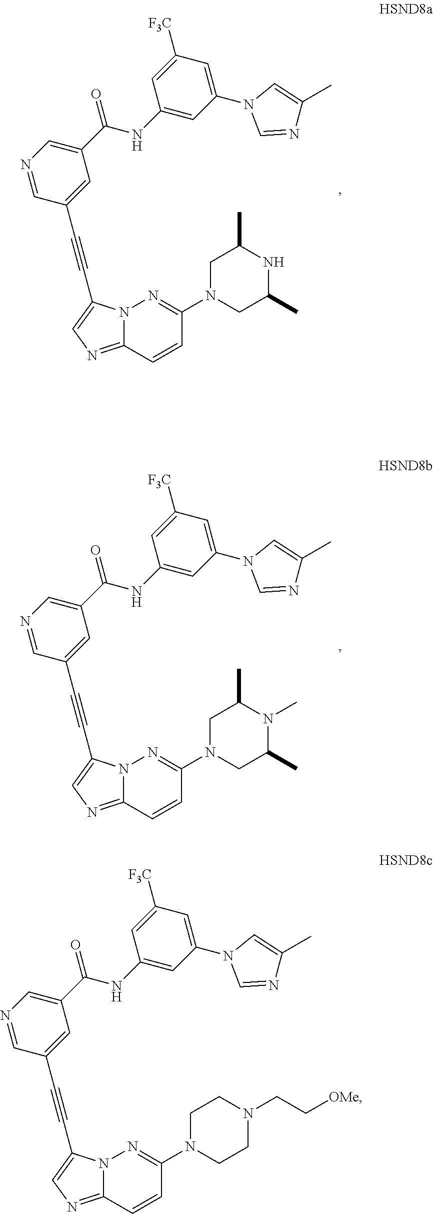 [SMILES: Cc1cn(-c2cc(NC(=O)c3cncc(C#Cc4cnc5ccc(N6C[C@@H](C)N[C@@H](C)C6)nn45)c3)cc(C(F)(F)F)c2)cn1 ; Cc1cn(-c2cc(NC(=O)c3cncc(C#Cc4cnc5ccc(N6C[C@@H](C)N(C)[C@@H](C)C6)nn45)c3)cc(C(F)(F)F)c2)cn1 ; COCCN1CCN(c2ccc3ncc(C#Cc4cncc(C(=O)Nc5cc(-n6cnc(C)c6)cc(C(F)(F)F)c5)c4)n3n2)CC1]

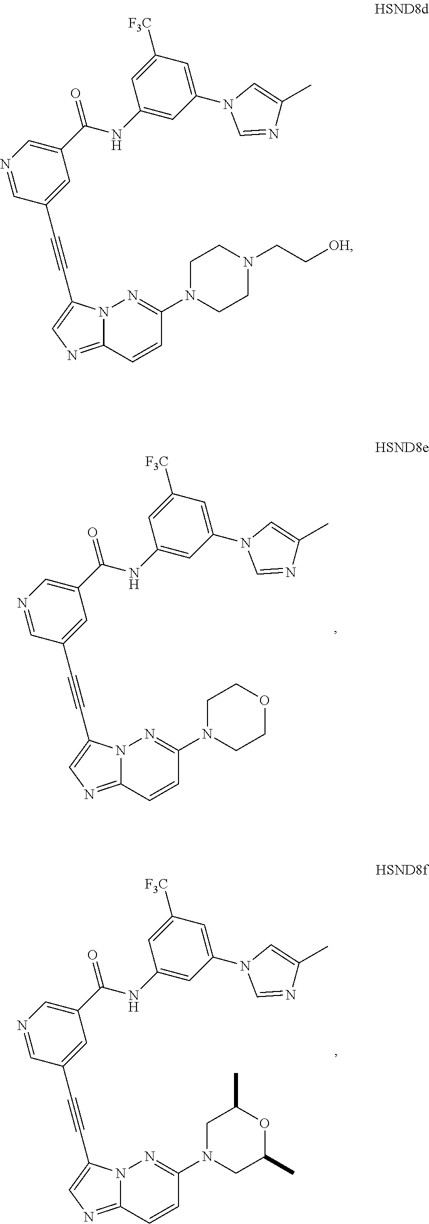 [SMILES: Cc1cn(-c2cc(NC(=O)c3cncc(C#Cc4cnc5ccc(N6CCN(CCO)CC6)nn45)c3)cc(C(F)(F)F)c2)cn1 ; Cc1cn(-c2cc(NC(=O)c3cncc(C#Cc4cnc5ccc(N6CCOCC6)nn45)c3)cc(C(F)(F)F)c2)cn1 ; Cc1cn(-c2cc(NC(=O)c3cncc(C#Cc4cnc5ccc(N6C[C@@H](C)O[C@@H](C)C6)nn45)c3)cc(C(F)(F)F)c2)cn1]

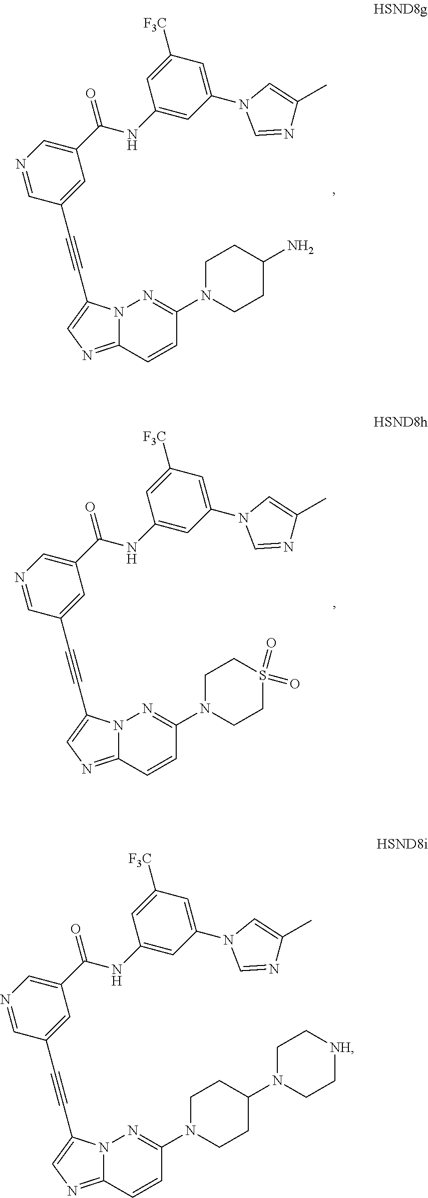 [SMILES: Cc1cn(-c2cc(NC(=O)c3cncc(C#Cc4cnc5ccc(N6CCC(N)CC6)nn45)c3)cc(C(F)(F)F)c2)cn1 ; Cc1cn(-c2cc(NC(=O)c3cncc(C#Cc4cnc5ccc(N6CCS(=O)(=O)CC6)nn45)c3)cc(C(F)(F)F)c2)cn1 ; Cc1cn(-c2cc(NC(=O)c3cncc(C#Cc4cnc5ccc(N6CCC(N7CCNCC7)CC6)nn45)c3)cc(C(F)(F)F)c2)cn1]

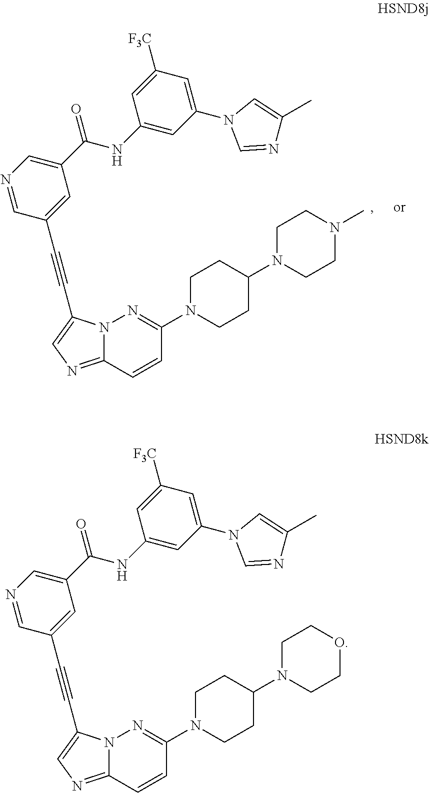 [SMILES: Cc1cn(-c2cc(NC(=O)c3cncc(C#Cc4cnc5ccc(N6CCC(N7CCN(C)CC7)CC6)nn45)c3)cc(C(F)(F)F)c2)cn1 ; Cc1cn(-c2cc(NC(=O)c3cncc(C#Cc4cnc5ccc(N6CCC(N7CCOCC7)CC6)nn45)c3)cc(C(F)(F)F)c2)cn1]

27. The compound of any one of claims 1, 2, or 9 having a structure:

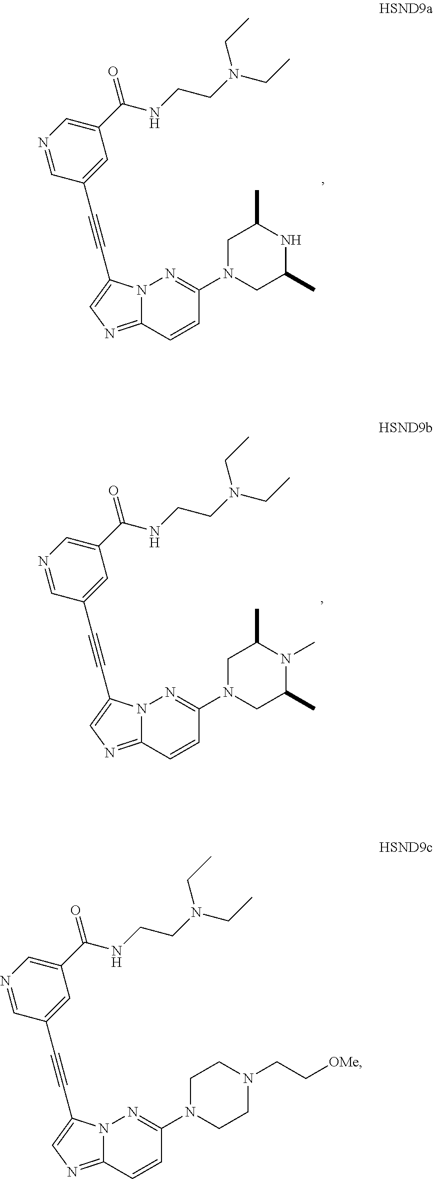 [SMILES: CCN(CC)CCNC(=O)c1cncc(C#Cc2cnc3ccc(N4C[C@@H](C)N[C@@H](C)C4)nn23)c1 ; CCN(CC)CCNC(=O)c1cncc(C#Cc2cnc3ccc(N4C[C@@H](C)N(C)[C@@H](C)C4)nn23)c1 ; CCN(CC)CCNC(=O)c1cncc(C#Cc2cnc3ccc(N4CCN(CCOC)CC4)nn23)c1]

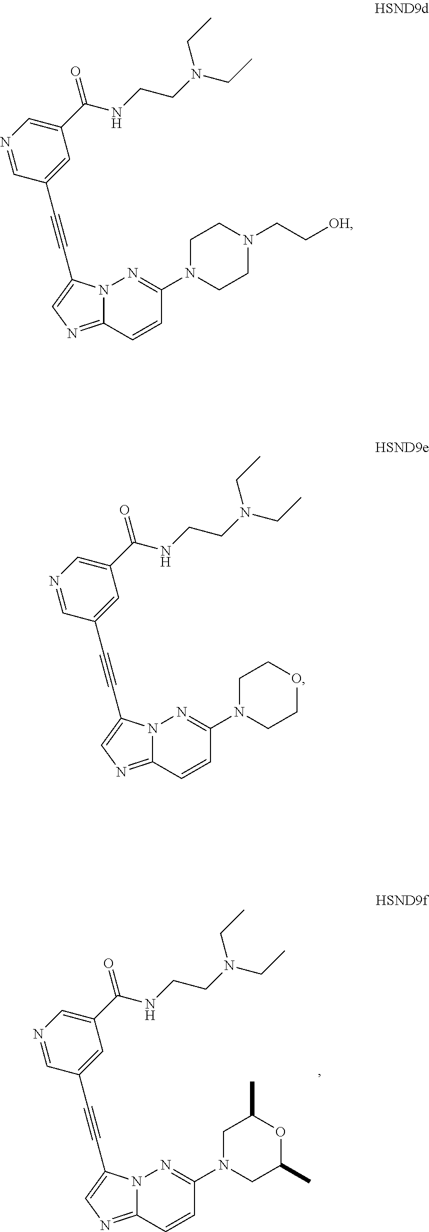 [SMILES: CCN(CC)CCNC(=O)c1cncc(C#Cc2cnc3ccc(N4CCN(CCO)CC4)nn23)c1 ; CCN(CC)CCNC(=O)c1cncc(C#Cc2cnc3ccc(N4CCOCC4)nn23)c1 ; CCN(CC)CCNC(=O)c1cncc(C#Cc2cnc3ccc(N4C[C@@H](C)O[C@@H](C)C4)nn23)c1]

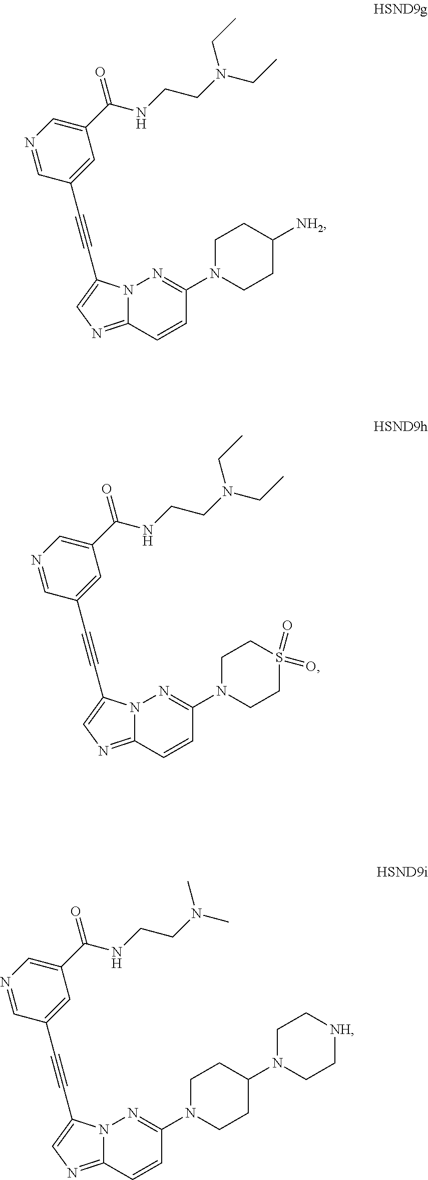 [SMILES: CCN(CC)CCNC(=O)c1cncc(C#Cc2cnc3ccc(N4CCC(N)CC4)nn23)c1 ; CCN(CC)CCNC(=O)c1cncc(C#Cc2cnc3ccc(N4CCS(=O)(=O)CC4)nn23)c1 ; CN(C)CCNC(=O)c1cncc(C#Cc2cnc3ccc(N4CCC(N5CCNCC5)CC4)nn23)c1]

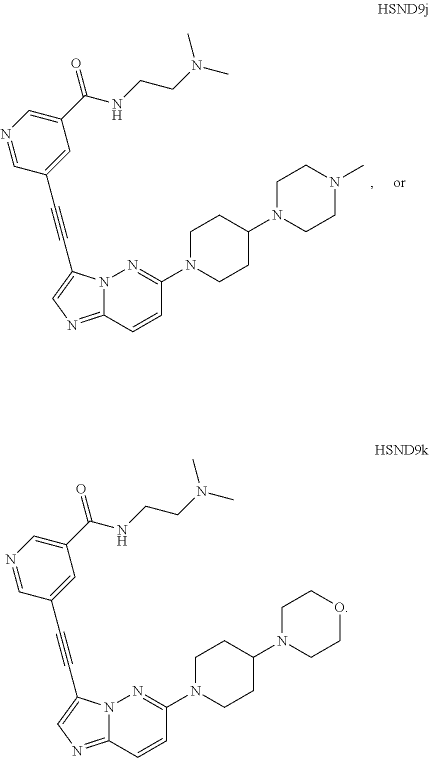 [SMILES: CN(C)CCNC(=O)c1cncc(C#Cc2cnc3ccc(N4CCC(N5CCN(C)CC5)CC4)nn23)c1 ; CN(C)CCNC(=O)c1cncc(C#Cc2cnc3ccc(N4CCC(N5CCOCC5)CC4)nn23)c1]

28. The compound of any one of claims 1, 2, or 9 having a structure:

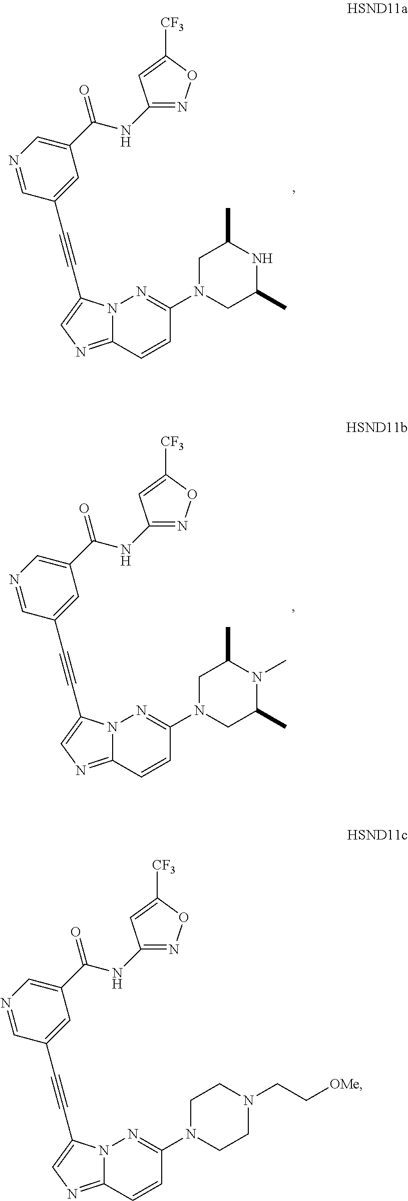 [SMILES: C[C@@H]1CN(c2ccc3ncc(C#Cc4cncc(C(=O)Nc5cc(C(F)(F)F)on5)c4)n3n2)C[C@H](C)N1 ; C[C@@H]1CN(c2ccc3ncc(C#Cc4cncc(C(=O)Nc5cc(C(F)(F)F)on5)c4)n3n2)C[C@H](C)N1C ; COCCN1CCN(c2ccc3ncc(C#Cc4cncc(C(=O)Nc5cc(C(F)(F)F)on5)c4)n3n2)CC1]

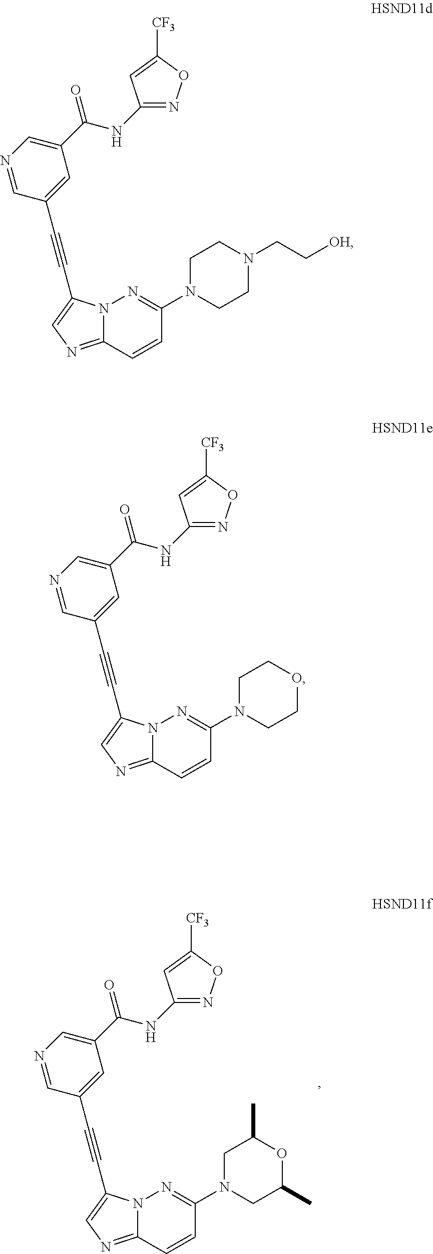 [SMILES: O=C(Nc1cc(C(F)(F)F)on1)c1cncc(C#Cc2cnc3ccc(N4CCN(CCO)CC4)nn23)c1 ; O=C(Nc1cc(C(F)(F)F)on1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)on5)c4)n3n2)C[C@H](C)O1]

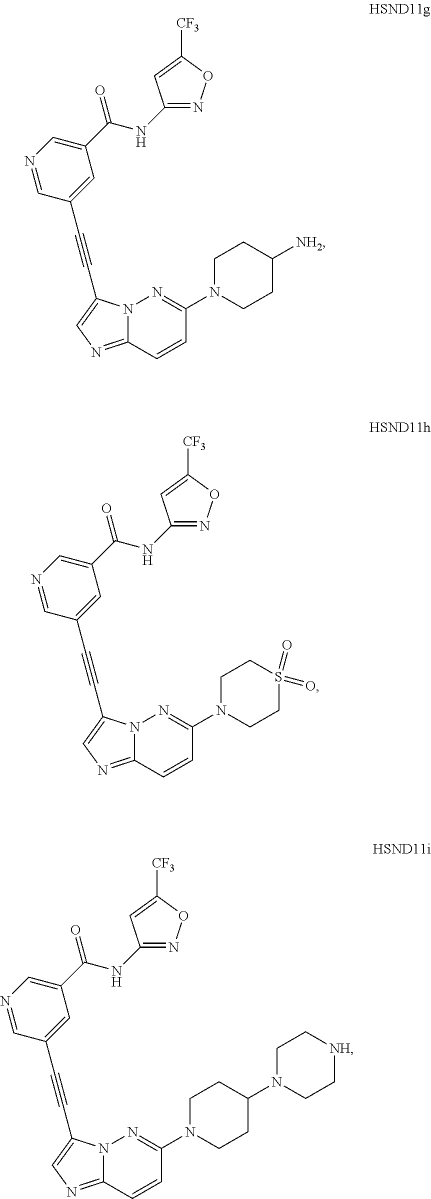 [SMILES: NC1CCN(c2ccc3ncc(C#Cc4cncc(C(=O)Nc5cc(C(F)(F)F)on5)c4)n3n2)CC1 ; O=C(Nc1cc(C(F)(F)F)on1)c1cncc(C#Cc2cnc3ccc(N4CCS(=O)(=O)CC4)nn23)c1 ; O=C(Nc1cc(C(F)(F)F)on1)c1cncc(C#Cc2cnc3ccc(N4CCC(N5CCNCC5)CC4)nn23)c1]

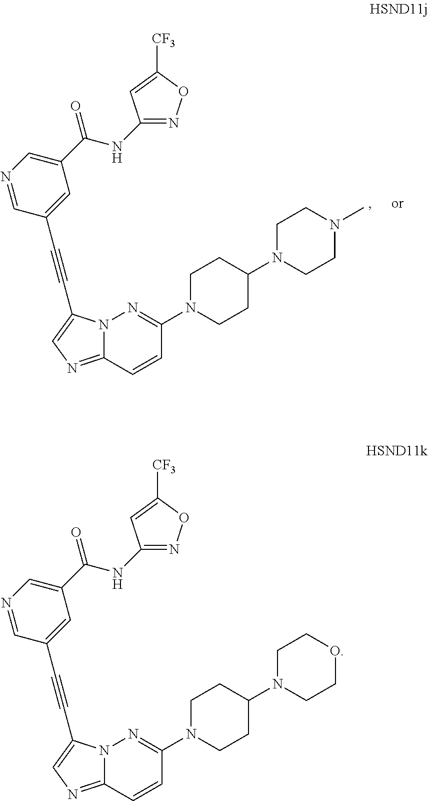 [SMILES: CN1CCN(C2CCN(c3ccc4ncc(C#Cc5cncc(C(=O)Nc6cc(C(F)(F)F)on6)c5)n4n3)CC2)CC1 ; O=C(Nc1cc(C(F)(F)F)on1)c1cncc(C#Cc2cnc3ccc(N4CCC(N5CCOCC5)CC4)nn23)c1]

29. The compound of any one of claims 1, 2, or 9 having a structure:

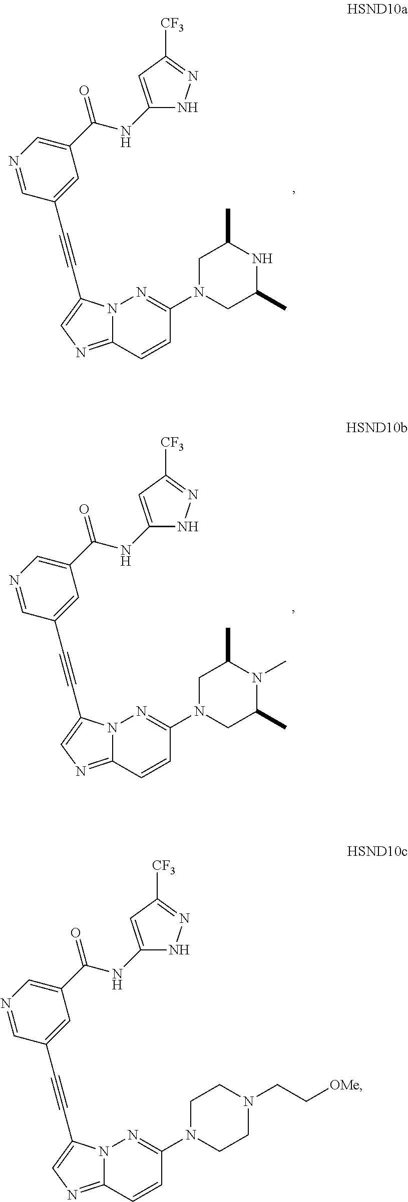 [SMILES: C[C@@H]1CN(c2ccc3ncc(C#Cc4cncc(C(=O)Nc5cc(C(F)(F)F)n[nH]5)c4)n3n2)C[C@H](C)N1 ; C[C@@H]1CN(c2ccc3ncc(C#Cc4cncc(C(=O)Nc5cc(C(F)(F)F)n[nH]5)c4)n3n2)C[C@H](C)N1C ; COCCN1CCN(c2ccc3ncc(C#Cc4cncc(C(=O)Nc5cc(C(F)(F)F)n[nH]5)c4)n3n2)CC1]

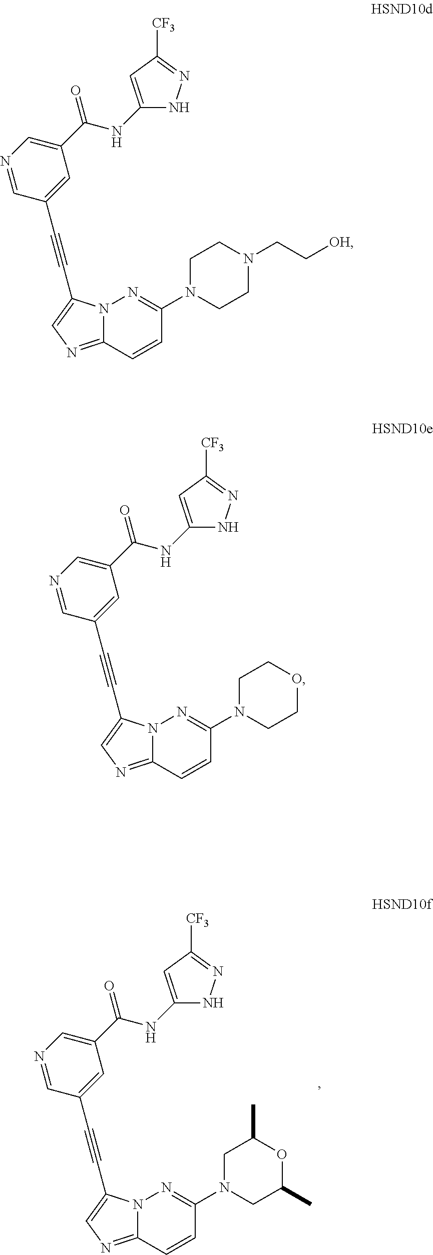 [SMILES: O=C(Nc1cc(C(F)(F)F)n[nH]1)c1cncc(C#Cc2cnc3ccc(N4CCN(CCO)CC4)nn23)c1 ; O=C(Nc1cc(C(F)(F)F)n[nH]1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)n[nH]5)c4)n3n2)C[C@H](C)O1]

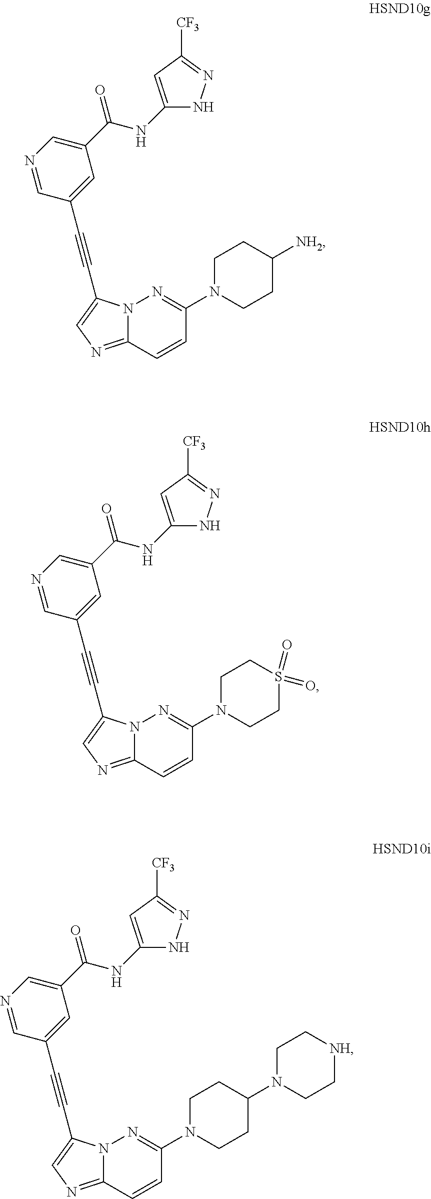 [SMILES: NC1CCN(c2ccc3ncc(C#Cc4cncc(C(=O)Nc5cc(C(F)(F)F)n[nH]5)c4)n3n2)CC1 ; O=C(Nc1cc(C(F)(F)F)n[nH]1)c1cncc(C#Cc2cnc3ccc(N4CCS(=O)(=O)CC4)nn23)c1 ; O=C(Nc1cc(C(F)(F)F)n[nH]1)c1cncc(C#Cc2cnc3ccc(N4CCC(N5CCNCC5)CC4)nn23)c1]

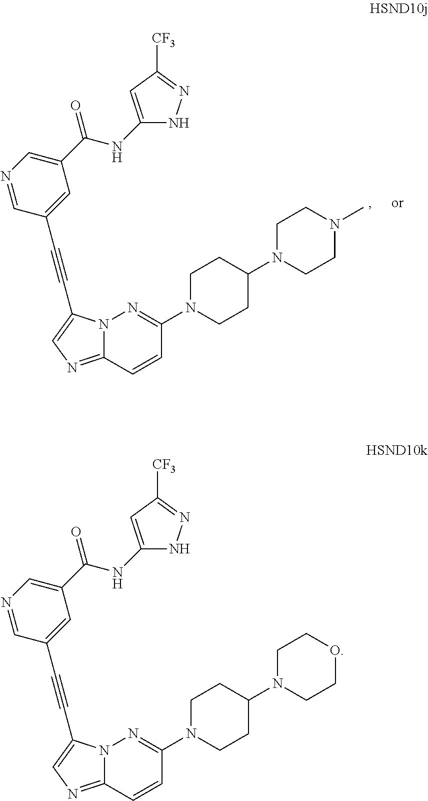 [SMILES: CN1CCN(C2CCN(c3ccc4ncc(C#Cc5cncc(C(=O)Nc6cc(C(F)(F)F)n[nH]6)c5)n4n3)CC2)CC1 ; O=C(Nc1cc(C(F)(F)F)n[nH]1)c1cncc(C#Cc2cnc3ccc(N4CCC(N5CCOCC5)CC4)nn23)c1]

30. The compound of any one of claims 1, 2, or 9 having a structure:

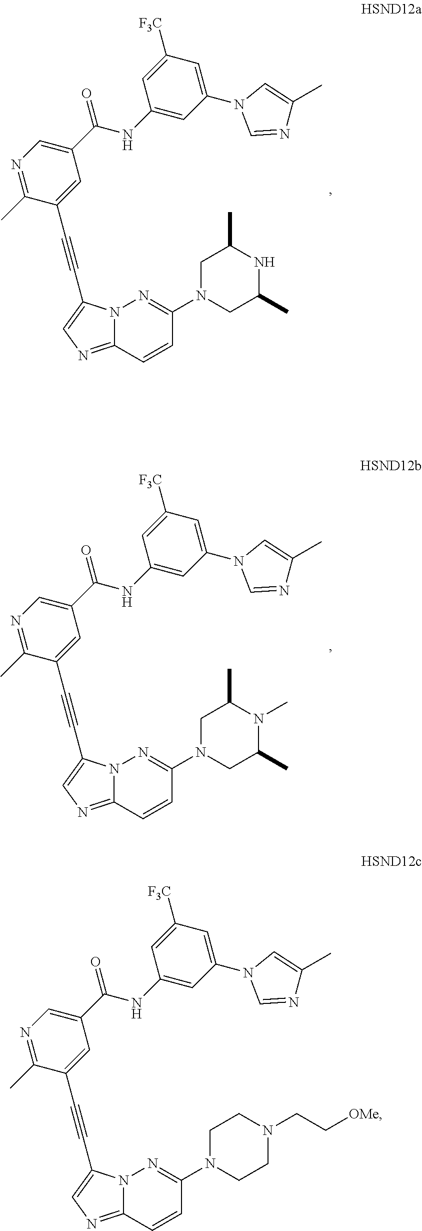

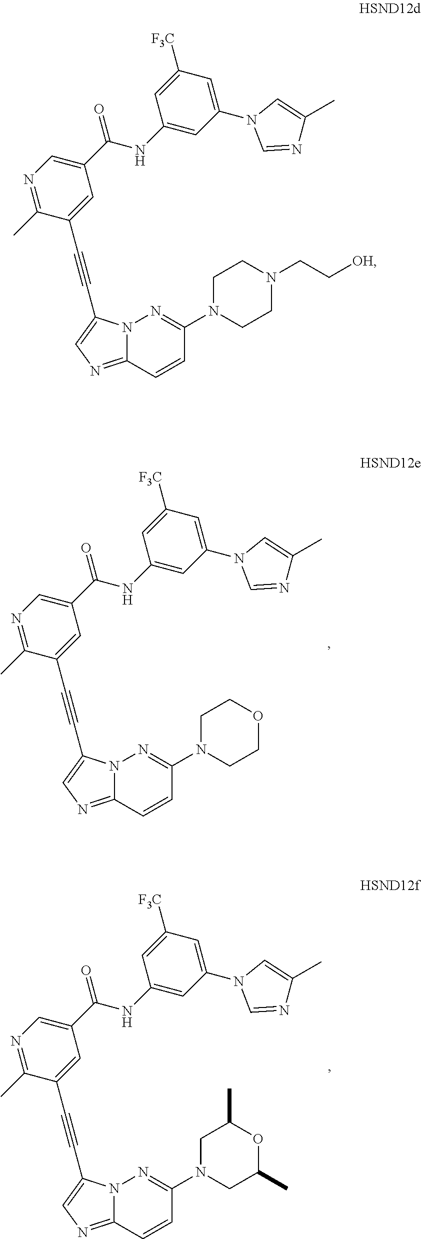

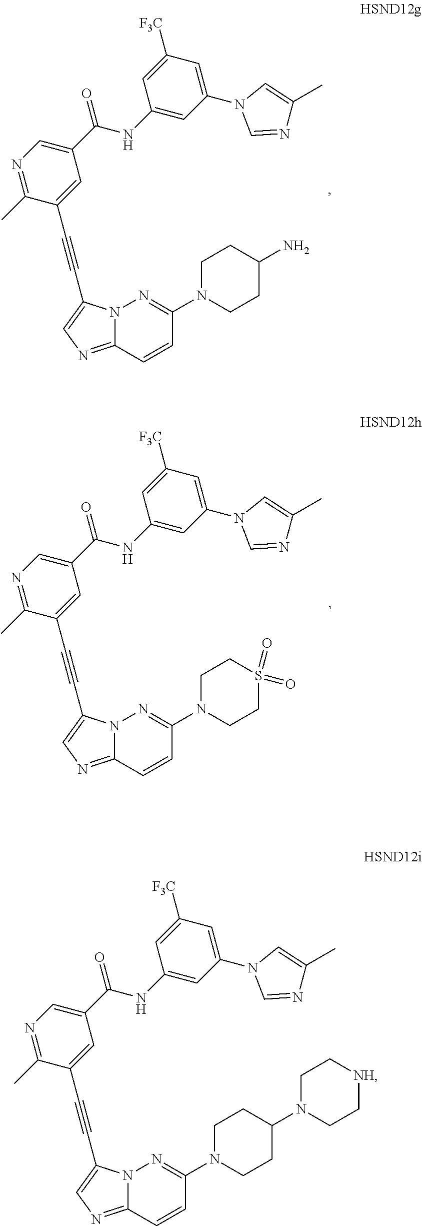

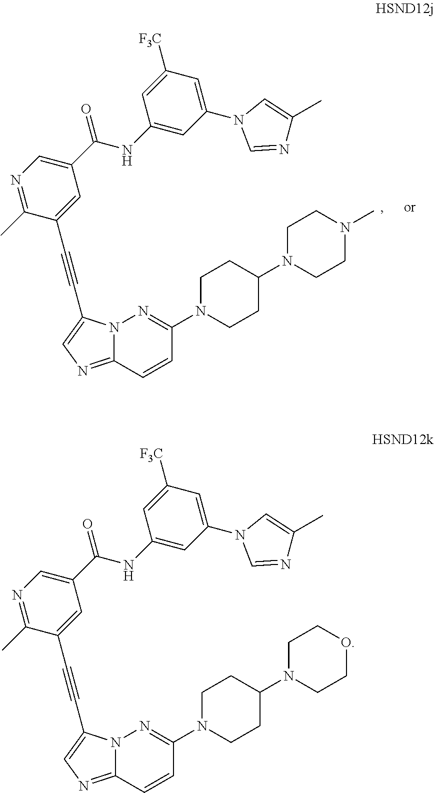

31. The compound of any one of claims 1, 2, or 9 having a structure:

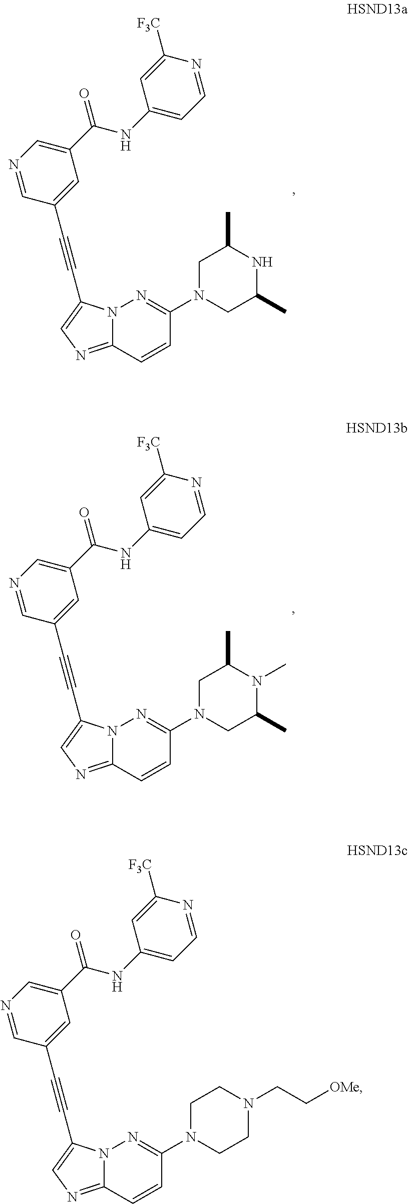 [SMILES: C[C@@H]1CN(c2ccc3ncc(C#Cc4cncc(C(=O)Nc5ccnc(C(F)(F)F)c5)c4)n3n2)C[C@H](C)N1 ; C[C@@H]1CN(c2ccc3ncc(C#Cc4cncc(C(=O)Nc5ccnc(C(F)(F)F)c5)c4)n3n2)C[C@H](C)N1C ; COCCN1CCN(c2ccc3ncc(C#Cc4cncc(C(=O)Nc5ccnc(C(F)(F)F)c5)c4)n3n2)CC1]

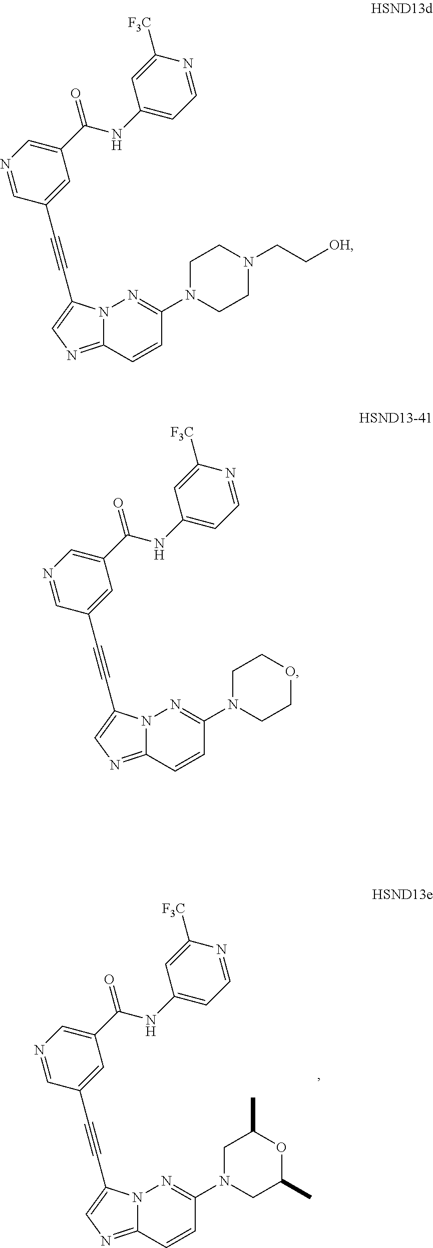 [SMILES: O=C(Nc1ccnc(C(F)(F)F)c1)c1cncc(C#Cc2cnc3ccc(N4CCN(CCO)CC4)nn23)c1 ; O=C(Nc1ccnc(C(F)(F)F)c1)c1cncc(C#Cc2cnc3ccc(N4CCOCC4)nn23)c1 ; C[C@@H]1CN(c2ccc3ncc(C#Cc4cncc(C(=O)Nc5ccnc(C(F)(F)F)c5)c4)n3n2)C[C@H](C)O1]

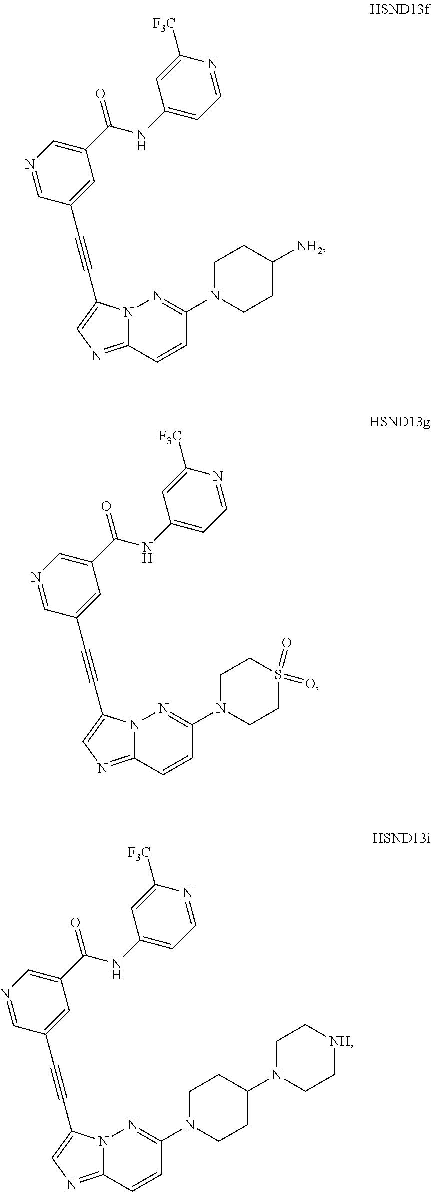 [SMILES: NC1CCN(c2ccc3ncc(C#Cc4cncc(C(=O)Nc5ccnc(C(F)(F)F)c5)c4)n3n2)CC1 ; O=C(Nc1ccnc(C(F)(F)F)c1)c1cncc(C#Cc2cnc3ccc(N4CCS(=O)(=O)CC4)nn23)c1 ; O=C(Nc1ccnc(C(F)(F)F)c1)c1cncc(C#Cc2cnc3ccc(N4CCC(N5CCNCC5)CC4)nn23)c1]

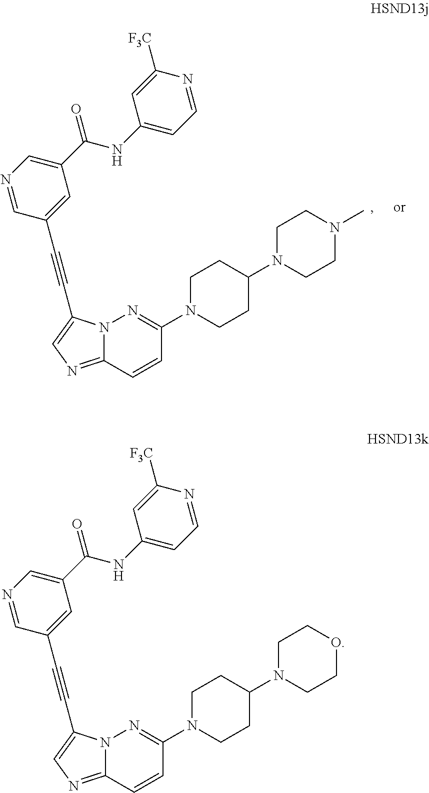 [SMILES: CN1CCN(C2CCN(c3ccc4ncc(C#Cc5cncc(C(=O)Nc6ccnc(C(F)(F)F)c6)c5)n4n3)CC2)CC1 ; O=C(Nc1ccnc(C(F)(F)F)c1)c1cncc(C#Cc2cnc3ccc(N4CCC(N5CCOCC5)CC4)nn23)c1]

32. The compound of any one of claims 1, 2, or 9 having a structure:

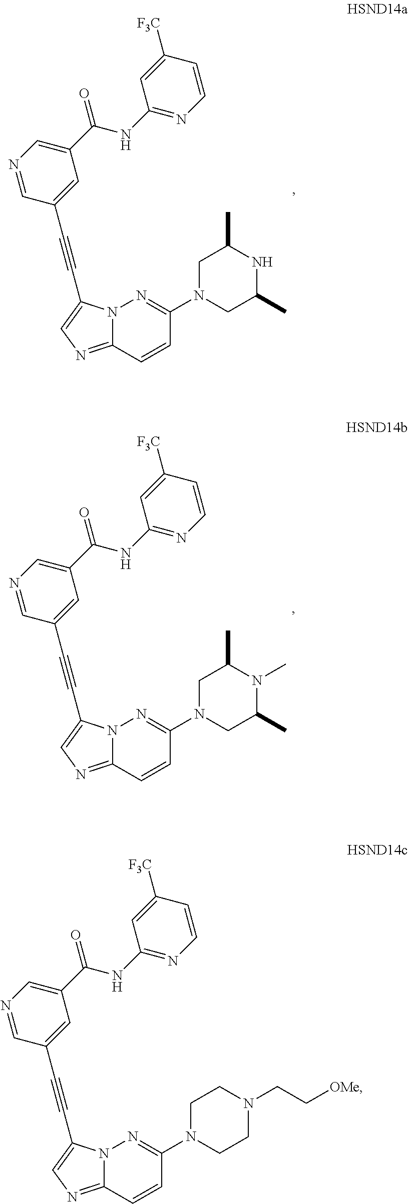 [SMILES: C[C@@H]1CN(c2ccc3ncc(C#Cc4cncc(C(=O)Nc5cc(C(F)(F)F)ccn5)c4)n3n2)C[C@H](C)N1 ; C[C@@H]1CN(c2ccc3ncc(C#Cc4cncc(C(=O)Nc5cc(C(F)(F)F)ccn5)c4)n3n2)C[C@H](C)N1C ; COCCN1CCN(c2ccc3ncc(C#Cc4cncc(C(=O)Nc5cc(C(F)(F)F)ccn5)c4)n3n2)CC1]

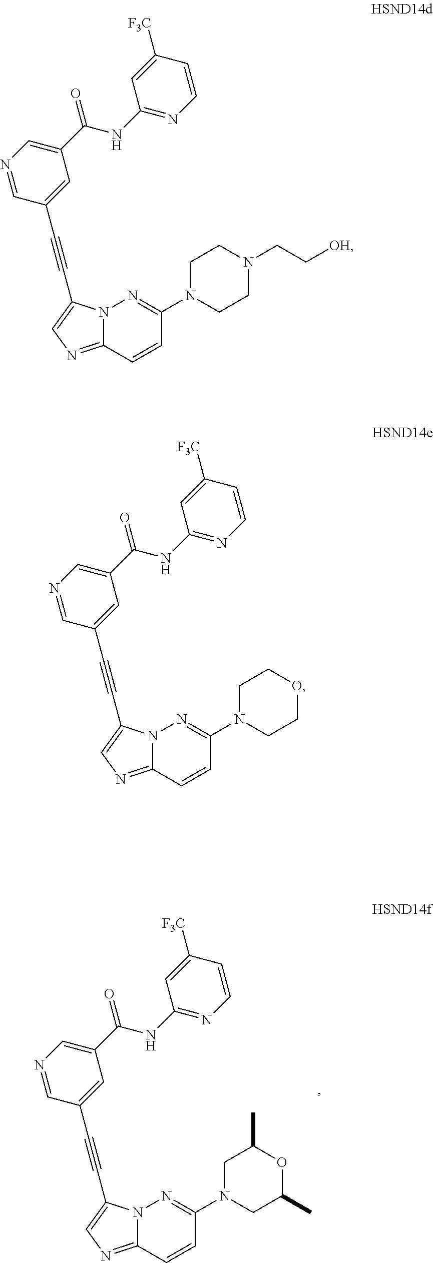 [SMILES: O=C(Nc1cc(C(F)(F)F)ccn1)c1cncc(C#Cc2cnc3ccc(N4CCN(CCO)CC4)nn23)c1 ; O=C(Nc1cc(C(F)(F)F)ccn1)c1cncc(C#Cc2cnc3ccc(N4CCOCC4)nn23)c1 ; C[C@@H]1CN(c2ccc3ncc(C#Cc4cncc(C(=O)Nc5cc(C(F)(F)F)ccn5)c4)n3n2)C[C@H](C)O1]

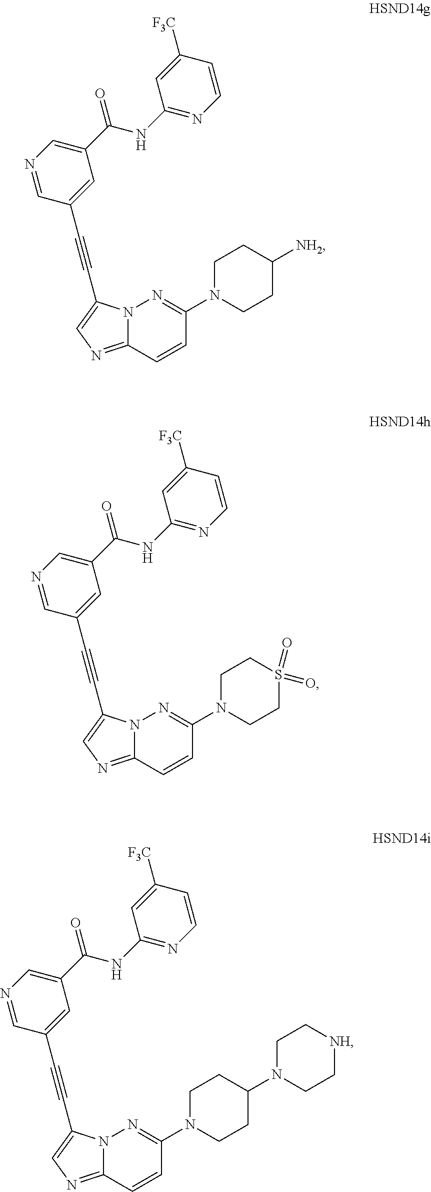 [SMILES: NC1CCN(c2ccc3ncc(C#Cc4cncc(C(=O)Nc5cc(C(F)(F)F)ccn5)c4)n3n2)CC1 ; O=C(Nc1cc(C(F)(F)F)ccn1)c1cncc(C#Cc2cnc3ccc(N4CCS(=O)(=O)CC4)nn23)c1 ; O=C(Nc1cc(C(F)(F)F)ccn1)c1cncc(C#Cc2cnc3ccc(N4CCC(N5CCNCC5)CC4)nn23)c1]

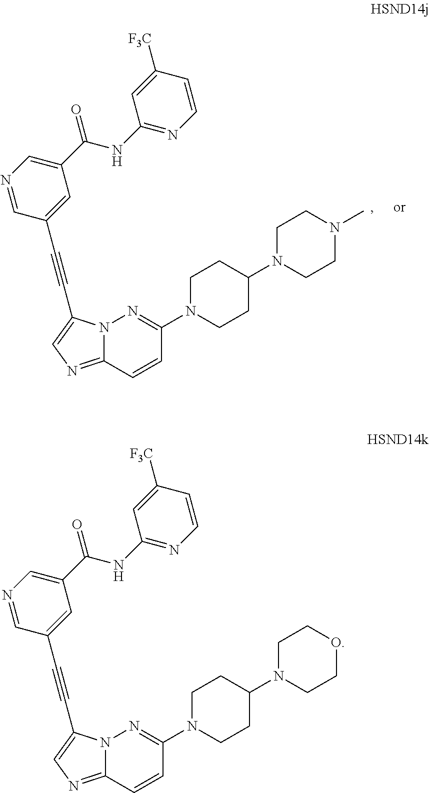 [SMILES: CN1CCN(C2CCN(c3ccc4ncc(C#Cc5cncc(C(=O)Nc6cc(C(F)(F)F)ccn6)c5)n4n3)CC2)CC1 ; O=C(Nc1cc(C(F)(F)F)ccn1)c1cncc(C#Cc2cnc3ccc(N4CCC(N5CCOCC5)CC4)nn23)c1]

33. The compound of any one of claims 1, 2, or 9 having a structure:

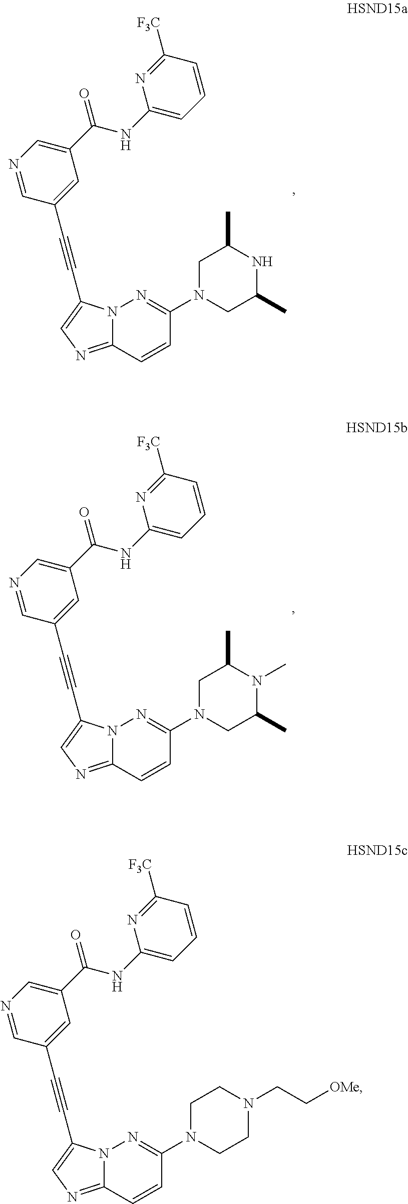 [SMILES: C[C@@H]1CN(c2ccc3ncc(C#Cc4cncc(C(=O)Nc5cccc(C(F)(F)F)n5)c4)n3n2)C[C@H](C)N1 ; C[C@@H]1CN(c2ccc3ncc(C#Cc4cncc(C(=O)Nc5cccc(C(F)(F)F)n5)c4)n3n2)C[C@H](C)N1C ; COCCN1CCN(c2ccc3ncc(C#Cc4cncc(C(=O)Nc5cccc(C(F)(F)F)n5)c4)n3n2)CC1]

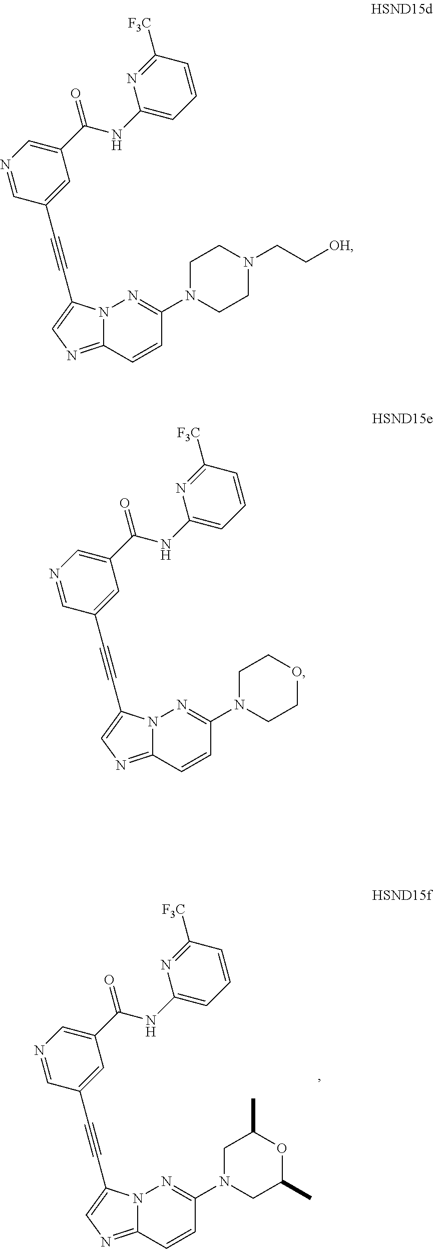 [SMILES: O=C(Nc1cccc(C(F)(F)F)n1)c1cncc(C#Cc2cnc3ccc(N4CCN(CCO)CC4)nn23)c1 ; O=C(Nc1cccc(C(F)(F)F)n1)c1cncc(C#Cc2cnc3ccc(N4CCOCC4)nn23)c1 ; C[C@@H]1CN(c2ccc3ncc(C#Cc4cncc(C(=O)Nc5cccc(C(F)(F)F)n5)c4)n3n2)C[C@H](C)O1]

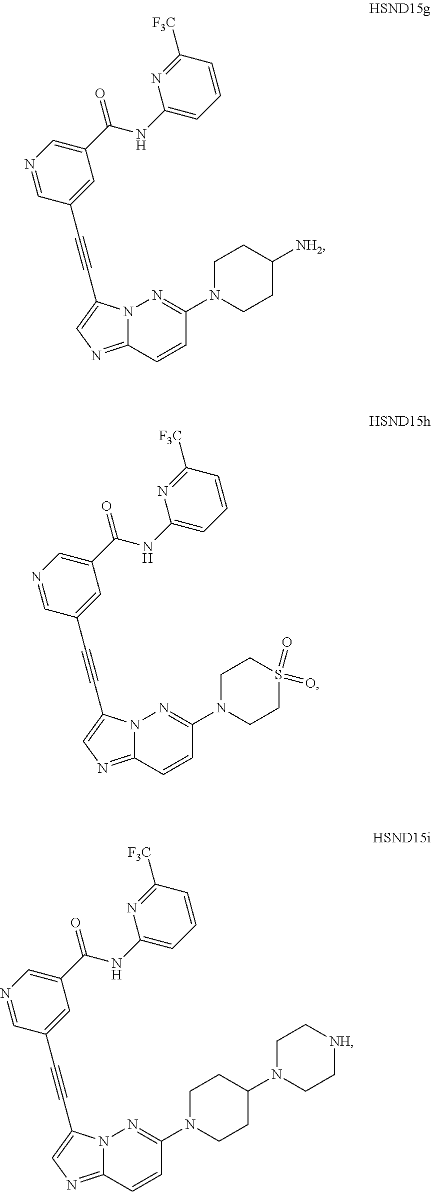 [SMILES: NC1CCN(c2ccc3ncc(C#Cc4cncc(C(=O)Nc5cccc(C(F)(F)F)n5)c4)n3n2)CC1 ; O=C(Nc1cccc(C(F)(F)F)n1)c1cncc(C#Cc2cnc3ccc(N4CCS(=O)(=O)CC4)nn23)c1 ; O=C(Nc1cccc(C(F)(F)F)n1)c1cncc(C#Cc2cnc3ccc(N4CCC(N5CCNCC5)CC4)nn23)c1]

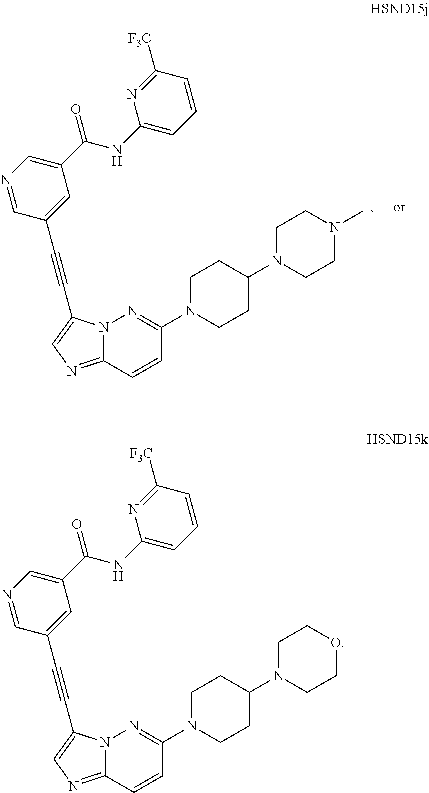 [SMILES: CN1CCN(C2CCN(c3ccc4ncc(C#Cc5cncc(C(=O)Nc6cccc(C(F)(F)F)n6)c5)n4n3)CC2)CC1 ; O=C(Nc1cccc(C(F)(F)F)n1)c1cncc(C#Cc2cnc3ccc(N4CCC(N5CCOCC5)CC4)nn23)c1]

34. The compound of any one of claims 1, 2, or 9 having a structure:

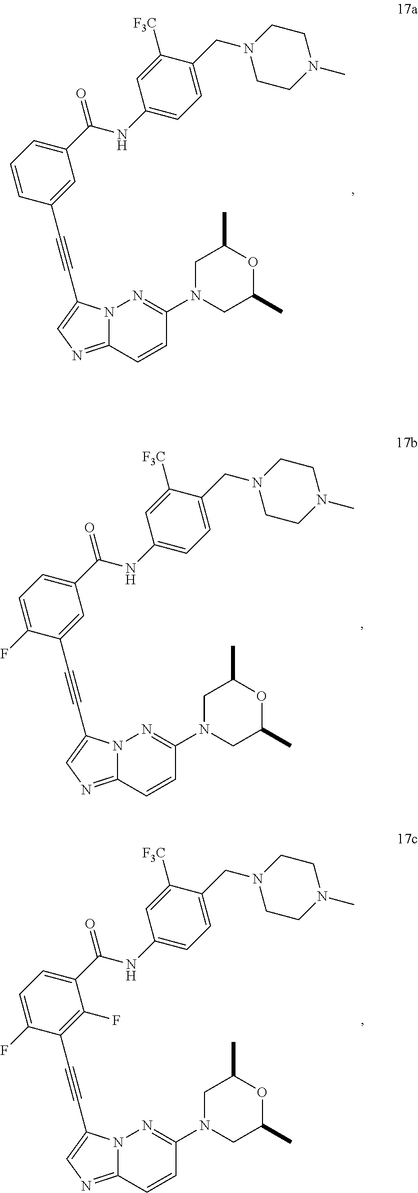 [SMILES: C[C@@H]1CN(c2ccc3ncc(C#Cc4cccc(C(=O)Nc5ccc(CN6CCN(C)CC6)c(C(F)(F)F)c5)c4)n3n2)C[C@H](C)O1 ; C[C@@H]1CN(c2ccc3ncc(C#Cc4cc(C(=O)Nc5ccc(CN6CCN(C)CC6)c(C(F)(F)F)c5)ccc4F)n3n2)C[C@H](C)O1 ; C[C@@H]1CN(c2ccc3ncc(C#Cc4c(F)ccc(C(=O)Nc5ccc(CN6CCN(C)CC6)c(C(F)(F)F)c5)c4F)n3n2)C[C@H](C)O1]

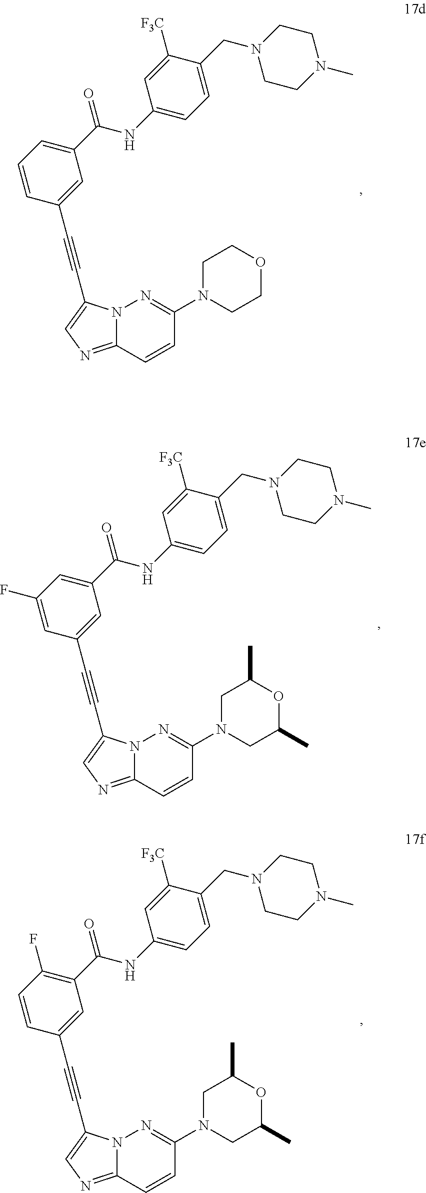 [SMILES: CN1CCN(Cc2ccc(NC(=O)c3cccc(C#Cc4cnc5ccc(N6CCOCC6)nn45)c3)cc2C(F)(F)F)CC1 ; C[C@@H]1CN(c2ccc3ncc(C#Cc4cc(F)cc(C(=O)Nc5ccc(CN6CCN(C)CC6)c(C(F)(F)F)c5)c4)n3n2)C[C@H](C)O1 ; C[C@@H]1CN(c2ccc3ncc(C#Cc4ccc(F)c(C(=O)Nc5ccc(CN6CCN(C)CC6)c(C(F)(F)F)c5)c4)n3n2)C[C@H](C)O1]

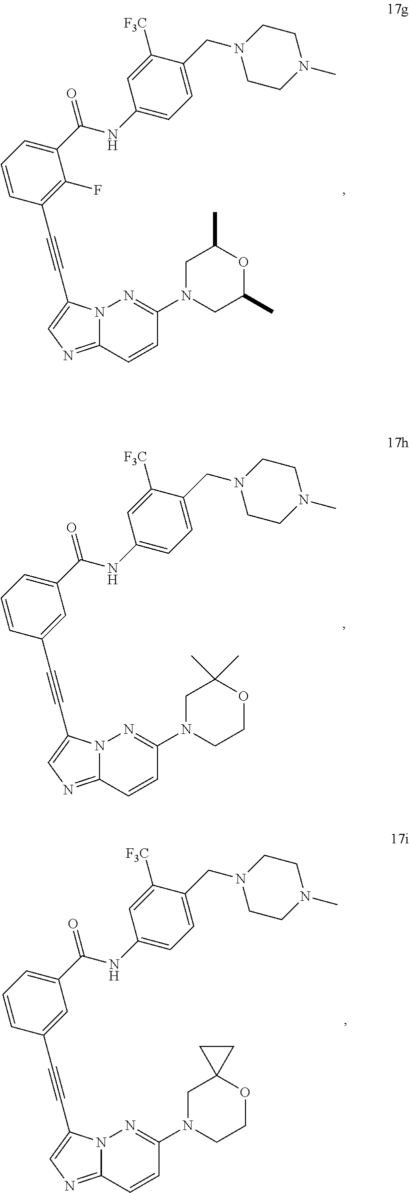 [SMILES: C[C@@H]1CN(c2ccc3ncc(C#Cc4cccc(C(=O)Nc5ccc(CN6CCN(C)CC6)c(C(F)(F)F)c5)c4F)n3n2)C[C@H](C)O1 ; CN1CCN(Cc2ccc(NC(=O)c3cccc(C#Cc4cnc5ccc(N6CCOC(C)(C)C6)nn45)c3)cc2C(F)(F)F)CC1 ; CN1CCN(Cc2ccc(NC(=O)c3cccc(C#Cc4cnc5ccc(N6CCOC7(CC7)C6)nn45)c3)cc2C(F)(F)F)CC1]

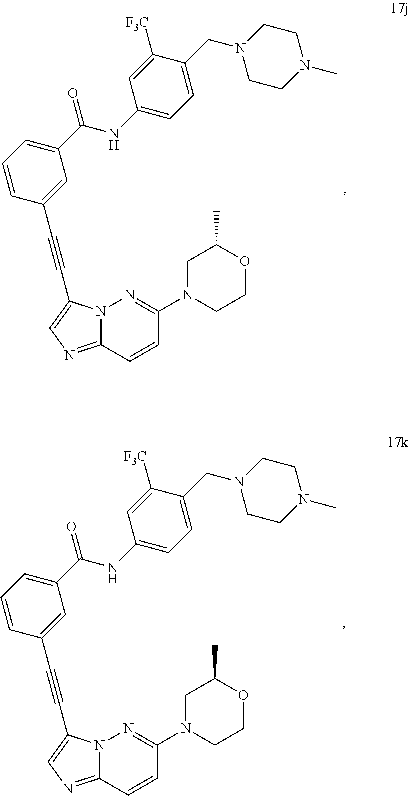 [SMILES: C[C@H]1CN(c2ccc3ncc(C#Cc4cccc(C(=O)Nc5ccc(CN6CCN(C)CC6)c(C(F)(F)F)c5)c4)n3n2)CCO1 ; C[C@@H]1CN(c2ccc3ncc(C#Cc4cccc(C(=O)Nc5ccc(CN6CCN(C)CC6)c(C(F)(F)F)c5)c4)n3n2)CCO1]

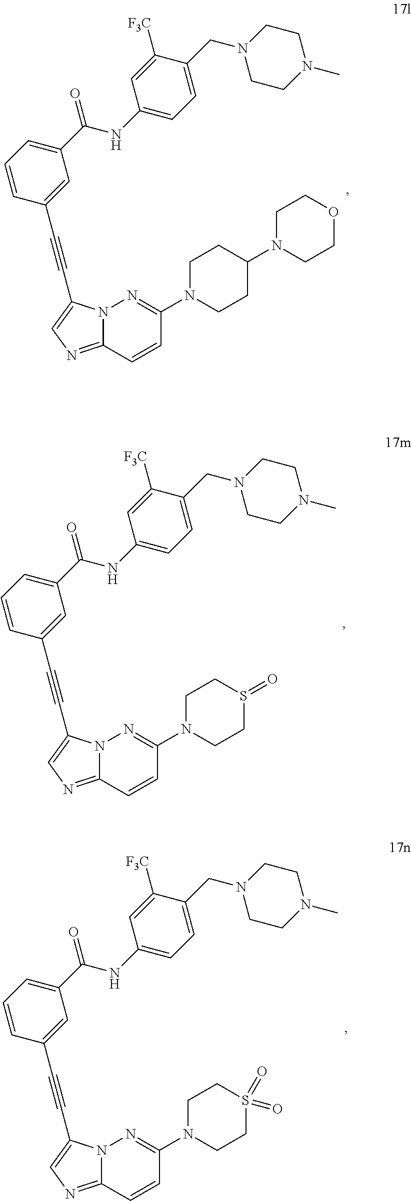 [SMILES: CN1CCN(Cc2ccc(NC(=O)c3cccc(C#Cc4cnc5ccc(N6CCC(N7CCOCC7)CC6)nn45)c3)cc2C(F)(F)F)CC1 ; CN1CCN(Cc2ccc(NC(=O)c3cccc(C#Cc4cnc5ccc(N6CCS(=O)CC6)nn45)c3)cc2C(F)(F)F)CC1 ; CN1CCN(Cc2ccc(NC(=O)c3cccc(C#Cc4cnc5ccc(N6CCS(=O)(=O)CC6)nn45)c3)cc2C(F)(F)F)CC1]

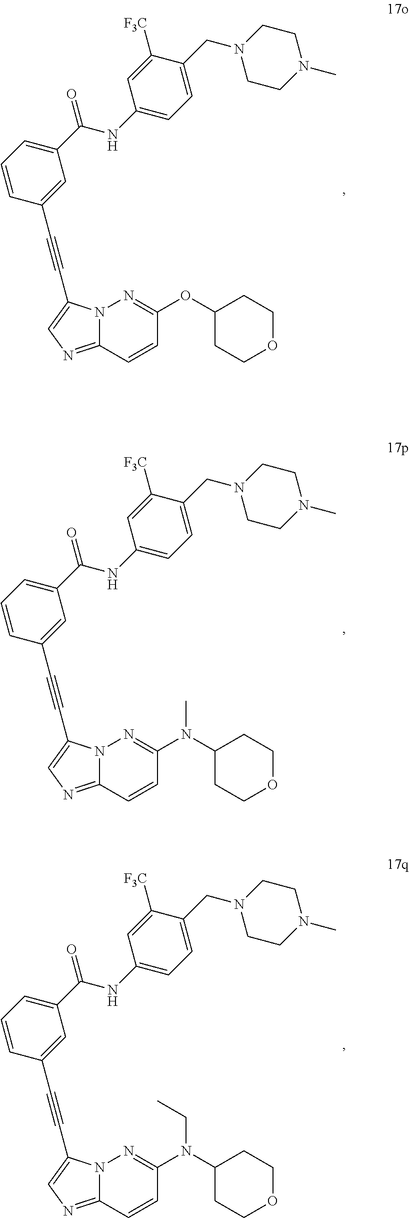 [SMILES: CN1CCN(Cc2ccc(NC(=O)c3cccc(C#Cc4cnc5ccc(OC6CCOCC6)nn45)c3)cc2C(F)(F)F)CC1 ; CN1CCN(Cc2ccc(NC(=O)c3cccc(C#Cc4cnc5ccc(N(C)C6CCOCC6)nn45)c3)cc2C(F)(F)F)CC1 ; CCN(c1ccc2ncc(C#Cc3cccc(C(=O)Nc4ccc(CN5CCN(C)CC5)c(C(F)(F)F)c4)c3)n2n1)C1CCOCC1]

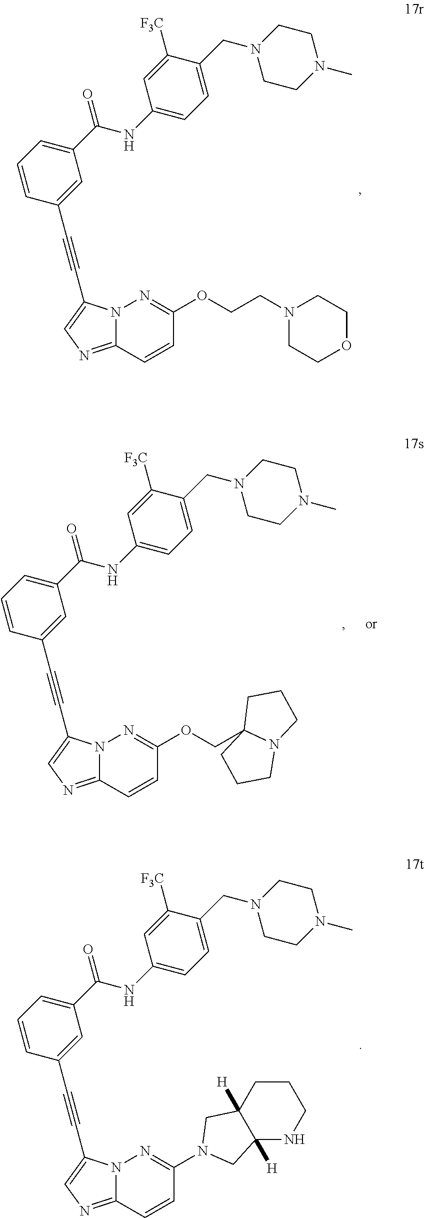 [SMILES: CN1CCN(Cc2ccc(NC(=O)c3cccc(C#Cc4cnc5ccc(OCCN6CCOCC6)nn45)c3)cc2C(F)(F)F)CC1 ; CN1CCN(Cc2ccc(NC(=O)c3cccc(C#Cc4cnc5ccc(OCC67CCCN6CCC7)nn45)c3)cc2C(F)(F)F)CC1 ; CN1CCN(Cc2ccc(NC(=O)c3cccc(C#Cc4cnc5ccc(N6C[C@@H]7CCCN[C@@H]7C6)nn45)c3)cc2C(F)(F)F)CC1]

35. The compound of any one of claims 1, 2, or 9 having a structure:

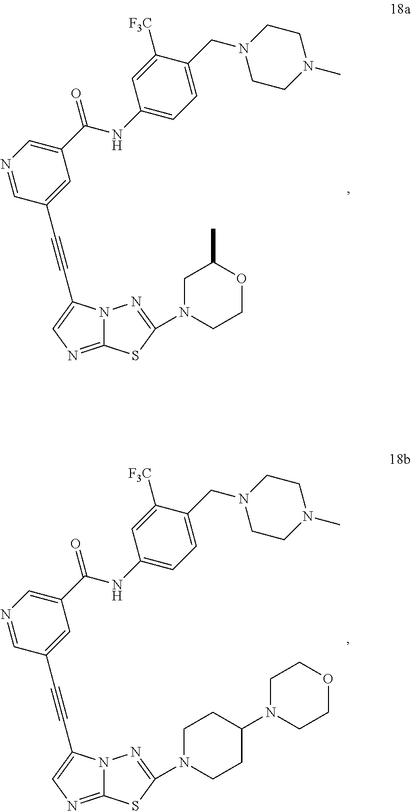

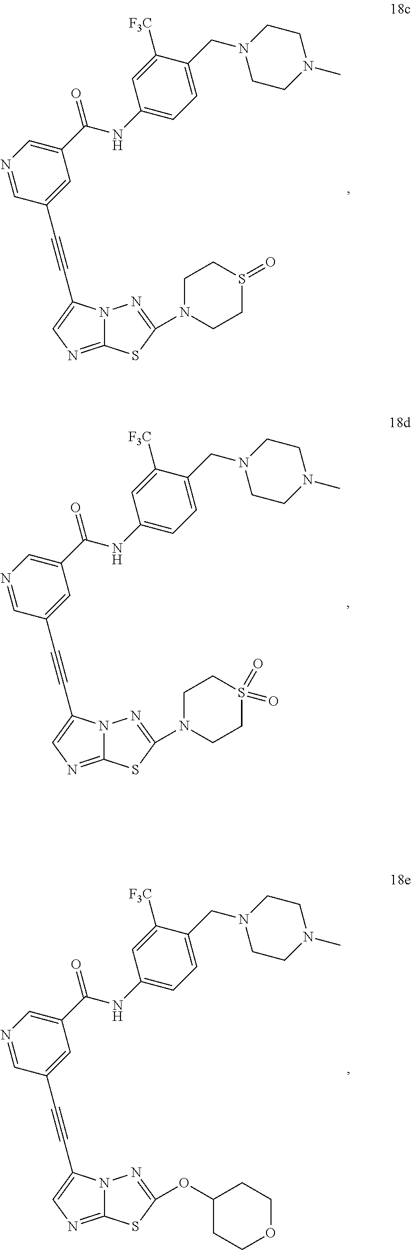

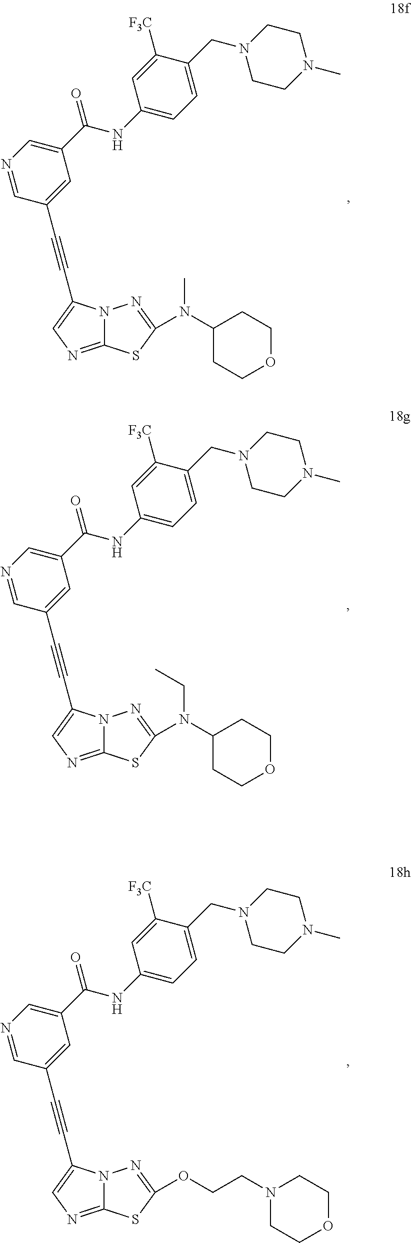

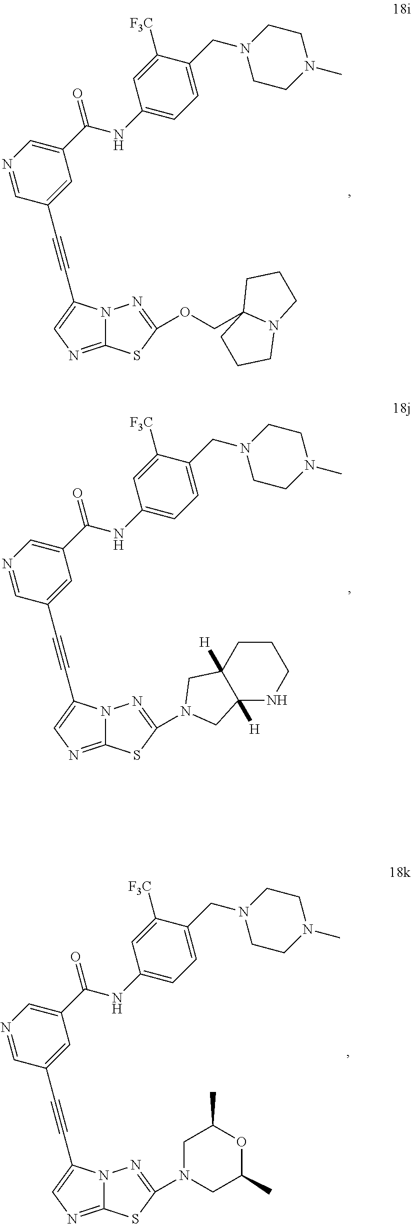

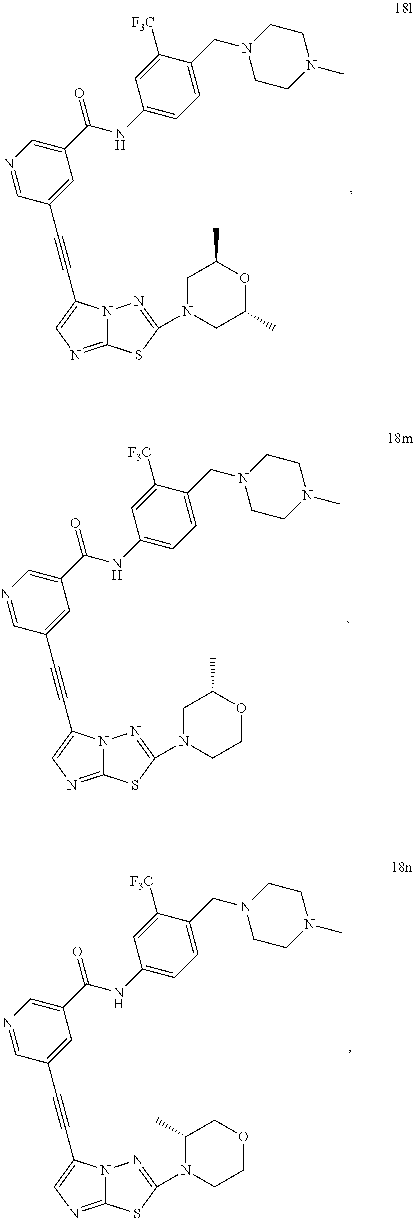

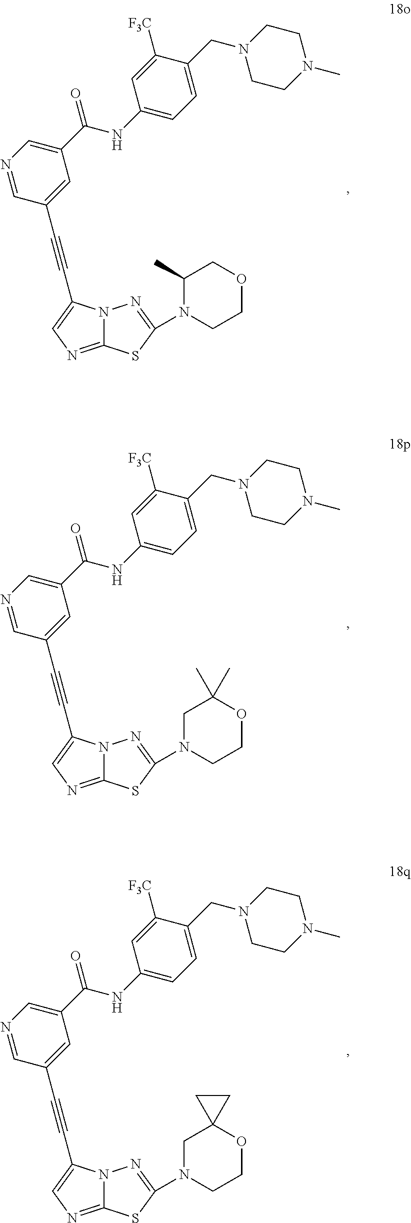

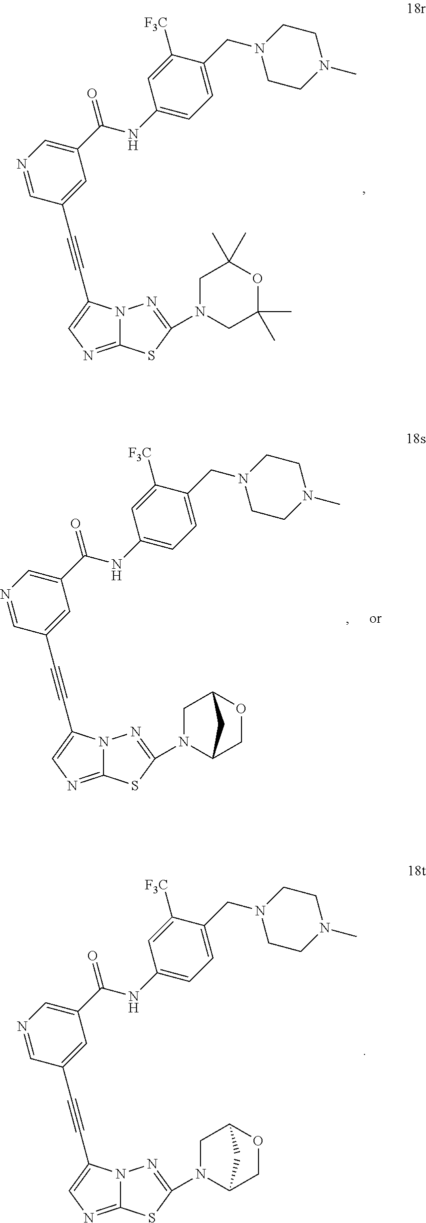

36. The compound of any one of claims 1, 2, or 9 having a structure:

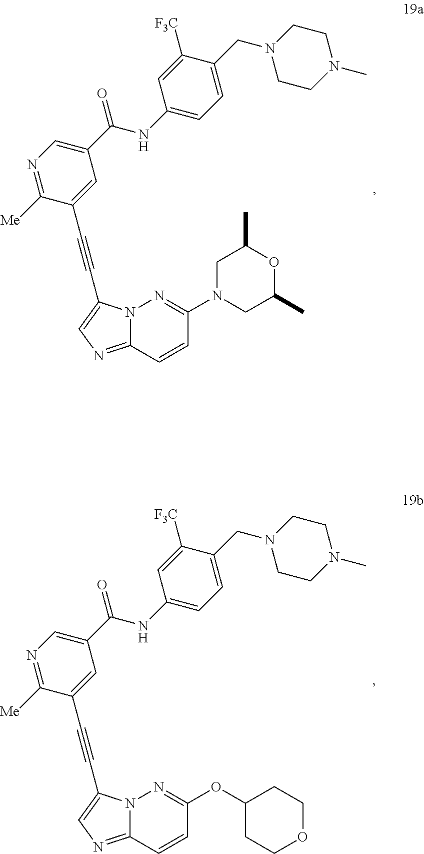

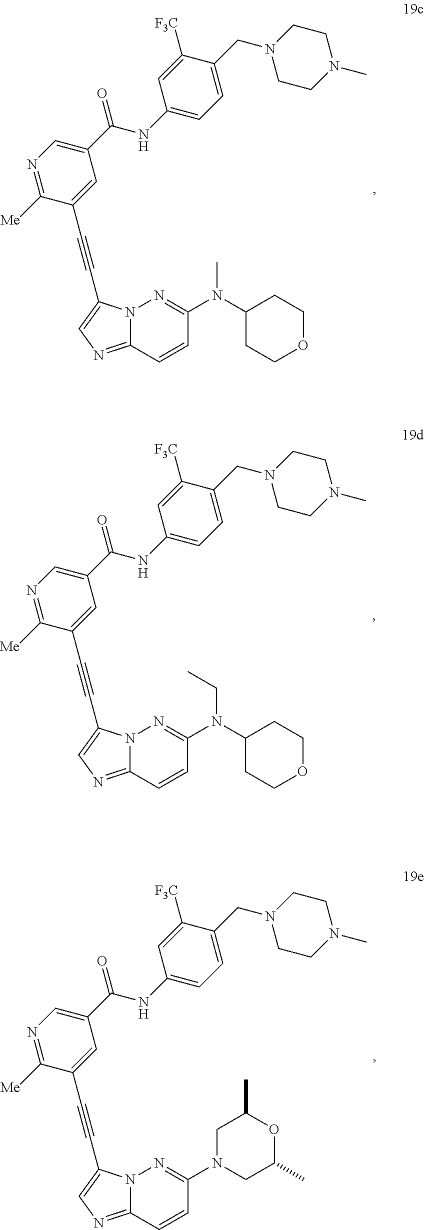

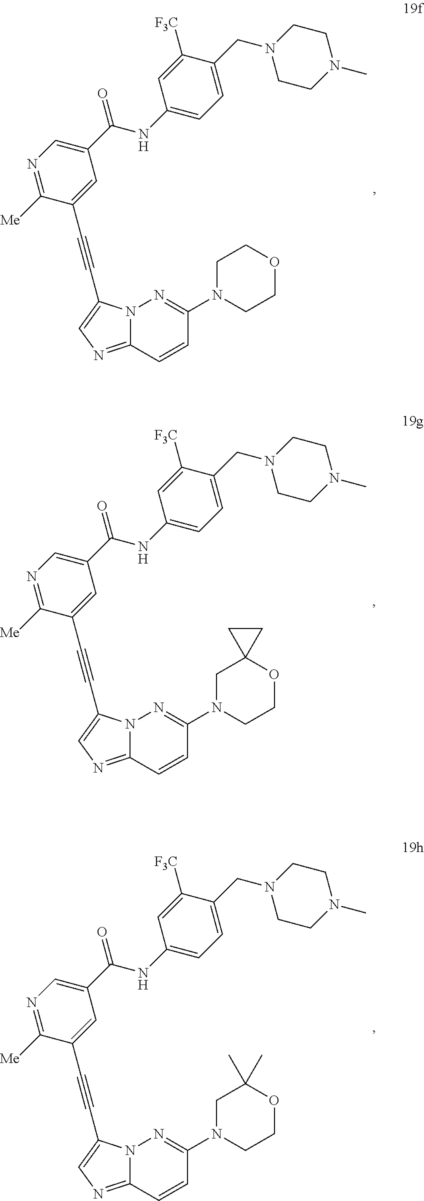

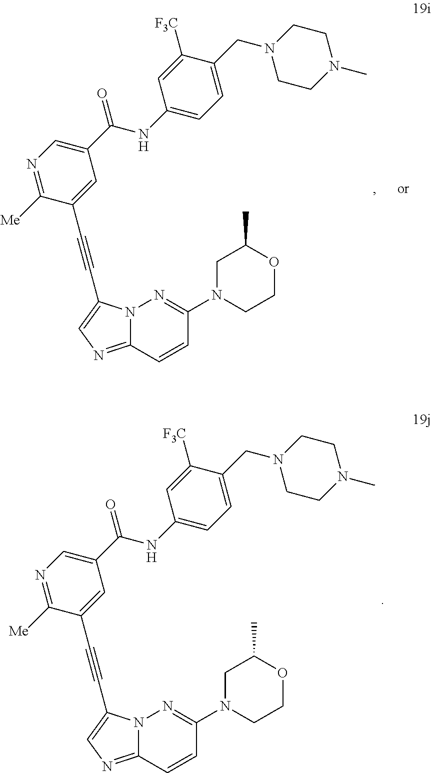

37. The compound of any one of claims 1, 2, 4, 7, 10-12, or 15, wherein R₁ is 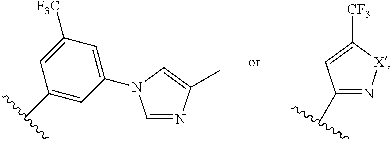
wherein 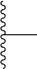
is a point of attachment and X′ is O, C, or N.

38. A compound having a structure of 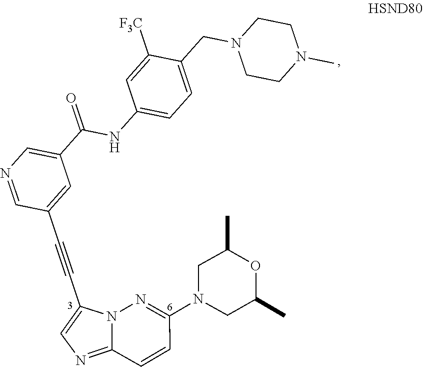 [SMILES: C[C@@H]1CN(c2ccc3ncc(C#Cc4cncc(C(=O)Nc5ccc(CN6CCN(C)CC6)c(C(F)(F)F)c5)c4)n3n2)C[C@H](C)O1]
or be a pharmaceutically acceptable salt thereof.

39. A compound having a structure of 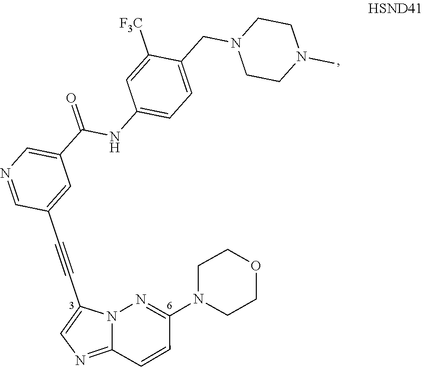 [SMILES: CN1CCN(Cc2ccc(NC(=O)c3cncc(C#Cc4cnc5ccc(N6CCOCC6)nn45)c3)cc2C(F)(F)F)CC1]
or a pharmaceutically acceptable salt thereof.

40. A compound having a structure of 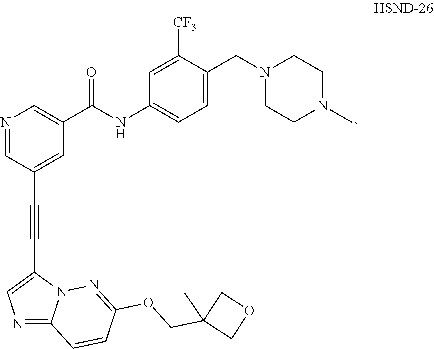 [SMILES: CN1CCN(Cc2ccc(NC(=O)c3cncc(C#Cc4cnc5ccc(OCC6(C)COC6)nn45)c3)cc2C(F)(F)F)CC1]
or a pharmaceutically acceptable salt thereof.

41. A compound having a structure of 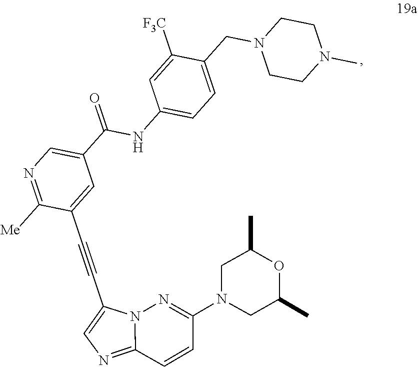 [SMILES: Cc1ncc(C(=O)Nc2ccc(CN3CCN(C)CC3)c(C(F)(F)F)c2)cc1C#Cc1cnc2ccc(N3C[C@@H](C)O[C@@H](C)C3)nn12]
or a pharmaceutically acceptable salt thereof.

42. A compound having a structure of 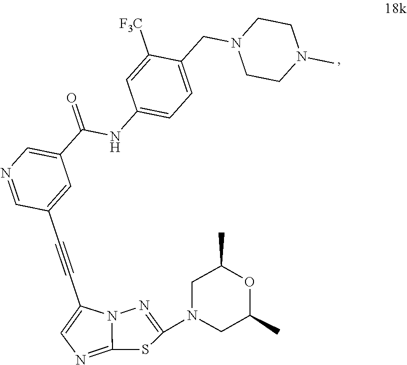 [SMILES: C[C@@H]1CN(c2nn3c(C#Cc4cncc(C(=O)Nc5ccc(CN6CCN(C)CC6)c(C(F)(F)F)c5)c4)cnc3s2)C[C@H](C)O1]
or a pharmaceutically acceptable salt thereof.

43. A compound having a structure of 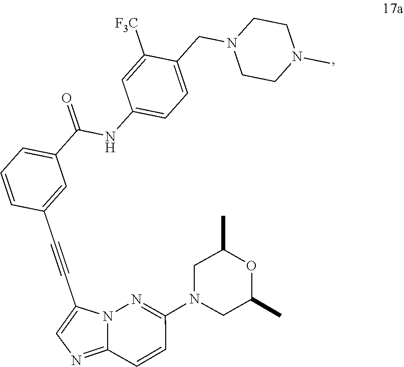 [SMILES: C[C@@H]1CN(c2ccc3ncc(C#Cc4cccc(C(=O)Nc5ccc(CN6CCN(C)CC6)c(C(F)(F)F)c5)c4)n3n2)C[C@H](C)O1]
or a pharmaceutically acceptable salt thereof.

44. A compound having a structure of 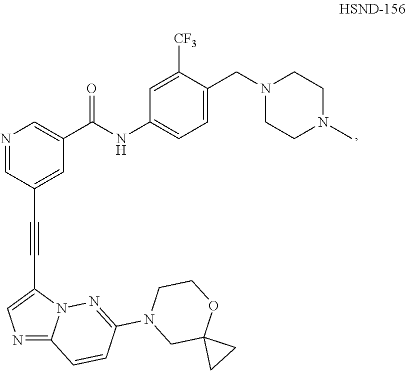 [SMILES: CN1CCN(Cc2ccc(NC(=O)c3cncc(C#Cc4cnc5ccc(N6CCOC7(CC7)C6)nn45)c3)cc2C(F)(F)F)CC1]
or a pharmaceutically acceptable salt thereof.

45. A compound having a structure of 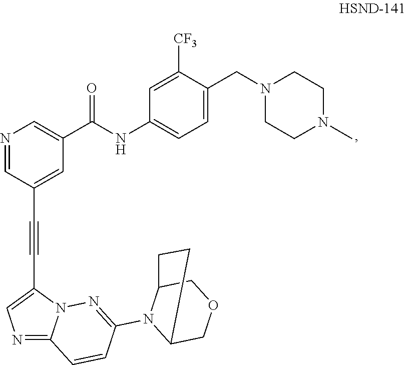 [SMILES: CN1CCN(Cc2ccc(NC(=O)c3cncc(C#Cc4cnc5ccc(N6C7CCC6COC7)nn45)c3)cc2C(F)(F)F)CC1]
or a pharmaceutically acceptable salt thereof.

46. A compound having a structure of 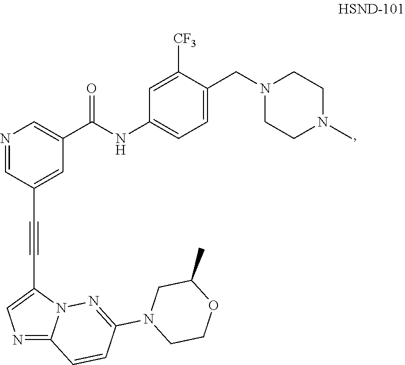 [SMILES: C[C@@H]1CN(c2ccc3ncc(C#Cc4cncc(C(=O)Nc5ccc(CN6CCN(C)CC6)c(C(F)(F)F)c5)c4)n3n2)CCO1]
or a pharmaceutically acceptable salt thereof.

47. A compound having a structure of 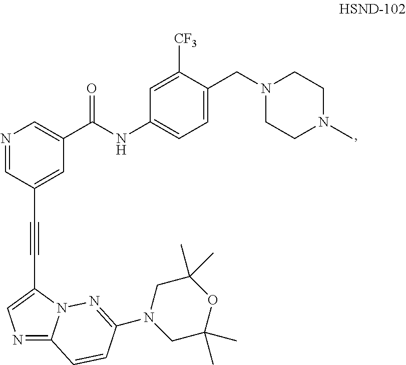 [SMILES: CN1CCN(Cc2ccc(NC(=O)c3cncc(C#Cc4cnc5ccc(N6CC(C)(C)OC(C)(C)C6)nn45)c3)cc2C(F)(F)F)CC1]
or a pharmaceutically acceptable salt thereof.

48. A compound having a structure of 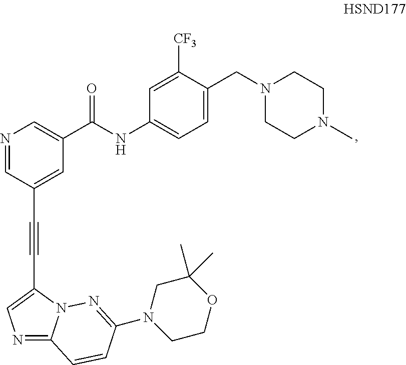 [SMILES: CN1CCN(Cc2ccc(NC(=O)c3cncc(C#Cc4cnc5ccc(N6CCOC(C)(C)C6)nn45)c3)cc2C(F)(F)F)CC1]
or a pharmaceutically acceptable salt thereof.

49. A PROTAC conjugate having a chemical structure of Formula (X):
A L′-D (X)
or a pharmaceutically acceptable salt thereof, wherein:
A is a radical of a compound of any one of claims 1-37;
L′ is a linker that binds A and D, or absent; and
D is a ubiquitin pathway protein binding moiety.

50. A pharmaceutical composition comprising:
a compound of any one of claims 1-48, a conjugate of claim 49, or a pharmaceutically acceptable salt, N-oxide, hydrate, solvent, tautomer, or optical isomer of the compound or conjugate; and
a pharmaceutically acceptable carrier or excipient.

51. A method of treating a disease state or disorder in a subject, the method comprising administering to the subject a first therapy comprising an effective amount of:
a compound of any one of claims 1-48, a conjugate of claim 49, or a pharmaceutically acceptable salt, N-oxide, hydrate, solvate, tautomer, or optical isomer of the compound or conjugate; or
a pharmaceutical composition comprising one or more of a compound of any one of claims 1-48, a conjugate of claim 49, or a pharmaceutically acceptable salt, N-oxide, hydrate, solvate, tautomer, or optical isomer of the compound(s) or conjugate.

52. The method of claim 51, further comprising administering to the subject a second therapy comprising: an effective amount of a chemotherapeutic agent, an immunotherapeutic agent, or a hormone therapeutic agent; or radiation therapy.

53. The method of claim 51, wherein the disease state or disorder of the subject is a cancer.

54. The method of claim 53, wherein the cancer is selected from the group consisting of acute myeloid leukemia, chronic myeloid leukemia, ovarian cancer, cervical cancer, pancreatic cancer, breast cancer, brain cancer, skin cancer, lung cancer, prostate cancer, lymphoma, leukemia, colon cancer, head cancer, neck cancer, thyroid cancer, kidney cancer, liver cancer, and stomach cancer.

55. The method of claim 54, further comprising imaging a tumor microenvironment, a population of cancer cells, or a solid tumor in the subject.

56. A method of suppressing T cell response in a tumor microenvironment of a subject, the method comprising administering to the subject an effective amount of:
a compound of any one of claims 1-48, a conjugate of claim 49, or a pharmaceutically acceptable salt, N-oxide, hydrate, solvate, tautomer, or optical isomer of the compound or conjugate; or
a pharmaceutical composition comprising one or more of a compound of any one of claims 1-48, a conjugate of claim 49, or a pharmaceutically acceptable salt, N-oxide, hydrate, solvate, tautomer, or optical isomer of the compound(s) or conjugate.

57. The method of claim 46, wherein the subject has cancer.

58. The method of claim 46, wherein administering the effective amount to the subject inhibits one or more of MERTK and AXL in the subject.

59. A compound of any one of claims 1-48 or a conjugate of claim 49 for use in the treatment of a disease state modulated by one or more kinases.

60. The compound or conjugate of claim 59, wherein the disease state is cancer.
